(19) United States
(12) Patent Application Publication
(10) Pub. No.: US 2026/0070902 A1
(43) Pub. Date: Mar. 12, 2026

(54) SULFONYL DERIVATIVES AS CCR6 INHIBITORS

(71) Applicant: Hoffmann-La Roche Inc., Little Falls, NJ (US)

(72) Inventors: Lady Johanna BAUTISTA SARMIENTO, Oranjestad (AW); Joseph Maria Gerardus Barbara CALS, Oss (NL); Johanna Hendrika Angelique DE BROUWER, Tilburg (NL); Vera DE KIMPE, Oss (NL); Eugen DERETEY, Wijchen (NL); Alger Ayrton LAZO MORI, Rijswijk (NL); Daan Marten Jorrit MANDERS, Berghem (NL); Sander Bernardus NABUURS, Nijmegen (NL)

(73) Assignee: Hoffmann-La Roche Inc., Little Falls, NJ (US)

(21) Appl. No.: 19/229,636

(22) Filed: Jun. 5, 2025

Related U.S. Application Data
(63) Continuation of application No. PCT/EP2023/084028, filed on Dec. 4, 2023.

(30) Foreign Application Priority Data
Dec. 6, 2022 (EP) 22211605.5
Jul. 26, 2023 (EP) 23187731.7

(51) Int. Cl. C07D 471/04; A61K 31/166; A61K 31/341; A61K 31/381; A61K 31/4035; A61K 31/404; A61K 31/416; A61K 31/4365; A61K 31/437; A61K 31/4418; A61K 31/444; A61K 31/4468; A61K 31/454; A61K 31/4545; A61K 31/455; A61K 31/472; A61K 31/4985; A61K 31/5025; A61K 31/519; A61K 31/53; A61K 31/551; A61K 31/66; C07C 323/22; C07C 381/10; C07D 209/34; C07D 209/42; C07D 209/46; C07D 209/96; C07D 211/96; C07D 213/64; C07D 213/82; C07D 217/24; C07D 231/56; C07D 237/14; C07D 307/68; C07D 333/38; C07D 401/12; C07D 471/10; C07D 487/04; C07D 491/08; C07D 491/107; C07D 495/04; C07D 498/10; C07F 9/53

(52) U.S. Cl. CPC C07D 471/04; A61K 31/166; A61K 31/341; A61K 31/381; A61K 31/4035; A61K 31/404; A61K 31/416; A61K 31/4365; A61K 31/437; A61K 31/4418; A61K 31/444; A61K 31/4468; A61K 31/454; A61K 31/4545; A61K 31/455; A61K 31/472; A61K 31/4985; A61K 31/5025; A61K 31/519; A61K 31/53; A61K 31/551; A61K 31/66; C07C 323/22; C07C 381/10; C07D 209/34; C07D 209/42; C07D 209/46; C07D 209/96; C07D 211/96; C07D 213/64; C07D 213/82; C07D 217/24; C07D 231/56; C07D 237/14; C07D 307/68; C07D 333/38; C07D 401/12; C07D 471/10; C07D 487/04; C07D 491/08; C07D 491/107; C07D 495/04; C07D 498/10; C07F 9/5325

(57) ABSTRACT

The present invention provides new derivatives having the general formula (I)

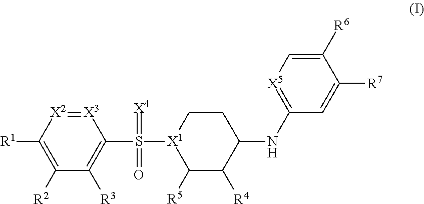

wherein R¹, R², R³, R⁴, R⁵, R⁶, R⁷, X¹, X², X³, X⁴, and X⁵ are as defined herein, compositions including the compounds, processes of manufacturing the compounds and methods of using the compounds.

### BACKGROUND

Immune surveillance, the migration of immune cells throughout the body is a tightly regulated process that is involved in many aspects of health and disease. Chemokines, and their corresponding receptors, play critical roles in these trafficking patterns, they are responsible for getting the right cells into the right tissues (Griffith, J. W., Sokol, C. L. & Luster, A. D. (2014). Chemokines and Chemokine Receptors: Positioning Cells for Host Defense and Immunity. Immunology, 32(1), 659-702 and Zlotnik, A. & Yoshie, O. (2012). The Chemokine Superfamily Revisited. Immunity, 36(5), 705-716).

Chemokines, or chemotactic cytokines, are a family of around 50 small signaling proteins secreted by a variety of cell populations (David, B. A. & Kubes, P. (2019). Exploring the complex role of chemokines and chemoattractants in vivo on leukocyte dynamics. Immunological Reviews, 289(1), 9-30 and Griffith, J. W., Sokol, C. L. & Luster, A. D. (2014). Chemokines and Chemokine Receptors: Positioning Cells for Host Defense and Immunity. Immunology, 32(1), 659-702). Chemokines are divided into four main subfamilies, called CC, CXC, CX3C and C, based on the location of the canonical cysteine residues in the N-terminal region. Chemokine secretion and diffusion create concentration gradients that direct the migration of cells expressing the corresponding receptors. Chemokine receptors are a family of around 20 seven transmembrane proteins differentially expressed on the surface of immune cells and can be divided into two main subfamilies, the first is called G protein-coupled chemokine receptors, which mediate immune cell trafficking, and the second is called atypical chemokine receptors, which seem to be chemokine scavengers that influence the chemokine gradients. They are also grouped into four subfamilies according to the subfamily of their major chemokine ligands. In some cases, one chemokine can signal through multiple receptors and, in many cases, one receptor can be stimulated by multiple chemokines. These promiscuous interactions make pharmacological intervention of signaling more complicated.

CCR6, also called CD196, is a chemokine receptor expressed on a variety of adaptive and innate immune cells including B cells, T cells, dendritic cells and neutrophils. For example, TH17 cells, which play a critical role in the pathogenesis of multiple autoimmune diseases, express CCR6 and this signal has been shown to recruit these cells into inflamed peripheral tissues (Esplugues, E., Huber, S., Gagliani, N., Hauser, A. E., Town, T., Wan, Y. Y., O'Connor, W., Rongvaux, A., Rooijen, N. V., Haberman, A. M., Iwakura, Y., Kuchroo, V. K., Kolls, J. K., Bluestone, J. A., Herold, K. C. & Flavell, R. A. (2011). Control of TH17 cells occurs in the Small Intestine. Nature, 475(7357), 514-518 and Singh, S. P., Zhang, H. H., Foley, J. F., Hedrick, M. N. & Farber, J. M. (2008). Human T Cells That Are Able to Produce IL-17 Express the Chemokine Receptor CCR6. The Journal of Immunology, 180(1), 214-221). The ligand for CCR6 is CCL20, also called macrophage inflammatory protein 3 alpha (MIP-3 alpha) and liver and activation-regulated chemokine (LARC). The CCR6/CCL20 pair is somewhat unique because they have only one binding partner and therefore form a pharmacologically selective receptor-ligand pair (Schutyser, E., Struyf, S. & Damme, J. V. (2003). The CC chemokine CCL20 and its receptor CCR6. Cytokine & Growth Factor Reviews, 14(5), 409-426).

CCL20 expression and secretion is increased in the presence of inflammatory stimuli. High levels of CCL20 can be found in the inflamed tissue associated with multiple inflammatory autoimmune diseases including psoriatic diseases, asthma, Crohn's disease, ulcerative colitis, rheumatoid arthritis, systemic lupus erythematosus and multiple sclerosis (Richmond, J. M., Strassner, J. P., Essien, K. I. & Harris, J. E. (2019). T-cell positioning by chemokines in autoimmune skin diseases. Immunological Reviews, 289(1), 186-204, Lee, A. Y. & Korner, H. (2014). CCR6 and CCL20: emerging players in the pathogenesis of rheumatoid arthritis. Immunology and Cell Biology, 92(4), 354-358, Raman, D., Sobolik-Delmaire, T. & Richmond, A. (2011). Chemokines in health and disease. Experimental Cell Research, 317(5), 575-589, Pene, J., Chevalier, S., Preisser, L., Venereau, E., Guilleux, M.-H., Ghannam, S., Moles, J.-P., Danger, Y., Ravon, E., Lesaux, S., Yssel, H. & Gascan, H. (2008). Chronically Inflamed Human Tissues Are Infiltrated by Highly Differentiated Th17 Lymphocytes. The Journal of Immunology, 180(11), 7423-7430 and Schutyser, E., Struyf, S. & Damme, J. V. (2003). The CC chemokine CCL20 and its receptor CCR6. Cytokine & Growth Factor Reviews, 14(5), 409-426).

Genetic linkage, clinical association and preclinical studies highlight a critical role for CCR6 in these inflammatory diseases (Hamburg, J. P. van & Tas, S. W. (2018). Molecular mechanisms underpinning T helper 17 cell heterogeneity and functions in rheumatoid arthritis. Journal of Autoimmunity, 87, 69-81 and Kurkó, J., Besenyei, T., Laki, J., Glant, T. T., Mikecz, K. & Szekanecz, Z. (2013). Genetics of Rheumatoid Arthritis A Comprehensive Review. Clinical Reviews in Allergy & Immunology, 45(2), 170-179). For example, CCR6 gene variants have the highest risk association for Crohn's disease (CD) amongst the chemokine receptor family (Lee, A. Y. S., Eri, R., Lyons, A. B., Grimm, M. C. & Korner, H. (2013). CC Chemokine Ligand 20 and Its Cognate Receptor CCR6 in Mucosal T Cell Immunology and Inflammatory Bowel Disease: Odd Couple or Axis of Evil?Frontiers in Immunology, 4, 194).

This high selectivity renders CCR6 an attractive drug target. Selective CCR6 inhibitors would only result in on-target pharmacology.

### BRIEF SUMMARY

A first object of the present invention is a compound of formula (I)

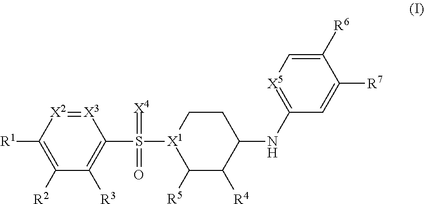

wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a};
R^{1a} is C₁₋₆alkyl, cyano, —NR^{1b}R^{1c}, —C₁₋₆alkyl-NR^{1b}R^{1c}, oxo, —CONR^{1b}R^{1c}, —PO(Me)₂, hydroxy, C₃₋₆cycloalkyl, —C₁₋₆alkyl-C₁₋₆alkoxy, C₃₋₆heterocyclyl, haloC₁₋₆alkyl, halo, C₁₋₆alkoxy;
R^{1b} is hydrogen, C₃₋₆cycloalkyl, or C₁₋₆alkyl;
R^{1c} is hydrogen, C₃₋₆cycloalkyl, or C₁₋₆alkyl;
R² is hydrogen or halogen;
R³ is hydrogen or halogen;
R⁴ is hydrogen, C₁₋₆alkyl, or halogen;
R⁵ is hydrogen, halogen, or C₁₋₆alkyl;
R⁶ is hydrogen or —SF₅, wherein R⁶ and R⁷ must be different;
R⁷ is hydrogen or —SF₅, wherein R⁶ and R⁷ must be different;
or a pharmaceutically acceptable salt thereof.

A second object of the present invention is a process of preparation of compound of formula (I) as described above, or a pharmaceutically acceptable salt thereof, wherein X¹ is N and X⁴ is O, comprising:
reacting compound of formula (VI), wherein R⁶, R⁷, and X⁵ are as defined above,

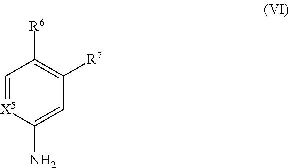

with compound of formula (VII), wherein R⁵ and R⁴ are as defined above,

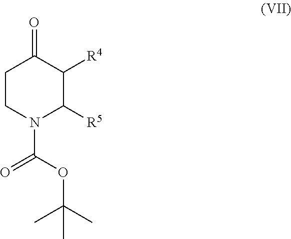

to form compound of formula (VIII), wherein R⁴, R⁵, X⁵, R⁶, R⁷ are as defined above,

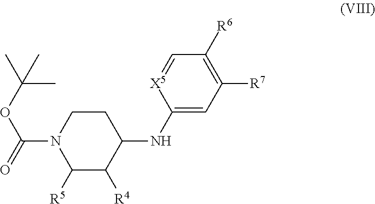

reacting said compound of formula (VIII) with acid to form compound of formula (IX), wherein R⁴, R⁵, X⁵, R⁶, R⁷ are as defined above,

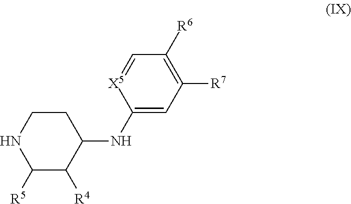

reacting said compound of formula (XI) with compound of formula (XI), wherein X², X³, R² and R³ are as defined above,

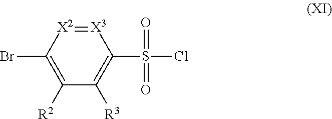

to form compound of formula (III), wherein R², R³, R⁴, R⁶, R⁷, R⁵, X², X⁵ and X³ are as defined above,

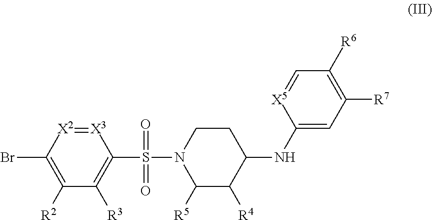

reacting said compound of formula (III) with compound of formula (X) wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

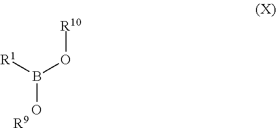

to form compound of formula (I).

A third object of the present invention is a process of preparation of compound of formula (I) as described above, or a pharmaceutically acceptable salt thereof, wherein X¹ is CH and X⁴ is O, comprising:
reacting compound of formula (XII), wherein R⁵ and R⁴ are as defined in above,

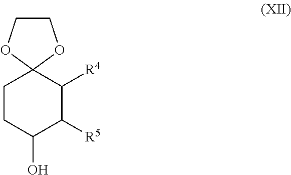

with CH₃SO₂Cl to form compound of formula (XIII), wherein R⁵ and R⁴ are as defined in above,

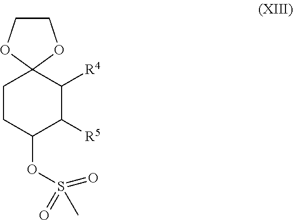

reacting said compound of formula (XIII) with compound of formula (XIV), wherein R³, R², X², and X³ are as defined above,

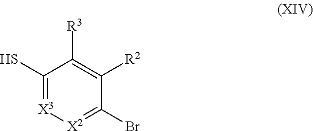

to form compound of formula (XV), wherein R³, R², R⁵, R⁴, X², and X³ are as defined above,

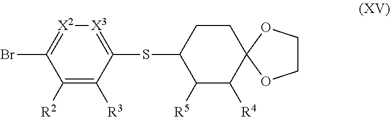

reacting said compound of formula (XV) with an oxidant to form compound of formula (XVI), wherein R³, R², R⁵, R⁴, X², and X³ are as defined above,

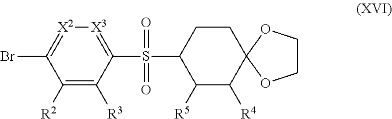

reacting said compound of formula (XVI) with an acid to form compound of formula (XVII), wherein R³, R², R⁵, R⁴, X², and X³ are as defined above,

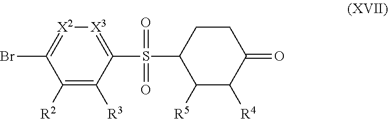

reacting said compound of formula (XVII) with compound of formula (VI), wherein R⁶, R⁷, and X⁵ are as defined above,

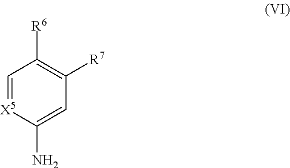

to form compound of formula (XVIII), wherein R², R³, R⁴, R⁶, R⁷, R⁵, X², X⁵ and X³ are as defined above,

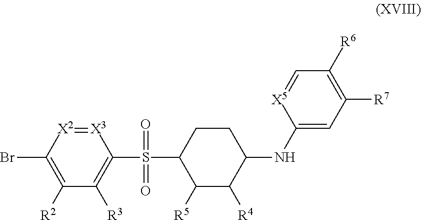

reacting said compound of formula (XVIII) with compound of formula (X) wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

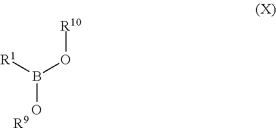

to form compound of formula (I).

A fourth object of the present invention is a process of preparation of compound of formula (I) as described above, or a pharmaceutically acceptable salt thereof, wherein X¹ is CH and X⁴ is NH, comprising:
reacting compound of formula (XV), wherein R³, R², R⁵, R⁴, X², and X³ are as defined above,

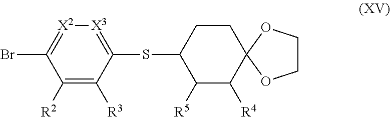

with an acid to form compound of formula (XX), wherein R³, R², R⁵, R⁴, X², and X³ are as defined above,

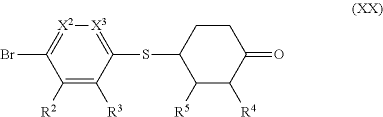

reacting said compound of formula (XX) with compound of formula (VI), wherein R⁶, R⁷, and X⁵ are as defined above,

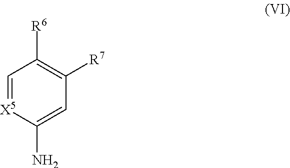

to form compound of formula (XXI), wherein R², R³, R⁴, R⁶, R⁷, R⁵, X², X⁵ and X³ are as defined above,

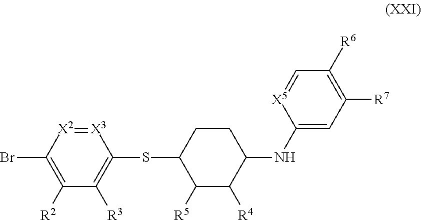

reacting said compound of formula (XXI) with ammonium carbamate and (diacetoxyiodo)benzene to form compound of formula (XXII), wherein R², R³, R⁴, R⁶, R⁷, R⁵, X², X⁵ and X³ are as defined above,

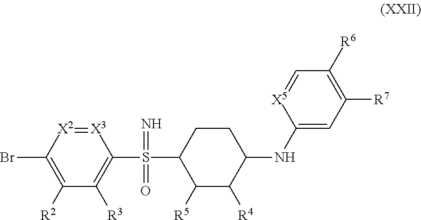

reacting said compound of formula (XXII) with compound of formula (X) wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

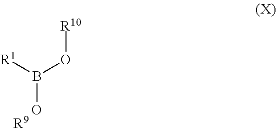

to form compound of formula (I).

A fifth object of the present invention is a process of preparation of compound of formula (I) as described above, or a pharmaceutically acceptable salt thereof, wherein X¹ is N and X⁴ is NH, comprising:
reacting compound of formula (IX), wherein R⁴, R⁵, X⁵, R⁶, R⁷ are as defined above,

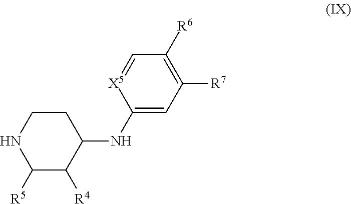

with compound of formula (XIV), wherein R² and R³ are as defined above,

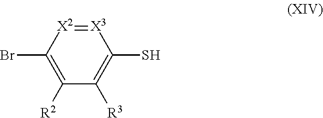

to form compound of formula (XXIV), wherein R², R³, R⁴, R⁶, R⁷, R⁵, X², X⁵ and X³ are as defined above,

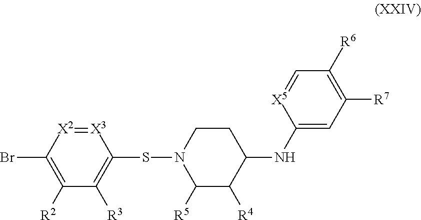

reacting said compound of formula (XXIV) with ammonium carbamate and (diacetoxyiodo)benzene to form compound of formula (XXV), wherein R², R³, R⁴, R⁶, R⁷, R⁵, X², X⁵ and X³ are as defined above,

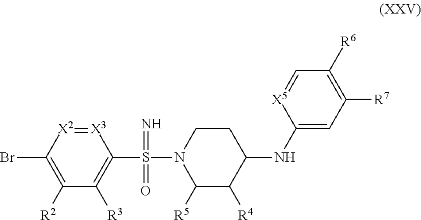

reacting said compound of formula (XXV) with di-tert-butyl dicarbonate and NaH to form compound of formula (XXVI), wherein R², R³, R⁴, R⁶, R⁷, R⁵, X², X⁵ and X³ are as defined above,

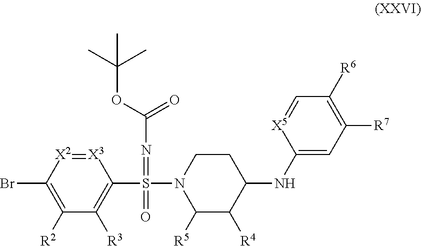

reacting said compound of formula (XXVI) with compound of formula (X), wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

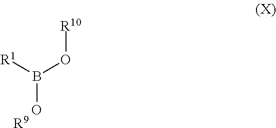

to form compound of formula (XXVII), wherein R¹, R², R³, R⁴, R⁶, R⁷, R⁵, X², X⁵ and X³ are as defined above,

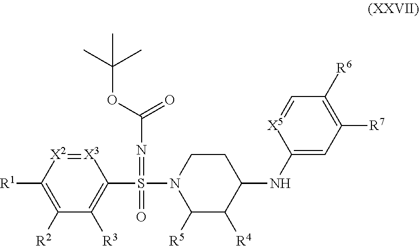

reacting said compound of formula (XXVII) with acid to form compound of formula (I).

A sixth object of the present invention is a pharmaceutical composition comprising a compound of formula (I) as described above, or a pharmaceutically acceptable salt thereof, and a pharmaceutically acceptable excipient.

A seventh object of the current invention is a compound of formula (I), as described above, or a pharmaceutically acceptable salt thereof, for use in the treatment, prevention and/or delay of inflammatory autoimmune disease.

An eighth object of the current invention is a method for the treatment, prevention and/or delay of progression of inflammatory autoimmune disease, which method comprises administering a therapeutically effective amount of a compound of formula (I), or a pharmaceutically acceptable salt thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below.

All publications, patent applications, patents, and other references mentioned herein are incorporated by reference in their entirety.

The nomenclature used in this application is based on IUPAC systematic nomenclature, unless indicated otherwise.

### Definitions

“—SF₅” refers to pentafluorosulfanyl.

“Acid” refers to a compound capable of giving proton of Brønsted's definition, dissociating into proton and counter ion in water at 25° C. and giving a solution having neutral pH or below. Concrete examples of the acid are phosphoric acid (orthophosphoric acid), sulfuric acid, nitric acid, phosphinic acid, phosphonic acid, diphosphonic acid, hydrochloric acid, pyrophosphoric acid, metaphosphoric acid and nitrous acid. These acids may be used in the form of metal salts, ammonium salts or the like, particularly acid means hydrochloric acid.

“Alkoxy” refers to a C₁₋₆alkyl group, as previously defined, attached to the parent molecular moiety via an oxygen atom. Unless otherwise specified, the alkoxy group contains 1 to 12 carbon atoms (“C₁₋₁₂-alkoxy”), preferably 1 to 10 carbon atoms (“C₁₋₁₀-alkoxy”), more preferably 1 to 6 carbon atoms (“C₁₋₆-alkoxy”). In some particular embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy.

“C₁₋₆alkyl” refers to a saturated linear (i.e. unbranched) or branched univalent hydrocarbon chain or combination thereof, having the number of carbon atoms designated (i.e., C₁₋₆ means one to ten carbon atoms). Particular C₁₋₆alkyl groups are those having 1 to 6 carbon atoms, having 2 to 6 carbon atoms (a “C₂₋₆alkyl”), or having 1 to 4 carbon atoms (a “C₁₋₄ alkyl”). Examples of C₁₋₆alkyl group include, but are not limited to, groups such as methyl, ethyl, n-propyl, isopropyl, n-butyl, t-butyl, isobutyl, sec-butyl, homologs and isomers of, for example, n-pentyl, n-hexyl, and the like.

“Amino”, alone or in combination with other groups, refers to NH₂.

“Aromatic” refers to the conventional idea of aromaticity as defined in the literature, in particular in IUPAC—Compendium of Chemical Terminology, 2^{nd} Edition, A. D. McNaught & A. Wilkinson (Eds). Blackwell Scientific Publications, Oxford (1997).

“Aryl” refers to a cyclic aromatic hydrocarbon moiety having a mono-, bi- or tricyclic aromatic ring of 5 to 14 carbon ring atoms (“C₅₋₁₄-aryl”). Bicyclic aryl ring systems include fused bicyclics having two fused five-membered aryl rings (denoted as 5-5), having a five-membered aryl ring and a fused six-membered aryl ring (denoted as 5-6 and as 6-5), and having two fused six-membered aryl rings (denoted as 6-6). The aryl group can be optionally substituted as defined herein. Examples of aryl moieties include, but are not limited to, phenyl, naphthyl, phenanthryl, fluorenyl, indenyl, pentalenyl, azulenyl, and the like. In particular aryl means phenyl.

“Autoimmune disease” or “inflammatory autoimmune disease” refers to a disease resulting from an immune response against a self tissue or tissue component, including both self antibody responses and cell-mediated responses. The term autoimmune disease or inflammatory autoimmune disease, as used herein, encompasses organ-specific autoimmune diseases. Examples of autoimmune diseases are psoriatic diseases, asthma, Inflammatory Bowel Diseases (IBD), Crohn's disease, Obstructive Lung Diseases ulcerative colitis, rheumatoid arthritis, systemic lupus erythematosus and multiple sclerosis.

“Cyano”, alone or in combination with other groups, refers to CN (i.e. nitrile).

“Cycloalkyl” refers to a saturated or partially unsaturated carbocyclic moiety having mono-, bi- (including bridged bicyclic and cycloalkyl spiro moieties) or tricyclic rings and 3 to 10 carbon atoms i.e., (C₃-C₁₀)cycloalkyl) in the ring. The cycloalkyl moiety can optionally be substituted with one or more substituents. In particular aspects cycloalkyl contains from 3 to 8 carbon atoms (i.e., (C₃-C₈)cycloalkyl). In other particular aspects cycloalkyl contains from 3 to 6 carbon atoms (i.e., (C₃-C₆)cycloalkyl). Examples of cycloalkyl moieties include, but are not limited to, cyclopropyl, cyclobutyl, cyclopentyl, cyclohexyl, cycloheptyl, and partially unsaturated (cycloalkenyl) derivatives thereof (e.g. cyclopentenyl, cyclohexenyl, and cycloheptenyl), bicyclo[3.1.0]hexanyl, bicyclo[3.1.0]hexenyl, bicyclo[3.1.1]heptanyl, bicyclo[3.1.1]heptenyl and bicyclo[1.1.1]pentane. The cycloalkyl moiety can be attached in a “spiro-cycloalkyl” or “cycloalkyl spiro” fashion such as “spirocyclopropyl”.

“Halo” or “Halogen” means fluoro, chloro, bromo or iodo, particularly chloro or fluoro.

“Halo-C₁₋₆C₁₋₆alkyl” refers to an C₁₋₆C₁₋₆alkyl, as defined above, substituted with one or more halogen atoms, particularly with one to three halogen atoms. More particularly halo-C₁₋₆C₁₋₆alkyl is the chloro- and fluoro-C₁₋₆C₁₋₆alkyl. In some particular embodiment halo-C₁₋₆C₁₋₆alkyl refers to perhaloC₁₋₃C₁₋₆alkyl as defined herein. More particularly halo-C₁₋₆C₁₋₆alkyl is trifluoromethyl, difluoromethyl or fluoromethyl. Most particularly halo-C₁-C₁₋₆alkyl is trifluoromethyl (—CF₃).

“Haloalkoxy” refers to an alkoxy group in which at least one Halogen takes the place of each H in the hydrocarbon making up the C₁₋₆alkyl moiety of the alkoxy group. An example of a haloalkoxy group is difluoromethoxy (—OCHF₂), trifluoromethoxy (—OCF₃).

“Heteroaryl” refers to an aromatic heterocyclic mono-, bi- or tricyclic ring system of 5 to 14 ring atoms, preferably from 5 to 10 ring atoms, more preferably from 5 to 6 ring atoms, comprising 1, 2, 3 or 4 heteroatoms selected from N, O and S, the remaining ring atoms being carbon. In some aspects, monocyclic heteroaryl rings may be 5-6 membered. Bicyclic heteroaryl ring systems include fused bicyclics having two fused five-membered heteroaryl rings (denoted as 5-5), having a five-membered heteroaryl ring and a fused six-membered heteroaryl ring (denoted as 5-6 and 6-5), and having two fused six-membered heteroaryl rings (denoted as 6-6). The heteroaryl group can be optionally substituted as defined herein. Examples of heteroaryl moieties include indazolyl, indolyl, triazolopyridinyl, imidazopyridinyl, imidazopyrazinyl, pyridyl, triazolopyridazinyl, pyridazinyl, triazolopyrazinyl, pyrrolotriazinyl, spirocyclopropaneindolinyl, pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, benzothiophenyl, indolyl, aza-indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, pyrrolopyridinyl, furopyridinyl, thienopyridinyl, pyrrolopyridazinyl, pyrrolopyrimidinyl, pyrrolopyrazinyl, thienopyridazinyl, thienopyrimidinyl, thienopyrazinyl, furopyridazinyl, furopyrimidinyl, and furopyrazinyl. More particularly heteroaryl are pyridinyl, pyrazinyl, pyridazinyl, pyrimidinyl.

“Heterocycle” or “heterocyclyl” refer to a 3, 4, 5, 6, 7, 8, 9, 10-membered monocyclic, 7, 8, 9 and 10-membered bicyclic (including bridged bicyclic and cycloalkyl spiro moieties) or 10, 11, 12, 13, 14 and 15-membered bicyclic heterocyclic moiety that is saturated or partially unsaturated, and has one or more (e.g., 1, 2, 3 or 4) heteroatoms selected from oxygen, nitrogen and sulfur in the ring with the remaining ring atoms being carbon. In some aspects, the heterocycle is a heterocycloalkyl. In particular aspects heterocycle or heterocyclyl refers to a 4, 5, 6 or 7-membered heterocycle. When used in reference to a ring atom of a heterocycle, a nitrogen or sulfur may also be in an oxidized form, and a nitrogen may be substituted with one or more (C₁-C₆)C₁₋₆alkyl or groups. The heterocycle can be attached to its pendant group at any heteroatom or carbon atom that results in a stable structure. Any of the heterocycle ring atoms can be optionally substituted with one or more substituents described herein. Examples of such saturated or partially unsaturated heterocycles include, without limitation, tetrahydrofuranyl, tetrahydrothienyl, pyrrolidinyl, pyrrolidonyl, piperidinyl, pyrrolinyl, isoindolinyl, quinolinyl, isoquinolinyl, tetrahydroquinolinyl, indolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, pyrrolidine 1-oxide, N-hydroxypiperidine, 1-methylpyrrolidine N-oxide, diazirinyl and quinuclidinyl. The term heterocycle also includes groups in which a heterocycle is fused to one or more aryl, heteroaryl, or cycloalkyl rings, such as indolinyl, 3H-indolyl, chromanyl, azabicyclo[2.2.1]heptanyl, azabicyclo[3.1.0]hexanyl, azabicyclo[3.1.1]heptanyl, octahydroindolyl, or tetrahydroquinolinyl.

“Hydroxy”, alone or in combination with other groups, refers to —OH.

“Hydroxyalkyl” refers to a C₁₋₆alkyl group wherein one or more of the hydrogen atoms of the C₁₋₆alkyl group have been replaced by a hydroxy moiety. Examples include alcohols and diols.

The terms “inflammatory bowel disease” or “IBD” means several diseases associated with inflammation of the small intestine, large intestine (colon), rectum or anus (anal sphincter), and may particularly include ulcerative colitis and Crohn's disease, including proctitis in both cases. As used in the present context, the term “IBD” also includes gastrointestinal (GI) tract cancer, which is a likely result of GI tract inflammation. As used in this specification, the terms “gastrointestinal tract” or “GI tract” mean the small intestine, large intestine (colon), rectum or anus (anal sphincter).

“Moiety” and “Substituent” refer to an atom or group of chemically bonded atoms that is attached to another atom or molecule by one or more chemical bonds thereby forming part of a molecule.

When indicating the number of substituents, the term “one or more” refers to the range from one substituent to the highest possible number of substitutions, i.e. replacement of one hydrogen up to replacement of all hydrogens by substituents, in particular wherein “one or more” refers to one, two or three, most particularly “one or more” refers to one or two.

“Obstructive pulmonary disease” refers to any disease that causes the airways of the lungs to become narrow or blocked so that a patient cannot exhale completely. Because of damage to the lungs or narrowing of the airways inside the lungs, exhaled air comes out more slowly than normal. At the end of a full exhalation, an abnormally high amount of air may still remain in the lungs. Examples of obstructive pulmonary diseases are asthma, bronchiectasis, bronchitis and chronic obstructive pulmonary disease (COPD).

“Optional” or “optionally” means that the subsequently described event or circumstance may but need not occur, and that the description includes instances where the event or circumstance occurs and instances in which it does not. For example, “aryl group optionally substituted with a C₁₋₆alkyl group” means that the C₁₋₆alkyl may but need not be present, and the description includes situations where the aryl group is substituted with a C₁₋₆alkyl group and situations where the aryl group is not substituted with the C₁₋₆alkyl group.

“Optionally substituted” means unsubstituted or substituted. Generally, these substituents can be the same or different.

“Oxidant” refers to one or more suitable electron acceptors or electron sharers and may be an element, combination of elements, a compound, or combination of compounds including reducible compounds, and is a vapor, solid or liquid at the process conditions. An example of oxidant is mCPBA (3-chloroperbenzoic acid).

“Oxo”, alone or in combination with other groups, refers to ═O.

“Pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein.

More particularly pharmaceutically acceptable salts of compounds of formula (I) are the salts of hydrochloric acid, hydrobromic acid, sulfuric acid, phosphoric acid and methanesulfonic acid.

“Prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

“Protecting group” refers to the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are Amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

“Substituted” refers to the replacement of at least one of hydrogen atoms of a compound or moiety with another substituent or moiety. Examples of such substituents include, without limitation, Halogen, —OH, —CN, oxo, alkoxy, C₁₋₆alkyl, alkylene, aryl, heteroaryl, haloalkyl, haloalkoxy, cycloalkyl and heterocycle. For example, the term “haloalkyl” refers to the fact that one or more hydrogen atoms of a C₁₋₆alkyl (as defined below) is replaced by one or more Halogen atoms (e.g., trifluoromethyl, difluoromethyl, fluoromethyl, chloromethyl, etc.). In one aspect, substituted as used herein can refer to replacement of at least one hydrogen atom of a compound or moiety described herein with Halogen or C₁₋₆alkyl.

“Therapeutically effective amount” refers to an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

“Therapeutically inert carrier” refers to any ingredient having no therapeutic activity and being non-toxic such as disintegrators, binders, fillers, solvents, buffers, tonicity agents, stabilizers, antioxidants, surfactants or lubricants used in formulating pharmaceutical products.

### DETAILED DESCRIPTION

In one embodiment, the present invention relates a compound of formula (I),

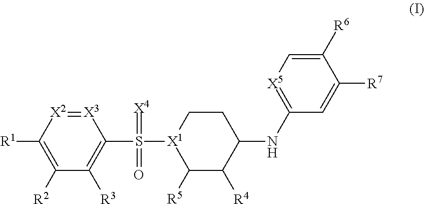

wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a};
R^{1a} is C₁₋₆alkyl, cyano, —NR^{1b}R^{1c}, —C₁₋₆alkylNR^{1b}R^{1c}, oxo, —CONR^{1b}R^{1c}, —PO(Me)₂, hydroxy, C₃₋₆cycloalkyl, —C₁₋₆alkylC₁₋₆alkoxy, C₃₋₆heterocyclyl, haloC₁₋₆alkyl, halo, C₁₋₆alkoxy;
R^{1b} is hydrogen, C₃₋₆cycloalkyl, or C₁₋₆alkyl;
R^{1c} is hydrogen, C₃₋₆cycloalkyl, or C₁₋₆alkyl;
R² is hydrogen or halogen;
R³ is hydrogen or halogen;
R⁴ is hydrogen, C₁₋₆alkyl, or halogen;
R⁶ is hydrogen or —SF₅, wherein R⁶ and R⁷ must be different;
R⁷ is hydrogen or —SF₅, wherein R⁶ and R⁷ must be different;
R⁵ is hydrogen, halogen, or C₁₋₆alkyl;
or a pharmaceutically acceptable salt thereof.

A particular embodiment of the present invention relates to a compound of formula (I) as described herein, wherein
R^{1a} is C₁₋₆alkyl, cyano, oxo, —CONR^{1b}R^{1c}, —PO(Me)₂, hydroxy, C₃₋₆cycloalkyl, —C₁₋₆alkylC₁₋₆alkoxy, haloC₁₋₆alkyl, halo, C₁₋₆alkoxy;
R^{1b} is hydrogen or C₁₋₆alkyl;
R^{1c} is hydrogen or C₁₋₆alkyl;
X⁵ is CH;
R⁵ is hydrogen;
or a pharmaceutically acceptable salt thereof.

A particular embodiment of the present invention relates to a compound of formula (I) or (I′) as described herein, or a pharmaceutically acceptable salt thereof, wherein X¹ is CH.

A particular embodiment of the present invention relates to a compound of formula (I) or (I′) as described herein, or a pharmaceutically acceptable salt thereof, wherein wherein X¹ is N.

A particular embodiment of the present invention relates to a compound of formula (I) or (I′) as described herein, or a pharmaceutically acceptable salt thereof, wherein X² is CH.

A particular embodiment of the present invention relates to a compound of formula (I) or (I′) as described herein, or a pharmaceutically acceptable salt thereof, wherein X² is N.

A particular embodiment of the present invention relates to a compound of formula (I) or (I′) as described herein, or a pharmaceutically acceptable salt thereof, wherein X³ is CH.

A particular embodiment of the present invention relates to a compound of formula (I) or (I′) as described herein, or a pharmaceutically acceptable salt thereof, wherein X³ is N.

A particular embodiment of the present invention relates to a compound of formula (I) or (I′) as described herein, or a pharmaceutically acceptable salt thereof, wherein X⁴ is O.

A particular embodiment of the present invention relates to a compound of formula (I) or (I′) as described herein, or a pharmaceutically acceptable salt thereof, wherein X⁴ is NH.

A particular embodiment of the present invention relates to a compound of formula (I) as described herein, or a pharmaceutically acceptable salt thereof, wherein X⁵ is N.

A particular embodiment of the present invention relates to a compound of formula (I) as described herein, or a pharmaceutically acceptable salt thereof, wherein X⁵ is CH.

A particular embodiment of the present invention relates to a compound of formula (I) or (I′) as described herein, or a pharmaceutically acceptable salt thereof, wherein X¹ is N, X² is CH, X³ is CH, X⁴ is O, and X⁵ is CH.

A particular embodiment of the present invention relates to a compound of formula (I) or (I′) as described herein, or a pharmaceutically acceptable salt thereof, wherein X¹ is CH, X² is CH, X³ is CH, X⁴ is O, and X⁵ is CH.

A particular embodiment of the present invention relates to a compound of formula (I) or (I′) as described herein, or a pharmaceutically acceptable salt thereof, wherein X¹ is N, X² is N, X³ is CH, X⁴ is O, and X⁵ is CH.

A particular embodiment of the present invention relates to a compound of formula (I) or (I′) as described herein, or a pharmaceutically acceptable salt thereof, wherein X¹ is N, X² is CH, X³ is N, X⁴ is O, and X⁵ is CH.

A particular embodiment of the present invention relates to a compound of formula (I) or (I′) as described herein, or a pharmaceutically acceptable salt thereof, wherein X¹ is CH, X² is CH, X³ is CH, X⁴ is NH, and X⁵ is CH.

A particular embodiment of the present invention relates to a compound of formula (I) or (I′) as described herein, or a pharmaceutically acceptable salt thereof, wherein X¹ is N, X² is CH, X³ is CH, X⁴ is NH, and X⁵ is CH.

A particular embodiment of the present invention relates to a compound of formula (I), (I′) or (I″) as described herein, or a pharmaceutically acceptable salt thereof, wherein R¹ is azaspirononanyl, triazaspirodecanyl, diazaspirodecanyl, oxadiazaspirodecanyl, oxaazaspirononanyl, oxaazabicyclooctanyl, pyrazolopyridinyl, pyrrolopyrimidinyl, pyrrolopyridinyl, thiophenyl, thienopyridinyl, pyrrolopyrazinyl, pyrrolodiazepinyl, furanyl, indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, or spirocyclopropaneindolinyl, wherein azaspirononanyl, triazaspirodecanyl, diazaspirodecanyl, oxadiazaspirodecanyl, oxaazaspirononanyl, oxaazabicyclooctanyl, pyrazolopyridinyl, pyrrolopyrimidinyl, pyrrolopyridinyl, thiophenyl, thienopyridinyl, pyrrolopyrazinyl, pyrrolodiazepinyl, furanyl, indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, and spirocyclopropaneindolinyl are optionally substituted with one or more R^{1a}.

A particular embodiment of the present invention relates to a compound of formula (I), (I′) or (I″) as described herein, or a pharmaceutically acceptable salt thereof, wherein R¹ is indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, or spirocyclopropaneindolinyl, wherein indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, and spirocyclopropaneindolinyl are optionally substituted with one or more R^{1a}.

A particular embodiment of the present invention relates to a compound of formula (I), (I′) or (I″) as described herein, or a pharmaceutically acceptable salt thereof, wherein R¹ is indazolyl, indolyl, pyridyl, triazolopyridinyl, phenyl, or imidazopyridinyl, wherein indazolyl, indolyl, pyridyl, triazolopyridinyl, phenyl, and imidazopyridinylare optionally substituted with one or more R^{1a}.

A particular embodiment of the present invention relates to a compound of formula (I), (I′) or (I″) as described herein, or a pharmaceutically acceptable salt thereof, wherein R¹ is methylindazolyl, cyano-indolyl, cyano-indazolyl, oxo-isoindolinyl, Carbamoylpyridyl, methyl-triazolopyridinyl, imidazopyridinyl, cyano-imidazopyridinyl, imidazopyrazinyl, methylimidazopyridinyl, carbamoylimidazopyridinyl, isopropyl-triazolopyridinyl, triazolopyridinyl, carbamoylindazolyl, oxo-indolinyl, carbamoyl-triazolopyridinyl, cyano-triazolopyridinyl, (dimethylcarbamoyl)phenyl, methylimidazopyrazinyl, (dimethylcarbamoyl)pyridyl, (methylcarbamoyl)phenyl, (methylcarbamoyl)pyridyl, oxo-triazolopyridinyl, carbamoylphenyl, isopropyl-triazolopyridazinyl, oxo-dihydro-isoquinolinyl, carbamoylpyridazinyl, dimethylphosphorylphenyl, isopropyl-triazolopyrazinyl, oxo-pyridinyl, oxo-pyrrolotriazinyl, hydroxypyridyl, (dimethylcarbamoyl)pyridyl, oxo-spirocyclopropaneindolineyl, methoxyazaspirononanyl, oxo-triazaspirodecanyl, oxo-diazaspirodecanyl, oxo-oxadiazaspirodecanyl, oxaazaspirononanyl, oxaazabicyclooctanyl, carbamoylfluorophenyl, propanyltriazolopyrazinyl, isopropycarbamoylphenyl, methylpyrazolopyridinyl, N,N-dimethylamineimidazopyridinyl, cyclopropylcarbamoylphenyl, pyrrolopyrimidinyl, pyrrolopyridinyl, pyrazolopyridinyl, carbamoylthiophenyl, methylimidazopyridinyl, (trifluoromethyl)imidazopyridinyl, fluoroimidazopyridinyl, oxo-thienopyridinyl, oxo-pyrrolopyrazinyl, oxo-pyrrolodiazepinyl, carbamoylimidazopyridinyl, furancarboxamide, cyano-imidazopyridinyl, pyrrolidinyl-triazolopyridinyl, propanyl-triazolopyrazinyl, or azetidinyl-triazolopyridinyl.

A particular embodiment of the present invention relates to a compound of formula (I), (I′) or (I″) as described herein, or a pharmaceutically acceptable salt thereof, wherein R¹ is methylindazolyl, cyanoindolyl, cyanoindazolyl, oxoisoindolinyl, carbamoylpyridyl, methyl-triazolopyridinyl, imidazopyridinyl, cyanoimidazopyridinyl, imidazopyrazinyl, methylimidazopyridinyl, carbamoylimidazopyridinyl, isopropyl-triazolopyridinyl, triazolopyridinyl, carbamoylindazolyl, oxoindolinyl, carbamoyl-triazolopyridinyl, methyl-triazolopyridinyl, methyl-triazolopyridinyl, cyano-triazolopyridinyl, (dimethylcarbamoyl)phenyl, methylimidazopyrazinyl, (dimethylcarbamoyl)pyridyl, (methylcarbamoyl)phenyl, (methylcarbamoyl)pyridyl, oxo-triazolopyridinyl, carbamoylphenyl, carbamoylpyridyl, isopropyl-triazolopyridazinyl, oxo-dihydro-isoquinolinyl, carbamoylpyridazinyl, dimethylphosphorylphenyl, oxoisoindolinyl, oxoindolinyl, oxo-dihydro-isoquinolinyl, isopropyl-triazolopyrazinyl, oxo-pyridinyl, oxo-dihydro-isoquinolinyl, oxo-pyrrolotriazinyl, hydroxypyridyl, methyl-triazolopyridinyl, imidazopyridinyl, triazolopyridinyl, carbamoylphenyl, carbamoylpyridyl, (dimethylcarbamoyl)pyridyl, oxospirocyclopropaneindolineyl.

A particular embodiment of the present invention relates to a compound of formula (I), (I′) or (I″) as described herein, or a pharmaceutically acceptable salt thereof, wherein R¹ is methyl-indazolyl, cyano-indolyl, carbamoyl-pyridyl, methyl-imidazopyridinyl, methyl-triazolopyridinyl, isopropyl-triazolopyridinyl, cyano-indazolyl, (methylcarbamoyl)phenyl, imidazopyridinyl, cyanoimidazopyridinyl.

A particular embodiment of the present invention relates to a compound of formula (I), (I′) or (I″) as described herein, or a pharmaceutically acceptable salt thereof, wherein R^{1a} is methoxy, fluoro, —CONH[(CH(CH3)₂], —CONH(cyclopropyl), dimethylamino, —CF₃, pyrrolidinyl, azetidinyl, methyl, cyano, oxo, —CONH₂, isopropyl, —CON(Me)₂, —CH₂—O—CH₃, —CONH(Me), —PO(Me)₂, —OH.

A particular embodiment of the present invention relates to a compound of formula (I), (I′) or (I″) as described herein, or a pharmaceutically acceptable salt thereof, wherein R^{1a} is methyl, cyano, oxo, —CONH₂, isopropyl, —CON(Me)₂, —CH₂—O—CH₃, —CONH(Me), —PO(Me)₂, —OH.

A particular embodiment of the present invention relates to a compound of formula (I), (I′) or (I″) as described herein, or a pharmaceutically acceptable salt thereof, wherein R^{1a} is methyl, cyano, —CONH₂, isopropyl, —CONH(Me).

A particular embodiment of the present invention relates to a compound of formula (I) or (I′) as described herein, or a pharmaceutically acceptable salt thereof, wherein R² is hydrogen, fluorine, or —CF₃.

A particular embodiment of the present invention relates to a compound of formula (I) or (I′) as described herein, or a pharmaceutically acceptable salt thereof, wherein R² is hydrogen.

A particular embodiment of the present invention relates to a compound of formula (I) or (I′) as described herein, or a pharmaceutically acceptable salt thereof, wherein R³ is hydrogen, fluorine, chlorine, or —CF₃.

A particular embodiment of the present invention relates to a compound of formula (I) or (I′) as described herein, or a pharmaceutically acceptable salt thereof, wherein R³ is hydrogen. A particular embodiment of the present invention relates to a compound of formula (I) or (I′) as described herein, or a pharmaceutically acceptable salt thereof, wherein R⁴ is hydrogen or fluorine.

A particular embodiment of the present invention relates to a compound of formula (I) or (I′) as described herein, or a pharmaceutically acceptable salt thereof, wherein R⁴ is hydrogen.

A particular embodiment of the present invention relates to a compound of formula (I) or (I′) as described herein, or a pharmaceutically acceptable salt thereof, wherein R⁶ is —SF₅.

A particular embodiment of the present invention relates to a compound of formula (I) or (I′) as described herein, or a pharmaceutically acceptable salt thereof, wherein R⁷ is hydrogen.

A particular embodiment of the present invention relates to a compound of formula (I) or (I′) as described herein, or a pharmaceutically acceptable salt thereof, wherein R⁵ is hydrogen.

A particular embodiment of the present invention relates to a compound of formula (I) as described herein, or a pharmaceutically acceptable salt thereof, wherein the compound is of formula (I′)

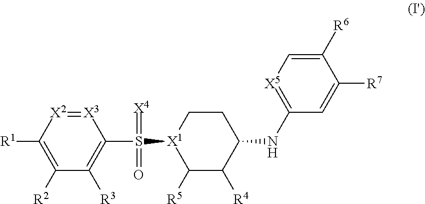

or a pharmaceutically acceptable salt thereof wherein R¹, R², R³, R⁴, R⁵, R⁶, R⁷, X¹, X², X³, X⁴, and X⁵ are as defined above.

A particular embodiment of the present invention relates to a compound of formula (I) as described herein, or a pharmaceutically acceptable salt thereof, wherein the compound is of formula (I″)

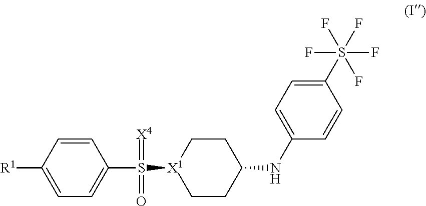

or a pharmaceutically acceptable salt thereof wherein R¹, X¹ and X⁴ are as defined above.

A particular embodiment of the present invention relates to a compound of formula (I) or (I′) is as described herein, or a pharmaceutically acceptable salt thereof, wherein
X¹ is CH or N,
X² is CH,
X³ is CH,
X⁴ is O or NH,
X⁵ is CH,
R¹ is indazolyl, indolyl, pyridyl, triazolopyridinyl, phenyl, or imidazopyridinyl, wherein indazolyl, indolyl, pyridyl, triazolopyridinyl, phenyl, and imidazopyridinylare optionally substituted with one or more R^{1a},
R^{1a} is methyl, cyano, —CONH₂, isopropyl, —CONH(Me),
R² is hydrogen,
R³ is hydrogen,
R⁴ is hydrogen,
R⁵ is hydrogen,
R⁶ is —SF₅,
R⁷ is hydrogen.

A particular embodiment of the present invention relates to a compound of formula (I) or (I′) as described herein, or a pharmaceutically acceptable salt thereof, wherein
X¹ is CH or N,
X² is CH,
X³ is CH,
X⁴ is O or NH,
X⁵ is CH,
R¹ is methyl-indazolyl, cyano-indolyl, carbamoyl-pyridyl, methyl-imidazopyridinyl, methyl-triazolopyridinyl, isopropyl-triazolopyridinyl, cyano-indazolyl, (methylcarbamoyl)phenyl, imidazopyridinyl, cyanoimidazopyridinyl,
R² is hydrogen,
R³ is hydrogen,
R⁴ is hydrogen,
R⁵ is hydrogen,
R⁶ is —SF₅,
R⁷ is hydrogen.

Further, it is to be understood that every embodiment relating to a specific X¹, X², X³, X⁴, R¹, R^{1a}, R^{1b}, R^{1c}, R², R³, R⁴, R⁵, R⁶ and R⁷ as disclosed herein may be combined with any other embodiment relating to another X¹, X², X³, X⁴, R¹, R^{1a}, R^{1b}, R^{1c}, R², R³, R⁴, R⁵, R⁶ and R⁷ as disclosed herein.

A particular embodiment of the present invention relates to a compound of formula (I) as described herein, or a pharmaceutically acceptable salt thereof, selected from:
1-(4-{imidazo[1,2-a]pyridin-6-yl}benzenesulfonyl)-N-[4-(pentafluoro-λ⁶-sulfanyl)phenyl]piperidin-4-amine
1-[4-(3-methyl-2H-indazol-5-yl)benzenesulfonyl]-N-[4-(pentafluoro-λ⁶-sulfanyl)phenyl]piperidin-4-amine
5-{4-[(4-{[4-(pentafluoro-λ⁶-sulfanyl)phenyl]Amino}piperidin-1-yl)sulfonyl]phenyl}-1H-indole-3-carbonitrile
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[cis-4-[4-(3-methyl-2H-indazol-5-yl)benzenesulfonyl]cyclohexyl]aniline
4-(pentafluoro-λ⁶-sulfanyl)-N-[trans-4-[4-(3-methyl-2H-indazol-5-yl)benzenesulfonyl]cyclohexyl]aniline
5-(4-{[trans-4-{[4-(pentafluoro-λ⁶-sulfanyl)phenyl]Amino}cyclohexyl]sulfonyl}phenyl)-1H-indole-3-carbonitrile
5-(4-{[cis-4-{[4-(pentafluoro-λ⁶-sulfanyl)phenyl]Amino}cyclohexyl]sulfonyl}phenyl)-1H-indole-3-carbonitrile
6-(4-{[trans-4-{[4-(pentafluoro-λ⁶-sulfanyl)phenyl]Amino}cyclohexyl]sulfonyl}phenyl)-2,3-dihydro-1H-isoindol-1-one
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(pentafluoro-λ⁶-sulfanyl)-N-[trans-4-(4-{imidazo[1,2-a]pyridin-6-yl}benzenesulfonyl)cyclohexyl]aniline
4-(pentafluoro-λ⁶-sulfanyl)-N-[trans-4-(4-{3-methylimidazo[1,2-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-indol-2-one
N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide
N-methyl-4′-{[trans-4-{[4-(pentafluoro-λ⁶-sulfanyl)phenyl]Amino}cyclohexyl]sulfonyl}-[1,1′-biphenyl]-4-carboxamide
6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile
4-(pentafluoro-λ⁶-sulfanyl)-N-[cis-4-(4-{imidazo[1,2-a]pyridin-6-yl}benzenesulfonyl)cyclohexyl]aniline
N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonyl}phenyl)pyridine-2-carboxamide
N-methyl-4′-{[trans-4-{[4-(pentafluoro-λ⁶-sulfanyl)phenyl]Amino}cyclohexyl]sulfonyl}-[1,1′-biphenyl]-3-carboxamide
N-methyl-5-(4-{[trans-4-{[4-(pentafluoro-λ⁶-sulfanyl)phenyl]Amino}cyclohexyl]sulfonyl}phenyl)pyridine-2-carboxamide
4′-{[trans-4-{[4-(pentafluoro-λ⁶-sulfanyl)phenyl]Amino}cyclohexyl]sulfonyl}-[1,1′-biphenyl]-3-carboxamide
1-(4-{imidazo[1,2-a]pyrazin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine
N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
N-[4-(pentafluoro-λ⁶-sulfanyl)phenyl]-1-{4-[3-(propan-2-yl)imidazo[1,2-a]pyridin-6-yl]benzenesulfonyl}piperidin-4-amine
1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine
1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile
6-(4-{[trans-4-{[4-(pentafluoro-λ⁶-sulfanyl)phenyl]Amino}cyclohexyl]sulfonyl}phenyl)imidazo[1,2-a]pyridine-3-carboxamide
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-(4-{[1,2,4]triazolo[4,3-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-1H-indazole-3-carboxamide
5-(4-{[trans-4-{[4-(pentafluoro-λ⁶-sulfanyl)phenyl]Amino}cyclohexyl]sulfonyl}phenyl)-1H-indazole-3-carbonitrile
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide
4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline
4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile
4-(pentafluoro-λ⁶-sulfanyl)-N-[trans-4-(4-{2-methylimidazo[1,2-a]pyrazin-6-yl}benzenesulfonyl)cyclohexyl]aniline
4-(pentafluoro-λ⁶-sulfanyl)-N-[trans-4-(4-{imidazo[1,2-a]pyrazin-6-yl}benzenesulfonyl)cyclohexyl]aniline
N,N-dimethyl-6-(4-{[cis-4-{[4-(pentafluoro-λ⁶-sulfanyl)phenyl]Amino}cyclohexyl]sulfonyl}phenyl)pyridine-3-carboxamide
N,N-dimethyl-6-(4-{[trans-4-{[4-(pentafluoro-λ⁶-sulfanyl)phenyl]Amino}cyclohexyl]sulfonyl}phenyl)pyridine-3-carboxamide
N-methyl-6-(4-{[trans-4-{[4-(pentafluoro-λ⁶-sulfanyl)phenyl]Amino}cyclohexyl]sulfonyl}phenyl)pyridine-3-carboxamide
N-methyl-4-(4-{[trans-4-{[4-(pentafluoro-λ⁶-sulfanyl)phenyl]Amino}cyclohexyl]sulfonyl}phenyl)pyridine-2-carboxamide
6-(4-{[trans-4-{[4-(pentafluoro-λ⁶-sulfanyl)phenyl]Amino}cyclohexyl]sulfonyl}phenyl)pyridine-3-carboxamide
4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
4-(4-{[trans-4-{[4-(pentafluoro-λ⁶-sulfanyl)phenyl]Amino}cyclohexyl]sulfonyl}phenyl)-1,2-dihydropyridin-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one
5-(4-{[trans-4-{[4-(pentafluoro-λ⁶-sulfanyl)phenyl]Amino}cyclohexyl]sulfonyl}phenyl)pyridin-2-ol
4-(pentafluoro-λ⁶-sulfanyl)-N-[trans-4-{[4′-(dimethylphosphoryl)-[1,1′-biphenyl]-4-yl]sulfonyl}cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-isoindol-1-one
6-(4-{[trans-4-{[4-(pentafluoro-λ⁶-sulfanyl)phenyl]Amino}cyclohexyl]sulfonyl}phenyl)-2,3-dihydro-1H-indol-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)pyridazine-3-carboxamide
4-(pentafluoro-λ⁶-sulfanyl)-N-[trans-4-{4-[3-(propan-2-yl)imidazo[1,2-a]pyridin-6-yl]benzenesulfonyl}cyclohexyl]aniline
4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline
5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one
6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one
1-(2-fluoro-4-{3-methylimidazo[1,2-a]pyridin-6-yl}benzenesulfonyl)-N-[4-(pentafluoro-λ⁶-sulfanyl)phenyl]piperidin-4-amine
1-(3-fluoro-4-{3-methylimidazo[1,2-a]pyridin-6-yl}benzenesulfonyl)-N-[4-(pentafluoro-λ⁶-sulfanyl)phenyl]piperidin-4-amine
1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine
1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine
6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(3-fluoro-4-{imidazo[1,2-a]pyridin-6-yl}benzenesulfonyl)-N-[4-(pentafluoro-λ⁶-sulfanyl)phenyl]piperidin-4-amine
6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(2-fluoro-4-{imidazo[1,2-a]pyridin-6-yl}benzenesulfonyl)-N-[4-(pentafluoro-λ⁶-sulfanyl)phenyl]piperidin-4-amine
1-[(5-{imidazo[1,2-a]pyridin-6-yl}pyridin-2-yl)sulfonyl]-N-[4-(pentafluoro-λ⁶-sulfanyl)phenyl]piperidin-4-amine
6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile
1-[(6-{imidazo[1,2-a]pyridin-6-yl}pyridin-3-yl)sulfonyl]-N-[4-(pentafluoro-λ⁶-sulfanyl)phenyl]piperidin-4-amine
6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile
1-(2-chloro-4-{imidazo[1,2-a]pyridin-6-yl}benzenesulfonyl)-N-[4-(pentafluoro-λ⁶-sulfanyl)phenyl]piperidin-4-amine
6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[3-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[3-(pentafluoro-λ⁶-sulfanyl)phenyl]piperidin-4-amine
4-(4-{[(3S,4S)-3-fluoro-4-{[4-(pentafluoro-λ⁶-sulfanyl)phenyl]Amino}piperidin-1-yl]sulfonyl}phenyl)pyridine-2-carboxamide
N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
5-(4-{[trans-4-{[4-(pentafluoro-λ⁶-sulfanyl)phenyl]Amino}cyclohexyl]sulfonimidoyl}phenyl)-1H-indole-3-carbonitrile
[4-(3-methyl-1H-indazol-5-yl)phenyl][trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
4-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide
(4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone
[trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone
N-methyl-4′-{[trans-4-{[4-(pentafluoro-λ⁶-sulfanyl)phenyl]Amino}cyclohexyl]sulfonimidoyl}-[1,1′-biphenyl]-4-carboxamide
4′-{[trans-4-{[4-(pentafluoro-λ⁶-sulfanyl)phenyl]Amino}cyclohexyl]sulfonimidoyl}-[1,1′-biphenyl]-4-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide
N-methyl-4′-{[trans-4-{[4-(pentafluoro-λ⁶-sulfanyl)phenyl]Amino}cyclohexyl]sulfonimidoyl}-[1,1′-biphenyl]-3-carboxamide
4′-{[trans-4-{[4-(pentafluoro-λ⁶-sulfanyl)phenyl]Amino}cyclohexyl]sulfonimidoyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide
N-methyl-5-(4-{[trans-4-{[4-(pentafluoro-λ⁶-sulfanyl)phenyl]Amino}cyclohexyl]sulfonimidoyl}phenyl)pyridine-2-carboxamide
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
(4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone
N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide
N-methyl-6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide
N-methyl-4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
5-(4-{[trans-4-{[4-(pentafluoro-λ⁶-sulfanyl)phenyl]Amino}cyclohexyl]sulfonimidoyl}phenyl)-2,3-dihydro-1H-indol-2-one
6-(4-{[trans-4-{[4-(pentafluoro-λ⁶-sulfanyl)phenyl]Amino}cyclohexyl]sulfonimidoyl}phenyl)-2,3-dihydro-1H-indol-2-one
{4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one
5-{4-[(4-{[4-(pentafluoro-λ⁶-sulfanyl)phenyl]Amino}piperidin-1-yl)sulfonimidoyl]phenyl}-1H-indole-3-carbonitrile
N-methyl-5-{4-[(4-{[4-(pentafluoro-λ⁶-sulfanyl)phenyl]Amino}piperidin-1-yl)sulfonimidoyl]phenyl}pyridine-2-carboxamide
4′-[(4-{[4-(pentafluoro-λ⁶-sulfanyl)phenyl]Amino}piperidin-1-yl)sulfonimidoyl]-[1,1′-biphenyl]-4-carbonitrile
5-{4-[(4-{[4-(pentafluoro-λ⁶-sulfanyl)phenyl]Amino}piperidin-1-yl)sulfonimidoyl]phenyl}-2,3-dihydro-1H-indol-2-one
(4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone
4-{4-[(4-{[4-(pentafluoro-λ⁶-sulfanyl)phenyl]Amino}piperidin-1-yl)sulfonimidoyl]phenyl}pyridine-2-carboxamide
(+)-5-(4-{[trans-4-{[4-(pentafluoro-λ⁶-sulfanyl)phenyl]Amino}cyclohexyl]sulfonimidoyl}phenyl)-1H-indole-3-carbonitrile
(−)-5-(4-{[trans-4-{[4-(pentafluoro-λ⁶-sulfanyl)phenyl]Amino}cyclohexyl]sulfonimidoyl}phenyl)-1H-indole-3-carbonitrile
(+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(+)-4-(4-{[trans-4-{[4-(pentafluoro-λ⁶-sulfanyl)phenyl]Amino}cyclohexyl]sulfonimidoyl}phenyl)pyridine-2-carboxamide
(−)-4-(4-{[trans-4-{[4-(pentafluoro-λ⁶-sulfanyl)phenyl]Amino}cyclohexyl]sulfonimidoyl}phenyl)pyridine-2-carboxamide
1-(4-{2-methoxy-7-azaspiro[3.5]nonan-7-yl}benzenesulfonyl)-N-[4-(pentafluoro-λ⁶-sulfanyl)phenyl]piperidin-4-amine,
8-{4-[(4-{[4-(pentafluoro-λ⁶-sulfanyl)phenyl]amino}piperidin-1-yl)sulfonyl]phenyl}-1,3,8-triazaspiro[4.5]decane-2,4-dione,
8-{4-[(4-{[4-(pentafluoro-λ⁶-sulfanyl)phenyl]amino}piperidin-1-yl)sulfonyl]phenyl}-2,8-diazaspiro[4.5]decan-1-one,
8-{4-[(4-{[4-(pentafluoro-λ⁶-sulfanyl)phenyl]amino}piperidin-1-yl)sulfonyl]phenyl}-3-oxa-1,8-diazaspiro[4.5]decan-2-one,
1-(4-{2-oxa-7-azaspiro[3.5]nonan-7-yl}benzenesulfonyl)-N-[4-(pentafluoro-λ⁶-sulfanyl)phenyl]piperidin-4-amine,
1-(4-{8-oxa-3-azabicyclo[3.2.1]octan-3-yl}benzenesulfonyl)-N-[4-(pentafluoro-λ⁶-sulfanyl)phenyl]piperidin-4-amine,
4-fluoro-N-methyl-4′-[(4-{[4-(pentafluoro-λ⁶-sulfanyl)phenyl]amino}piperidin-1-yl)sulfonyl]-[1,1′-biphenyl]-3-carboxamide,
3-fluoro-N-methyl-4′-[(4-{[4-(pentafluoro-λ⁶-sulfanyl)phenyl]amino}piperidin-1-yl)sulfonyl]-[1,1′-biphenyl]-4-carboxamide,
N-[4-(pentafluoro-λ⁶-sulfanyl)phenyl]-1-{4-[3-(propan-2-yl)-[1,2,4]triazolo[4,3-a]pyrazin-6-yl]benzenesulfonyl}piperidin-4-amine,
N-(propan-2-yl)-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{3-methyl-1H-pyrazolo[3,4-c]pyridin-5-yl}benzenesulfonyl)cyclohexyl]aniline,
N,N-dimethyl-6-(4-{[trans-4-{[4-(pentafluoro-λ⁶-sulfanyl)phenyl]amino}cyclohexyl]sulfonyl}phenyl)imidazo[1,2-a]pyridin-3-amine,
N-cyclopropyl-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{5H-pyrrolo[3,2-d]pyrimidin-2-yl}benzenesulfonyl)cyclohexyl]aniline,
4-(pentafluoro-λ⁶-sulfanyl)-N-[trans-4-(4-{1H-pyrrolo[2,3-c]pyridin-5-yl}benzenesulfonyl)cyclohexyl]aniline,
4-(pentafluoro-λ⁶-sulfanyl)-N-[trans-4-(4-{1H-pyrazolo[3,4-c]pyridin-5-yl}benzenesulfonyl)cyclohexyl]aniline,
4-(4-{[trans-4-{[4-(pentafluoro-λ⁶-sulfanyl)phenyl]amino}cyclohexyl]sulfonyl}phenyl)thiophene-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
4-(pentafluoro-λ⁶-sulfanyl)-N-[trans-4-(4-{3-methyl-1H-pyrazolo[3,4-b]pyridin-5-yl}benzenesulfonyl)cyclohexyl]aniline,
4-(pentafluoro-λ⁶-sulfanyl)-N-[trans-4-{4-[8-(trifluoromethyl)imidazo[1,2-a]pyridin-6-yl]benzenesulfonyl}cyclohexyl]aniline,
4-(pentafluoro-λ⁶-sulfanyl)-N-[trans-4-(4-{8-fluoroimidazo[1,2-a]pyridin-6-yl}benzenesulfonyl)cyclohexyl]aniline,
2-(4-{[trans-4-{[4-(pentafluoro-λ⁶-sulfanyl)phenyl]amino}cyclohexyl]sulfonyl}phenyl)-4H,5H,6H,7H-thieno[3,2-c]pyridin-4-one,
7-(4-{[trans-4-{[4-(pentafluoro-λ⁶-sulfanyl)phenyl]amino}cyclohexyl]sulfonyl}phenyl)-1H,2H-pyrrolo[1,2-a]pyrazin-1-one,
8-(4-{[trans-4-{[4-(pentafluoro-λ⁶-sulfanyl)phenyl]amino}cyclohexyl]sulfonyl}phenyl)-1H,2H,3H,4H,5H-pyrrolo[1,2-a][1,4]diazepin-1-one,
7-(4-{[trans-4-{[4-(pentafluoro-λ⁶-sulfanyl)phenyl]amino}cyclohexyl]sulfonyl}phenyl)-1H,2H,3H,4H-pyrrolo[1,2-a]pyrazin-1-one,
6-(4-{[trans-4-{[4-(pentafluoro-λ⁶-sulfanyl)phenyl]amino}cyclohexyl]sulfonyl}phenyl)imidazo[1,2-a]pyridine-8-carboxamide,
4-(4-{[trans-4-{[4-(pentafluoro-λ⁶-sulfanyl)phenyl]amino}cyclohexyl]sulfonyl}phenyl)furan-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
6-(4-{[trans-4-{[4-(pentafluoro-λ⁶-sulfanyl)phenyl]amino}cyclohexyl]sulfonyl}phenyl)imidazo[1,2-a]pyridine-8-carbonitrile,
4-(pentafluoro-λ⁶-sulfanyl)-N-[trans-4-(4-{3-[(2R)-pyrrolidin-2-yl]-[1,2,4]triazolo[4,3-a]pyridin-6-yl}benzenesulfonyl)cyclohexyl]aniline,
{4-[3-(propan-2-yl)-[1,2,4]triazolo[4,3-a]pyrazin-6-yl]phenyl}[trans-4-{[4-(pentafluoro-λ⁶-sulfanyl)phenyl]amino}cyclohexyl](imino)-λ⁶-sulfanone,
(+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
(−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
N-[4-(pentafluoro-λ⁶-sulfanyl)phenyl]-1-(4-{3-[(3R)-pyrrolidin-3-yl]-[1,2,4]triazolo[4,3-a]pyridin-6-yl}benzenesulfonyl)piperidin-4-amine,
N-[4-(pentafluoro-λ⁶-sulfanyl)phenyl]-1-(4-{3-[(3S)-pyrrolidin-3-yl]-[1,2,4]triazolo[4,3-a]pyridin-6-yl}benzenesulfonyl)piperidin-4-amine,
1-{4-[3-(azetidin-3-yl)-[1,2,4]triazolo[4,3-a]pyridin-6-yl]benzenesulfonyl}-N-[4-(pentafluoro-λ⁶-sulfanyl)phenyl]piperidin-4-amine,
4-(pentafluoro-λ⁶-sulfanyl)-N-[trans-4-{4-[3-(azetidin-3-yl)-[1,2,4]triazolo[4,3-a]pyridin-6-yl]benzenesulfonyl}cyclohexyl]aniline;
{4-[3-(difluoromethyl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone;
(4-{3-methyl-[1,2,4]triazolo[4,3-a]pyrazin-6-yl}phenyl)[trans-4-{[4-(pentafluoro-λ⁶-sulfanyl)phenyl]amino}cyclohexyl](imino)-λ⁶-sulfanone;
{4-[3-(propan-2-yl)-[1,2,4]triazolo[4,3-a]pyridin-6-yl]phenyl}[trans-4-{[5-(pentafluoro-)⁶-sulfanyl)pyridin-2-yl]amino}cyclohexyl](imino)-λ⁶-sulfanone;
5-(pentafluoro-λ⁶-sulfanyl)-N-(1-{4-[3-(propan-2-yl)-[1,2,4]triazolo[4,3-a]pyridin-6-yl]benzenesulfonyl}piperidin-4-yl)pyridin-2-amine;
N-[1-(4-{imidazo[1,2-a]pyridin-6-yl}benzenesulfonyl)piperidin-4-yl]-5-(pentafluoro-λ⁶-sulfanyl)pyridin-2-amine;
N-[1-(4-{3-methyl-[1,2,4]triazolo[4,3-a]pyridin-6-yl}benzenesulfonyl)piperidin-4-yl]-5-(pentafluoro-λ⁶-sulfanyl)pyridin-2-amine;
4-{4-[(4-{[5-(pentafluoro-λ⁶-sulfanyl)pyridin-2-yl]amino}piperidin-1-yl)sulfonyl]phenyl}pyridine-2-carboxamide;
4-(pentafluoro-λ⁶-sulfanyl)-N-[trans-4-{4-[3-(difluoromethyl)-[1,2,4]triazolo[4,3-a]pyridin-6-yl]benzenesulfonyl}cyclohexyl]aniline;
4-{4-[(4-{[4-(pentafluoro-λ⁶-sulfanyl)phenyl]amino}piperidin-1-yl)sulfonyl]phenyl}pyridine-2-carboxamide;
5-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]pyridin-2-amine;
5-(pentafluoro-λ⁶-sulfanyl)-N-[trans-4-(4-{3-methyl-[1,2,4]triazolo[4,3-a]pyridin-6-yl}benzenesulfonyl)cyclohexyl]pyridin-2-amine;
5-(pentafluoro-λ⁶-sulfanyl)-N-[trans-4-(4-{imidazo[1,2-a]pyridin-6-yl}benzenesulfonyl)cyclohexyl]pyridin-2-amine;
or
5-(pentafluoro-λ⁶-sulfanyl)-N-[trans-4-{4-[8-methyl-3-(propan-2-yl)-[1,2,4]triazolo[4,3-a]pyridin-6-yl]benzenesulfonyl}cyclohexyl]pyridin-2-amine.
A particular embodiment of the present invention relates to a compound of formula (I) as described herein, or a pharmaceutically acceptable salt thereof, selected from:
1-(4-{imidazo[1,2-a]pyridin-6-yl}benzenesulfonyl)-N-[4-(pentafluoro-λ⁶-sulfanyl)phenyl]piperidin-4-amine
1-[4-(3-methyl-2H-indazol-5-yl)benzenesulfonyl]-N-[4-(pentafluoro-λ⁶-sulfanyl)phenyl]piperidin-4-amine
5-{4-[(4-{[4-(pentafluoro-λ⁶-sulfanyl)phenyl]Amino}piperidin-1-yl)sulfonyl]phenyl}-1H-indole-3-carbonitrile
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[cis-4-[4-(3-methyl-2H-indazol-5-yl)benzenesulfonyl]cyclohexyl]aniline
4-(pentafluoro-λ⁶-sulfanyl)-N-[trans-4-[4-(3-methyl-2H-indazol-5-yl)benzenesulfonyl]cyclohexyl]aniline
5-(4-{[trans-4-{[4-(pentafluoro-λ⁶-sulfanyl)phenyl]Amino}cyclohexyl]sulfonyl}phenyl)-1H-indole-3-carbonitrile
5-(4-{[cis-4-{[4-(pentafluoro-λ⁶-sulfanyl)phenyl]Amino}cyclohexyl]sulfonyl}phenyl)-1H-indole-3-carbonitrile
6-(4-{[trans-4-{[4-(pentafluoro-λ⁶-sulfanyl)phenyl]Amino}cyclohexyl]sulfonyl}phenyl)-2,3-dihydro-1H-isoindol-1-one
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(pentafluoro-λ⁶-sulfanyl)-N-[trans-4-(4-{imidazo[1,2-a]pyridin-6-yl}benzenesulfonyl)cyclohexyl]aniline
4-(pentafluoro-λ⁶-sulfanyl)-N-[trans-4-(4-{3-methylimidazo[1,2-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-indol-2-one
N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide
N-methyl-4′-{[trans-4-{[4-(pentafluoro-λ⁶-sulfanyl)phenyl]Amino}cyclohexyl]sulfonyl}-[1,1′-biphenyl]-4-carboxamide
6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile
4-(pentafluoro-λ⁶-sulfanyl)-N-[cis-4-(4-{imidazo[1,2-a]pyridin-6-yl}benzenesulfonyl)cyclohexyl]aniline
N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonyl}phenyl)pyridine-2-carboxamide
N-methyl-4′-{[trans-4-{[4-(pentafluoro-λ⁶-sulfanyl)phenyl]Amino}cyclohexyl]sulfonyl}-[1,1′-biphenyl]-3-carboxamide
N-methyl-5-(4-{[trans-4-{[4-(pentafluoro-λ⁶-sulfanyl)phenyl]Amino}cyclohexyl]sulfonyl}phenyl)pyridine-2-carboxamide
4′-{[trans-4-{[4-(pentafluoro-λ⁶-sulfanyl)phenyl]Amino}cyclohexyl]sulfonyl}-[1,1′-biphenyl]-3-carboxamide
1-(4-{imidazo[1,2-a]pyrazin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-)⁶-sulfanyl)phenyl]piperidin-4-amine
N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
N-[4-(pentafluoro-λ⁶-sulfanyl)phenyl]-1-{4-[3-(propan-2-yl)imidazo[1,2-a]pyridin-6-yl]benzenesulfonyl}piperidin-4-amine
1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine
1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile
6-(4-{[trans-4-{[4-(pentafluoro-λ⁶-sulfanyl)phenyl]Amino}cyclohexyl]sulfonyl}phenyl)imidazo[1,2-a]pyridine-3-carboxamide
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-(4-{[1,2,4]triazolo[4,3-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-1H-indazole-3-carboxamide
5-(4-{[trans-4-{[4-(pentafluoro-λ⁶-sulfanyl)phenyl]Amino}cyclohexyl]sulfonyl}phenyl)-1H-indazole-3-carbonitrile
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide
4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline
4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile
4-(pentafluoro-λ⁶-sulfanyl)-N-[trans-4-(4-{2-methylimidazo[1,2-a]pyrazin-6-yl}benzenesulfonyl)cyclohexyl]aniline
4-(pentafluoro-λ⁶-sulfanyl)-N-[trans-4-(4-{imidazo[1,2-a]pyrazin-6-yl}benzenesulfonyl)cyclohexyl]aniline
N,N-dimethyl-6-(4-{[cis-4-{[4-(pentafluoro-λ⁶-sulfanyl)phenyl]Amino}cyclohexyl]sulfonyl}phenyl)pyridine-3-carboxamide
N,N-dimethyl-6-(4-{[trans-4-{[4-(pentafluoro-λ⁶-sulfanyl)phenyl]Amino}cyclohexyl]sulfonyl}phenyl)pyridine-3-carboxamide
N-methyl-6-(4-{[trans-4-{[4-(pentafluoro-λ⁶-sulfanyl)phenyl]Amino}cyclohexyl]sulfonyl}phenyl)pyridine-3-carboxamide
N-methyl-4-(4-{[trans-4-{[4-(pentafluoro-λ⁶-sulfanyl)phenyl]Amino}cyclohexyl]sulfonyl}phenyl)pyridine-2-carboxamide
6-(4-{[trans-4-{[4-(pentafluoro-λ⁶-sulfanyl)phenyl]Amino}cyclohexyl]sulfonyl}phenyl)pyridine-3-carboxamide
4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
4-(4-{[trans-4-{[4-(pentafluoro-λ⁶-sulfanyl)phenyl]Amino}cyclohexyl]sulfonyl}phenyl)-1,2-dihydropyridin-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one
5-(4-{[trans-4-{[4-(pentafluoro-λ⁶-sulfanyl)phenyl]Amino}cyclohexyl]sulfonyl}phenyl)pyridin-2-ol
4-(pentafluoro-λ⁶-sulfanyl)-N-[trans-4-{[4′-(dimethylphosphoryl)-[1,1′-biphenyl]-4-yl]sulfonyl}cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-isoindol-1-one
6-(4-{[trans-4-{[4-(pentafluoro-λ⁶-sulfanyl)phenyl]Amino}cyclohexyl]sulfonyl}phenyl)-2,3-dihydro-1H-indol-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)pyridazine-3-carboxamide
4-(pentafluoro-λ⁶-sulfanyl)-N-[trans-4-{4-[3-(propan-2-yl)imidazo[1,2-a]pyridin-6-yl]benzenesulfonyl}cyclohexyl]aniline
4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline
5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one
6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one
1-(2-fluoro-4-{3-methylimidazo[1,2-a]pyridin-6-yl}benzenesulfonyl)-N-[4-(pentafluoro-λ⁶-sulfanyl)phenyl]piperidin-4-amine
1-(3-fluoro-4-{3-methylimidazo[1,2-a]pyridin-6-yl}benzenesulfonyl)-N-[4-(pentafluoro-λ⁶-sulfanyl)phenyl]piperidin-4-amine
1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine
1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine
6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(3-fluoro-4-{imidazo[1,2-a]pyridin-6-yl}benzenesulfonyl)-N-[4-(pentafluoro-λ⁶-sulfanyl)phenyl]piperidin-4-amine
6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(2-fluoro-4-{imidazo[1,2-a]pyridin-6-yl}benzenesulfonyl)-N-[4-(pentafluoro-λ⁶-sulfanyl)phenyl]piperidin-4-amine
1-[(5-{imidazo[1,2-a]pyridin-6-yl}pyridin-2-yl)sulfonyl]-N-[4-(pentafluoro-λ⁶-sulfanyl)phenyl]piperidin-4-amine
6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile
1-[(6-{imidazo[1,2-a]pyridin-6-yl}pyridin-3-yl)sulfonyl]-N-[4-(pentafluoro-λ⁶-sulfanyl)phenyl]piperidin-4-amine
6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile
1-(2-chloro-4-{imidazo[1,2-a]pyridin-6-yl}benzenesulfonyl)-N-[4-(pentafluoro-λ⁶-sulfanyl)phenyl]piperidin-4-amine
6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[3-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[3-(pentafluoro-λ⁶-sulfanyl)phenyl]piperidin-4-amine
4-(4-{[(3S,4S)-3-fluoro-4-{[4-(pentafluoro-λ⁶-sulfanyl)phenyl]Amino}piperidin-1-yl]sulfonyl}phenyl)pyridine-2-carboxamide
N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
5-(4-{[trans-4-{[4-(pentafluoro-λ⁶-sulfanyl)phenyl]Amino}cyclohexyl]sulfonimidoyl}phenyl)-1H-indole-3-carbonitrile
[4-(3-methyl-1H-indazol-5-yl)phenyl][trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
4-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide
(4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone
[trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone
N-methyl-4′-{[trans-4-{[4-(pentafluoro-λ⁶-sulfanyl)phenyl]Amino}cyclohexyl]sulfonimidoyl}-[1,1′-biphenyl]-4-carboxamide
4′-{[trans-4-{[4-(pentafluoro-λ⁶-sulfanyl)phenyl]Amino}cyclohexyl]sulfonimidoyl}-[1,1′-biphenyl]-4-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide
N-methyl-4′-{[trans-4-{[4-(pentafluoro-λ⁶-sulfanyl)phenyl]Amino}cyclohexyl]sulfonimidoyl}-[1,1′-biphenyl]-3-carboxamide
4′-{[trans-4-{[4-(pentafluoro-λ⁶-sulfanyl)phenyl]Amino}cyclohexyl]sulfonimidoyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide
N-methyl-5-(4-{[trans-4-{[4-(pentafluoro-λ⁶-sulfanyl)phenyl]Amino}cyclohexyl]sulfonimidoyl}phenyl)pyridine-2-carboxamide
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
(4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone
N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide
N-methyl-6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide
N-methyl-4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
5-(4-{[trans-4-{[4-(pentafluoro-λ⁶-sulfanyl)phenyl]Amino}cyclohexyl]sulfonimidoyl}phenyl)-2,3-dihydro-1H-indol-2-one
6-(4-{[trans-4-{[4-(pentafluoro-λ⁶-sulfanyl)phenyl]Amino}cyclohexyl]sulfonimidoyl}phenyl)-2,3-dihydro-1H-indol-2-one
{4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one
5-{4-[(4-{[4-(pentafluoro-λ⁶-sulfanyl)phenyl]Amino}piperidin-1-yl)sulfonimidoyl]phenyl}-1H-indole-3-carbonitrile
N-methyl-5-{4-[(4-{[4-(pentafluoro-λ⁶-sulfanyl)phenyl]Amino}piperidin-1-yl)sulfonimidoyl]phenyl}pyridine-2-carboxamide
4′-[(4-{[4-(pentafluoro-λ⁶-sulfanyl)phenyl]Amino}piperidin-1-yl)sulfonimidoyl]-[1,1′-biphenyl]-4-carbonitrile
5-{4-[(4-{[4-(pentafluoro-λ⁶-sulfanyl)phenyl]Amino}piperidin-1-yl)sulfonimidoyl]phenyl}-2,3-dihydro-1H-indol-2-one
(4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone
4-{4-[(4-{[4-(pentafluoro-λ⁶-sulfanyl)phenyl]Amino}piperidin-1-yl)sulfonimidoyl]phenyl}pyridine-2-carboxamide
(+)-5-(4-{[trans-4-{[4-(pentafluoro-λ⁶-sulfanyl)phenyl]Amino}cyclohexyl]sulfonimidoyl}phenyl)-1H-indole-3-carbonitrile
(−)-5-(4-{[trans-4-{[4-(pentafluoro-λ⁶-sulfanyl)phenyl]Amino}cyclohexyl]sulfonimidoyl}phenyl)-1H-indole-3-carbonitrile
(+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(+)-4-(4-{[trans-4-{[4-(pentafluoro-λ⁶-sulfanyl)phenyl]Amino}cyclohexyl]sulfonimidoyl}phenyl)pyridine-2-carboxamide
(−)-4-(4-{[trans-4-{[4-(pentafluoro-λ⁶-sulfanyl)phenyl]Amino}cyclohexyl]sulfonimidoyl}phenyl)pyridine-2-carboxamide
1-(4-{2-methoxy-7-azaspiro[3.5]nonan-7-yl}benzenesulfonyl)-N-[4-(pentafluoro-λ⁶-sulfanyl)phenyl]piperidin-4-amine,
8-{4-[(4-{[4-(pentafluoro-λ⁶-sulfanyl)phenyl]amino}piperidin-1-yl)sulfonyl]phenyl}-1,3,8-triazaspiro[4.5]decane-2,4-dione,
8-{4-[(4-{[4-(pentafluoro-λ⁶-sulfanyl)phenyl]amino}piperidin-1-yl)sulfonyl]phenyl}-2,8-diazaspiro[4.5]decan-1-one,
8-{4-[(4-{[4-(pentafluoro-λ⁶-sulfanyl)phenyl]amino}piperidin-1-yl)sulfonyl]phenyl}-3-oxa-1,8-diazaspiro[4.5]decan-2-one,
1-(4-{2-oxa-7-azaspiro[3.5]nonan-7-yl}benzenesulfonyl)-N-[4-(pentafluoro-λ⁶-sulfanyl)phenyl]piperidin-4-amine,
1-(4-{8-oxa-3-azabicyclo[3.2.1]octan-3-yl}benzenesulfonyl)-N-[4-(pentafluoro-λ⁶-sulfanyl)phenyl]piperidin-4-amine,
4-fluoro-N-methyl-4′-[(4-{[4-(pentafluoro-λ⁶-sulfanyl)phenyl]amino}piperidin-1-yl)sulfonyl]-[1,1′-biphenyl]-3-carboxamide,
3-fluoro-N-methyl-4′-[(4-{[4-(pentafluoro-λ⁶-sulfanyl)phenyl]amino}piperidin-1-yl)sulfonyl]-[1,1′-biphenyl]-4-carboxamide,
N-[4-(pentafluoro-λ⁶-sulfanyl)phenyl]-1-{4-[3-(propan-2-yl)-[1,2,4]triazolo[4,3-a]pyrazin-6-yl]benzenesulfonyl}piperidin-4-amine,
N-(propan-2-yl)-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{3-methyl-1H-pyrazolo[3,4-c]pyridin-5-yl}benzenesulfonyl)cyclohexyl]aniline,
N,N-dimethyl-6-(4-{[trans-4-{[4-(pentafluoro-λ⁶-sulfanyl)phenyl]amino}cyclohexyl]sulfonyl}phenyl)imidazo[1,2-a]pyridin-3-amine,
N-cyclopropyl-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{5H-pyrrolo[3,2-d]pyrimidin-2-yl}benzenesulfonyl)cyclohexyl]aniline,
4-(pentafluoro-λ⁶-sulfanyl)-N-[trans-4-(4-{1H-pyrrolo[2,3-c]pyridin-5-yl}benzenesulfonyl)cyclohexyl]aniline,
4-(pentafluoro-λ⁶-sulfanyl)-N-[trans-4-(4-{1H-pyrazolo[3,4-c]pyridin-5-yl}benzenesulfonyl)cyclohexyl]aniline,
4-(4-{[trans-4-{[4-(pentafluoro-λ⁶-sulfanyl)phenyl]amino}cyclohexyl]sulfonyl}phenyl)thiophene-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
4-(pentafluoro-λ⁶-sulfanyl)-N-[trans-4-(4-{3-methyl-1H-pyrazolo[3,4-b]pyridin-5-yl}benzenesulfonyl)cyclohexyl]aniline,
4-(pentafluoro-λ⁶-sulfanyl)-N-[trans-4-{4-[8-(trifluoromethyl)imidazo[1,2-a]pyridin-6-yl]benzenesulfonyl}cyclohexyl]aniline,
4-(pentafluoro-λ⁶-sulfanyl)-N-[trans-4-(4-{8-fluoroimidazo[1,2-a]pyridin-6-yl}benzenesulfonyl)cyclohexyl]aniline,
2-(4-{[trans-4-{[4-(pentafluoro-λ⁶-sulfanyl)phenyl]amino}cyclohexyl]sulfonyl}phenyl)-4H,5H,6H,7H-thieno[3,2-c]pyridin-4-one,
7-(4-{[trans-4-{[4-(pentafluoro-λ⁶-sulfanyl)phenyl]amino}cyclohexyl]sulfonyl}phenyl)-1H,2H-pyrrolo[1,2-a]pyrazin-1-one,
8-(4-{[trans-4-{[4-(pentafluoro-λ⁶-sulfanyl)phenyl]amino}cyclohexyl]sulfonyl}phenyl)-1H,2H,3H,4H,5H-pyrrolo[1,2-a][1,4]diazepin-1-one,
7-(4-{[trans-4-{[4-(pentafluoro-λ⁶-sulfanyl)phenyl]amino}cyclohexyl]sulfonyl}phenyl)-1H,2H,3H,4H-pyrrolo[1,2-a]pyrazin-1-one,
6-(4-{[trans-4-{[4-(pentafluoro-λ⁶-sulfanyl)phenyl]amino}cyclohexyl]sulfonyl}phenyl)imidazo[1,2-a]pyridine-8-carboxamide,
4-(4-{[trans-4-{[4-(pentafluoro-λ⁶-sulfanyl)phenyl]amino}cyclohexyl]sulfonyl}phenyl)furan-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
6-(4-{[trans-4-{[4-(pentafluoro-λ⁶-sulfanyl)phenyl]amino}cyclohexyl]sulfonyl}phenyl)imidazo[1,2-a]pyridine-8-carbonitrile,
4-(pentafluoro-λ⁶-sulfanyl)-N-[trans-4-(4-{3-[(2R)-pyrrolidin-2-yl]-[1,2,4]triazolo[4,3-a]pyridin-6-yl}benzenesulfonyl)cyclohexyl]aniline,
{4-[3-(propan-2-yl)-[1,2,4]triazolo[4,3-a]pyrazin-6-yl]phenyl}[trans-4-{[4-(pentafluoro-λ⁶-sulfanyl)phenyl]amino}cyclohexyl](imino)-λ⁶-sulfanone,
(+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
(−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
N-[4-(pentafluoro-λ⁶-sulfanyl)phenyl]-1-(4-{3-[(3R)-pyrrolidin-3-yl]-[1,2,4]triazolo[4,3-a]pyridin-6-yl}benzenesulfonyl)piperidin-4-amine,
N-[4-(pentafluoro-λ⁶-sulfanyl)phenyl]-1-(4-{3-[(3S)-pyrrolidin-3-yl]-[1,2,4]triazolo[4,3-a]pyridin-6-yl}benzenesulfonyl)piperidin-4-amine,
1-{4-[3-(azetidin-3-yl)-[1,2,4]triazolo[4,3-a]pyridin-6-yl]benzenesulfonyl}-N-[4-(pentafluoro-λ⁶-sulfanyl)phenyl]piperidin-4-amine, or
4-(pentafluoro-λ⁶-sulfanyl)-N-[trans-4-{4-[3-(azetidin-3-yl)-[1,2,4]triazolo[4,3-a]pyridin-6-yl]benzenesulfonyl}cyclohexyl]aniline.

A particular embodiment of the present invention relates to a compound of formula (I) as described herein, or a pharmaceutically acceptable salt thereof, selected from:
1-(4-{imidazo[1,2-a]pyridin-6-yl}benzenesulfonyl)-N-[4-(pentafluoro-λ⁶-sulfanyl)phenyl]piperidin-4-amine
1-[4-(3-methyl-2H-indazol-5-yl)benzenesulfonyl]-N-[4-(pentafluoro-λ⁶-sulfanyl)phenyl]piperidin-4-amine
5-{4-[(4-{[4-(pentafluoro-λ⁶-sulfanyl)phenyl]Amino}piperidin-1-yl)sulfonyl]phenyl}-1H-indole-3-carbonitrile
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[cis-4-[4-(3-methyl-2H-indazol-5-yl)benzenesulfonyl]cyclohexyl]aniline
4-(pentafluoro-λ⁶-sulfanyl)-N-[trans-4-[4-(3-methyl-2H-indazol-5-yl)benzenesulfonyl]cyclohexyl]aniline
5-(4-{[trans-4-{[4-(pentafluoro-λ⁶-sulfanyl)phenyl]Amino}cyclohexyl]sulfonyl}phenyl)-1H-indole-3-carbonitrile
5-(4-{[cis-4-{[4-(pentafluoro-λ⁶-sulfanyl)phenyl]Amino}cyclohexyl]sulfonyl}phenyl)-1H-indole-3-carbonitrile
6-(4-{[trans-4-{[4-(pentafluoro-λ⁶-sulfanyl)phenyl]Amino}cyclohexyl]sulfonyl}phenyl)-2,3-dihydro-1H-isoindol-1-one
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(pentafluoro-λ⁶-sulfanyl)-N-[trans-4-(4-{imidazo[1,2-a]pyridin-6-yl}benzenesulfonyl)cyclohexyl]aniline
4-(pentafluoro-λ⁶-sulfanyl)-N-[trans-4-(4-{3-methylimidazo[1,2-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-indol-2-one
N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide
N-methyl-4′-{[trans-4-{[4-(pentafluoro-λ⁶-sulfanyl)phenyl]Amino}cyclohexyl]sulfonyl}-[1,1′-biphenyl]-4-carboxamide
6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile
4-(pentafluoro-λ⁶-sulfanyl)-N-[cis-4-(4-{imidazo[1,2-a]pyridin-6-yl}benzenesulfonyl)cyclohexyl]aniline
N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonyl}phenyl)pyridine-2-carboxamide
N-methyl-4′-{[trans-4-{[4-(pentafluoro-λ⁶-sulfanyl)phenyl]Amino}cyclohexyl]sulfonyl}-[1,1′-biphenyl]-3-carboxamide
N-methyl-5-(4-{[trans-4-{[4-(pentafluoro-λ⁶-sulfanyl)phenyl]Amino}cyclohexyl]sulfonyl}phenyl)pyridine-2-carboxamide
4′-{[trans-4-{[4-(pentafluoro-λ⁶-sulfanyl)phenyl]Amino}cyclohexyl]sulfonyl}-[1,1′-biphenyl]-3-carboxamide
1-(4-{imidazo[1,2-a]pyrazin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine
N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
N-[4-(pentafluoro-λ⁶-sulfanyl)phenyl]-1-{4-[3-(propan-2-yl)imidazo[1,2-a]pyridin-6-yl]benzenesulfonyl}piperidin-4-amine
1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine
1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile
6-(4-{[trans-4-{[4-(pentafluoro-λ⁶-sulfanyl)phenyl]Amino}cyclohexyl]sulfonyl}phenyl)imidazo[1,2-a]pyridine-3-carboxamide
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-(4-{[1,2,4]triazolo[4,3-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-1H-indazole-3-carboxamide
5-(4-{[trans-4-{[4-(pentafluoro-λ⁶-sulfanyl)phenyl]Amino}cyclohexyl]sulfonyl}phenyl)-1H-indazole-3-carbonitrile
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide
4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline
4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile
4-(pentafluoro-λ⁶-sulfanyl)-N-[trans-4-(4-{2-methylimidazo[1,2-a]pyrazin-6-yl}benzenesulfonyl)cyclohexyl]aniline
4-(pentafluoro-λ⁶-sulfanyl)-N-[trans-4-(4-{imidazo[1,2-a]pyrazin-6-yl}benzenesulfonyl)cyclohexyl]aniline
N,N-dimethyl-6-(4-{[cis-4-{[4-(pentafluoro-λ⁶-sulfanyl)phenyl]Amino}cyclohexyl]sulfonyl}phenyl)pyridine-3-carboxamide
N,N-dimethyl-6-(4-{[trans-4-{[4-(pentafluoro-λ⁶-sulfanyl)phenyl]Amino}cyclohexyl]sulfonyl}phenyl)pyridine-3-carboxamide
N-methyl-6-(4-{[trans-4-{[4-(pentafluoro-λ⁶-sulfanyl)phenyl]Amino}cyclohexyl]sulfonyl}phenyl)pyridine-3-carboxamide
N-methyl-4-(4-{[trans-4-{[4-(pentafluoro-λ⁶-sulfanyl)phenyl]Amino}cyclohexyl]sulfonyl}phenyl)pyridine-2-carboxamide
6-(4-{[trans-4-{[4-(pentafluoro-λ⁶-sulfanyl)phenyl]Amino}cyclohexyl]sulfonyl}phenyl)pyridine-3-carboxamide
4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
4-(4-{[trans-4-{[4-(pentafluoro-λ⁶-sulfanyl)phenyl]Amino}cyclohexyl]sulfonyl}phenyl)-1,2-dihydropyridin-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one
5-(4-{[trans-4-{[4-(pentafluoro-λ⁶-sulfanyl)phenyl]Amino}cyclohexyl]sulfonyl}phenyl)pyridin-2-ol
4-(pentafluoro-λ⁶-sulfanyl)-N-[trans-4-{[4′-(dimethylphosphoryl)-[1,1′-biphenyl]-4-yl]sulfonyl}cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-isoindol-1-one
6-(4-{[trans-4-{[4-(pentafluoro-λ⁶-sulfanyl)phenyl]Amino}cyclohexyl]sulfonyl}phenyl)-2,3-dihydro-1H-indol-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)pyridazine-3-carboxamide
4-(pentafluoro-λ⁶-sulfanyl)-N-[trans-4-{4-[3-(propan-2-yl)imidazo[1,2-a]pyridin-6-yl]benzenesulfonyl}cyclohexyl]aniline
4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline
5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one
6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one
1-(2-fluoro-4-{3-methylimidazo[1,2-a]pyridin-6-yl}benzenesulfonyl)-N-[4-(pentafluoro-λ⁶-sulfanyl)phenyl]piperidin-4-amine
1-(3-fluoro-4-{3-methylimidazo[1,2-a]pyridin-6-yl}benzenesulfonyl)-N-[4-(pentafluoro-λ⁶-sulfanyl)phenyl]piperidin-4-amine
1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine
1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine
6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(3-fluoro-4-{imidazo[1,2-a]pyridin-6-yl}benzenesulfonyl)-N-[4-(pentafluoro-λ⁶-sulfanyl)phenyl]piperidin-4-amine
6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(2-fluoro-4-{imidazo[1,2-a]pyridin-6-yl}benzenesulfonyl)-N-[4-(pentafluoro-λ⁶-sulfanyl)phenyl]piperidin-4-amine
1-[(5-{imidazo[1,2-a]pyridin-6-yl}pyridin-2-yl)sulfonyl]-N-[4-(pentafluoro-λ⁶-sulfanyl)phenyl]piperidin-4-amine
6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile
1-[(6-{imidazo[1,2-a]pyridin-6-yl}pyridin-3-yl)sulfonyl]-N-[4-(pentafluoro-λ⁶-sulfanyl)phenyl]piperidin-4-amine
6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile
1-(2-chloro-4-{imidazo[1,2-a]pyridin-6-yl}benzenesulfonyl)-N-[4-(pentafluoro-λ⁶-sulfanyl)phenyl]piperidin-4-amine
6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[3-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[3-(pentafluoro-λ⁶-sulfanyl)phenyl]piperidin-4-amine
4-(4-{[(3S,4S)-3-fluoro-4-{[4-(pentafluoro-λ⁶-sulfanyl)phenyl]Amino}piperidin-1-yl]sulfonyl}phenyl)pyridine-2-carboxamide
N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
5-(4-{[trans-4-{[4-(pentafluoro-λ⁶-sulfanyl)phenyl]Amino}cyclohexyl]sulfonimidoyl}phenyl)-1H-indole-3-carbonitrile
[4-(3-methyl-1H-indazol-5-yl)phenyl][trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
4-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide
(4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone
[trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone
N-methyl-4′-{[trans-4-{[4-(pentafluoro-λ⁶-sulfanyl)phenyl]Amino}cyclohexyl]sulfonimidoyl}-[1,1′-biphenyl]-4-carboxamide
4′-{[trans-4-{[4-(pentafluoro-λ⁶-sulfanyl)phenyl]Amino}cyclohexyl]sulfonimidoyl}-[1,1′-biphenyl]-4-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide
N-methyl-4′-{[trans-4-{[4-(pentafluoro-λ⁶-sulfanyl)phenyl]Amino}cyclohexyl]sulfonimidoyl}-[1,1′-biphenyl]-3-carboxamide
4′-{[trans-4-{[4-(pentafluoro-λ⁶-sulfanyl)phenyl]Amino}cyclohexyl]sulfonimidoyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide
N-methyl-5-(4-{[trans-4-{[4-(pentafluoro-λ⁶-sulfanyl)phenyl]Amino}cyclohexyl]sulfonimidoyl}phenyl)pyridine-2-carboxamide
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
(4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone
N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide
N-methyl-6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide
N-methyl-4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
5-(4-{[trans-4-{[4-(pentafluoro-λ⁶-sulfanyl)phenyl]Amino}cyclohexyl]sulfonimidoyl}phenyl)-2,3-dihydro-1H-indol-2-one
6-(4-{[trans-4-{[4-(pentafluoro-λ⁶-sulfanyl)phenyl]Amino}cyclohexyl]sulfonimidoyl}phenyl)-2,3-dihydro-1H-indol-2-one
{4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one
5-{4-[(4-{[4-(pentafluoro-λ⁶-sulfanyl)phenyl]Amino}piperidin-1-yl)sulfonimidoyl]phenyl}-1H-indole-3-carbonitrile
N-methyl-5-{4-[(4-{[4-(pentafluoro-λ⁶-sulfanyl)phenyl]Amino}piperidin-1-yl)sulfonimidoyl]phenyl}pyridine-2-carboxamide
4′-[(4-{[4-(pentafluoro-λ⁶-sulfanyl)phenyl]Amino}piperidin-1-yl)sulfonimidoyl]-[1,1′-biphenyl]-4-carbonitrile
5-{4-[(4-{[4-(pentafluoro-λ⁶-sulfanyl)phenyl]Amino}piperidin-1-yl)sulfonimidoyl]phenyl}-2,3-dihydro-1H-indol-2-one
(4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone
4-{4-[(4-{[4-(pentafluoro-λ⁶-sulfanyl)phenyl]Amino}piperidin-1-yl)sulfonimidoyl]phenyl}pyridine-2-carboxamide
(+)-5-(4-{[trans-4-{[4-(pentafluoro-λ⁶-sulfanyl)phenyl]Amino}cyclohexyl]sulfonimidoyl}phenyl)-1H-indole-3-carbonitrile
(−)-5-(4-{[trans-4-{[4-(pentafluoro-λ⁶-sulfanyl)phenyl]Amino}cyclohexyl]sulfonimidoyl}phenyl)-1H-indole-3-carbonitrile
(+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(+)-4-(4-{[trans-4-{[4-(pentafluoro-λ⁶-sulfanyl)phenyl]Amino}cyclohexyl]sulfonimidoyl}phenyl)pyridine-2-carboxamide
(−)-4-(4-{[trans-4-{[4-(pentafluoro-λ⁶-sulfanyl)phenyl]Amino}cyclohexyl]sulfonimidoyl}phenyl)pyridine-2-carboxamide.

In a more particular embodiment of the present invention relates to a compound of formula (I) as described herein, or a pharmaceutically acceptable salt thereof, selected from:
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
N-methyl-4′-{[trans-4-{[4-(pentafluoro-λ⁶-sulfanyl)phenyl]Amino}cyclohexyl]sulfonyl}-[1,1′-biphenyl]-4-carboxamide
4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
5-(4-{[trans-4-{[4-(pentafluoro-λ⁶-sulfanyl)phenyl]Amino}cyclohexyl]sulfonyl}phenyl)-1H-indazole-3-carbonitrile
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(4-{[trans-4-{[4-(pentafluoro-λ⁶-sulfanyl)phenyl]Amino}cyclohexyl]sulfonyl}phenyl)pyridine-2-carboxamide
5-(4-{[trans-4-{[4-(pentafluoro-λ⁶-sulfanyl)phenyl]Amino}cyclohexyl]sulfonyl}phenyl)-1H-indole-3-carbonitrile
4-(pentafluoro-λ⁶-sulfanyl)-N-[trans-4-[4-(3-methyl-2H-indazol-5-yl)benzenesulfonyl]cyclohexyl]aniline
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[trans-4-(4-{3-methylimidazo[1,2-a]pyridin-6-yl}benzenesulfonyl)cyclohexyl]aniline
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[4-(pentafluoro-λ⁶-sulfanyl)phenyl]piperidin-4-amine.

In a more particular embodiment of the present invention relates to a compound of formula (I) as described herein, or a pharmaceutically acceptable salt thereof, selected from:
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
N-methyl-4′-{[trans-4-{[4-(pentafluoro-λ⁶-sulfanyl)phenyl]Amino}cyclohexyl]sulfonyl}-[1,1′-biphenyl]-4-carboxamide
4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
5-(4-{[trans-4-{[4-(pentafluoro-λ⁶-sulfanyl)phenyl]Amino}cyclohexyl]sulfonyl}phenyl)-1H-indazole-3-carbonitrile
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(4-{[trans-4-{[4-(pentafluoro-λ⁶-sulfanyl)phenyl]Amino}cyclohexyl]sulfonyl}phenyl)pyridine-2-carboxamide
5-(4-{[trans-4-{[4-(pentafluoro-λ⁶-sulfanyl)phenyl]Amino}cyclohexyl]sulfonyl}phenyl)-1H-indole-3-carbonitrile
4-(pentafluoro-λ⁶-sulfanyl)-N-[trans-4-[4-(3-methyl-2H-indazol-5-yl)benzenesulfonyl]cyclohexyl]aniline
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[trans-4-(4-{3-methylimidazo[1,2-a]pyridin-6-yl}benzenesulfonyl)cyclohexyl]aniline
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[4-(pentafluoro-λ⁶-sulfanyl)phenyl]piperidin-4-amine;
5-(pentafluoro-λ⁶-sulfanyl)-N-(1-{4-[3-(propan-2-yl)-[1,2,4]triazolo[4,3-a]pyridin-6-yl]benzenesulfonyl}piperidin-4-yl)pyridin-2-amine;
N-[1-(4-{imidazo[1,2-a]pyridin-6-yl}benzenesulfonyl)piperidin-4-yl]-5-(pentafluoro-λ⁶-sulfanyl)pyridin-2-amine;
or
N-[1-(4-{3-methyl-[1,2,4]triazolo[4,3-a]pyridin-6-yl}benzenesulfonyl)piperidin-4-yl]-5-(pentafluoro-λ⁶-sulfanyl)pyridin-2-amine.

In a more particular embodiment of the present invention relates to a compound of formula (I) as described herein, or a pharmaceutically acceptable salt thereof, selected from:
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
(+)-4-(4-{[trans-4-{[4-(pentafluoro-λ⁶-sulfanyl)phenyl]Amino}cyclohexyl]sulfonimidoyl}phenyl)pyridine-2-carboxamide
(+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone
(−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone

Further objects of the present invention are all forms of optically pure enantiomers, racemates or diastereomeric mixtures for compounds of formula (I) or (I′).

Process of manufacturing Processes for the manufacture of compounds of formula (I), or pharmaceutically acceptable salt thereof, as described herein are also an object of the invention.

The present invention provides a process of preparation of a compound as described herein, or a pharmaceutically acceptable salt thereof, wherein X¹ is N and X⁴ is O, comprising:
reacting compound of formula (VI), wherein R⁶, R⁷, and X⁵ are as defined above,

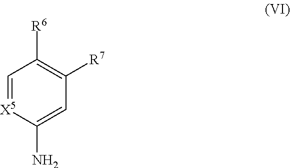

with compound of formula (VII), wherein R⁵ and R⁴ are as defined above,

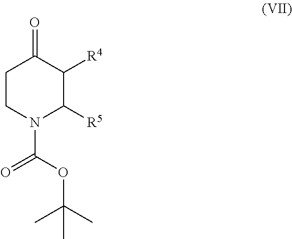

to form compound of formula (VIII), wherein R⁴, R⁵, X⁵, R⁶, R⁷ are as defined above,

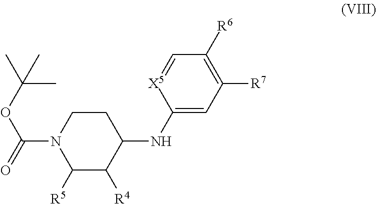

reacting said compound of formula (VIII) with acid to form compound of formula (IX), wherein R⁴, R⁵, X⁵, R⁶, R⁷ are as defined above,

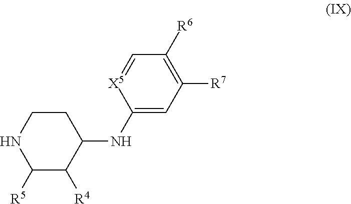

reacting said compound of formula (IX) with compound of formula (XI), wherein X², X³, R² and R³ are as defined above,

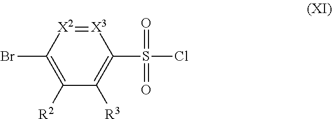

to form compound of formula (III), wherein R², R³, R⁴, R⁶, R⁷, R⁵, X², X⁵ and X³ are as defined above,

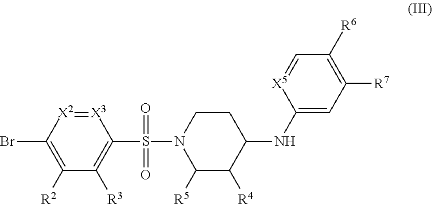

reacting said compound of formula (III) with compound of formula (X) wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

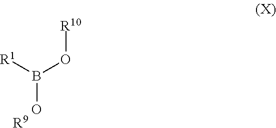

to form compound of formula (I).

The present invention provides a process of preparation of a compound as described herein, or a pharmaceutically acceptable salt thereof, wherein X¹ is CH and X⁴ is O, comprising:
reacting compound of formula (XII), wherein R⁵ and R⁴ are as defined in above,

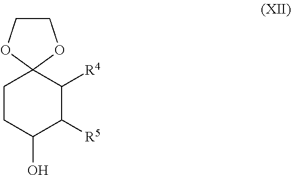

with CH₃SO₂Cl to form compound of formula (XIII), wherein R⁵ and R⁴ are as defined in above,

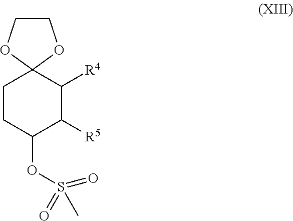

reacting said compound of formula (XIII) with compound of formula (XIV), wherein R³, R², X², and X³ are as defined above,

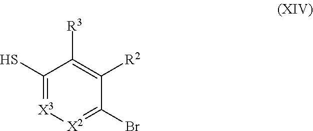

to form compound of formula (XV), wherein R³, R², R⁵, R⁴, X², and X³ are as defined above,

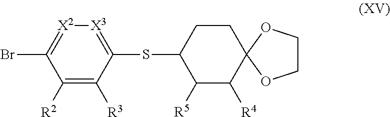

reacting said compound of formula (XV) with an oxidant to form compound of formula (XVI), wherein R³, R², R⁵, R⁴, X², and X³ are as defined above,

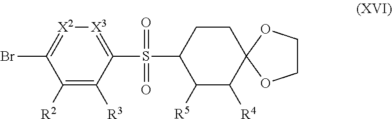

reacting said compound of formula (XVI) with an acid to form compound of formula (XVII), wherein R³, R², R⁵, R⁴, X², and X³ are as defined above,

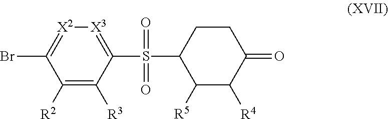

reacting said compound of formula (XVII) with compound of formula (VI), wherein R⁶, R⁷, and X⁵ are as defined above,

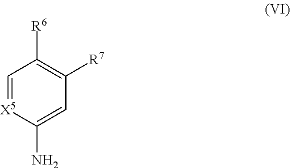

to form compound of formula (XVIII), wherein R², R³, R⁴, R⁶, R⁷, R⁵, X², X⁵ and X³ are as defined above,

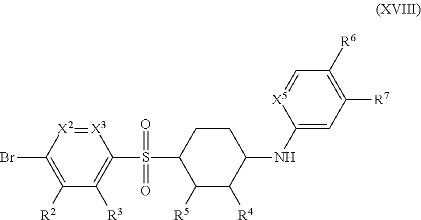

reacting said compound of formula (XVIII) with compound of formula (X) wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

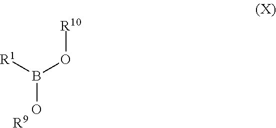

to form compound of formula (I).

The present invention provides a process of preparation of a compound as described herein, or a pharmaceutically acceptable salt thereof, wherein X¹ is CH and X⁴ is NH, comprising:
reacting compound of formula (XV), wherein R³, R², R⁵, R⁴, X², and X³ are as defined above,

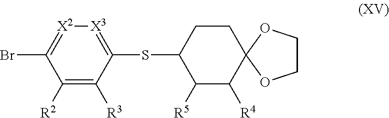

with an acid to form compound of formula (XX), wherein R³, R², R⁵, R⁴, X², and X³ are as defined above,

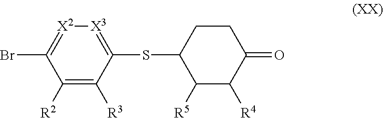

reacting said compound of formula (XX) with compound of formula (VI), wherein R⁶, R⁷, and X⁵ are as defined above,

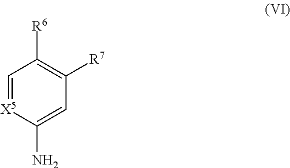

to form compound of formula (XXI), wherein R², R³, R⁴, R⁶, R⁷, R⁵, X², X⁵ and X³ are as defined above,

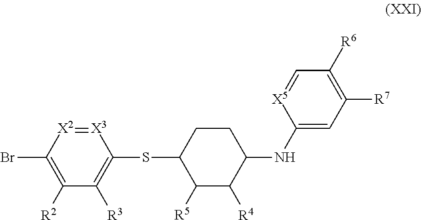

reacting said compound of formula (XXI) with ammonium carbamate and (diacetoxyiodo)benzene to form compound of formula (XXII), wherein R², R³, R⁴, R⁶, R⁷, R⁵, X², X⁵ and X³ are as defined above,

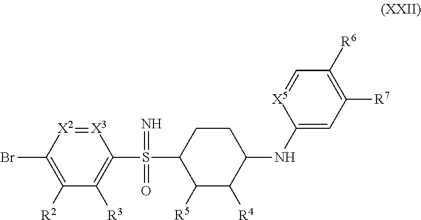

reacting said compound of formula (XXII) with compound of formula (X) wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

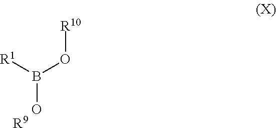

to form compound of formula (I).

The present invention provides a process of preparation of a compound as described herein, or a pharmaceutically acceptable salt thereof, wherein X¹ is N and X⁴ is NH, comprising:
reacting compound of formula (IX), wherein R⁴, R⁵, X⁵, R⁶, R⁷ are as defined above,

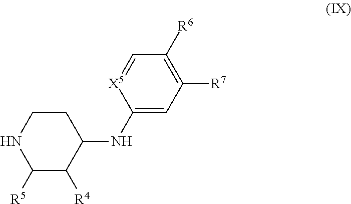

with compound of formula (XIV), wherein R² and R³ are as defined above,

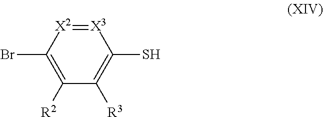

to form compound of formula (XXIV), wherein R², R³, R⁴, R⁶, R⁷, R⁵, X², X⁵ and X³ are as defined above,

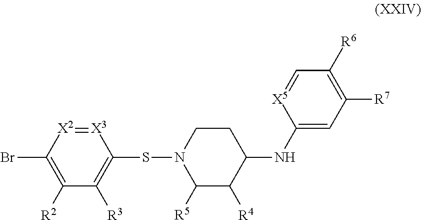

reacting said compound of formula (XXIV) with ammonium carbamate and (diacetoxyiodo)benzene to form compound of formula (XXV), wherein R², R³, R⁴, R⁶, R⁷, R⁵, X², X⁵ and X³ are as defined above,

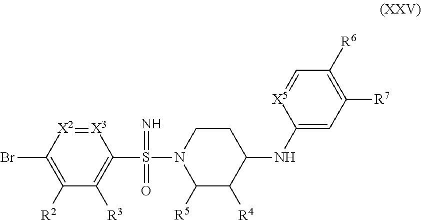

reacting said compound of formula (XXV) with di-tert-butyl dicarbonate and NaH to form compound of formula (XXVI), wherein R², R³, R⁴, R⁶, R⁷, R⁵, X², X⁵ and X³ are as defined above,

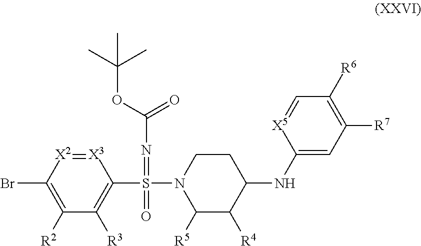

reacting said compound of formula (XXVI) with compound of formula (X), wherein R¹ is as defined above, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

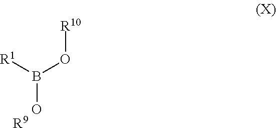

to form compound of formula (XXVII), wherein R¹, R², R³, R⁴, R⁶, R⁷, R⁵, X², X⁵ and X³ are as defined above,

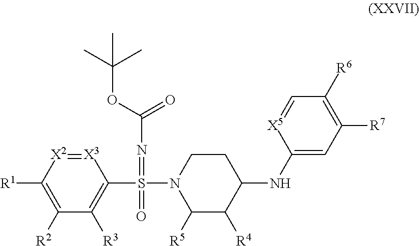

reacting said compound of formula (XXVII) with acid to form compound of formula (I).

### General Methods of Preparation

The compounds described herein, including compounds of general Formula (I), can be readily prepared according to the following reaction schemes and Examples, or modifications thereof, using readily available starting materials, reagent and conventional synthesis procedures. Many of the reactions can also be carried out under microwave conditions or using conventional heating or utilizing other technologies such as solid phase reagents/scavengers or flow chemistry. In these reactions, it is also possible to make use of variants which are themselves known to those skilled in the art, but are not mentioned in greater detail. For Example, where specific acids, bases, reagents, coupling agents, solvents, etc. are mentioned, it is understood that other suitable acids, bases, reagents, coupling agents, solvents etc. may be used and are included within the scope of the present invention. Furthermore, other methods for preparing compounds of the invention will be readily apparent to a person of ordinary skill in the art in light of the following reaction schemes and Examples. In cases where synthetic intermediates and final products contain potentially reactive functional groups, for Example amino, hydroxyl, thiol and carboxylic acid groups that may interfere with the desired reaction, it may be advantageous to employ protected forms of the intermediate. Methods for the selection, introduction and subsequent removal of protecting groups are well known to those skilled in the art. The compounds obtained by using the general reaction sequences may be of insufficient purity. The compounds can be purified by using any of the methods of purification of organic compounds, for Example, crystallization or silica gel, alumina or C18 column chromatography, using different solvents in suitable ratios. All possible stereoisomers are envisioned within the scope of the invention. In the discussion below variables have the meaning indicated above unless otherwise indicated. All final compounds have been characterized using for example LC-MS, NMR and/or Specific Optical Rotation.

The abbreviations used in these experimental details are listed below and additional ones should be considered known to a person skilled in the art of synthetic chemistry.

Abbreviations used herein are as follow: r.t.: room temperature; DMF: Dimethyl formamide; DiPEA: Diisopropylethylamine; DMAP: 4-(dimethylamino)pyridine; TFA: Trifluoroacetic acid; THF: Tetrahydrofuran; DMSO: Dimethylsulfoxide; EtOH: Ethanol; TLC: Thin Layer Chromatography; mCPBA: 3-chloroperbenzoic acid; MeOH: Methanol; HOAc: Acetic acid; NMP: N-methylpyrrolidinone, KOAc: Potassium acetate; N: normal; Josiphos SL J009-1: cyclopentane; ditert-butyl-[(1R)-1-(2-dicyclohexylphosphanylcyclopentyl)ethyl]phosphane; iron.

If a chemical compound is referred to using both a chemical structure and a chemical name, and an ambiguity exists between the structure and the name, the structure predominates.

### General Procedures

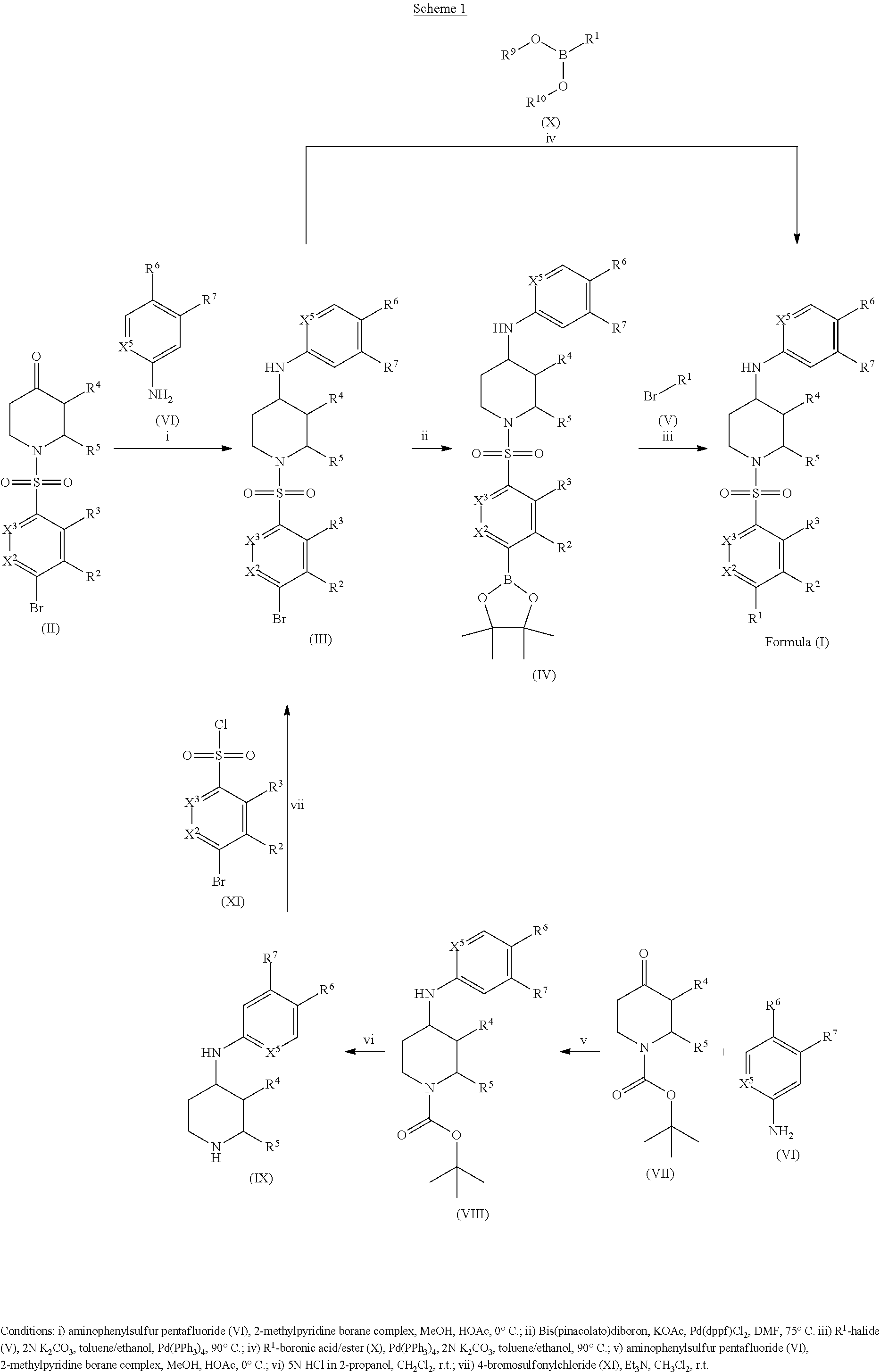

As depicted in scheme 1, the derivatives of the invention having Formula (I), when X₁═N and X₄═O, can be prepared by methods known in the art of organic chemistry. Compounds of the invention can for example be obtained by coupling of commercially available ketone derivatives of formula (II), wherein X₂, X₃, R₂, R₃, R₄ and R₅ have the meaning as previously described, and an aniline derivative of formula (VI), wherein X₅, R₆ and R₇ have the meaning as previously described, under reductive amination conditions to give the corresponding N-phenylpiperidin-4-amine of formula derivatives (III).

In an alternative way, N-phenylpiperidin-4-amine derivatives of formula (III) can be prepared starting from the N-protected piperidin-4-one derivatives of formula (VII), wherein R₄ and R₅ have the meaning as previously described, and an aniline derivative of formula (VI), wherein X₅, R₆ and R₇ have the meaning as previously described. Under reductive amination conditions, compounds of formula (VI) and (VII) can be converted to the corresponding piperidine derivatives of formula (VIII) which, after deprotection under acidic conditions, using for example HCl or TFA as the acid, give the piperidin-4-amine derivatives of formula (IX). These piperidine derivatives of formula (IX) can be converted into the derivatives of formula (III) by reaction with 4-bromobenzene-1-sulfonyl chloride derivatives of formula (XI), wherein X₂, X₃, R₂ and R₃ have the meaning as previously described.

Derivatives of Formula (I) can be prepared, either by direct coupling of derivatives of formula (III) with commercially available boronic acids or boronic esters of formula (X), wherein R₁, R⁹ and R¹⁰ are as defined previously under Suzuki conditions using for example Pd(PPh₃)₄ and NaHCO₃ in dioxane/water mixture or, by first converting piperidin derivatives of formula (III) into the corresponding boronic acids or boronic esters of formula (IV), which then can be reacted under Suzuki conditions, with R₁-halides of formula (V), wherein R₁ has the meaning as previously described.

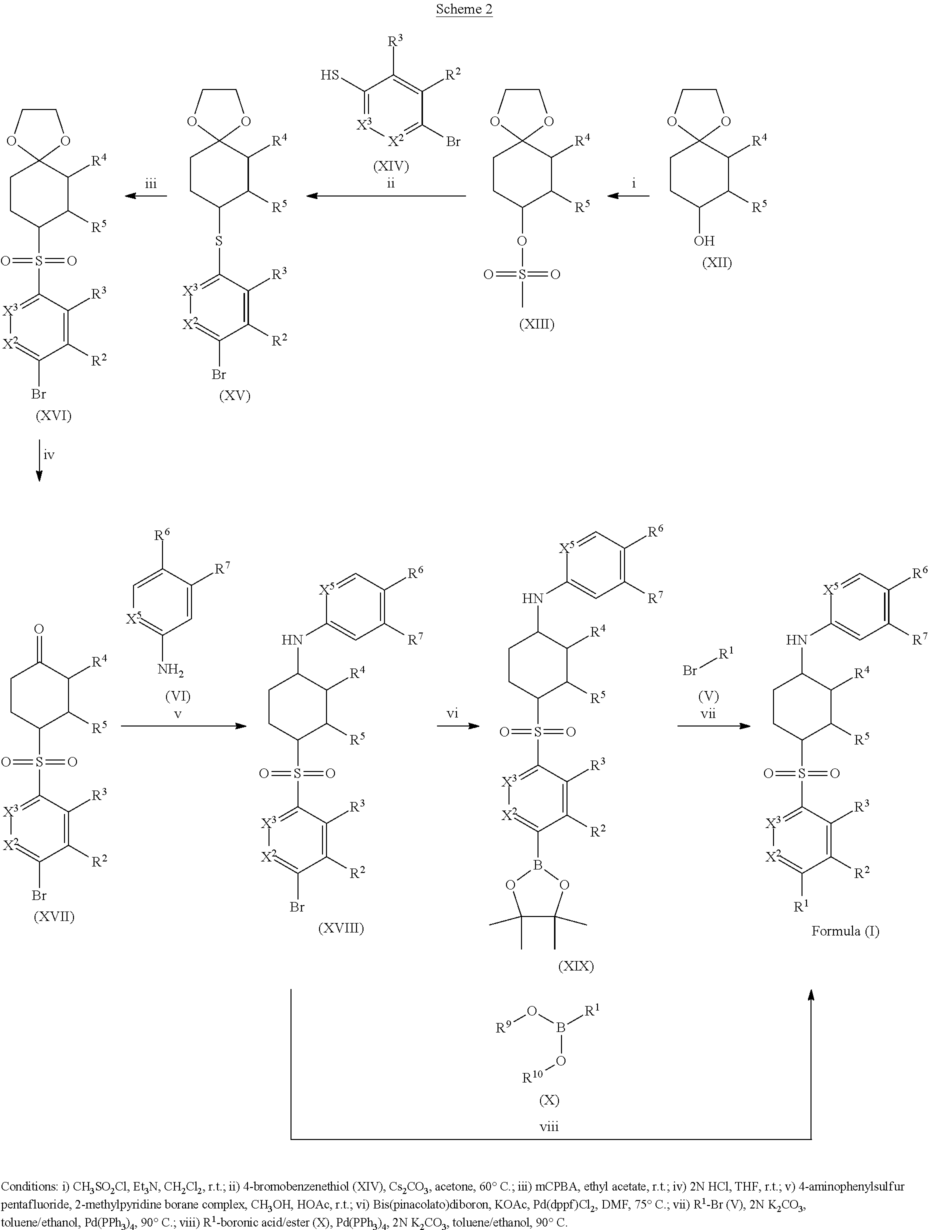

Scheme 2 describes a route to synthesize derivatives of the invention having Formula (I), when X₁═C and X₄═O.

These compounds can for example be obtained by starting from readily available keto-protected 4-hydroxycyclohexan-1-ones of formula (XII), wherein R₄ and R₅ have the meaning as previously described, in which the hydroxyl group is converted into a suitable leaving group, e.g. a mesyl or tosyl group, to give derivates of formula (XIII), which can react with 4-bromobenzenethiol derivatives of formula (XIV), to form the phenylsulfanyl derivatives of formula (XV). After oxidation of the sulfur, using for example mCPBA, the formed sulfonyl derivatives of formula (XVI) can then be deprotected under acid conditions to get the sulfonylcyclohexanone derivatives of formula (XVII). These sulfonylcyclohexanone derivatives of formula (XVII) can be coupled to an aniline of formula (VI), wherein X₅, R₆ and R₇ have the meaning as previously described, under reductive amination conditions to give the corresponding N-phenylpiperidin-4-amine derivatives of formula (XVIII).

Derivatives of Formula (I) can be prepared, either by direct coupling of derivatives of formula (XVIII) with commercially available boronic acids or boronic esters of formula (X), wherein R₁, R⁹ and R¹⁰ are as defined previously under Suzuki conditions using for example Pd(PPh₃)₄ and NaHCO₃ in dioxane/water mixture or, by first converting piperidin derivatives of formula (XVIII) into the corresponding boronic acids or boronic esters of formula (XIX), which then can be reacted under Suzuki conditions, with R₁-halides of formula (V), wherein R₁ has the meaning as previously described.

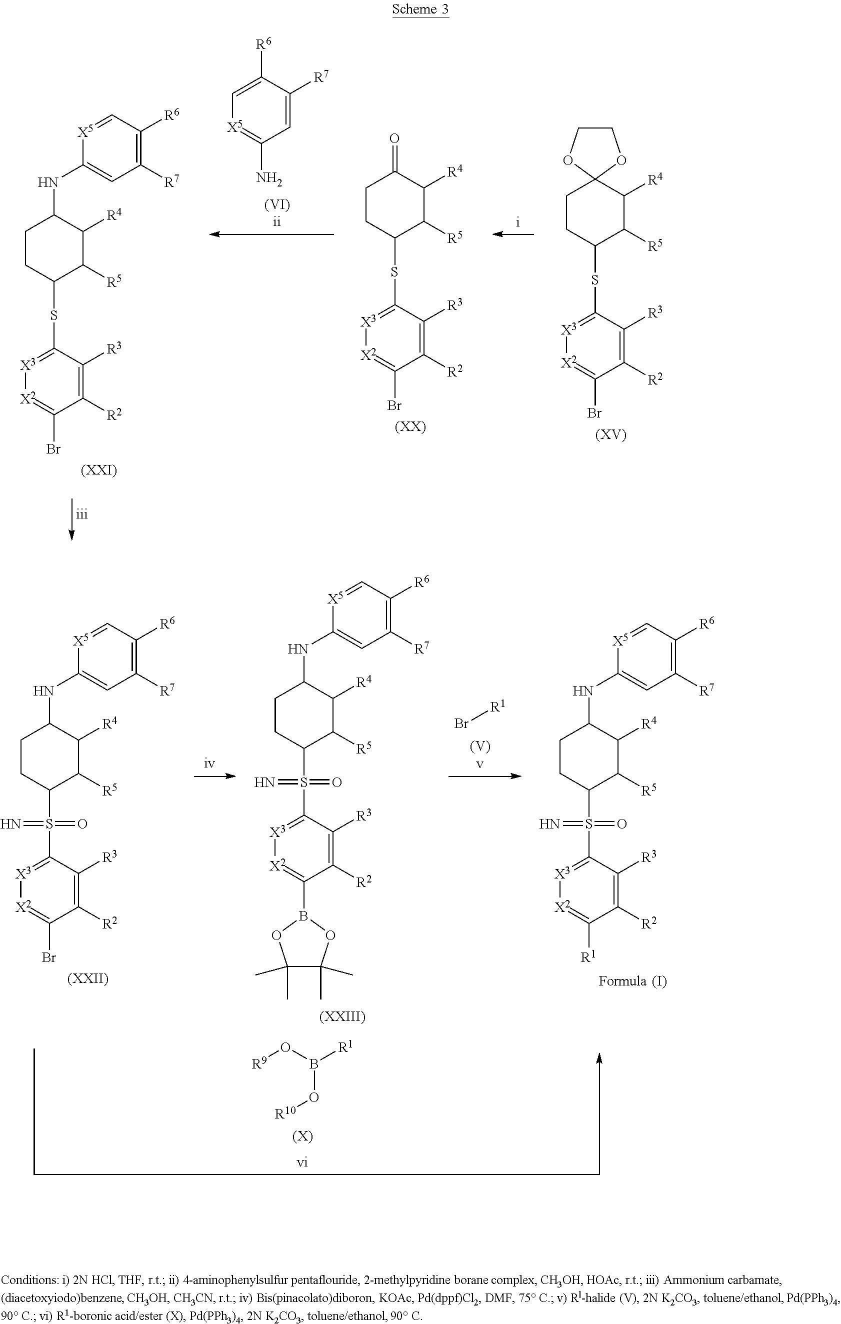

Scheme 3 describes a route to synthesize derivatives of the invention having Formula (I), when X₁═C and X₄═N.

Keto-protected sulfanyl derivatives of formula (XV) can be deprotected under acidic conditions, using for example HCl or TFA, to give 4-(phenylsulfanyl)cyclohexan-1-one derivatives of formula (XX). Under reductive amination conditions, derivatives of formula (XX) can be coupled with suitable aniline derivatives of formula (VI), wherein X₅, R₆ and R₇ have the meaning as previously described, to give the corresponding N-[4-(phenylsulfanyl)cyclohexyl]aniline derivatives of formula (XXI). Derivatives of formula (XXII) can be prepared by first converting derivatives of formula (XXI) into the corresponding sulfoximines derivatives, by using ammonium carbamate, (diacetoxyiodo)benzene and CH₃CN in CH₃OH. Finally, derivatives of formula (XXII) can be converted into derivatives of compound Formula (I) either by reaction with suitable boronic acids or boronic esters of formula (X), wherein R₁, R⁹ and R¹⁰ are as defined previously under Suzuki conditions using for example Pd(PPh₃)₄ as the catalyst or by first converting derivatives of formula (XXII) into their corresponding boronic acids/esters of formula (XXIII), which can further be reacted with suitable bromo aryl derivatives of (V) under Suzuki conditions, as previously described.

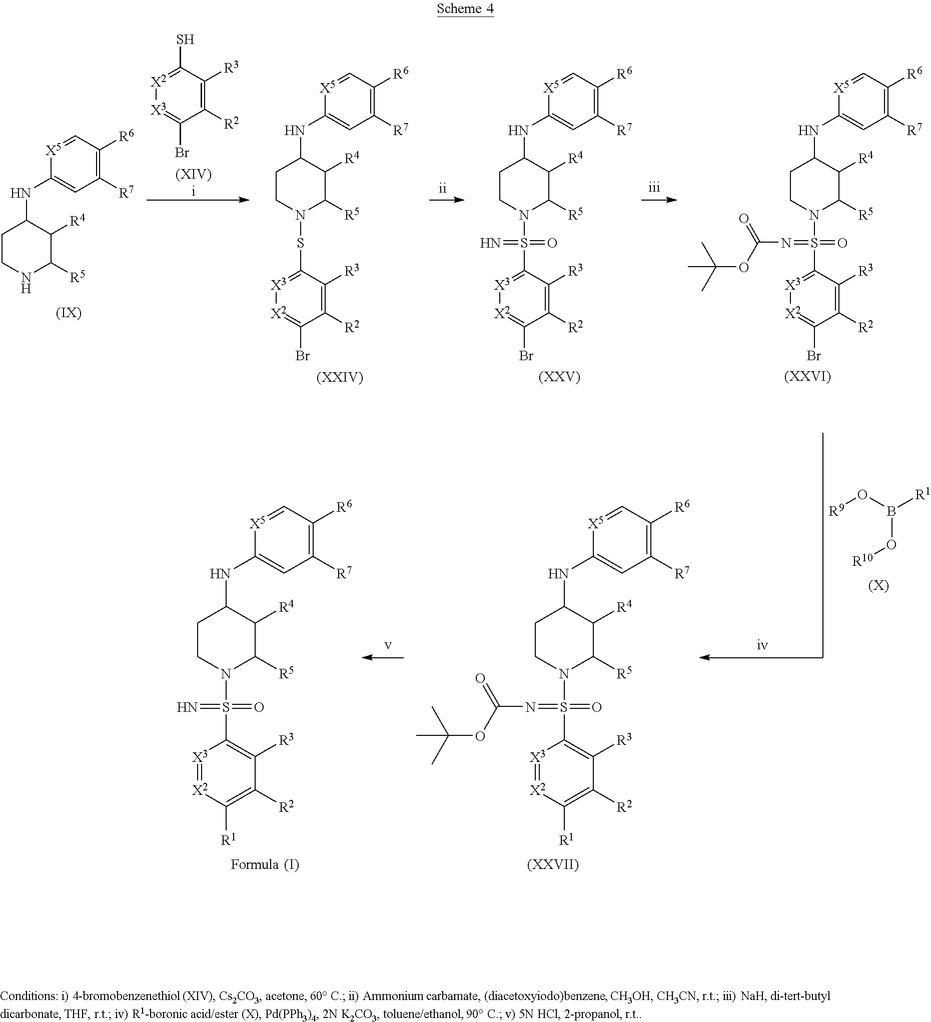

Scheme 4 describes a route to synthesize derivatives of the invention having Formula (I), when X₁ and X₄ are N.

N-phenylpiperidin-4-amine derivatives of formula (IX), wherein X₅, R₄, R₅, R₆ and R₇ have the meaning as previously described, can be converted into the N-phenyl-1-(phenylsulfanyl)piperidin-4-amine derivatives of formula (XXIV) by reaction with 4-bromobenzene-1-thiol derivatives of formula (XIV), wherein X₂, X₃, R₂ and R₃ have the meaning as previously described, using for example Cs₂CO₃ as the base. Derivatives of formula (XXIV) can be converted into the corresponding sulfoniminamide derivatives of formula (XXV), by using ammonium carbamate, (diacetoxyiodo)benzene and CH₃CN in CH₃OH. After protecting the sulfonimidamide nitrogen with for example a Boc-group, derivatives of formula (XXVI) can react with suitable boronic acids or boronic esters of formula (X) under Suzuki conditions, as previously described, to give derivatives of formula (XXVII). In the final step, the Boc-group can be removed, under acidic conditions, to give the corresponding derivatives of Formula (I), wherein X¹ and X₄ are N, X₂, X₃, X₅, R₁, R₂, R₃, R₄, R₅, R₆ and R₇ have the meaning as previously described.

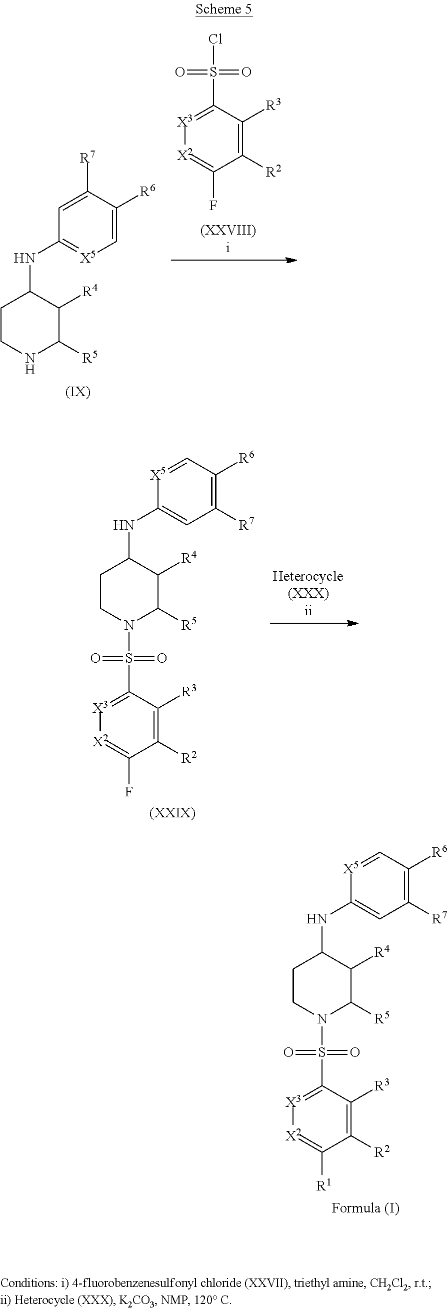

Scheme 5 describes a route to synthesize derivatives of the invention having Formula (I), when R₁ is a N-connected heterocyclic group.

N-phenylpiperidin-4-amine derivatives of formula (IX), wherein X₅, R₄, R₅, R₆ and R₇ have the meaning as previously described, can be converted into the 1-(4-fluorobenzenesulfonyl)-N-phenylpiperidin-4-amine derivatives of formula (XXIX) by reaction with 4-fluorobenzene-1-sulfonyl chloride derivatives of formula (XXVIII), wherein X₂, X₃, R₂ and R₃ have the meaning as previously described, using for example triethyl amine as the base. In the final step, an NH-heterocyclic group (XXX) can be coupled, under basic conditions, to give the corresponding derivatives of Formula (I), wherein X¹ is N, X₄ is O and X₂, X₃, X₅, R₁, R₂, R₃, R₄, R₅, R₆ and R₇ have the meaning as previously described.

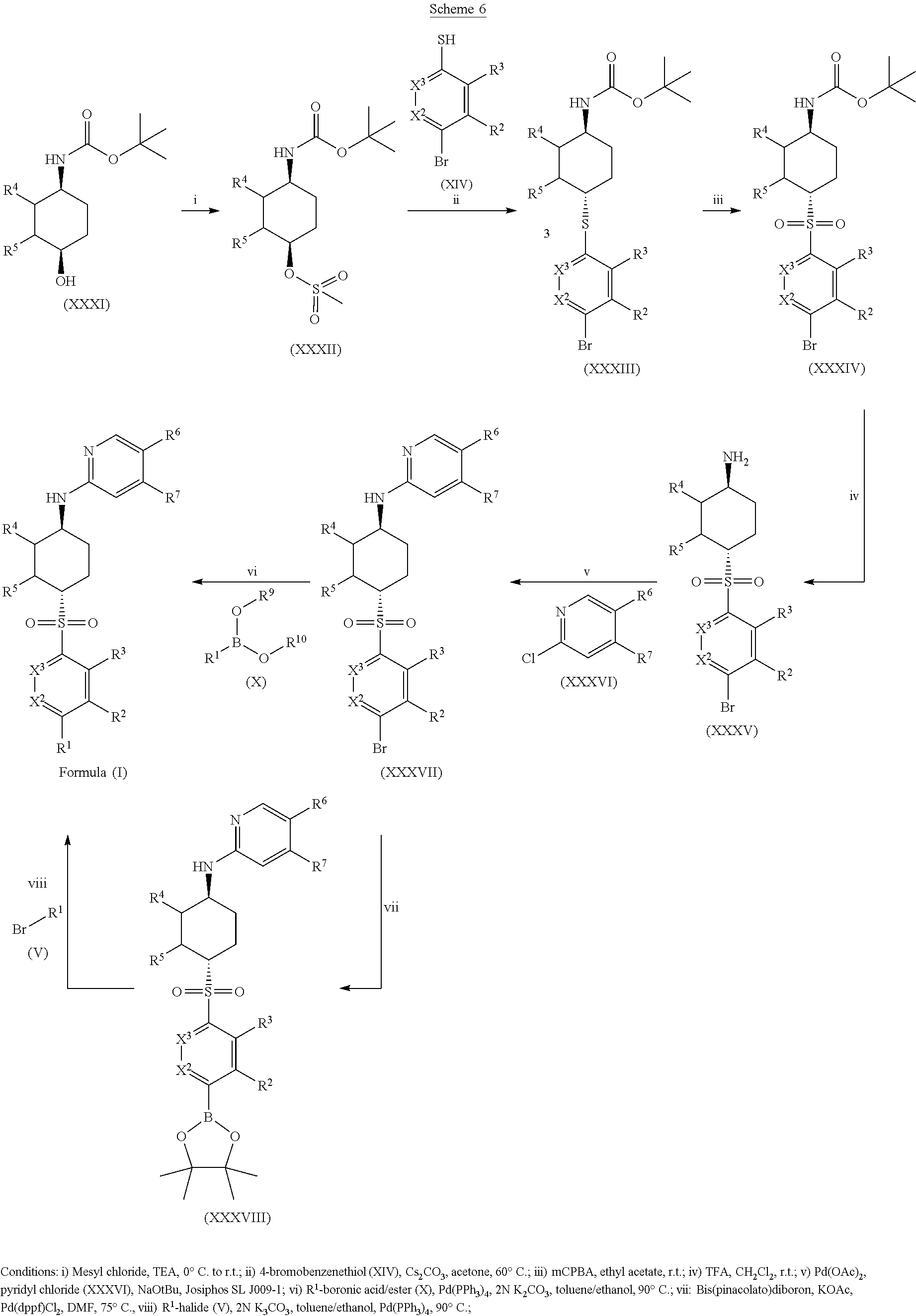

Scheme 6 describes a route to synthesize derivatives of the invention having Formula (I), when X¹ is O, X₄ is O and X₂, X₃, X₅, R₁, R₂, R₃, R₄, R₅, R₆ and R₇ have the meaning as previously described.

As depicted in scheme 5, the derivatives of the invention having Formula (I) can be obtained by coupling of N-Boc protected cis-4-aminocyclohexan-1-ol derivatives of formula (XXXI), wherein R⁴ and R⁵ have the meaning as previously described, which can easily be prepared by someone skilled in the art of organic chemistry, and mesylchloride under basic conditions, to give the corresponding N-Boc protected cis-4-aminocyclohexyl methanesulfonate derivatives of formula (XXXII). These sulfonates derivatives of formula (XXXII) can be converted into N-Boc protected 4-[(4-bromophenyl)sulfanyl]cyclohexan-1-amine derivatives of formula (XXXIII) via a nucleophilic substitution reaction with 4-bromobenzene-1-thiol derivatives of formula (XIV), using a suitable base e.g. Cs₂CO₃, which can be oxidized to the corresponding N-Boc protected sulfonyl derivatives of formula (XXXIV) using for example mCPBA as the oxidizing agent. After removing the Boc-group of derivatives of formula (XXXIV) under acidic conditions, using for example TFA, the obtained derivatives of formula (XXXV) can be converted, for example, under Buchwald conditions, using the appropriate pyridyl halide (XXXVI), wherein R⁶ and R⁷ have the meaning as previously described, using for example Josiphos SL J009-1, Pd(OAc)₂ and a suitable base e.g. NaOtBu, into derivatives of formula (XXXVII), wherein X², X³, X⁵, R¹, R², R³, R⁴, R⁵, R⁶ and R⁷ have the meaning as previously described. Finally, derivatives of formula (XXXVII) can be converted into derivatives of compound Formula (I) either by reaction with suitable boronic acids or boronic esters of formula (X), wherein R₁, R⁹ and R¹⁰ are as defined previously under Suzuki conditions using for example Pd(PPh₃)₄ as the catalyst or by first converting derivatives of formula (XXXVII) into their corresponding boronic acids/esters of formula (XXXVIII), which can further be reacted with suitable bromo aryl derivatives of (V) under Suzuki conditions, as previously described.

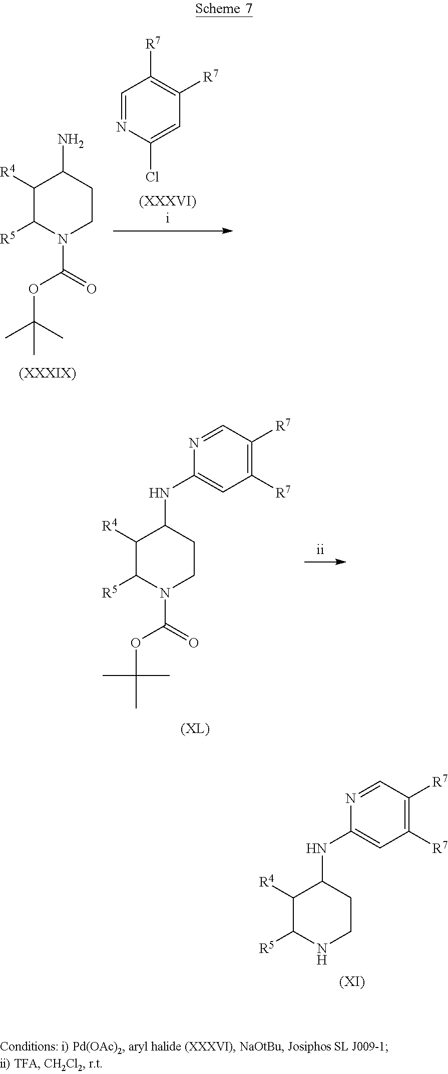

Scheme 7 describes a different way to synthesize derivatives of formula (IX), when X₅ is N.

t-Butyl 4-aminopiperidine-1-carboxylate derivatives of formula (XXXIX) can be converted for example, under Buchwald conditions, using the appropriate pyridyl chloride (XXXVI), wherein R⁶ and R⁷ have the meaning as previously described, using for example Josiphos SL J009-1, Pd(OAc)₂ and a suitable base e.g. NaOtBu, into derivatives of formula (XL), which after removing the Boc-group under acidic conditions, using for example TFA, give the corresponding pyridyl derivatives of formula (XI), wherein R⁴, R⁵, R⁶ and R⁷ have the meaning as previously described.

Another embodiment of the present invention is the compound of formula (I), as described herein, when manufactured according to the process described herein.

### Pharmaceutical Compositions and Administration

Another object of the present invention is a pharmaceutical composition comprising a compound of formula (I) as described herein, or a pharmaceutically acceptable salt thereof, and a pharmaceutically acceptable excipient.

Another embodiment of the present invention is a pharmaceutical composition comprising a compound as described herein, or a pharmaceutically acceptable salt thereof, and a pharmaceutically acceptable excipient.

Another embodiment of the present invention is the pharmaceutical composition as described herein, further comprising an additional therapeutic agent.

### Indications

The compounds of formula (I), (I′) or (I″) as described herein, can be used in an effective amount to treat a subject, in particular a human, affected by an inflammatory autoimmune disease.

In one embodiment of the present invention is the compound as described herein, or a pharmaceutically acceptable salt thereof, for use as therapeutically active substance.

In one embodiment of the present invention is the compound as described herein, or a pharmaceutically acceptable salt thereof, for use in the treatment, prevention and/or delay of an inflammatory autoimmune disease.

In one embodiment of the present invention is the compound as described herein, or a pharmaceutically acceptable salt thereof, for use in the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus and multiple sclerosis.

In one embodiment of the present invention is the compound as described herein, or a pharmaceutically acceptable salt thereof, for use in the treatment, prevention and/or delay of progression of psoriatic diseases, asthma, Crohn's disease, ulcerative colitis, rheumatoid arthritis, systemic lupus erythematosus and multiple sclerosis.

In one embodiment of the present invention is the compound as described herein, or a pharmaceutically acceptable salt thereof, for the preparation of a medicament for the treatment, prevention and/or delay of progression of psoriatic diseases, asthma, Crohn's disease, ulcerative colitis, rheumatoid arthritis, systemic lupus erythematosus and multiple sclerosis.

In one embodiment of the present invention is the compound as described herein, or a pharmaceutically acceptable salt thereof, for the preparation of a medicament for the treatment, prevention and/or delay of an inflammatory autoimmune disease.

In a further embodiment, the present invention provides a method for the treatment, prevention and/or delay of progression of an inflammatory autoimmune disease, which method comprises administering a therapeutically effective amount of a compound as described herein, or a pharmaceutically acceptable salt thereof.

In a further embodiment, the present invention provides a method for the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus or multiple, which method comprises administering a therapeutically effective amount of a compound as described herein, or a pharmaceutically acceptable salt thereof.

By the term “treatment” or “treating” and grammatical variations thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy using the methods and/or compositions of the invention is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing psoriatic diseases, asthma, Inflammatory Bowel Diseases (IBD), Crohn's disease, Obstructive Lung Diseases ulcerative colitis, rheumatoid arthritis, systemic lupus erythematosus or multiple, such as when a subject has a strong family history of psoriatic diseases, asthma, Inflammatory Bowel Diseases (IBD), Crohn's disease, Obstructive Lung Diseases ulcerative colitis, rheumatoid arthritis, systemic lupus erythematosus or multiple.

### EXPERIMENTAL PART

As depicted in the Examples below, in certain exemplary embodiments, compounds are prepared according to the following general procedures. It will be appreciated that, although the general methods depict the synthesis of certain compounds of the invention, the following general methods, and other methods known to one skilled in the art, can be applied to all compounds and subclasses and species of each of these compounds, as described herein.

### Example 1: 1-(4-{imidazo[1,2-a]pyridin-6-yl}benzenesulfonyl)-N-[4-(pentafluoro-λ⁶-sulfanyl)phenyl]piperidin-4-amine

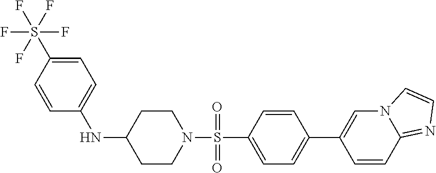 [SMILES: O=S(=O)(c1ccc(-c2ccc3nccn3c2)cc1)N1CCC(Nc2ccc(S(F)(F)(F)(F)F)cc2)CC1]

i) To a suspension of 1-(4-bromobenzenesulfonyl)piperidin-4-one (500 mg) and 4-(pentafluoro-λ⁶-sulfanyl)aniline (1.38 g) in a mixture of 10% HOAc in CH₃OH (16.5 mL) was added 2-methylpyridine borane complex (168 mg) at 0° C. After removal of the ice bath the reaction mixture was stirred overnight at room temperature. The reaction mixture was concentrated under reduced pressure and an aqueous 2N HCl solution (10 mL) was added. After stirring for 1 h at room temperature an aqueous 5N NaOH solution (20 mL) was added, and the product was extracted into CH₂Cl₂. The organic layer was washed with brine, dried over MgSO₄ and concentrated under reduced pressure. The residue was purified on SiO₂, using 30% ethyl acetate in heptane as the eluent, giving 1-(4-bromobenzenesulfonyl)-N-[4-(pentafluoro-λ⁶-sulfanyl)phenyl]piperidin-4-amine (1.75 g) as a white solid.
ii) To a solution of the product obtained in the previous step (200 mg), 6-(4,4,5,5-tetramethyl-1,3,2-dioxaborolan-2-yl)imidazo[1,2-a]pyridine (112 mg) and NaHCO₃ (193 mg) in a dioxane/water (4/1) mixture (5 mL), purged with N₂ gas, was added Pd(PPh₃)₄ (22 mg). The reaction mixture was heated for 2 h at 110° C. in a microwave. After cooling to room temperature, the product was extracted into ethyl acetate and the organic layer was washed with water, brine, dried over MgSO₄ and concentrated under reduced pressure. The residue was purified on C18, using 10% to 100% CH₃CN in water as the eluent, giving the title compound 1-(4-{imidazo[1,2a]pyridin-6-yl}benzenesulfonyl)-N-[4-(pentafluoro-λ⁶-sulfanyl)phenyl]piperidin-4-amine (59 mg) as a white solid. MS(ES⁺) m/z 559.2 (M+H)⁺.

¹H NMR (400 MHz, DMSO) δ 9.10 (dd, J=1.9, 1.0 Hz, 1H), 8.06-7.98 (m, 3H), 7.91-7.84 (m, 2H), 7.72 (dt, J=9.4, 0.9, 0.9 Hz, 1H), 7.67 (dd, J=9.1, 1.6 Hz, 2H), 7.52-7.43 (m, 2H), 6.61 (d, J=9.0 Hz, 2H), 6.47 (d, J=7.9 Hz, 1H), 3.62 (d, J=11.9 Hz, 2H), 3.44-3.34 (m, 1H), 2.63-2.52 (m, 2H), 1.97 (d, J=12.8 Hz, 2H), 1.52-1.38 (m, 2H).

Following a procedure analogous to that described for Example 1, using in step ii the appropriate boronic esters or boronic acids, Examples 2-4 have been prepared.

### Examples 2-4

### Example 2: 1-[4-(3-methyl-2H-indazol-5-yl)benzenesulfonyl]-N-[4-(pentafluoro-λ⁶-sulfanyl)phenyl]piperidin-4-amine

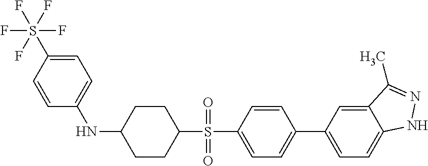 [SMILES: Cc1n[nH]c2ccc(-c3ccc(S(=O)(=O)C4CCC(Nc5ccc(S(F)(F)(F)(F)F)cc5)CC4)cc3)cc12]

MS(ES⁺) m/z 573.3 (M+H)⁺

Building block: Step ii: (3-methyl-1H-indazol-5-yl)boronic acid.

### Example 3: 5-{4-[(4-{[4-(pentafluoro-λ⁶-sulfanyl)phenyl]Amino}piperidin-1-yl)sulfonyl]phenyl}-1H-indole-3-carbonitrile

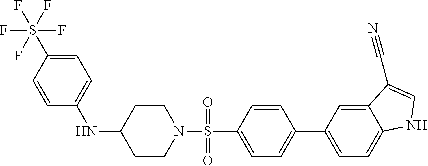 [SMILES: N#Cc1c[nH]c2ccc(-c3ccc(S(=O)(=O)N4CCC(Nc5ccc(S(F)(F)(F)(F)F)cc5)CC4)cc3)cc12]

MS(ES⁺) m/z 583.2 (M+H)⁺

Building block: Step ii: 5-(4,4,5,5-tetramethyl-1,3,2-dioxaborolan-2-yl)-1H-indole-3-carbonitrile.

### Example 4: 5-{4-[(4-{[4-(pentafluoro-λ⁶-sulfanyl)phenyl]Amino}piperidin-1-yl)sulfonyl]phenyl}-1H-indazole-3-carbonitrile

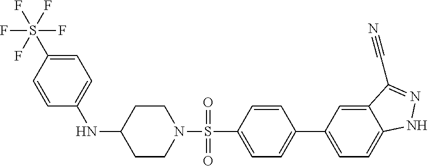 [SMILES: N#Cc1n[nH]c2ccc(-c3ccc(S(=O)(=O)N4CCC(Nc5ccc(S(F)(F)(F)(F)F)cc5)CC4)cc3)cc12]

MS(ES⁺) m/z 584.3 (M+H)⁺

Building block: Step ii: 5-(4,4,5,5-tetramethyl-1,3,2-dioxaborolan-2-yl)-1H-indazole-3-carbonitrile.

### Example 5: 4-(pentafluoro-λ⁶-sulfanyl)-N-[cis-4-[4-(3-methyl-2H-indazol-5-yl)benzenesulfonyl]cyclohexyl]aniline

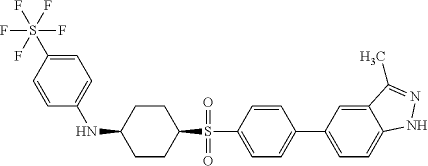 [SMILES: Cc1n[nH]c2ccc(-c3ccc(S(=O)(=O)[C@H]4CC[C@@H](Nc5ccc(S(F)(F)(F)(F)F)cc5)CC4)cc3)cc12]

i) A solution of methanesulfonyl chloride (1.3 mL) in CH₂Cl₂ (10 mL) was added dropwise to a solution of 4-hydroxycyclohexanone monoethylene ketal (2.0 g) and triethyl amine (2.3 mL) in CH₂Cl₂ (15 mL) at 0° C. The reaction mixture was stirred overnight at room temperature. After completion the reaction mixture was quenched by pouring into a saturated aqueous NaHCO₃ solution. The layers were separated, and the aqueous layers were extracted with CH₂Cl₂. The combined organic layers were washed brine, dried over MgSO₄, filtered, and concentrated under reduced pressure. The obtained yellow oil was dissolved in heptane, the precipitated solid was filtered off and the filtrate was reduced under reduced pressure to give 1,4-dioxaspiro[4.5]decan-8-yl methanesulfonate (3.3 g) as an oil, which slowly solidified. The product was used in the next step without further purification.
ii) 4-bromobenzene-1-thiol (2.64 g) was added to a suspension of the product obtained in the previous step (3.3 g) and cesium carbonate (5.46 g) in acetone (50 mL). The reaction mixture was stirred overnight at 60° C. After cooling to room temperature, the reaction mixture was filtered, and the solids were washed with ethyl acetate. The filtrate was concentrated under reduced pressure and the obtained oil was purified on SiO₂, using 0% to 60% ethyl acetate in heptane as the eluent, giving 8-[(4-bromophenyl)sulfanyl]-1,4-dioxaspiro[4.5]decane (3.68 g) as a white solid.
iii) 3-chloroperbenzoic acid (9.64 g) was added to a solution of the product obtained in the previous step (3.68 g) in ethyl acetate (50 mL) and the reaction mixture was stirred overnight at room temperature. The reaction mixture was washed with a saturated NaHCO₃ solution, and the combined organic layers were washed with brine, dried over MgSO₄, filtered, and concentrated under reduced pressure. The residue was purified on SiO₂, using 0% to 100% ethyl acetate in heptane as the eluent, giving 8-(4-bromobenzenesulfonyl)-1,4-dioxaspiro[4.5]decane (3.68 g) as a white solid.
iv) To a solution of the product obtained in the previous step (3.5 g) in THF (50 mL) was added at room temperature an aqueous 2N HCl solution (39 mL) and the reaction mixture was stirred overnight at room temperature. After completion, the reaction mixture was extracted with ethyl acetate and the combined organic layers were washed with brine, dried over MgSO₄, filtered and concentrated under reduced pressure to give 4-(4-bromobenzenesulfonyl)cyclohexan-1-one (3.0 g) as a white solid, which was used in the next step without further purification.
v) To a suspension of the product obtained in the previous step (500 mg) and 4-(pentafluoro-λ⁶-sulfanyl)aniline (0.35 ml g) in a mixture of 10% HOAc in CH₃OH (11 mL) was added 2-methylpyridine borane complex (219 mg) at 0° C. After removal of the ice bath the reaction mixture was stirred overnight at room temperature. The reaction mixture was concentrated under reduced pressure and an aqueous 2N HCl solution (5 mL) was added at 0° C. After stirring for 1 h at 0° C., an aqueous 5N NaOH solution (12 mL) was added, and the product was extracted into CH₂Cl₂. The organic layer was washed with brine, dried over MgSO₄, and concentrated under reduced pressure. The residue was purified on SiO₂, using 0% to 40% ethyl acetate in heptane as the eluent, giving N-[4-(4-bromobenzenesulfonyl)cyclohexyl]-4-(pentafluoro-λ⁶-sulfanyl)aniline (0.5 g) as a white solid.
vi) Under a nitrogen atmosphere, Pd(PPh₃)₄ (28 mg) was added to a solution of the product obtained in the previous step (250 mg), 3-methyl-5-(4,4,5,5-tetramethyl-1,3,2-dioxaborolan-2-yl)indazole (148 mg) and NaHCO₃ (242 mg) in a mixture of 1,4-dioxane (8 mL) and water (2 mL). The reaction mixture was stirred for 2 hours at 110° C. in a microwave. After cooling to room temperature, the reaction mixture was poured into water and the product was extracted into ethyl acetate. The combined organic layers were washed with water, brine, dried over MgSO₄, filtered, and concentrated under reduced pressure. The obtained brown oil was purified on C18, using 10% to 90% CH₃CN in water as the eluent, to give the title compound 4-(pentafluoro-λ⁶-sulfanyl)-N-[cis-4-[4-(3-methylindazol-5-yl)benzenesulfonyl]cyclohexyl]aniline (92 mg) as a white solid. MS(ES⁺) m/z 572.2 (M+H)⁺.

¹H NMR (400 MHz, DMSO) δ 12.78 (s, 1H), 8.15 (dd, J=1.8, 0.8 Hz, 1H), 8.08-8.00 (m, 2H), 7.95-7.88 (m, 2H), 7.75 (dd, J=8.8, 1.7 Hz, 1H), 7.59 (d, J=8.7 Hz, 1H), 7.54-7.49 (m, 2H), 6.67 (d, J=8.9 Hz, 2H), 6.58 (d, J=6.4 Hz, 1H), 3.59 (s, 1H), 3.41-3.34 (m, 1H), 2.56 (s, 3H), 1.94-1.56 (m, 8H).

Following a procedure analogous to that described for Example 5, using in step vi the appropriate boronic esters or boronic acids, Examples 6-23 have been prepared.

### Examples 6-23

### Example 6: 4-(pentafluoro-λ⁶-sulfanyl)-N-[trans-4-[4-(3-methyl-2H-indazol-5-yl)benzenesulfonyl]cyclohexyl]aniline

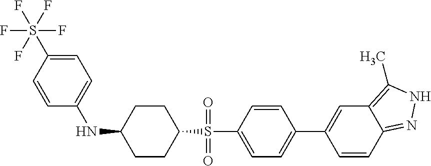 [SMILES: Cc1[nH]nc2ccc(-c3ccc(S(=O)(=O)[C@H]4CC[C@H](Nc5ccc(S(F)(F)(F)(F)F)cc5)CC4)cc3)cc12]

MS(ES⁺) m/z 572.2 (M+H)⁺

Building block: Step vi: 3-methyl-5-(4,4,5,5-tetramethyl-1,3,2-dioxaborolan-2-yl)indazole.

### Example 7: 5-(4-{[trans-4-{[4-(pentafluoro-λ⁶-sulfanyl)phenyl]Amino}cyclohexyl]sulfonyl}phenyl)-1H-indole-3-carbonitrile

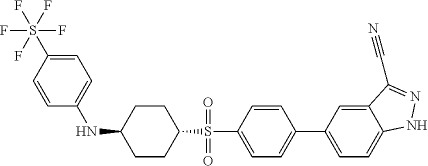 [SMILES: N#Cc1n[nH]c2ccc(-c3ccc(S(=O)(=O)[C@H]4CC[C@H](Nc5ccc(S(F)(F)(F)(F)F)cc5)CC4)cc3)cc12]

MS(ES⁺) m/z 582.2 (M+H)⁺

Building block: Step vi: 5-(4,4,5,5-tetramethyl-1,3,2-dioxaborolan-2-yl)-1H-indole-3-carbonitrile.

### Example 8: 5-(4-{[cis-4-{[4-(pentafluoro-λ⁶-sulfanyl)phenyl]Amino}cyclohexyl]sulfonyl}phenyl)-1H-indole-3-carbonitrile

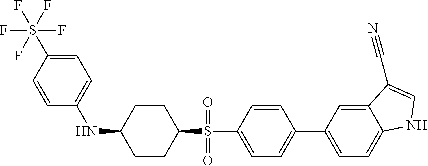 [SMILES: N#Cc1c[nH]c2ccc(-c3ccc(S(=O)(=O)[C@H]4CC[C@@H](Nc5ccc(S(F)(F)(F)(F)F)cc5)CC4)cc3)cc12]

MS(ES⁺) m/z 582.2 (M+H)⁺

Building block: Step vi: 5-(4,4,5,5-tetramethyl-1,3,2-dioxaborolan-2-yl)-1H-indole-3-carbonitrile.

### Example 9: 6-(4-{[trans-4-{[4-(pentafluoro-λ⁶-sulfanyl)phenyl]Amino}cyclohexyl]sulfonyl}phenyl)-2,3-dihydro-1H-isoindol-1-one

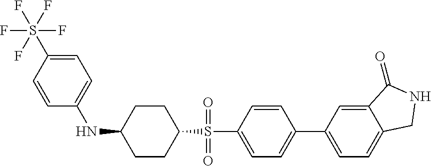 [SMILES: O=C1NCc2ccc(-c3ccc(S(=O)(=O)[C@H]4CC[C@H](Nc5ccc(S(F)(F)(F)(F)F)cc5)CC4)cc3)cc21]

MS(ES⁺) m/z 573.4 (M+H)⁺

Building block: Step vi: 6-(4,4,5,5-tetramethyl-1,3,2-dioxaborolan-2-yl)-2,3-dihydro-1H-isoindol-1-one.

### Example 10: 4-(4-{[trans-4-{[4-(pentafluoro-λ⁶-sulfanyl)phenyl]Amino}cyclohexyl]sulfonyl}phenyl)pyridine-2-carboxamide

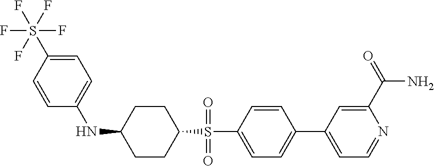 [SMILES: NC(=O)c1cc(-c2ccc(S(=O)(=O)[C@H]3CC[C@H](Nc4ccc(S(F)(F)(F)(F)F)cc4)CC3)cc2)ccn1]

MS(ES⁺) m/z 562.3 (M+H)⁺

Building block: Step vi: 4-(4,4,5,5-tetramethyl-1,3,2-dioxaborolan-2-yl)pyridine-2-carboxamide.

### Example 11: 4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline

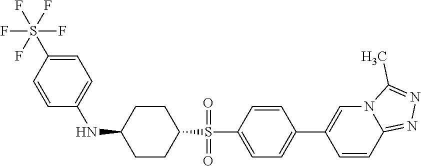 [SMILES: Cc1nnc2ccc(-c3ccc(S(=O)(=O)[C@H]4CC[C@H](Nc5ccc(S(F)(F)(F)(F)F)cc5)CC4)cc3)cn12]

MS(ES⁺) m/z 573.3 (M+H)⁺

Building block: Step vi: {3-methyl-[1,2,4]triazolo[4,3-a]pyridin-6-yl}boronic acid.

### Example 12: 4-(pentafluoro-λ⁶-sulfanyl)-N-[trans-4-(4-{imidazo[1,2-a]pyridin-6-yl}benzenesulfonyl)cyclohexyl]aniline

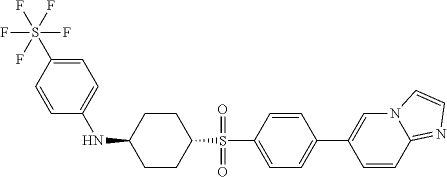 [SMILES: O=S(=O)(c1ccc(-c2ccc3nccn3c2)cc1)[C@H]1CC[C@H](Nc2ccc(S(F)(F)(F)(F)F)cc2)CC1]

MS(ES⁺) m/z 558.2 (M+H)⁺

Building block: Step vi: {imidazo[1,2-a]pyridin-6-yl}boronic acid.

### Example 13: 4-(pentafluoro-λ⁶-sulfanyl)-N-[trans-4-(4-{3-methylimidazo[1,2-a]pyridin-6-yl}benzenesulfonyl)cyclohexyl]aniline

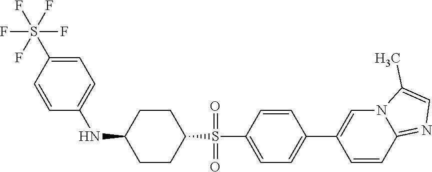 [SMILES: Cc1cnc2ccc(-c3ccc(S(=O)(=O)[C@H]4CC[C@H](Nc5ccc(S(F)(F)(F)(F)F)cc5)CC4)cc3)cn12]

MS(ES⁺) m/z 572 (M+H)⁺

Building block: Step vi: 3-methyl-6-(4,4,5,5-tetramethyl-1,3,2-dioxaborolan-2-yl)imidazo[1,2-a]pyridine.

### Example 14: 5-(4-{[trans-4-{[4-(pentafluoro-λ⁶-sulfanyl)phenyl]Amino}cyclohexyl]sulfonyl}phenyl)-2,3-dihydro-1H-indol-2-one

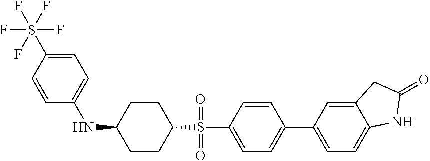 [SMILES: O=C1Cc2cc(-c3ccc(S(=O)(=O)[C@H]4CC[C@H](Nc5ccc(S(F)(F)(F)(F)F)cc5)CC4)cc3)ccc2N1]

MS(ES⁺) m/z 573.2 (M+H)⁺

Building block: Step vi: 5-(4,4,5,5-tetramethyl-1,3,2-dioxaborolan-2-yl)-2,3-dihydro-1H-indol-2-one.

### Example 15: N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide

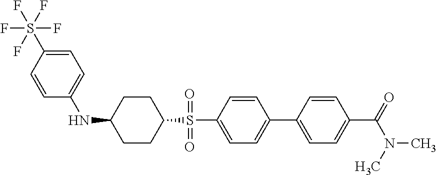 [SMILES: CN(C)C(=O)c1ccc(-c2ccc(S(=O)(=O)[C@H]3CC[C@H](Nc4ccc(S(F)(F)(F)(F)F)cc4)CC3)cc2)cc1]

MS(ES⁺) m/z 589.3 (M+H)⁺

Building block: Step vi: N,N-dimethyl-4-(4,4,5,5-tetramethyl-1,3,2-dioxaborolan-2-yl)benzamide.

### Example 16: N-methyl-4′-{[trans-4-{[4-(pentafluoro-λ⁶-sulfanyl)phenyl]Amino}cyclohexyl]sulfonyl}-[1,1′-biphenyl]-4-carboxamide

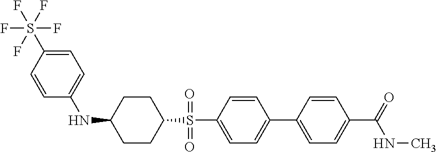 [SMILES: CNC(=O)c1ccc(-c2ccc(S(=O)(=O)[C@H]3CC[C@H](Nc4ccc(S(F)(F)(F)(F)F)cc4)CC3)cc2)cc1]

MS(ES⁺) m/z 575.3 (M+H)⁺

Building block: Step vi: N-methyl-4-(4,4,5,5-tetramethyl-1,3,2-dioxaborolan-2-yl)benzamide.

### Example 17: 6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile

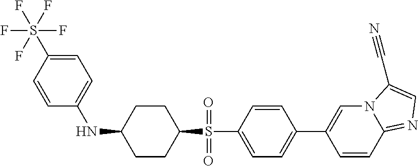 [SMILES: N#Cc1cnc2ccc(-c3ccc(S(=O)(=O)[C@H]4CC[C@@H](Nc5ccc(S(F)(F)(F)(F)F)cc5)CC4)cc3)cn12]

MS(ES⁺) m/z 583.3 (M+H)⁺

Building block: Step vi: 6-(4,4,5,5-tetramethyl-1,3,2-dioxaborolan-2-yl)imidazo[1,2-a]pyridine-3-carbonitrile.

### Example 18: 4-(pentafluoro-λ⁶-sulfanyl)-N-[cis-4-(4-{imidazo[1,2-a]pyridin-6-yl}benzenesulfonyl)cyclohexyl]aniline

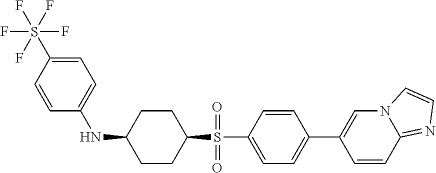 [SMILES: O=S(=O)(c1ccc(-c2ccc3nccn3c2)cc1)[C@H]1CC[C@@H](Nc2ccc(S(F)(F)(F)(F)F)cc2)CC1]

MS(ES⁺) m/z 558.3 (M+H)⁺

Building block: Step vi: 6-(4,4,5,5-tetramethyl-1,3,2-dioxaborolan-2-yl)imidazo[1,2-a]pyridine.

### Example 19: N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide

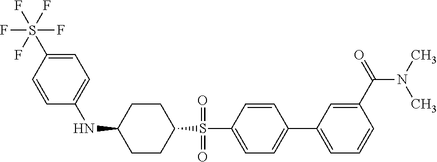 [SMILES: CN(C)C(=O)c1cccc(-c2ccc(S(=O)(=O)[C@H]3CC[C@H](Nc4ccc(S(F)(F)(F)(F)F)cc4)CC3)cc2)c1]

MS(ES⁺) m/z 587.3 (M−H)⁺

Building block: Step vi: N,N-dimethyl-3-(4,4,5,5-tetramethyl-1,3,2-dioxaborolan-2-yl)benzamide.

### Example 20: N,N-dimethyl-5-(4-{[trans-4-{[4-(pentafluoro-λ⁶-sulfanyl)phenyl]Amino}cyclohexyl]sulfonyl}phenyl)pyridine-2-carboxamide

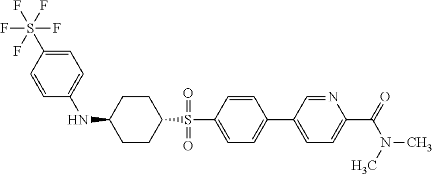 [SMILES: CN(C)C(=O)c1ccc(-c2ccc(S(=O)(=O)[C@H]3CC[C@H](Nc4ccc(S(F)(F)(F)(F)F)cc4)CC3)cc2)cn1]

MS(ES⁺) m/z 590.3 (M+H)⁺

Building block: Step vi: N,N-dimethyl-5-(4,4,5,5-tetramethyl-1,3,2-dioxaborolan-2-yl)pyridine-2-carboxamide.

### Example 21: N-methyl-4′-{[trans-4-{[4-(pentafluoro-λ⁶-sulfanyl)phenyl]Amino}cyclohexyl]sulfonyl}-[1,1′-biphenyl]-3-carboxamide

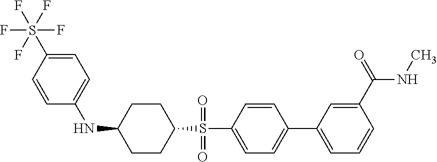 [SMILES: CNC(=O)c1cccc(-c2ccc(S(=O)(=O)[C@H]3CC[C@H](Nc4ccc(S(F)(F)(F)(F)F)cc4)CC3)cc2)c1]

MS(ES⁺) m/z 575 (M+H)⁺

Building block: Step vi: [3-(methylcarbamoyl)phenyl]boronic acid.

### Example 22: N-methyl-5-(4-{[trans-4-{[4-(pentafluoro-λ⁶-sulfanyl)phenyl]Amino}cyclohexyl]sulfonyl}phenyl)pyridine-2-carboxamide

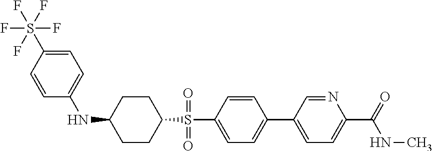 [SMILES: CNC(=O)c1ccc(-c2ccc(S(=O)(=O)[C@H]3CC[C@H](Nc4ccc(S(F)(F)(F)(F)F)cc4)CC3)cc2)cn1]

MS(ES⁺) m/z 576.3 (M+H)⁺

Building block: Step vi: N-methyl-5-(4,4,5,5-tetramethyl-1,3,2-dioxaborolan-2-yl)pyridine-2-carboxamide.

### Example 23: 4′-{[trans-4-{[4-(pentafluoro-λ⁶-sulfanyl)phenyl]Amino}cyclohexyl]sulfonyl}-[1,1′-biphenyl]-3-carboxamide

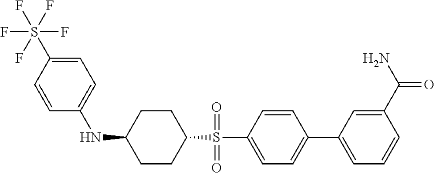 [SMILES: NC(=O)c1cccc(-c2ccc(S(=O)(=O)[C@H]3CC[C@H](Nc4ccc(S(F)(F)(F)(F)F)cc4)CC3)cc2)c1]

MS(ES⁺) m/z 561.3 (M+H)⁺

Building block: Step vi: (3-carbamoylphenyl)boronic acid.

### Example 24: 1-(4-{imidazo[1,2-a]pyrazin-6-yl}benzenesulfonyl)-N-[4-(pentafluoro-λ⁶-sulfanyl)phenyl]piperidin-4-amine

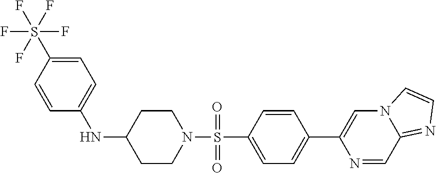 [SMILES: O=S(=O)(c1ccc(-c2cn3ccnc3cn2)cc1)N1CCC(Nc2ccc(S(F)(F)(F)(F)F)cc2)CC1]

i) To a suspension of 1-(4-bromobenzenesulfonyl)-N-[4-(pentafluoro-λ⁶-sulfanyl)phenyl]piperidin-4-amine (Example 1, step i, 500 mg), bis(pinacolato)diboron (365 mg) and potassium acetate (235 mg) in cyclopentyl methyl ether (5 mL), purged with N₂ gas, was added PdCl₂(dppf)·CH₂Cl₂ (39 mg). The reaction mixture was heated for 1 h at 100° C. in a microwave. After cooling to room temperature, the reaction mixture was poured into water and the product was extracted into ethyl acetate. The organic layer was washed with brine, dried over MgSO₄ and concentrated under reduced pressure giving N-[4-(pentafluoro-λ⁶-sulfanyl)phenyl]-1-[4-(4,4,5,5-tetramethyl-1,3,2-dioxaborolan-2-yl)benzenesulfonyl]piperidin-4-amine as a brown oil (558 mg), which was used in the next step without further purification.
ii) Following a procedure described for Example 1, step ii, the product obtained in the previous step (150 mg) and 6-bromoimidazo[1,2-a]pyrazine (63 mg) were converted to the title compound 1-(4-{imidazo[1,2-a]pyrazin-6-yl}benzenesulfonyl)-N-[4-(pentafluoro-λ⁶-sulfanyl)phenyl]piperidin-4-amine (48 mg) as a white solid. MS(ES⁺) m/z 560.3 (M+H)⁺.

¹H NMR (400 MHz, DMSO) δ 9.43 (d, J=1.5 Hz, 1H), 9.25-9.20 (m, 1H), 8.36-8.28 (m, 2H), 8.20 (t, J=0.9, 0.9 Hz, 1H), 7.94-7.85 (m, 3H), 7.51-7.42 (m, 2H), 6.60 (d, J=8.9 Hz, 2H), 6.46 (d, J=7.9 Hz, 1H), 3.61 (d, J=12.0 Hz, 2H), 3.43-3.34 (m, 1H), 2.59 (td, J=11.7, 11.6, 2.7 Hz, 2H), 2.01-1.93 (m, 2H), 1.52-1.38 (m, 2H).

Following a procedure analogous to that described for Example 24, using in step ii the appropriate aryl halide, Example 25-31 have been prepared.

### Examples 25-31

### Example 25: 6-{4-[(4-{[4-(pentafluoro-λ⁶-sulfanyl)phenyl]Amino}piperidin-1-yl)sulfonyl]phenyl}imidazo[1,2-a]pyridine-3-carbonitrile

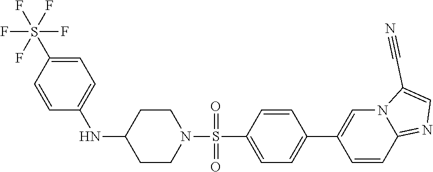 [SMILES: N#Cc1cnc2ccc(-c3ccc(S(=O)(=O)N4CCC(Nc5ccc(S(F)(F)(F)(F)F)cc5)CC4)cc3)cn12]

MS(ES⁺) m/z 584.3 (M+H)⁺

Building block: Step ii: 6-bromoimidazo[1,2-a]pyridine-3-carbonitrile.

### Example 26: 6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide

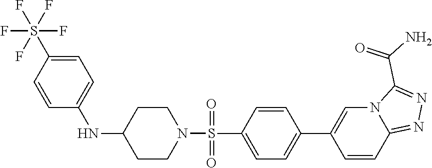 [SMILES: NC(=O)c1nnc2ccc(-c3ccc(S(=O)(=O)N4CCC(Nc5ccc(S(F)(F)(F)(F)F)cc5)CC4)cc3)cn12]

MS(ES⁺) m/z 603.3 (M+H)⁺

Building block: Step ii: 6-bromo-[1,2,4]triazolo[4,3-a]pyridine-3-carboxamide.

### Example 27: 1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine

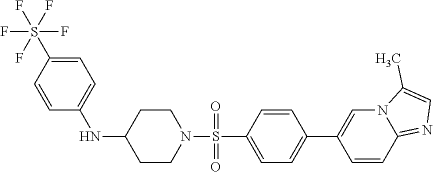 [SMILES: Cc1cnc2ccc(-c3ccc(S(=O)(=O)N4CCC(Nc5ccc(S(F)(F)(F)(F)F)cc5)CC4)cc3)cn12]

MS(ES⁺) m/z 574.3 (M+H)⁺

Building block: Step ii: 6-bromo-3-methyl-[1,2,4]triazolo[4,3-a]pyridine.

### Example 28: N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine

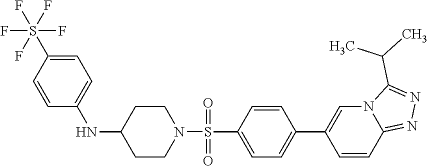 [SMILES: CC(C)c1nnc2ccc(-c3ccc(S(=O)(=O)N4CCC(Nc5ccc(S(F)(F)(F)(F)F)cc5)CC4)cc3)cn12]

MS(ES⁺) m/z 602.3 (M+H)⁺

Building block: Step ii: 6-bromo-3-(propan-2-yl)-[1,2,4]triazolo[4,3-a]pyridine.

### Example 29: N-[4-(pentafluoro-λ⁶-sulfanyl)phenyl]-1-{4-[3-(propan-2-yl)imidazo[1,2-a]pyridin-6-yl]benzenesulfonyl}piperidin-4-amine

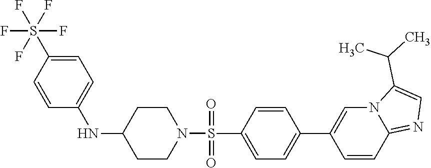 [SMILES: CC(C)c1cnc2ccc(-c3ccc(S(=O)(=O)N4CCC(Nc5ccc(S(F)(F)(F)(F)F)cc5)CC4)cc3)cn12]

MS(ES⁺) m/z 601.4 (M+H)⁺

Building block: Step ii: 6-bromo-3-(propan-2-yl)imidazo[1,2-a]pyridine.

### Example 30: 1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine

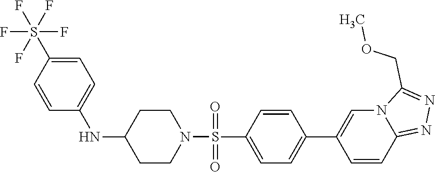 [SMILES: COCc1nnc2ccc(-c3ccc(S(=O)(=O)N4CCC(Nc5ccc(S(F)(F)(F)(F)F)cc5)CC4)cc3)cn12]

MS(ES⁺) m/z 604.4 (M+H)⁺

Building block: Step ii: 6-bromo-3-(methoxymethyl)-[1,2,4]triazolo[4,3-a]pyridine.

### Example 31: 1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine

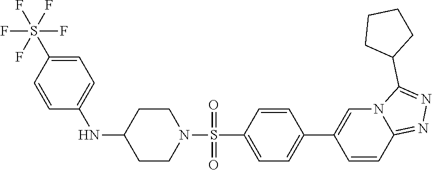 [SMILES: O=S(=O)(c1ccc(-c2ccc3nnc(C4CCCC4)n3c2)cc1)N1CCC(Nc2ccc(S(F)(F)(F)(F)F)cc2)CC1]

MS(ES⁺) m/z 628.4 (M+H)⁺

Building block: Step ii: 6-bromo-3-cyclopentyl-[1,2,4]triazolo[4,3-a]pyridine.

### Example 32: 6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one

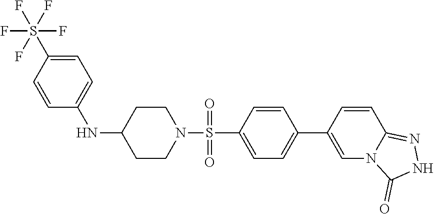 [SMILES: O=c1[nH]nc2ccc(-c3ccc(S(=O)(=O)N4CCC(Nc5ccc(S(F)(F)(F)(F)F)cc5)CC4)cc3)cn12]

i) Following a procedure analogous to that described for Example 24, using in step ii 6-bromo-2-[(4-methoxyphenyl)methyl]-2H,3H-[1,2,4]triazolo[4,3-a]pyridin-3-one (97 mg), 2-[(4-methoxyphenyl)methyl]-6-{4-[(4-{[4-(pentafluoro-4-sulfanyl)phenyl]Amino}piperidin-1-yl)sulfonyl]phenyl}-2H,3H-[1,2,4]triazolo[4,3-a]pyridin-3-one (48 mg) has been prepared as a white solid.
ii) A solution of the product obtained in the previous step (48 mg) and L-cycteine (17 mg) in trifluoroacetic acid (604 uL) was stirred for 4 hours at 70° C. After cooling to room temperature the reaction mixture was concentrated under reduced pressure and the residue was purified on C18, using 10% to 60% acetonitril in water as the eluent, giving the title compound 6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one (6 mg) as a white solid. MS(ES⁺) m/z 576.3 (M+H)⁺.

¹H NMR (400 MHz, DMSO) δ 12.61 (s, 1H), 8.23 (t, J=1.4, 1.4 Hz, 1H), 8.07-8.00 (m, 2H), 7.86-7.78 (m, 2H), 7.66 (dd, J=9.8, 1.8 Hz, 1H), 7.51-7.44 (m, 2H), 7.40 (dd, J=9.8, 1.1 Hz, 1H), 6.60 (d, J=9.0 Hz, 2H), 6.46 (d, J=7.8 Hz, 1H), 3.60 (d, J=11.9 Hz, 2H), 3.42-3.34 (m, 1H), 2.61-2.53 (m, 2H), 2.02-1.93 (m, 2H), 1.51-1.37 (m, 2H).

### Example 33: 6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile

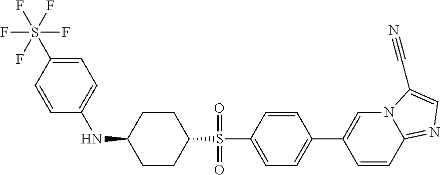 [SMILES: N#Cc1cnc2ccc(-c3ccc(S(=O)(=O)[C@H]4CC[C@H](Nc5ccc(S(F)(F)(F)(F)F)cc5)CC4)cc3)cn12]

i) Following a procedure described for Example 24, step i, N-[4-(4-bromobenzenesulfonyl)cyclohexyl]-4-(pentafluoro-λ⁶-sulfanyl)aniline (Example 5, step v, 500 mg) was converted to 4-(pentafluoro-λ⁶-sulfanyl)-N-{4-[4-(4,4,5,5-tetramethyl-1,3,2-dioxaborolan-2-yl)benzenesulfonyl]cyclohexyl}aniline (916 mg) as a brown oil, which was used in the next step without further purification.
ii) Following a procedure described for Example 1, step ii, the product obtained in the previous step (126 mg) and 6-bromoimidazo[1,2-a]pyridine-3-carbonitrile (60 mg) were converted to the title compound 6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile (10 mg) as a white solid. MS(ES⁺) m/z 583.3 (M+H)⁺.

¹H NMR (400 MHz, DMSO) δ 9.00 (t, J=1.4, 1.4 Hz, 1H), 8.54 (s, 1H), 8.19-8.12 (m, 2H), 8.07-7.96 (m, 4H), 7.52-7.45 (m, 2H), 6.62 (d, J=8.9 Hz, 2H), 6.43 (d, J=7.8 Hz, 1H), 3.45-3.35 (m, 1H), 3.29-3.20 (m, 1H), 2.02 (t, J=15.8, 15.8 Hz, 4H), 1.56 (q, J=12.2, 12.1, 12.1 Hz, 2H), 1.21 (q, J=11.9, 11.6, 11.6 Hz, 2H).

Following a procedure analogous to that described for Example 33, using in step ii the appropriate aryl halide, Example 34-65 have been prepared.

### Example 34-65

### Example 34: 6-(4-{[trans-4-{[4-(pentafluoro-λ⁶-sulfanyl)phenyl]Amino}cyclohexyl]sulfonyl}phenyl)imidazo[1,2-a]pyridine-3-carboxamide

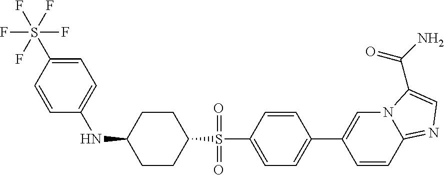 [SMILES: NC(=O)c1cnc2ccc(-c3ccc(S(=O)(=O)[C@H]4CC[C@H](Nc5ccc(S(F)(F)(F)(F)F)cc5)CC4)cc3)cn12]

MS(ES⁺) m/z 601.3 (M+H)⁺

Building block: Step ii: 6-bromoimidazo[1,2-a]pyridine-3-carboxamide.

### Example 35: 4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline

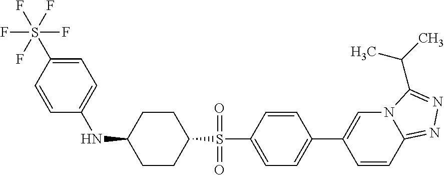 [SMILES: CC(C)c1nnc2ccc(-c3ccc(S(=O)(=O)[C@H]4CC[C@H](Nc5ccc(S(F)(F)(F)(F)F)cc5)CC4)cc3)cn12]

MS(ES⁺) m/z 601.4 (M+H)⁺

Building block: Step ii: 6-bromo-3-(propan-2-yl)-[1,2,4]triazolo[4,3-a]pyridine.

### Example 36: 4-(pentafluoro-λ⁶-sulfanyl)-N-[trans-4-(4-{[1,2,4]triazolo[4,3-a]pyridin-6-yl}benzenesulfonyl)cyclohexyl]aniline

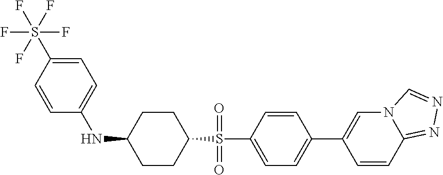 [SMILES: O=S(=O)(c1ccc(-c2ccc3nncn3c2)cc1)[C@H]1CC[C@H](Nc2ccc(S(F)(F)(F)(F)F)cc2)CC1]

MS(ES⁺) m/z 559.3 (M+H)⁺

Building block: Step ii: 6-bromo-[1,2,4]triazolo[4,3-a]pyridine.

### Example 37: 5-(4-{[trans-4-{[4-(pentafluoro-λ⁶-sulfanyl)phenyl]Amino}cyclohexyl]sulfonyl}phenyl)-1H-indazole-3-carboxamide

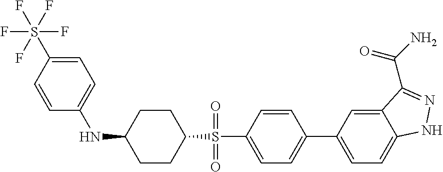 [SMILES: NC(=O)c1n[nH]c2ccc(-c3ccc(S(=O)(=O)[C@H]4CC[C@H](Nc5ccc(S(F)(F)(F)(F)F)cc5)CC4)cc3)cc12]

MS(ES⁺) m/z 601.3 (M+H)⁺

Building block: Step ii: 5-bromo-1H-indazole-3-carboxamide.

### Example 38: 5-(4-{[trans-4-{[4-(pentafluoro-λ⁶-sulfanyl)phenyl]Amino}cyclohexyl]sulfonyl}phenyl)-1H-indazole-3-carbonitrile

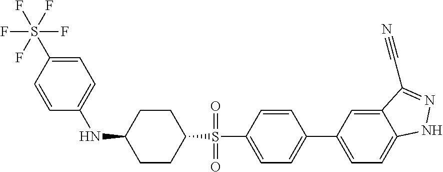 [SMILES: N#Cc1n[nH]c2ccc(-c3ccc(S(=O)(=O)[C@H]4CC[C@H](Nc5ccc(S(F)(F)(F)(F)F)cc5)CC4)cc3)cc12]

MS(ES⁺) m/z 583.3 (M+H)⁺

Building block: Step ii: 5-bromo-1H-indazole-3-carbonitrile.

### Example 39: 6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide

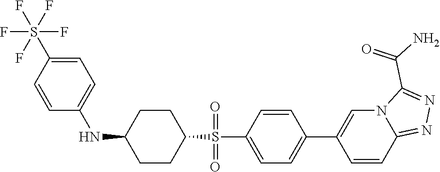 [SMILES: NC(=O)c1nnc2ccc(-c3ccc(S(=O)(=O)[C@H]4CC[C@H](Nc5ccc(S(F)(F)(F)(F)F)cc5)CC4)cc3)cn12]

MS(ES⁺) m/z 602.3 (M+H)⁺

Building block: Step ii: 6-bromo-[1,2,4]triazolo[4,3-a]pyridine-3-carboxamide.

### Example 40: 4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline

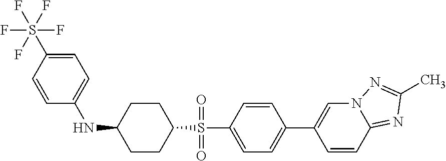 [SMILES: Cc1nc2ccc(-c3ccc(S(=O)(=O)[C@H]4CC[C@H](Nc5ccc(S(F)(F)(F)(F)F)cc5)CC4)cc3)cn2n1]

MS(ES⁺) m/z 573.3 (M+H)⁺

Building block: Step ii: 6-bromo-2-methyl-[1,2,4]triazolo[1,5-a]pyridine.

### Example 41: 4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline

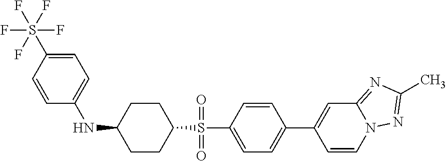 [SMILES: Cc1nc2cc(-c3ccc(S(=O)(=O)[C@H]4CC[C@H](Nc5ccc(S(F)(F)(F)(F)F)cc5)CC4)cc3)ccn2n1]

MS(ES⁺) m/z 573.3 (M+H)⁺

Building block: Step ii: 7-bromo-2-methyl-[1,2,4]triazolo[1,5-a]pyridine.

### Example 42: 6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile

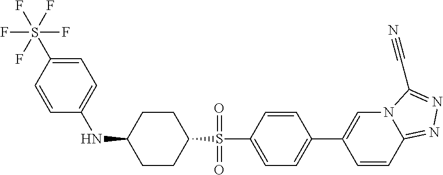 [SMILES: N#Cc1nnc2ccc(-c3ccc(S(=O)(=O)[C@H]4CC[C@H](Nc5ccc(S(F)(F)(F)(F)F)cc5)CC4)cc3)cn12]

MS(ES⁺) m/z 584.3 (M+H)⁺

Building block: Step ii: 6-bromo-[1,2,4]triazolo[4,3-a]pyridine-3-carbonitrile.

### Example 43: 4-(pentafluoro-λ⁶-sulfanyl)-N-[trans-4-(4-{2-methylimidazo[1,2-a]pyrazin-6-yl}benzenesulfonyl)cyclohexyl]aniline

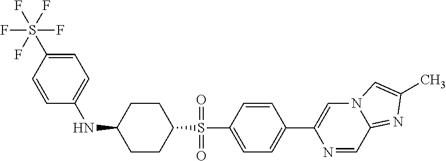 [SMILES: Cc1cn2cc(-c3ccc(S(=O)(=O)[C@H]4CC[C@H](Nc5ccc(S(F)(F)(F)(F)F)cc5)CC4)cc3)ncc2n1]

MS(ES⁺) m/z 573.3 (M+H)⁺

Building block: Step ii: 6-bromo-2-methylimidazo[1,2-a]pyrazine.

### Example 44: 4-(pentafluoro-λ⁶-sulfanyl)-N-[trans-4-(4-{imidazo[1,2-a]pyrazin-6-yl}benzenesulfonyl)cyclohexyl]aniline

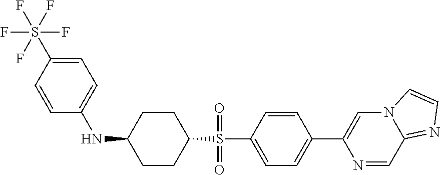 [SMILES: O=S(=O)(c1ccc(-c2cn3ccnc3cn2)cc1)[C@H]1CC[C@H](Nc2ccc(S(F)(F)(F)(F)F)cc2)CC1]

MS(ES⁺) m/z 559.3 (M+H)⁺

Building block: Step ii: 6-bromoimidazo[1,2-a]pyrazine.

### Example 45: N,N-dimethyl-6-(4-{[cis-4-{[4-(pentafluoro-λ⁶-sulfanyl)phenyl]Amino}cyclohexyl]sulfonyl}phenyl)pyridine-3-carboxamide

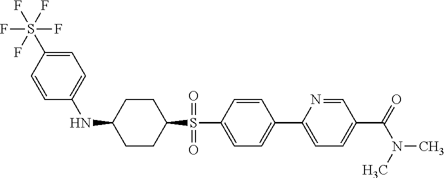 [SMILES: CN(C)C(=O)c1ccc(-c2ccc(S(=O)(=O)[C@H]3CC[C@@H](Nc4ccc(S(F)(F)(F)(F)F)cc4)CC3)cc2)nc1]

MS(ES⁺) m/z 590.3 (M+H)⁺

Building block: Step ii: 6-chloro-N,N-dimethylpyridine-3-carboxamide.

### Example 46: N,N-dimethyl-6-(4-{[trans-4-{[4-(pentafluoro-λ⁶-sulfanyl)phenyl]Amino}cyclohexyl]sulfonyl}phenyl)pyridine-3-carboxamide

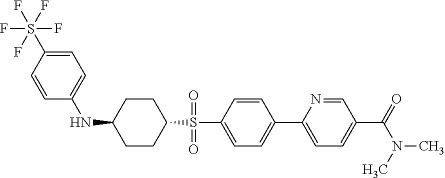 [SMILES: CN(C)C(=O)c1ccc(-c2ccc(S(=O)(=O)[C@H]3CC[C@H](Nc4ccc(S(F)(F)(F)(F)F)cc4)CC3)cc2)nc1]

MS(ES⁺) m/z 590.3 (M+H)⁺

Building block: Step ii: 6-chloro-N,N-dimethylpyridine-3-carboxamide.

### Example 47: N-methyl-6-(4-{[trans-4-{[4-(pentafluoro-λ⁶-sulfanyl)phenyl]Amino}cyclohexyl]sulfonyl}phenyl)pyridine-3-carboxamide

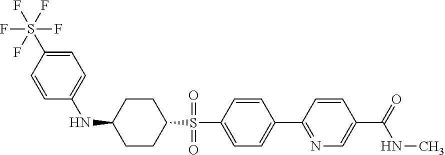 [SMILES: CNC(=O)c1ccc(-c2ccc(S(=O)(=O)[C@H]3CC[C@H](Nc4ccc(S(F)(F)(F)(F)F)cc4)CC3)cc2)nc1]

MS(ES⁺) m/z 576.3 (M+H)⁺

Building block: Step ii: 6-chloro-N-methylpyridine-3-carboxamide.

### Example 48: N-methyl-4-(4-{[trans-4-{[4-(pentafluoro-λ⁶-sulfanyl)phenyl]Amino}cyclohexyl]sulfonyl}phenyl)pyridine-2-carboxamide

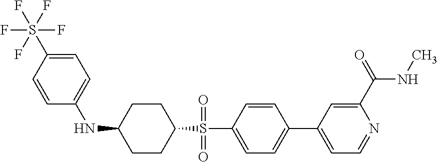 [SMILES: CNC(=O)c1cc(-c2ccc(S(=O)(=O)[C@H]3CC[C@H](Nc4ccc(S(F)(F)(F)(F)F)cc4)CC3)cc2)ccn1]

MS(ES⁺) m/z 576.3 (M+H)⁺

Building block: Step ii: 4-chloro-N-methylpyridine-2-carboxamide.

### Example 49: 6-(4-{[trans-4-{[4-(pentafluoro-λ⁶-sulfanyl)phenyl]Amino}cyclohexyl]sulfonyl}phenyl)pyridine-3-carboxamide

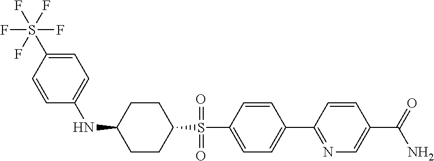 [SMILES: NC(=O)c1ccc(-c2ccc(S(=O)(=O)[C@H]3CC[C@H](Nc4ccc(S(F)(F)(F)(F)F)cc4)CC3)cc2)nc1]

Building block: Step ii: 6-chloropyridine-3-carboxamide.

### Example 50: 4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline

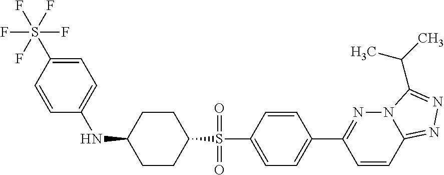 [SMILES: CC(C)c1nnc2ccc(-c3ccc(S(=O)(=O)[C@H]4CC[C@H](Nc5ccc(S(F)(F)(F)(F)F)cc5)CC4)cc3)nn12]

MS(ES⁺) m/z 602.3 (M+H)⁺

Building block: Step ii: 6-chloro-3-(propan-2-yl)-[1,2,4]triazolo[4,3-b]pyridazine.

### Example 51: 4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline

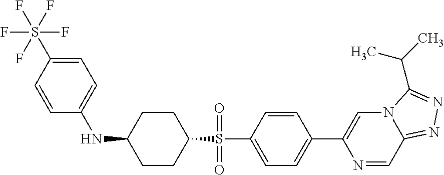 [SMILES: CC(C)c1nnc2cnc(-c3ccc(S(=O)(=O)[C@H]4CC[C@H](Nc5ccc(S(F)(F)(F)(F)F)cc5)CC4)cc3)cn12]

MS(ES⁺) m/z 602.4 (M+H)⁺

Building block: Step ii: 6-bromo-3-(propan-2-yl)-[1,2,4]triazolo[4,3-a]pyrazine.

### Example 52: 4-(4-{[trans-4-{[4-(pentafluoro-λ⁶-sulfanyl)phenyl]Amino}cyclohexyl]sulfonyl}phenyl)-1,2-dihydropyridin-2-one

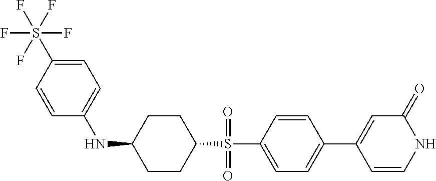 [SMILES: O=c1cc(-c2ccc(S(=O)(=O)[C@H]3CC[C@H](Nc4ccc(S(F)(F)(F)(F)F)cc4)CC3)cc2)cc[nH]1]

MS(ES⁺) m/z 533.2 (M−H)⁺

Building block: Step ii: 4-bromo-1,2-dihydropyridin-2-one.

### Example 53: 7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one

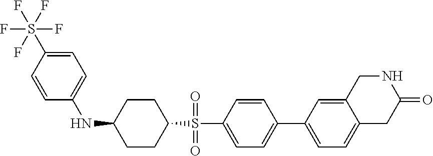 [SMILES: O=C1Cc2ccc(-c3ccc(S(=O)(=O)[C@H]4CC[C@H](Nc5ccc(S(F)(F)(F)(F)F)cc5)CC4)cc3)cc2CN1]

MS(ES⁺) m/z 587.4 (M+H)⁺

Building block: Step ii: 7-bromo-1,2,3,4-tetrahydroisoquinolin-3-one.

### Example 54: 6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one

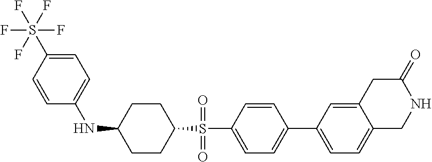 [SMILES: O=C1Cc2cc(-c3ccc(S(=O)(=O)[C@H]4CC[C@H](Nc5ccc(S(F)(F)(F)(F)F)cc5)CC4)cc3)ccc2CN1]

MS(ES⁺) m/z 587.4 (M+H)⁺

Building block: Step ii: 6-bromo-1,2,3,4-tetrahydroisoquinolin-3-one.

### Example 55: 6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one

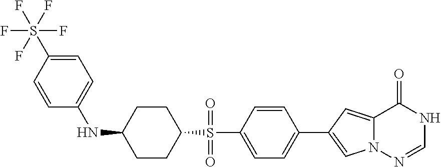 [SMILES: O=c1[nH]cnn2cc(-c3ccc(S(=O)(=O)[C@H]4CC[C@H](Nc5ccc(S(F)(F)(F)(F)F)cc5)CC4)cc3)cc12]

MS(ES⁺) m/z 575.3 (M+H)⁺

Building block: Step ii: 6-bromo-3H,4H-pyrrolo[2,1-f][1,2,4]triazin-4-one.

### Example 56: 5-(4-{[trans-4-{[4-(pentafluoro-λ⁶-sulfanyl)phenyl]Amino}cyclohexyl]sulfonyl}phenyl)pyridin-2-ol

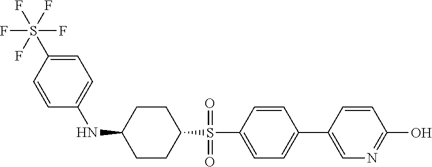 [SMILES: O=S(=O)(c1ccc(-c2ccc(O)nc2)cc1)[C@H]1CC[C@H](Nc2ccc(S(F)(F)(F)(F)F)cc2)CC1]

MS(ES⁺) m/z 535.2 (M+H)⁺

Building block: Step ii: 5-bromopyridin-2-ol.

### Example 57: 4-(pentafluoro-λ⁶-sulfanyl)-N-[trans-4-{[4′-(dimethylphosphoryl)-[1,1′-biphenyl]-4-yl]sulfonyl}cyclohexyl]aniline

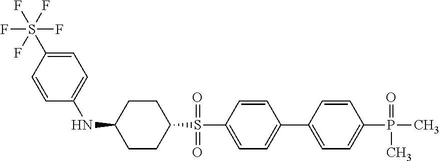 [SMILES: CP(C)(=O)c1ccc(-c2ccc(S(=O)(=O)[C@H]3CC[C@H](Nc4ccc(S(F)(F)(F)(F)F)cc4)CC3)cc2)cc1]

MS(ES⁺) m/z 594.3 (M+H)⁺

Building block: Step ii: 1-bromo-4-(dimethylphosphoryl)benzene.

### Example 58: 5-(4-{[trans-4-{[4-(pentafluoro-λ⁶-sulfanyl)phenyl]Amino}cyclohexyl]sulfonyl}phenyl)-2,3-dihydro-1H-isoindol-1-one

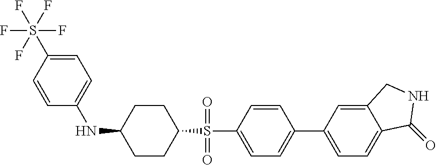 [SMILES: O=C1NCc2cc(-c3ccc(S(=O)(=O)[C@H]4CC[C@H](Nc5ccc(S(F)(F)(F)(F)F)cc5)CC4)cc3)ccc21]

MS(ES⁺) m/z 573.3 (M+H)⁺

Building block: Step ii: 5-bromo-2,3-dihydro-1H-isoindol-1-one.

### Example 59: 6-(4-{[trans-4-{[4-(pentafluoro-λ⁶-sulfanyl)phenyl]Amino}cyclohexyl]sulfonyl}phenyl)-2,3-dihydro-1H-indol-2-one

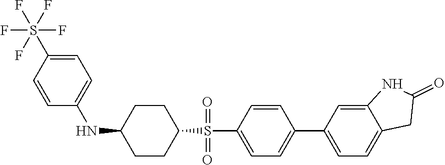 [SMILES: O=C1Cc2ccc(-c3ccc(S(=O)(=O)[C@H]4CC[C@H](Nc5ccc(S(F)(F)(F)(F)F)cc5)CC4)cc3)cc2N1]

MS(ES⁺) m/z 573.3 (M+H)⁺

Building block: Step ii: 6-bromo-2,3-dihydro-1H-indol-2-one.

### Example 60: 7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one

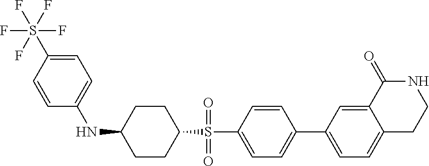 [SMILES: O=C1NCCc2ccc(-c3ccc(S(=O)(=O)[C@H]4CC[C@H](Nc5ccc(S(F)(F)(F)(F)F)cc5)CC4)cc3)cc21]

MS(ES⁺) m/z 585.3 (M−H)⁺

Building block: Step ii: 7-bromo-1,2,3,4-tetrahydroisoquinolin-1-one.

### Example 61: 6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one

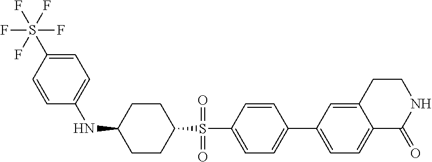 [SMILES: O=C1NCCc2cc(-c3ccc(S(=O)(=O)[C@H]4CC[C@H](Nc5ccc(S(F)(F)(F)(F)F)cc5)CC4)cc3)ccc21]

MS(ES⁺) m/z 587.4 (M+H)⁺

Building block: Step ii: 6-bromo-1,2,3,4-tetrahydroisoquinolin-1-one.

### Example 62: 6-(4-{[trans-4-{[4-(pentafluoro-λ⁶-sulfanyl)phenyl]Amino}cyclohexyl]sulfonyl}phenyl)pyridazine-3-carboxamide

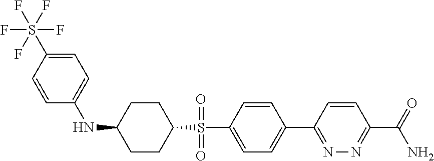 [SMILES: NC(=O)c1ccc(-c2ccc(S(=O)(=O)[C@H]3CC[C@H](Nc4ccc(S(F)(F)(F)(F)F)cc4)CC3)cc2)nn1]

MS(ES⁺) m/z 563.3 (M+H)⁺

Building block: Step ii: 6-chloropyridazine-3-carboxamide.

### Example 63: 4-(pentafluoro-λ⁶-sulfanyl)-N-[trans-4-{4-[3-(propan-2-yl)imidazo[1,2-a]pyridin-6-yl]benzenesulfonyl}cyclohexyl]aniline

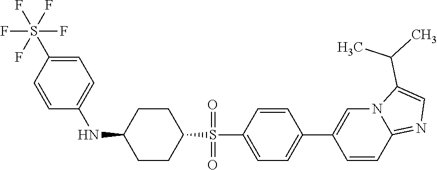 [SMILES: CC(C)c1cnc2ccc(-c3ccc(S(=O)(=O)[C@H]4CC[C@H](Nc5ccc(S(F)(F)(F)(F)F)cc5)CC4)cc3)cn12]

MS(ES⁺) m/z 563.3 (M+H)⁺

Building block: Step ii: 6-bromo-3-(propan-2-yl)imidazo[1,2-a]pyridine.

### Example 64: 4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline

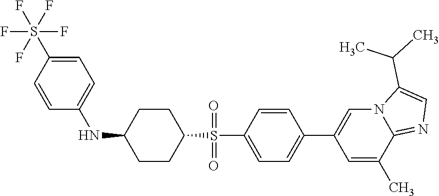 [SMILES: Cc1cc(-c2ccc(S(=O)(=O)[C@H]3CC[C@H](Nc4ccc(S(F)(F)(F)(F)F)cc4)CC3)cc2)cn2c(C(C)C)cnc12]

MS(ES⁺) m/z 614.4 (M+H)⁺

Building block: Step ii: 6-bromo-8-methyl-3-(propan-2-yl)imidazo[1,2-a]pyridine.

### Example 65: 5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one

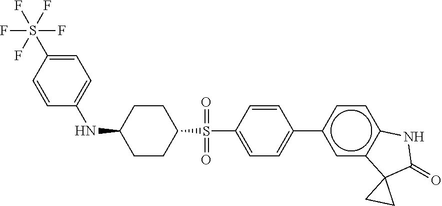

MS(ES⁺) m/z 599.3 (M+H)⁺

Building block: step vi: 5′-bromo-1′,2′-dihydrospiro[cyclopropane-1,3′-indol]-2′-one.

### Example 66: 6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one

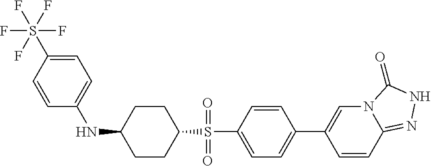 [SMILES: O=c1[nH]nc2ccc(-c3ccc(S(=O)(=O)[C@H]4CC[C@H](Nc5ccc(S(F)(F)(F)(F)F)cc5)CC4)cc3)cn12]

i) Following a procedure analogous to that described for Example 32, using in step ii 6-bromo-2-[(4-methoxyphenyl)methyl]-2H,3H-[1,2,4]triazolo[4,3-a]pyridin-3-one (447 mg), 2-[(4-methoxyphenyl)methyl]-6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one (400 mg) has been prepared as a white solid.
ii) A solution of the product obtained in the previous step (400 mg) and L-cycteine (104 mg) in trifluoroacetic acid (3.7 mL) was stirred for 6 hours at 70° C. After cooling to room temperature the reaction mixture was concentrated under reduced pressure and the residue was purified on C18, using 30% to 50% acetonitril in water as the eluent, giving the title compound 6-(4-{[trans-4-{[4-pentafluoro-λ⁶-sulfanyl)phenyl]Amino}cyclohexyl]sulfonyl}phenyl)-2H,3H-[1,2,4]triazolo[4,3-a]pyridin-3-one (170 mg) as a white solid. MS(ES⁺) m/z 575.3 (M+H)⁺.

¹H NMR (400 MHz, DMSO) δ 12.62 (s, 1H), 8.24 (t, J=1.5, 1.5 Hz, 1H), 8.10-8.00 (m, 2H), 7.95-7.87 (m, 2H), 7.66 (dd, J=9.9, 1.8 Hz, 1H), 7.52-7.43 (m, 2H), 7.39 (dd, J=9.8, 1.1 Hz, 1H), 6.62 (d, J=8.9 Hz, 2H), 6.42 (d, J=7.8 Hz, 1H), 3.40-3.31 (m, 1H), 3.30-3.19 (m, 1H), 2.10-1.95 (m, 4H), 1.61-1.47 (m, 2H), 1.20 (q, J=12.0, 11.4, 11.4 Hz, 2H).

### Example 67: 1-(2-fluoro-4-{3-methylimidazo[1,2-a]pyridin-6-yl}benzenesulfonyl)-N-[4-(pentafluoro-λ⁶-sulfanyl)phenyl]piperidin-4-amine

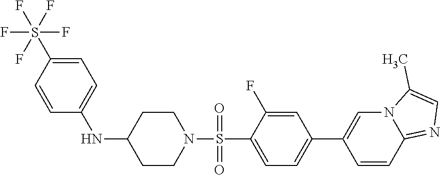 [SMILES: Cc1cnc2ccc(-c3ccc(S(=O)(=O)N4CCC(Nc5ccc(S(F)(F)(F)(F)F)cc5)CC4)c(F)c3)cn12]

i) Following a procedure analogous to that described for Example 1, Step i, tert-butyl 4-oxopiperidine-1-carboxylate (6 g) and 4-(pentafluoro-λ⁶-sulfanyl)aniline (7.9) were converted to tert-butyl 4-{[4-(pentafluoro-λ⁶-sulfanyl)phenyl]Amino}piperidine-1-carboxylate (15 g), obtained as a white solid, which was used in the next step without further purification.
ii) To a solution of the product obtained in the previous step (15 g) in CH₂Cl₂ (50 mL) was added a 5N HCl solution in 2-propanol (75 mL). After stirring overnight at room temperature the reaction mixture was concentrated under vacuo to give N-[4-(pentafluoro-λ⁶-sulfanyl)phenyl]piperidin-4-amine hydrochloride (14.2 g) as a white solid which was used in the next step without any further purification.
iii) To a solution of the product obtained in the previous step (1 g) and Et₃N (2.1 mL) in CH₂Cl₂ (20 mL) was added 4-bromo-2-fluorobenzene-1-sulfonyl chloride (0.89 g). The reaction mixture was stirred overnight at room temperature. The reaction mixture was quenched by pouring it into water. The product was extracted into CH₂Cl₂ and the combined organic layers were dried over MgSO₄, filtered and concentrated under vacuo to give 1-(4-bromo-2-fluorobenzenesulfonyl)-N-[4-pentafluoro-λ⁶-sulfanyl)phenyl]piperidin-4-amine (1.6 g) as an off white solid, which was used in the next step without any further purification.
iv) Following a procedure described for Example 1, step ii, the product obtained in the previous step (100 mg) and 3-methyl-6-(4,4,5,5-tetramethyl-1,3,2-dioxaborolan-2-yl)imidazo[1,2-a]pyridine (57 mg) were converted to the title compound 1-Q-fluoro-4-{3-methylimidazo[1,2-a]pyridin-6-yl}benzenesulfonyl)-N-[4-(pentafluoro-λ⁶-sulfanyl)phenyl]piperidin-4-amine (57 mg) as a white solid. MS(ES⁺) m/z 591.3 (M+H)⁺.

¹H NMR (400 MHz, DMSO) δ 8.75 (t, J=1.4, 1.4 Hz, 1H), 8.07 (dd, J=12.1, 1.6 Hz, 1H), 7.95-7.84 (m, 2H), 7.68 (d, J=1.3 Hz, 2H), 7.52-7.47 (m, 2H), 7.45 (t, J=0.9, 0.9 Hz, 1H), 6.62 (d, J=8.9 Hz, 2H), 6.50 (d, J=7.9 Hz, 1H), 3.67 (d, J=12.3 Hz, 2H), 3.51-3.41 (m, 1H), 2.83 (t, J=11.4, 11.4 Hz, 2H), 2.57 (d, J=1.0 Hz, 3H), 2.03-1.95 (m, 2H), 1.52-1.38 (m, 2H).

Following a procedure analogous to that described for Example 67, using in step iii the appropriate sulfonyl chloride and in step iv the appropriate boronic ester or boronic acid, compounds 68-80 have been prepared.

### Examples 68-80

### Example 68: 1-(3-fluoro-4-{3-methylimidazo[1,2-a]pyridin-6-yl}benzenesulfonyl)-N-[4-(pentafluoro-λ⁶-sulfanyl)phenyl]piperidin-4-amine

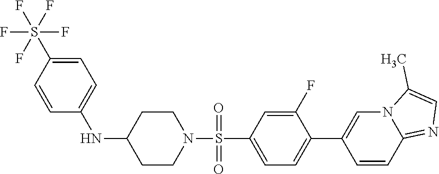 [SMILES: Cc1cnc2ccc(-c3ccc(S(=O)(=O)N4CCC(Nc5ccc(S(F)(F)(F)(F)F)cc5)CC4)cc3F)cn12]

MS(ES⁺) m/z 591.3 (M+H)⁺

Building blocks: Step iii: 4-bromo-3-fluorobenzene-1-sulfonyl chloride; step iv: 3-methyl-6-(4,4,5,5-tetramethyl-1,3,2-dioxaborolan-2-yl)imidazo[1,2-a]pyridine.

### Example 69: 1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine

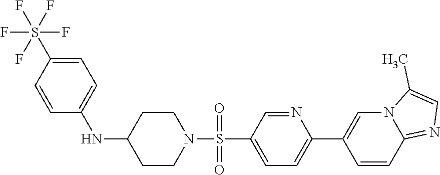 [SMILES: Cc1cnc2ccc(-c3ccc(S(=O)(=O)N4CCC(Nc5ccc(S(F)(F)(F)(F)F)cc5)CC4)cn3)cn12]

MS(ES⁺) m/z 574.3 (M+H)⁺

Building blocks: Step iii: 6-bromopyridine-3-sulfonyl chloride; step iv: 3-methyl-6-(4,4,5,5-tetramethyl-1,3,2-dioxaborolan-2-yl)imidazo[1,2-a]pyridine.

### Example 70: 1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine

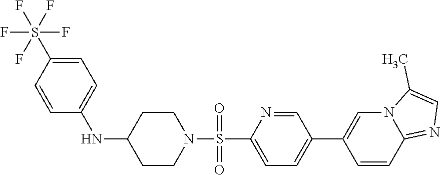 [SMILES: Cc1cnc2ccc(-c3ccc(S(=O)(=O)N4CCC(Nc5ccc(S(F)(F)(F)(F)F)cc5)CC4)nc3)cn12]

MS(ES⁺) m/z 574.3 (M+H)⁺

Building blocks: Step iii: 5-bromopyridine-2-sulfonyl chloride; step iv: 3-methyl-6-(4,4,5,5-tetramethyl-1,3,2-dioxaborolan-2-yl)imidazo[1,2-a]pyridine.

### Example 71: 6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile

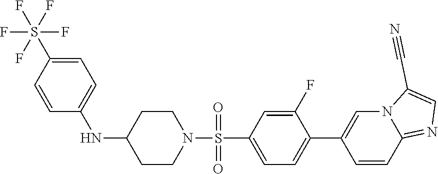 [SMILES: N#Cc1cnc2ccc(-c3ccc(S(=O)(=O)N4CCC(Nc5ccc(S(F)(F)(F)(F)F)cc5)CC4)cc3F)cn12]

MS(ES⁺) m/z 602.3 (M+H)⁺

Building blocks: Step iii: 4-bromo-3-fluorobenzene-1-sulfonyl chloride; step iv: 6-(4,4,5,5-tetramethyl-1,3,2-dioxaborolan-2-yl)imidazo[1,2-a]pyridine-3-carbonitrile.

### Example 72: 1-(3-fluoro-4-{imidazo[1,2-a]pyridin-6-yl}benzenesulfonyl)-N-[4-(pentafluoro-λ⁶-sulfanyl)phenyl]piperidin-4-amine

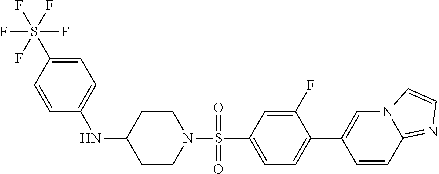 [SMILES: O=S(=O)(c1ccc(-c2ccc3nccn3c2)c(F)c1)N1CCC(Nc2ccc(S(F)(F)(F)(F)F)cc2)CC1]

MS(ES⁺) m/z 577.3 (M+H)⁺

Building blocks: Step iii: 4-bromo-3-fluorobenzene-1-sulfonyl chloride; step iv: 6-(4,4,5,5-tetramethyl-1,3,2-dioxaborolan-2-yl)imidazo[1,2-a]pyridine.

### Example 73: 6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile

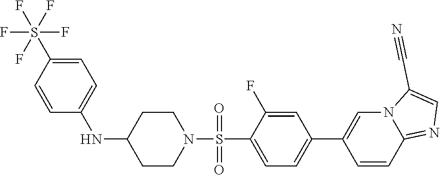 [SMILES: N#Cc1cnc2ccc(-c3ccc(S(=O)(=O)N4CCC(Nc5ccc(S(F)(F)(F)(F)F)cc5)CC4)c(F)c3)cn12]

MS(ES⁺) m/z 602 (M+H)⁺

Building blocks: Step iii: 4-bromo-2-fluorobenzene-1-sulfonyl chloride; step iv: 6-(4,4,5,5-tetramethyl-1,3,2-dioxaborolan-2-yl)imidazo[1,2-a]pyridine-3-carbonitrile.

### Example 74: 1-(2-fluoro-4-{imidazo[1,2-a]pyridin-6-yl}benzenesulfonyl)-N-[4-(pentafluoro-λ⁶-sulfanyl)phenyl]piperidin-4-amine

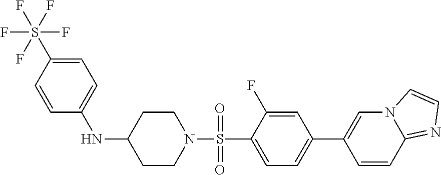 [SMILES: O=S(=O)(c1ccc(-c2ccc3nccn3c2)cc1F)N1CCC(Nc2ccc(S(F)(F)(F)(F)F)cc2)CC1]

MS(ES⁺) m/z 577.3 (M+H)⁺

Building blocks: Step iii: 4-bromo-2-fluorobenzene-1-sulfonyl chloride; step iv: 6-(4,4,5,5-tetramethyl-1,3,2-dioxaborolan-2-yl)imidazo[1,2-a]pyridine.

### Example 75: 1-[(5-{imidazo[1,2-a]pyridin-6-yl}pyridin-2-yl)sulfonyl]-N-[4-(pentafluoro-λ⁶-sulfanyl)phenyl]piperidin-4-amine

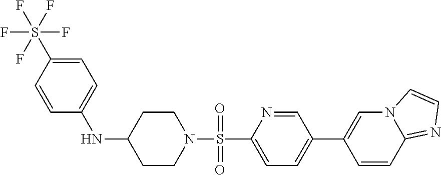 [SMILES: O=S(=O)(c1ccc(-c2ccc3nccn3c2)cn1)N1CCC(Nc2ccc(S(F)(F)(F)(F)F)cc2)CC1]

MS(ES⁺) m/z 560.3 (M+H)⁺

Building blocks: Step iii: 5-bromopyridine-2-sulfonyl chloride; step iv: 6-(4,4,5,5-tetramethyl-1,3,2-dioxaborolan-2-yl)imidazo[1,2-a]pyridine.

### Example 76: 6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile

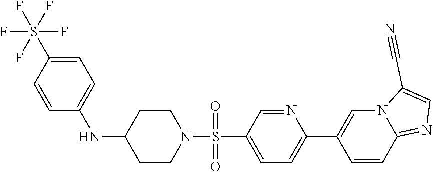 [SMILES: N#Cc1cnc2ccc(-c3ccc(S(=O)(=O)N4CCC(Nc5ccc(S(F)(F)(F)(F)F)cc5)CC4)cn3)cn12]

MS(ES⁺) m/z 585.3 (M+H)⁺

Building blocks: Step iii: 6-bromopyridine-3-sulfonyl chloride; step iv: 6-(4,4,5,5-tetramethyl-1,3,2-dioxaborolan-2-yl)imidazo[1,2-a]pyridine-3-carbonitrile.

### Example 77: 1-[(6-{imidazo[1,2-a]pyridin-6-yl}pyridin-3-yl)sulfonyl]-N-[4-(pentafluoro-λ⁶-sulfanyl)phenyl]piperidin-4-amine

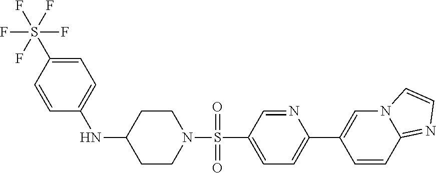 [SMILES: O=S(=O)(c1ccc(-c2ccc3nccn3c2)nc1)N1CCC(Nc2ccc(S(F)(F)(F)(F)F)cc2)CC1]

MS(ES⁺) m/z 560.3 (M+H)⁺

Building blocks: Step iii: 6-bromopyridine-3-sulfonyl chloride; step iv: 6-(4,4,5,5-tetramethyl-1,3,2-dioxaborolan-2-yl)imidazo[1,2-a]pyridine.

### Example 78: 6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile

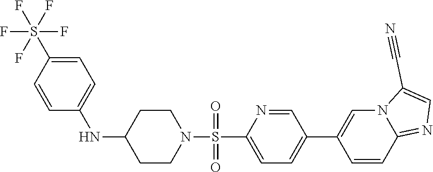 [SMILES: N#Cc1cnc2ccc(-c3ccc(S(=O)(=O)N4CCC(Nc5ccc(S(F)(F)(F)(F)F)cc5)CC4)nc3)cn12]

MS(ES⁺) m/z 585.3 (M+H)⁺

Building blocks: Step iii: 5-bromopyridine-2-sulfonyl chloride; step iv: 6-(4,4,5,5-tetramethyl-1,3,2-dioxaborolan-2-yl)imidazo[1,2-a]pyridine-3-carbonitrile.

### Example 79: 1-(2-chloro-4-{imidazo[1,2-a]pyridin-6-yl}benzenesulfonyl)-N-[4-(pentafluoro-λ⁶-sulfanyl)phenyl]piperidin-4-amine

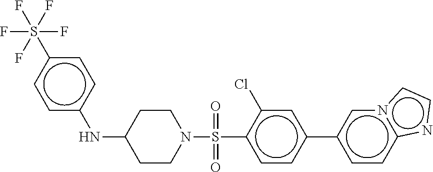

MS(ES⁺) m/z 593.3 (M+H)⁺

Building blocks: Step iii: 4-bromo-2-chlorobenzene-1-sulfonyl chloride; step iv: 6-(4,4,5,5-tetramethyl-1,3,2-dioxaborolan-2-yl)imidazo[1,2-a]pyridine.

### Example 80: 6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile

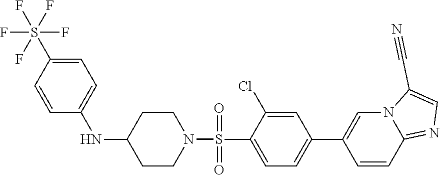 [SMILES: N#Cc1cnc2ccc(-c3ccc(S(=O)(=O)N4CCC(Nc5ccc(S(F)(F)(F)(F)F)cc5)CC4)c(Cl)c3)cn12]

MS(ES⁺) m/z 618.3 (M+H)⁺

Building blocks: Step iii: 4-bromo-2-chlorobenzene-1-sulfonyl chloride; step iv: 6-(4,4,5,5-tetramethyl-1,3,2-dioxaborolan-2-yl)imidazo[1,2-a]pyridine-3-carbonitrile.

Following a procedure analogous to that described for Example 67, using in step i the appropriate SF₅-aniline and tert-butyl 4-oxopiperidine-1-carboxylate, in step iii the appropriate sulfonyl chloride and in step iv the appropriate boronic ester or boronic acid, compounds 81-83 have been prepared.

### Examples 81-83

### Example 81: 6-{4-[(4-{[3-(pentafluoro-λ⁶-sulfanyl)phenyl]Amino}piperidin-1-yl)sulfonyl]phenyl}imidazo[1,2-a]pyridine-3-carbonitrile

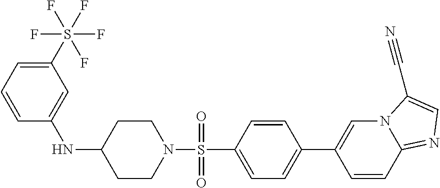 [SMILES: N#Cc1cnc2ccc(-c3ccc(S(=O)(=O)N4CCC(Nc5cccc(S(F)(F)(F)(F)F)c5)CC4)cc3)cn12]

MS(ES⁺) m/z 584.3 (M+H)⁺

Building blocks: Step i: 3-(pentafluoro-λ⁶-sulfanyl)aniline; Step iii: 4-bromobenzene-1-sulfonyl chloride; step iv: 6-(4,4,5,5-tetramethyl-1,3,2-dioxaborolan-2-yl)imidazo[1,2-a]pyridine-3-carbonitrile.

### Example 82: 1-(4-{imidazo[1,2-a]pyridin-6-yl}benzenesulfonyl)-N-[3-(pentafluoro-λ⁶-sulfanyl)phenyl]piperidin-4-amine

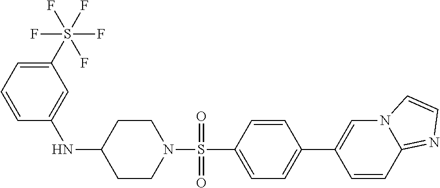 [SMILES: O=S(=O)(c1ccc(-c2ccc3nccn3c2)cc1)N1CCC(Nc2cccc(S(F)(F)(F)(F)F)c2)CC1]

MS(ES⁺) m/z 584.3 (M+H)⁺

Building blocks: Step i: 3-(pentafluoro-λ⁶-sulfanyl)aniline; Step iii: 4-bromobenzene-1-sulfonyl chloride; step iv: 6-(4,4,5,5-tetramethyl-1,3,2-dioxaborolan-2-yl)imidazo[1,2-a]pyridine.

### Example 83: 4-(4-{[(3S,4S)-3-fluoro-4-{[4-(pentafluoro-λ⁶-sulfanyl)phenyl]Amino}piperidin-1-yl]sulfonyl}phenyl)pyridine-2-carboxamide

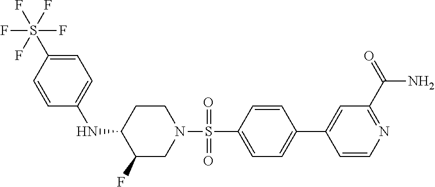 [SMILES: NC(=O)c1cc(-c2ccc(S(=O)(=O)N3CC[C@@H](Nc4ccc(S(F)(F)(F)(F)F)cc4)[C@H](F)C3)cc2)ccn1]

MS(ES⁺) m/z 579.3 (M+H)⁺

Building blocks: Step i: 4-(pentafluoro-λ⁶-sulfanyl)aniline and tert-butyl 3-fluoro-4-oxopiperidine-1-carboxylate; Step iii: 4-bromobenzene-1-sulfonyl chloride; step iv: 4-(4,4,5,5-tetramethyl-1,3,2-dioxaborolan-2-yl)pyridine-2-carboxamide.

### Example 84: N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine

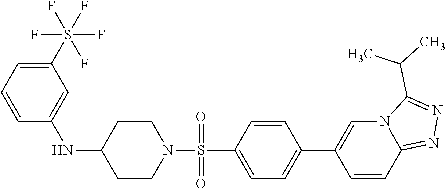 [SMILES: CC(C)c1nnc2ccc(-c3ccc(S(=O)(=O)N4CCC(Nc5cccc(S(F)(F)(F)(F)F)c5)CC4)cc3)cn12]

MS(ES⁺) m/z 584.3 (M+H)⁺
i) Following a procedure analogous to that described for Example 67, step i to step iii, using in step i the appropriate SF₅-aniline, and in step iii the appropriate sulfonyl chloride, 1-(4-bromobenzenesulfonyl)-N-[3-(pentafluoro-λ⁶-sulfanyl)phenyl]piperidin-4-amine was synthesized.
ii) To a suspension of the product obtained in the previous step (200 mg), bis(pinacolato)diboron (107 mg) and potassium acetate (94 mg) in cyclopentyl methyl ether (1.5 mL), purged with N₂ gas, was added PdCl₂(dppf)·CH₂Cl₂ (19 mg). The reaction mixture was heated for 2 hours at 100° C. in a microwave. After cooling to room temperature, the reaction mixture was poured into water and the product was extracted into ethyl acetate. The organic layer was washed with brine, dried over MgSO₄ and concentrated under reduced pressure giving N-[3-(pentafluoro-λ⁶-sulfanyl)phenyl]-1-[4-(4,4,5,5-tetramethyl-1,3,2-dioxaborolan-2-yl)benzenesulfonyl]piperidin-4-amine as a brown oil (290 mg), which was used in the next step without further purification.
iii) Following a procedure described for Example 1, step ii, the product obtained in the previous step (290 mg) and 6-bromo-3-(propan-2-yl)-[1,2,4]triazolo[4,3-a]pyridine (135 mg) were converted to the title compound N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine (159 mg) as a white solid. MS(ES⁺) m/z 602.3 (M+H)⁺.

¹H NMR (400 MHz, DMSO) δ 8.84 (d, J=1.7 Hz, 1H), 8.14-8.06 (m, 2H), 7.92-7.84 (m, 3H), 7.77 (dd, J=9.6, 1.7 Hz, 1H), 7.23 (t, J=8.2, 8.2 Hz, 1H), 7.00 (t, J=2.3, 2.3 Hz, 1H), 6.91 (dd, J=8.0, 2.2 Hz, 1H), 6.78 (dd, J=8.2, 2.2 Hz, 1H), 6.15 (d, J=8.1 Hz, 1H), 3.74 (p, J=6.9, 6.9, 6.8, 6.8 Hz, 1H), 3.60 (dd, J=10.1, 6.2 Hz, 2H), 3.40 (s, 1H), 2.64-2.52 (m, 2H), 1.97 (dd, J=13.4, 3.7 Hz, 2H), 1.43 (d, J=6.9 Hz, 8H).

### Example 85: 5-(4-{[trans-4-{[4-(pentafluoro-λ⁶-sulfanyl)phenyl]Amino}cyclohexyl]sulfonimidoyl}phenyl)-1H-indole-3-carbonitrile

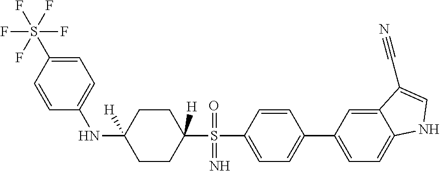 [SMILES: N#Cc1c[nH]c2ccc(-c3ccc(S(=N)(=O)[C@H]4CC[C@H](Nc5ccc(S(F)(F)(F)(F)F)cc5)CC4)cc3)cc12]

i) To a 0° C. cold solution of 4-hydroxycyclohexanone monoethylene ketal (25 g) and Et₃N (29 mL) in CH₂Cl₂ (55 mL) was added dropwise a solution of methanesulfonyl chloride (16 mL) in CH₂Cl₂ (20 mL). The reaction mixture was stirred overnight at room temperature. After completion the reaction mixture was poured into a saturated aqueous NaHCO₃ solution, and the product was extracted into CH₂Cl₂. The combined organic layers were washed with brine, dried over MgSO₄, filtered and concentrated under reduced pressure to give 1,4-dioxaspiro[4.5]decan-8-yl methanesulfonate as a yellow oil (40 g), which was used in the next step without further purification.
ii) To a suspension of the product obtained in the previous step (37.3 g) and Cs₂CO₃ (128.7 g) in acetone (400 mL) was added at room temperature 4-bromothiophenol (44.8 g). The reaction mixture was stirred overnight at 60° C. After the reaction mixture was concentrated under reduced pressure, water and ethyl acetate were added. The product was extracted into ethyl acetate and the combined organic layers were washed with water, brine, dried over MgSO₄, filtered, and concentrated under reduced pressure. The obtained brown oil (59 g) was purified on SiO₂, using 0% to 100% EtOAc in heptane as the eluent, giving 8-[(4-bromophenyl)sulfanyl]-1,4-dioxaspiro[4.5]decane as a clear oil (49 g), which slowly solidified.
iii) To a solution of the product obtained in the previous step (13.2 g) in THF (200 mL) was added an aqueous 2N HCl solution (160 mL) and the reaction mixture was stirred overnight at room temperature. After completion, the organic solvent was removed under reduced pressure and the remaining aqueous layer was extracted with EtOAc. The combined organic layers were washed with water, brine, dried over MgSO₄, filtered and concentrated under reduced pressure to give 4-[(4-bromophenyl)sulfanyl]cyclohexan-1-one as an yellowish oil (11 g), which was used in the next step without further purification.
iv) To a solution of the product obtained in the previous step (3 g), 4-(pentafluoro-λ⁶-sulfanyl)aniline (2.3 mL) and HOAc (1 mL) in methanol (20 mL) was added at room temperature 2-methylpyridine borane complex (1.46 g). The reaction mixture was stirred overnight at room temperature. After completion the reaction mixture was concentrated under reduced pressure and after cooling to 0° C. an aqueous solution of 2N HCl (5 mL) was added. After stirring for 1 hour at 0° C., an aqueous solution of 5N NaOH was added. The product was extracted into EtOAc and the combined organic layers were washed with water, brine, dried over MgSO₄, filtered and concentrated under reduced pressure to give N-{4-[(4-bromophenyl)sulfanyl]cyclohexyl}-4-(pentafluoro-λ⁶-sulfanyl)aniline as a white solid (3.2 g), which was used in the next step without further purification.
v) To a suspension of the product obtained in the previous step (1.0 g mg) and ammonium carbamate (240 mg) in a mixture of methanol (20 mL) and acetonitrile (20 mL) was added at room temperature (diacetoxyiodo)benzene (1.39 g). The reaction mixture was stirred at room temperature for 30 minutes in a flask open to the atmosphere. After completion, the reaction mixture was concentrated under reduced pressure. At this stage, the cis/trans mixture could be separated on C18, using 10% to 100% acetonitrile in water, to give (4-bromophenyl)trans-4-{[4-(pentafluoro-λ⁶-sulfanyl)phenyl]Amino}cyclohexyl](imino)-λ⁶-sulfanone (0.47 g) and (4-bromophenyl)[cis-4-{[4-(pentafluoro-λ⁶-sulfanyl)phenyl]Amino}cyclohexyl](imino)-λ⁶-sulfanone (0.58 g).
vi) Under a nitrogen atmosphere, Pd(PPh₃)₄ (11 mg) was added to a suspension of (4-bromophenyl)[trans-4-{[4-(pentafluoro-λ⁶-sulfanyl)phenyl]Amino}cyclohexyl](imino)-λ⁶-sulfanone (100 mg), 5-(4,4,5,5-tetramethyl-1,3,2-dioxaborolan-2-yl)-1H-indole-3-carbonitrile (62 mg) and NaHCO₃ (97 mg) in a mixture of 1,4-dioxane (4 mL) and water (1 mL). The reaction mixture was stirred for 1 h at 110° C. in a microwave. After cooling to room temperature, the product was extracted into EtOAc and the organic layer was dried over MgSO₄, filtered and concentrated under reduced pressure. The obtained brown solid was purified on C18, using 10% to 90% acetonitrile in water as the eluent, giving the title compound 5-(4-{[trans-4-{[4-(pentafluoro-λ⁶-sulfanyl)phenyl]Amino}cyclohexyl]sulfonimidoyl}phenyl)-1H-indole-3-carbonitrile as a white solid (27 mg). MS(ES⁺) m/z 581.3 (M+H)⁺

¹H NMR (400 MHz, DMSO) δ 12.29 (s, 1H), 8.34 (s, 1H), 8.04-7.97 (m, 3H), 7.96-7.90 (m, 2H), 7.69 (d, J=1.3 Hz, 2H), 7.52-7.43 (m, 2H), 6.60 (d, J=8.9 Hz, 2H), 6.40 (d, J 20=7.8 Hz, 1H), 4.22 (s, 1H), 3.25-3.15 (m, H), 3.14-3.04 (m, 1H), 2.12-1.99 (m, 4H), 1.59-1.42 (m, 2H), 1.19 (q, J=12.4, 12.4, 12.4 Hz, 2H).

Following a procedure analogous to that described for Example 85, using the appropriate boronic ester or boronic acid in step vi, Examples 86-101 have been prepared.

### Examples 86-101

### Example 86: [4-(3-methyl-1H-indazol-5-yl)phenyl][trans-4-{[4-(pentafluoro-λ⁶-sulfanyl)phenyl]Amino}cyclohexyl](imino)-λ⁶-sulfanone

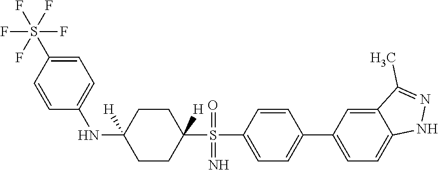 [SMILES: Cc1n[nH]c2ccc(-c3ccc(S(=N)(=O)[C@H]4CC[C@H](Nc5ccc(S(F)(F)(F)(F)F)cc5)CC4)cc3)cc12]

MS(ES⁺) m/z 571.3 (M+H)⁺

Building block: step vi: (3-methyl-1H-indazol-5-yl)boronic acid.

### Example 87: (4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone

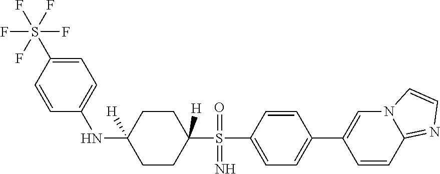 [SMILES: N=S(=O)(c1ccc(-c2ccc3nccn3c2)cc1)[C@H]1CC[C@H](Nc2ccc(S(F)(F)(F)(F)F)cc2)CC1]

MS(ES⁺) m/z 557.3 (M+H)⁺

Building block: step vi: 6-(4,4,5,5-tetramethyl-1,3,2-dioxaborolan-2-yl)imidazo[1,2-a]pyridine.

### Example 88: (4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone

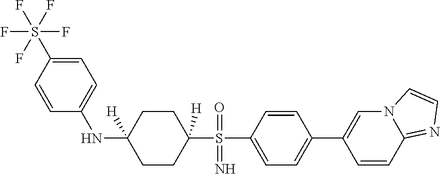 [SMILES: N=S(=O)(c1ccc(-c2ccc3nccn3c2)cc1)[C@H]1CC[C@@H](Nc2ccc(S(F)(F)(F)(F)F)cc2)CC1]

MS(ES⁺) m/z 557.3 (M+H)⁺

Building block: step vi: 6-(4,4,5,5-tetramethyl-1,3,2-dioxaborolan-2-yl)imidazo[1,2-a]pyridine.

### Example 89: (4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone

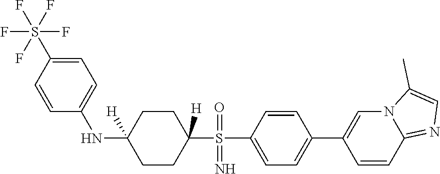 [SMILES: Cc1cnc2ccc(-c3ccc(S(=N)(=O)[C@H]4CC[C@H](Nc5ccc(S(F)(F)(F)(F)F)cc5)CC4)cc3)cn12]

MS(ES⁺) m/z 571.3 (M+H)⁺

Building block: step vi: 3-methyl-6-(4,4,5,5-tetramethyl-1,3,2-dioxaborolan-2-yl)imidazo[1,2-a]pyridine.

### Example 90: 6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile

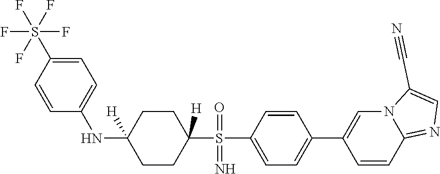 [SMILES: N#Cc1cnc2ccc(-c3ccc(S(=N)(=O)[C@H]4CC[C@H](Nc5ccc(S(F)(F)(F)(F)F)cc5)CC4)cc3)cn12]

MS(ES⁺) m/z 582.3 (M+H)⁺

Building block: step vi: 6-(4,4,5,5-tetramethyl-1,3,2-dioxaborolan-2-yl)imidazo[1,2-a]pyridine-3-carbonitrile.

### Example 91: 4-(4-{[trans-4-{[4-(pentafluoro-λ⁶-sulfanyl)phenyl]Amino}cyclohexyl]sulfonimidoyl}phenyl)pyridine-2-carboxamide

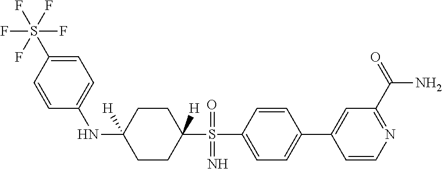 [SMILES: N=S(=O)(c1ccc(-c2ccnc(C(N)=O)c2)cc1)[C@H]1CC[C@H](Nc2ccc(S(F)(F)(F)(F)F)cc2)CC1]

MS(ES⁺) m/z 561.3 (M+H)⁺

Building block: step vi: 4-(4,4,5,5-tetramethyl-1,3,2-dioxaborolan-2-yl)pyridine-2-carboxamide.

### Example 92: N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide

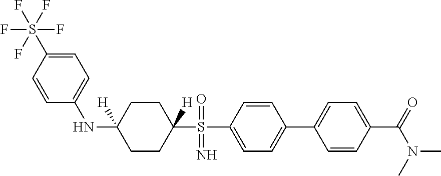 [SMILES: CN(C)C(=O)c1ccc(-c2ccc(S(=N)(=O)[C@H]3CC[C@H](Nc4ccc(S(F)(F)(F)(F)F)cc4)CC3)cc2)cc1]

MS(ES⁺) m/z 588.3 (M+H)⁺

Building block: N,N-dimethyl-4-(4,4,5,5-tetramethyl-1,3,2-dioxaborolan-2-yl)benzamide.

### Example 93: (4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone

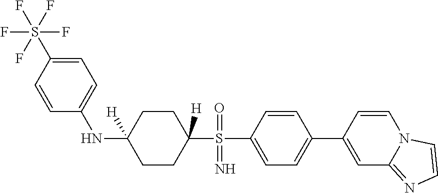 [SMILES: N=S(=O)(c1ccc(-c2ccn3ccnc3c2)cc1)[C@H]1CC[C@H](Nc2ccc(S(F)(F)(F)(F)F)cc2)CC1]

MS(ES⁺) m/z 557.3 (M+H)⁺

Building block: step vi: 7-(4,4,5,5-tetramethyl-1,3,2-dioxaborolan-2-yl)imidazo[1,2-a]pyridine.

### Example 94: [trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone

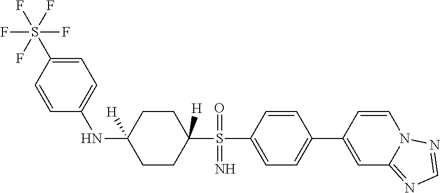 [SMILES: N=S(=O)(c1ccc(-c2ccn3ncnc3c2)cc1)[C@H]1CC[C@H](Nc2ccc(S(F)(F)(F)(F)F)cc2)CC1]

MS(ES⁺) m/z 558.3 (M+H)⁺

Building block: step vi: 7-(4,4,5,5-tetramethyl-1,3,2-dioxaborolan-2-yl)-[1,2,4]triazolo[1,5-a]pyridine.

### Example 95: N-methyl-4′-{[trans-4-{[4-(pentafluoro-λ⁶-sulfanyl)phenyl]Amino}cyclohexyl]sulfonimidoyl}-[1,1′-biphenyl]-4-carboxamide

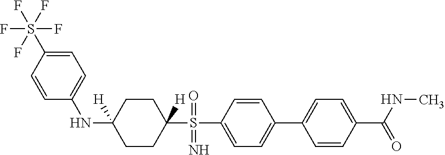 [SMILES: CNC(=O)c1ccc(-c2ccc(S(=N)(=O)[C@H]3CC[C@H](Nc4ccc(S(F)(F)(F)(F)F)cc4)CC3)cc2)cc1]

MS(ES⁺) m/z 574.3 (M+H)⁺

Building block: step vi: [4-(methylcarbamoyl)phenyl]boronic acid.

### Example 96: 4′-{[trans-4-{[4-(pentafluoro-λ⁶-sulfanyl)phenyl]Amino}cyclohexyl]sulfonimidoyl}-[1,1′-biphenyl]-4-carboxamide

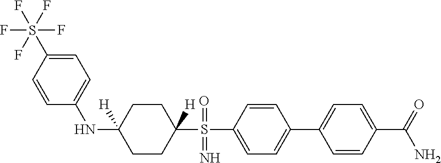 [SMILES: N=S(=O)(c1ccc(-c2ccc(C(N)=O)cc2)cc1)[C@H]1CC[C@H](Nc2ccc(S(F)(F)(F)(F)F)cc2)CC1]

MS(ES⁺) m/z 560.3 (M+H)⁺

Building block: step vi: (4-carbamoylphenyl)boronic acid.

### Example 97: N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide

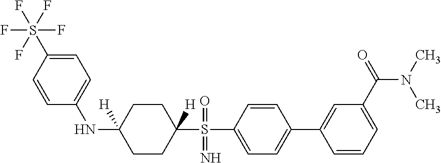 [SMILES: CN(C)C(=O)c1cccc(-c2ccc(S(=N)(=O)[C@H]3CC[C@H](Nc4ccc(S(F)(F)(F)(F)F)cc4)CC3)cc2)c1]

MS(ES⁺) m/z 588.3 (M+H)⁺

Building block: step vi: [3-(dimethylcarbamoyl)phenyl]boronic acid.

### Example 98: N-methyl-4′-{[trans-4-{[4-(pentafluoro-λ⁶-sulfanyl)phenyl]Amino}cyclohexyl]sulfonimidoyl}-[1,1′-biphenyl]-3-carboxamide

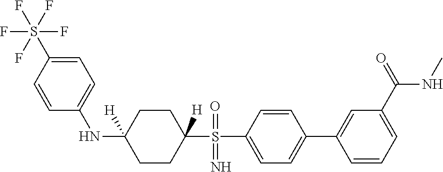 [SMILES: CNC(=O)c1cccc(-c2ccc(S(=N)(=O)[C@H]3CC[C@H](Nc4ccc(S(F)(F)(F)(F)F)cc4)CC3)cc2)c1]

MS(ES⁺) m/z 574.3 (M+H)⁺

Building block: step vi: [3-(methylcarbamoyl)phenyl]boronic acid.

### Example 99: 4′-{[trans-4-{[4-(pentafluoro-λ⁶-sulfanyl)phenyl]Amino}cyclohexyl]sulfonimidoyl}-[1,1′-biphenyl]-3-carboxamide

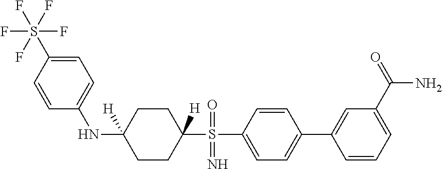 [SMILES: N=S(=O)(c1ccc(-c2cccc(C(N)=O)c2)cc1)[C@H]1CC[C@H](Nc2ccc(S(F)(F)(F)(F)F)cc2)CC1]

MS(ES⁺) m/z 560.3 (M+H)⁺

Building block: step vi: (3-carbamoylphenyl)boronic acid.

### Example 100: N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide

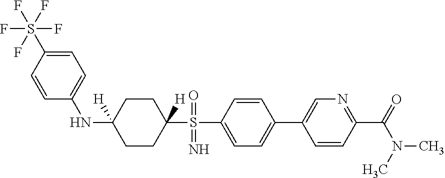 [SMILES: CN(C)C(=O)c1ccc(-c2ccc(S(=N)(=O)[C@H]3CC[C@H](Nc4ccc(S(F)(F)(F)(F)F)cc4)CC3)cc2)cn1]

MS(ES⁺) m/z 589.3 (M+H)⁺

Building block: N,N-dimethyl-5-(4,4,5,5-tetramethyl-1,3,2-dioxaborolan-2-yl)pyridine-2-carboxamide.

### Example 101: N-methyl-5-(4-{[trans-4-{[4-(pentafluoro-λ⁶-sulfanyl)phenyl]Amino}cyclohexyl]sulfonimidoyl}phenyl)pyridine-2-carboxamide

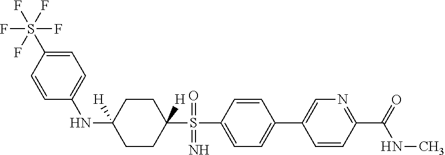 [SMILES: CNC(=O)c1ccc(-c2ccc(S(=N)(=O)[C@H]3CC[C@H](Nc4ccc(S(F)(F)(F)(F)F)cc4)CC3)cc2)cn1]

MS(ES⁺) m/z 575.3 (M+H)⁺

Building block: step vi: N-methyl-5-(4,4,5,5-tetramethyl-1,3,2-dioxaborolan-2-yl)pyridine-2-carboxamide.

### Example 102: {4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone

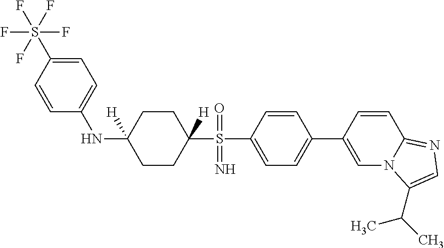 [SMILES: CC(C)c1cnc2ccc(-c3ccc(S(=N)(=O)[C@H]4CC[C@H](Nc5ccc(S(F)(F)(F)(F)F)cc5)CC4)cc3)cn12]

i) Following a procedure analogous to that described for Example 24, step i, (4-bromophenyl)[trans-4-{[4-(pentafluoro-λ⁶-sulfanyl)phenyl]Amino}cyclohexyl](imino)-λ⁶-sulfanone (Example 85, step v, 411 mg) was converted to [trans-4-{[4-(pentafluoro-λ⁶-sulfanyl)phenyl]Amino}cyclohexyl][4-(4,4,5,5-tetramethyl-1,3,2-dioxaborolan-2-yl)phenyl]imino-λ⁶-sulfanone (800 mg) as a brown oil, which was used in the next step without further purification.
ii) Following a procedure analogous to that described for Example 1, step ii, the product obtained in the previous step (125 mg) and 6-bromo-3-(propan-2-yl)-[1,2,4]triazolo[4,3-a]pyridine (64 mg) were converted to the title compound {4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone (15 mg) as a white solid. MS(ES⁺) m/z 600.4 (M+H)⁺.

¹H NMR (400 MHz, DMSO) δ 8.84 (t, J=1.4, 1.4 Hz, 1H), 8.10-8.02 (m, 2H), 8.01-7.94 (m, 2H), 7.87 (dd, J=9.6, 1.1 Hz, 1H), 7.77 (dd, J=9.6, 1.6 Hz, 1H), 7.52-7.43 (m, 2H), 6.60 (d, J=8.9 Hz, 2H), 6.40 (d, J=7.8 Hz, 1H), 4.30 (s, 1H), 3.74 (hept, J=6.7, 6.7, 6.5, 6.5, 6.5, 6.5 Hz, 1H), 3.26-3.15 (m, 1H), 3.15-3.07 (m, 1H), 2.03 (q, J=14.8, 14.1, 14.1 Hz, 4H), 1.52 (t, J=14.9, 14.9 Hz, 2H), 1.43 (d, J=6.8 Hz, 6H), 1.20 (t, J=13.2, 13.2 Hz, 2H).

Following a procedure analogous to that described for Example 102, using the appropriate aryl halide, Examples 103-118 have been prepared.

### Examples 103-118

### Example 103: (4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone

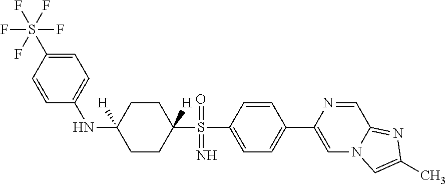 [SMILES: Cc1cn2cc(-c3ccc(S(=N)(=O)[C@H]4CC[C@H](Nc5ccc(S(F)(F)(F)(F)F)cc5)CC4)cc3)ncc2n1]

MS(ES⁺) m/z 572.3 (M+H)⁺

Building block: step ii: 6-bromo-2-methylimidazo[1,2-a]pyrazine.

### Example 104: (4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone

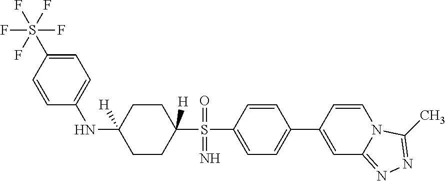 [SMILES: Cc1nnc2cc(-c3ccc(S(=N)(=O)[C@H]4CC[C@H](Nc5ccc(S(F)(F)(F)(F)F)cc5)CC4)cc3)ccn12]

MS(ES⁺) m/z 572.3 (M+H)⁺

Building block: step ii: 7-bromo-3-methyl-[1,2,4]triazolo[4,3-a]pyridine.

### Example 105: (4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone

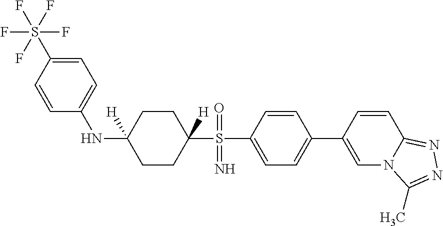 [SMILES: Cc1nnc2ccc(-c3ccc(S(=N)(=O)[C@H]4CC[C@H](Nc5ccc(S(F)(F)(F)(F)F)cc5)CC4)cc3)cn12]

MS(ES⁺) m/z 572.4 (M+H)⁺

Building block: step vi: 6-bromo-3-methyl-[1,2,4]triazolo[4,3-a]pyridine.

### Example 106: N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide

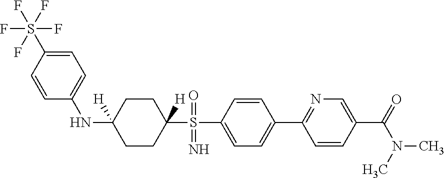 [SMILES: CN(C)C(=O)c1ccc(-c2ccc(S(=N)(=O)[C@H]3CC[C@H](Nc4ccc(S(F)(F)(F)(F)F)cc4)CC3)cc2)nc1]

MS(ES⁺) m/z 589.4 (M+H)⁺

Building block: step vi: 6-chloro-N,N-dimethylpyridine-3-carboxamide.

### Example 107: N-methyl-6-(4-{[trans-4-{[4-(pentafluoro-λ⁶-sulfanyl)phenyl]Amino}cyclohexyl]sulfonimidoyl}phenyl)pyridine-3-carboxamide

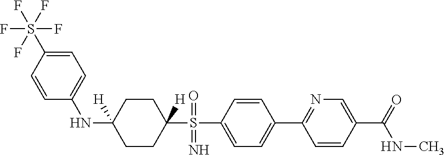 [SMILES: CNC(=O)c1ccc(-c2ccc(S(=N)(=O)[C@H]3CC[C@H](Nc4ccc(S(F)(F)(F)(F)F)cc4)CC3)cc2)nc1]

MS(ES⁺) m/z 575.3 (M+H)⁺

Building block: step vi: 6-chloro-N-methylpyridine-3-carboxamide.

### Example 108: 5-(4-{[trans-4-{[4-(pentafluoro-λ⁶-sulfanyl)phenyl]Amino}cyclohexyl]sulfonimidoyl}phenyl)pyridine-2-carboxamide

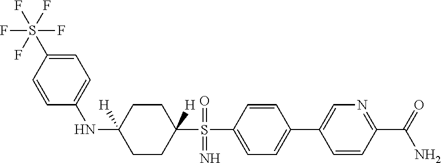 [SMILES: N=S(=O)(c1ccc(-c2ccc(C(N)=O)nc2)cc1)[C@H]1CC[C@H](Nc2ccc(S(F)(F)(F)(F)F)cc2)CC1]

MS(ES⁺) m/z 561.3 (M+H)⁺

Building block: step vi: 5-bromopyridine-2-carboxamide.

### Example 109: N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide

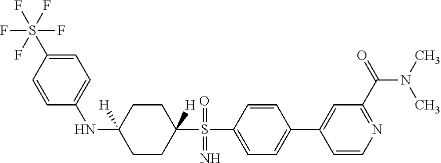 [SMILES: CN(C)C(=O)c1cc(-c2ccc(S(=N)(=O)[C@H]3CC[C@H](Nc4ccc(S(F)(F)(F)(F)F)cc4)CC3)cc2)ccn1]

MS(ES⁺) m/z 589.3 (M+H)⁺

Building block: step vi: 4-chloro-N,N-dimethylpyridine-2-carboxamide.

### Example 110: N-methyl-4-(4-{[trans-4-{[4-(pentafluoro-λ⁶-sulfanyl)phenyl]Amino}cyclohexyl]sulfonimidoyl}phenyl)pyridine-2-carboxamide

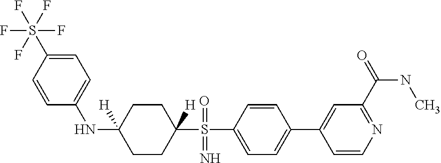 [SMILES: CNC(=O)c1cc(-c2ccc(S(=N)(=O)[C@H]3CC[C@H](Nc4ccc(S(F)(F)(F)(F)F)cc4)CC3)cc2)ccn1]

MS(ES⁺) m/z 575.3 (M+H)⁺

Building block: step vi: 4-chloro-N-methylpyridine-2-carboxamide.

### Example 111: 6-(4-{[trans-4-{[4-(pentafluoro-λ⁶-sulfanyl)phenyl]Amino}cyclohexyl]sulfonimidoyl}phenyl)pyridine-3-carboxamide

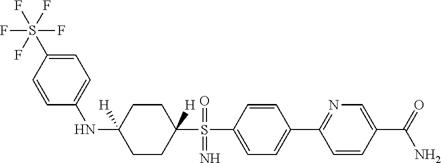 [SMILES: N=S(=O)(c1ccc(-c2ccc(C(N)=O)cn2)cc1)[C@H]1CC[C@H](Nc2ccc(S(F)(F)(F)(F)F)cc2)CC1]

MS(ES⁺) m/z 561.4 (M+H)⁺

Building block: step vi: 6-chloropyridine-3-carboxamide.

### Example 112: 5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one

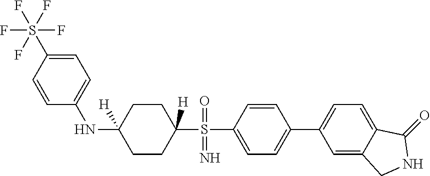 [SMILES: N=S(=O)(c1ccc(-c2ccc3c(c2)CNC3=O)cc1)[C@H]1CC[C@H](Nc2ccc(S(F)(F)(F)(F)F)cc2)CC1]

MS(ES⁺) m/z 572.3 (M+H)⁺

Building block: step vi: 5-bromo-2,3-dihydro-1H-isoindol-1-one.

### Example 113: 6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one

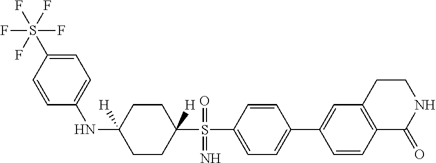 [SMILES: N=S(=O)(c1ccc(-c2ccc3c(c2)CCNC3=O)cc1)[C@H]1CC[C@H](Nc2ccc(S(F)(F)(F)(F)F)cc2)CC1]

MS(ES⁺) m/z 586.3 (M+H)⁺

Building block: step vi: 6-bromo-1,2,3,4-tetrahydroisoquinolin-1-one.

### Example 114: 7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one

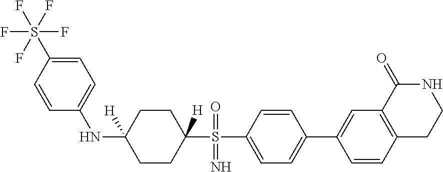 [SMILES: N=S(=O)(c1ccc(-c2ccc3c(c2)C(=O)NCC3)cc1)[C@H]1CC[C@H](Nc2ccc(S(F)(F)(F)(F)F)cc2)CC1]

MS(ES⁺) m/z 586.3 (M+H)⁺

Building block: step vi: 7-bromo-1,2,3,4-tetrahydroisoquinolin-1-one.

### Example 115: 6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one

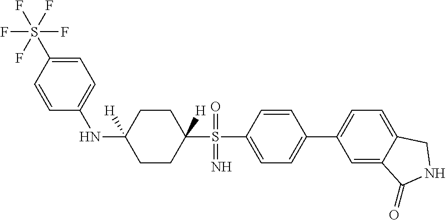 [SMILES: N=S(=O)(c1ccc(-c2ccc3c(c2)C(=O)NC3)cc1)[C@H]1CC[C@H](Nc2ccc(S(F)(F)(F)(F)F)cc2)CC1]

MS(ES⁺) m/z 572.3 (M+H)⁺

Building block: step vi: 6-bromo-2,3-dihydro-1H-isoindol-1-one.

### Example 116: 5-(4-{[trans-4-{[4-(pentafluoro-λ⁶-sulfanyl)phenyl]Amino}cyclohexyl]sulfonimidoyl}phenyl)-2,3-dihydro-1H-indol-2-one

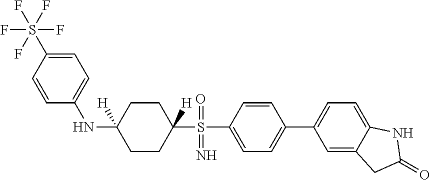 [SMILES: N=S(=O)(c1ccc(-c2ccc3c(c2)CC(=O)N3)cc1)[C@H]1CC[C@H](Nc2ccc(S(F)(F)(F)(F)F)cc2)CC1]

MS(ES⁺) m/z 572.3 (M+H)⁺

Building block: step vi: 5-bromo-2,3-dihydro-1H-indol-2-one.

### Example 117: 6-(4-{[trans-4-{[4-(pentafluoro-λ⁶-sulfanyl)phenyl]Amino}cyclohexyl]sulfonimidoyl}phenyl)-2,3-dihydro-1H-indol-2-one

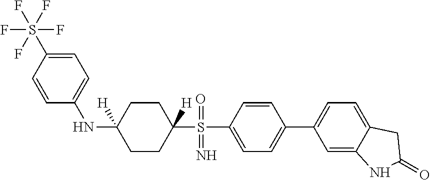 [SMILES: N=S(=O)(c1ccc(-c2ccc3c(c2)NC(=O)C3)cc1)[C@H]1CC[C@H](Nc2ccc(S(F)(F)(F)(F)F)cc2)CC1]

MS(ES⁺) m/z 572.3 (M+H)⁺

Building block: step vi: 6-bromo-2,3-dihydro-1H-isoindol-1-one.

### Example 118: {4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone

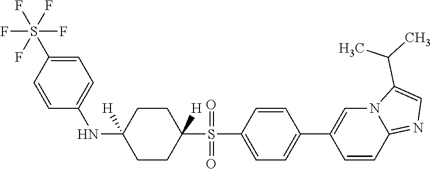 [SMILES: CC(C)c1cnc2ccc(-c3ccc(S(=O)(=O)[C@H]4CC[C@H](Nc5ccc(S(F)(F)(F)(F)F)cc5)CC4)cc3)cn12]

MS(ES⁺) m/z 599.3 (M+H)⁺

Building block: step vi: 6-bromo-3-(propan-2-yl)imidazo[1,2-a]pyridine.

### Example 119: 6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one

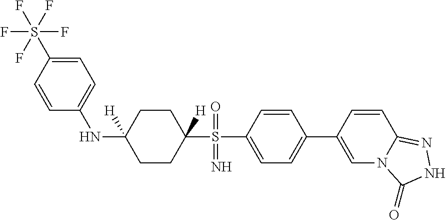 [SMILES: N=S(=O)(c1ccc(-c2ccc3n[nH]c(=O)n3c2)cc1)[C@H]1CC[C@H](Nc2ccc(S(F)(F)(F)(F)F)cc2)CC1]

i) Following a procedure analogous to that described for Example 67, using in step i, [trans-4-{[4-(pentafluoro-λ⁶-sulfanyl)phenyl]Amino}cyclohexyl][4-(4,4,5,5-tetramethyl-1,3,2-dioxaborolan-2-yl)phenyl](imino)-λ⁶-sulfanone (Example 102, step i, 300 mg) the title compound 6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one (75 mg) has been prepared as a white solid. MS(ES⁺) m/z 574 (M+H)⁺

¹H NMR (400 MHz, DMSO) δ 12.61 (s, 1H), 8.23 (t, J=1.4, 1.4 Hz, 1H), 8.07-8.00 (m, 2H), 7.86-7.78 (m, 2H), 7.66 (dd, J=9.8, 1.8 Hz, 1H), 7.51-7.44 (m, 2H), 7.40 (dd, J=9.8, 1.1 Hz, 1H), 6.60 (d, J=9.0 Hz, 2H), 6.46 (d, J=7.8 Hz, 1H), 3.60 (d, J=11.9 Hz, 2H), 3.42-3.34 (m, 1H), 2.61-2.53 (m, 2H), 2.02-1.93 (m, 2H), 1.51-1.37 (m, 2H).

### Example 120: 5-{4-[(4-{[4-(pentafluoro-λ⁶-sulfanyl)phenyl]Amino}piperidin-1-yl)sulfonimidoyl]phenyl}-1H-indole-3-carbonitrile

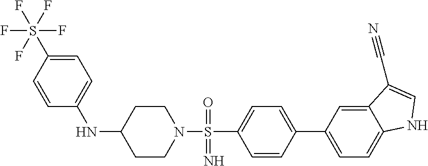 [SMILES: N#Cc1c[nH]c2ccc(-c3ccc(S(=N)(=O)N4CCC(Nc5ccc(S(F)(F)(F)(F)F)cc5)CC4)cc3)cc12]

i) A suspension of N-[4-(pentafluoro-λ⁶-sulfanyl)phenyl]piperidin-4-amine hydrochloride, Example 67, step ii (5 g) in a mixture of an aqueous 2N NaOH (400 mL) and ethyl acetate (100 mL) was stirred for 1 hour at room temperature. The product was extracted into ethyl acetate and the combined organic layers were washed with water, brine, dried over MgSO₄, filtered and concentrated under reduced pressure to give N-[4-(pentafluoro-λ⁶-sulfanyl)phenyl]piperidin-4-amine (4.2 g) as a white solid.
ii) The product obtained in the previous step (3.55 g) and 4-bromobenzene-1-thiol (2.0 g) were added, at room temperature, to a mixture of Cu(I)Br (118 mg) and 2,2′-bipyridine (83 mg) in DMSO (10 mL). the reaction mixture was stirred at 60° C. After 20 hours more Cu(I)Br (118 mg) and 2,2′-bipyridine (83 mg) was added and the reaction mixture was stirred for another 20 hours at 60° C. After cooling to room temperature, the reaction mixture poured in a water, and the product was extracted into ethyl acetate. The combined organic layers were washed with brine, dried over Mg SO₄, filtered and concentrated under reduced pressure. The obtained brown oil (6.4 g) was purified on SiO₂, using 0% to 25% EtOAc in heptane as the eluent, giving 1-[(4-bromophenyl)sulfanyl]-N-[4-(pentafluoro-λ⁶-sulfanyl)phenyl]piperidin-4-amine (3.9 g) as an off white solid.
iii) Following a procedure analogous to that described for Example 85, step v, the product obtained in the previous step (1.8 g) was converted to (4-bromophenyl)(4-{[4-(pentafluoro-λ⁶-sulfanyl)phenyl]Amino}piperidin-1-yl)(imino)-λ⁶-sulfanone (1.18 g) as a light brown oil.
iv) To a solution of the product obtained in the previous step (1.0 g) in THF (10 mL) was added NaH (152 mg) at 0° C. The reaction mixture was stirred until room temperature had been reached before di-tert-butyl dicarbonate (830 mg) was added. The reaction mixture was stirred at room temperature and after 20 hours one more portion of NaH (152 mg) was added. The reaction was stirred for another 20 hours at room temperature. After completion, the reaction mixture was quenched by adding a saturated aqueous NH₄Cl solution. The THF was removed under reduced pressure and the product was extracted into ethyl acetate. The combined organic layers were washed with water, brine, dried over MgSO₄, filtered and concentrated under reduced pressure. The obtained yellow oil (4.2 g) was purified on SiO₂, using 0% to 5% CH₃OH in CH₂Cl₂ as the eluent, giving tert-butyl N-[(4-bromophenyl)(oxo)(4-{[4-(pentafluoro-λ⁶-sulfanyl)phenyl]Amino}piperidin-1-yl)-λ⁶-sulfanylidene]carbamate (1.08 g) as an off white gum.
v) Following a procedure analogous to that described for Example 85, step vi, the product obtained in the previous step (205 mg) and 5-(4,4,5,5-tetramethyl-1,3,2-dioxaborolan-2-yl)-1H-indole-3-carbonitrile (86 mg) were converted to tert-butyl N-{[4-(3-cyano-1H-indol 5-yl)phenyl](oxo)(4-{[4-(pentafluoro-λ⁶-sulfanyl)phenyl]Amino}piperidin-1-yl)-λ⁶-sulfanylidene}carbamate (175 mg) as a yellow solid.
vi) To a solution of the product obtained in the previous step (175 mg) in ethyl acetate (10 mL) was added a 5N HCl solution in 2-propanol (5 mL) and the reaction mixture was stirred overnight at room temperature. After completion, the reaction mixture was concentrated under reduced pressure. The obtained yellowish oil (100 mg) was purified on C18, using 30% to 70% acetonitrile with 0.1% TFA in water as the eluent. The obtained white solid was dissolved in a saturated aqueous NaHCO₃ solution and the product was extracted into ethyl acetate. The combined organic layers were washed with brine, dried over MgSO4, filtered, and concentrated under reduced pressure. The obtained residue was dissolved in a acetonitrile/water mixture and freeze dried to give the title compound 5-{4-[(4-{[4-pentafluoro-λ⁶-sulfanyl)phenyl]Amino}piperidin-1-yl)sulfonimidoyl]phenyl}-1H-indole-3-carbonitrile (81 mg) as a white solid. MS(ES⁺) m/z 582.4 (M+H)⁺

¹H NMR (400 MHz, DMSO) δ 12.35 (s, 1H), 8.34 (s, 1H), 8.02-7.95 (m, 3H), 7.90-7.84 (m, 2H), 7.69 (d, J=1.2 Hz, 2H), 7.50-7.44 (m, 2H), 6.59 (d, J=9.0 Hz, 2H), 6.44 (d, J=7.8 Hz, 1H), 4.45 (s, 1H), 3.73-3.61 (m, 2H), 3.31-3.24 (m, 1H), 2.41 (t, J=11.3, 11.3 Hz, 2H), 1.95 (d, J=12.5 Hz, 2H), 1.50-1.37 (m, 2H).

Following a procedure analogous to that described for Example 120, using in step v the appropriate boronic ester or boronic acid, Examples 121-125 have been prepared.

### Examples 121-125

### Example 121: N-methyl-5-{4-[(4-{[4-(pentafluoro-λ⁶-sulfanyl)phenyl]Amino}piperidin-1-yl)sulfonimidoyl]phenyl}pyridine-2-carboxamide

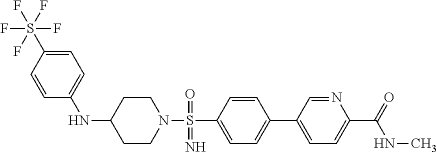 [SMILES: CNC(=O)c1ccc(-c2ccc(S(=N)(=O)N3CCC(Nc4ccc(S(F)(F)(F)(F)F)cc4)CC3)cc2)cn1]

MS(ES⁺) m/z 576.3 (M+H)⁺

Building block: step v: N-methyl-5-(4,4,5,5-tetramethyl-1,3,2-dioxaborolan-2-yl)pyridine-2-carboxamide.

### Example 122: 4′-[(4-{[4-(pentafluoro-λ⁶-sulfanyl)phenyl]Amino}piperidin-1-yl)sulfonimidoyl]-[1,1′-biphenyl]-4-carbonitrile

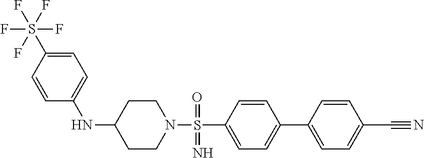 [SMILES: N#Cc1ccc(-c2ccc(S(=N)(=O)N3CCC(Nc4ccc(S(F)(F)(F)(F)F)cc4)CC3)cc2)cc1]

MS(ES⁺) m/z 543.3 (M+H)⁺

Building block: step v: (4-cyanophenyl)boronic acid.

### Example 123: 5-{4-[(4-{[4-(pentafluoro-λ⁶-sulfanyl)phenyl]Amino}piperidin-1-yl)sulfonimidoyl]phenyl}-2,3-dihydro-1H-indol-2-one

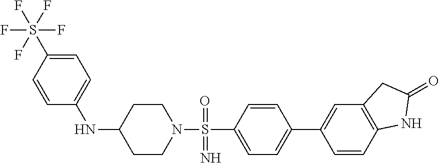 [SMILES: N=S(=O)(c1ccc(-c2ccc3c(c2)CC(=O)N3)cc1)N1CCC(Nc2ccc(S(F)(F)(F)(F)F)cc2)CC1]

MS(ES⁺) m/z 573.3 (M+H)⁺

Building block: step v: 5-(4,4,5,5-tetramethyl-1,3,2-dioxaborolan-2-yl)-2,3-dihydro-1H-indol-2-one.

### Example 124: (4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone

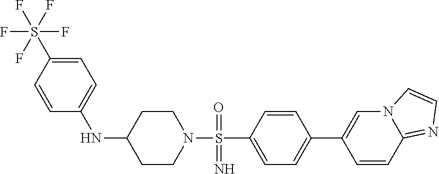 [SMILES: N=S(=O)(c1ccc(-c2ccc3nccn3c2)cc1)N1CCC(Nc2ccc(S(F)(F)(F)(F)F)cc2)CC1]

MS(ES⁺) m/z 558.3 (M+H)⁺

Building block: step v: 6-(4,4,5,5-tetramethyl-1,3,2-dioxaborolan-2-yl)imidazo[1,2-a]pyridine.

### Example 125: 4-{4-[(4-{[4-(pentafluoro-λ⁶-sulfanyl)phenyl]Amino}piperidin-1-yl)sulfonimidoyl]phenyl}pyridine-2-carboxamide

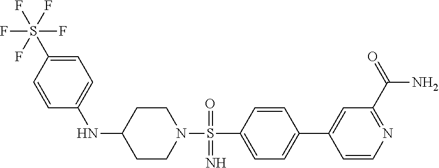 [SMILES: N=S(=O)(c1ccc(-c2ccnc(C(N)=O)c2)cc1)N1CCC(Nc2ccc(S(F)(F)(F)(F)F)cc2)CC1]

MS(ES⁺) m/z 562.3 (M+H)⁺

Building block: step v: 4-(4,4,5,5-tetramethyl-1,3,2-dioxaborolan-2-yl)pyridine-2-carboxamide.

### Example 126: (+)-5-(4-{[trans-4-{[4-(pentafluoro-λ⁶-sulfanyl)phenyl]Amino}cyclohexyl]sulfonimidoyl}phenyl)-1H-indole-3-carbonitrile

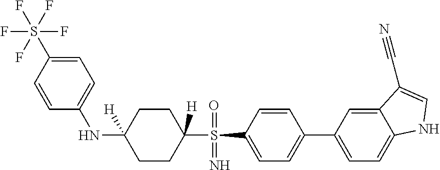 [SMILES: N#Cc1c[nH]c2ccc(-c3ccc([S@](=N)(=O)[C@H]4CC[C@H](Nc5ccc(S(F)(F)(F)(F)F)cc5)CC4)cc3)cc12]

[α]_{D}²⁰°=+7.2°; MS(ES⁺) m/z 581.3 (M+H)⁺.

### Example 127: (−)-5-(4-{[trans-4-{[4-(pentafluoro-λ⁶-sulfanyl)phenyl]Amino}cyclohexyl]sulfonimidoyl}phenyl)-1H-indole-3-carbonitrile

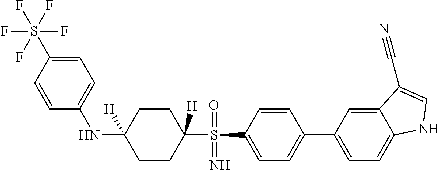 [SMILES: N#Cc1c[nH]c2ccc(-c3ccc([S@](=N)(=O)[C@H]4CC[C@H](Nc5ccc(S(F)(F)(F)(F)F)cc5)CC4)cc3)cc12]

[α]_{D}²⁰°=−6.6°; MS(ES⁺) m/z 581.3 (M+H)⁺.

The single enantiomers Example 126 and Example 127 of the racemic mixture Example 85 can be obtained by chiral separation. 60 mg of racemic Example 85 was dissolved in ethanol (4 mg/mL). The solution was injected on a chiral semi-preparative HPLC, using a semi-preparative Chiralpak AD-H column (available from Daicel) and an isocratic gradient of 20% EtOH, 15% 2-propanol in heptane as the eluent, to obtain: 33 mg of the (+)-enantiomer (Example 126): [α]_{D}²⁰°=+7.20, and 34 mg of the (−)-enantiomer (Example 127): [α]_{D}²⁰°=−6.60.

The absolute configuration of Example 126 and Example 127 are not known. These compounds were characterized by their optical rotation, using a Jasco P-2000 Polarimeter having the following configuration: Tungsten-Halogen (WI) light source; Glan-Taylor Prism Polarizer; Quartz Faraday Cell; and a monitor wavelength of 589 nm.

Following a procedure analogous to that described for Examples 126 and 127, using appropriate starting materials and suitable Chiralpak column, Examples 128-131 have been prepared.

### Examples 128-131

### Example 128: (+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone

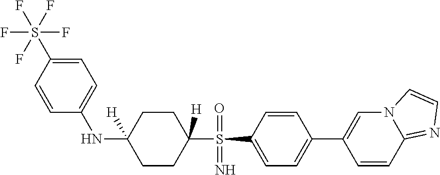 [SMILES: N=[S@](=O)(c1ccc(-c2ccc3nccn3c2)cc1)[C@H]1CC[C@H](Nc2ccc(S(F)(F)(F)(F)F)cc2)CC1]

Chiral Column: Chiralpak IA. [α]_{D}²⁰°=+4.7°; MS(ES⁺) m/z 557.3 (M+H)⁺.

### Example 129: (−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone

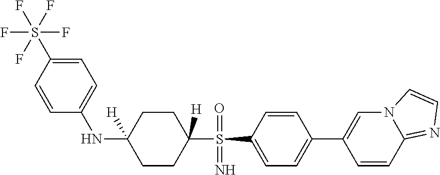 [SMILES: N=[S@](=O)(c1ccc(-c2ccc3nccn3c2)cc1)[C@H]1CC[C@H](Nc2ccc(S(F)(F)(F)(F)F)cc2)CC1]

Chiral Column: Chiralpak IA. [α]_{D}²⁰°=−5.4°; MS(ES⁺) m/z 557.3 (M+H)⁺.

### Example 130: (+)-4-(4-{[trans-4-{[4-(pentafluoro-λ⁶-sulfanyl)phenyl]Amino}cyclohexyl]sulfonimidoyl}phenyl)pyridine-2-carboxamide

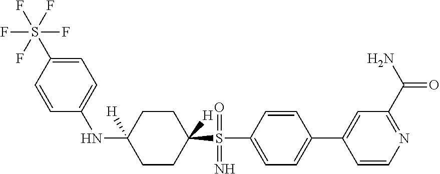 [SMILES: N=S(=O)(c1ccc(-c2ccnc(C(N)=O)c2)cc1)[C@H]1CC[C@@H](Nc2ccc(S(F)(F)(F)(F)F)cc2)CC1]

Chiral Column: Chiralpak AD-H. [α]_{D}²⁰=+2.6°; MS(ES⁺) m/z 561.3 (M+H).

### Example 131: (−)-4-(4-{[trans-4-{[4-(pentafluoro-λ⁶-sulfanyl)phenyl]Amino}cyclohexyl]sulfonimidoyl}phenyl)pyridine-2-carboxamide

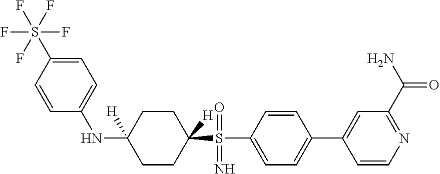 [SMILES: N=S(=O)(c1ccc(-c2ccnc(C(N)=O)c2)cc1)[C@H]1CC[C@@H](Nc2ccc(S(F)(F)(F)(F)F)cc2)CC1]

Chiral Column: Chiralpak AD-H. [α]_{D}²⁰°=−4.5°; MS(ES⁺) m/z 561.3 (M+H)⁺. Biological essays

### Example 132: 1-(4-{2-methoxy-7-azaspiro[3.5]nonan-7-yl}benzenesulfonyl)-N-[4-(pentafluoro-λ⁶-sulfanyl)phenyl]piperidin-4-amine

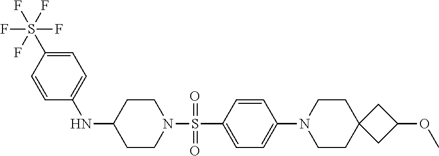 [SMILES: COC1CC2(CCN(c3ccc(S(=O)(=O)N4CCC(Nc5ccc(S(F)(F)(F)(F)F)cc5)CC4)cc3)CC2)C1]

i) To a solution of N-[4-(pentafluoro-λ⁶-sulfanyl)phenyl]piperidin-4-amine hydrochloride, Example 67, step ii, (1.07 g) and Et₃N (1.5 mL) in CH₂Cl₂ (40 mL) was added 4-fluorobenzene-1-sulfonyl chloride (0.70 g). The reaction mixture was stirred overnight at room temperature. The reaction mixture was quenched by pouring it into water. The product was extracted into CH₂Cl₂ and the combined organic layers were dried over MgSO₄, filtered and concentrated under vacuo to give 1-(4-fluorobenzenesulfonyl)-N-[4-(pentafluoro-λ⁶-sulfanyl)phenyl]piperidin-4-amine (1.36 g) as an off white solid, which was used in the next step without any further purification.
ii) In a capped microwave vial, a mixture of the product obtained in the previous step (100 mg), 2-methoxy-7-azaspiro[3.5]nonane hydrochloride (77 mg) and K₂CO in NMP (2 mL) was stirred overnight at 120° C. After cooling down to r.t., the reaction mixture was poured into water and the product was extracted into ethyl acetate. The combined organic layers were dried over MgSO₄, filtered and concentrated under vacuo and the obtained oil was purified on C18, using 20% to 60% acetonitrile in water as the eluent to give the title compound 1-(4-{2-methoxy-7-azaspiro[3.5]nonan-7-yl}benzenesulfonyl)-N-[4-(pentafluoro-λ⁶-sulfanyl)phenyl]piperidin-4-amine 9 (24 mg) as a white solid. MS(ES⁺) m/z 596.4 (M+H)⁺

¹H NMR (400 MHz, DMSO) δ 7.52-7.44 (t, J=9.1 Hz, 4H), 7.10-7.03 (d, J=9.1 Hz, 2H), 6.64-6.56 (d, J=8.9 Hz, 2H), 6.48-6.41 (d, J=7.9 Hz, 1H), 3.93-3.85 (p, J=6.9 Hz, 1H), 3.53-3.45 (d, J=11.6 Hz, 2H), 3.32-3.29 (m, 3H), 3.29-3.23 (m, 2H), 3.14-3.11 (s, 3H), 2.45-2.36 (t, 2H), 2.20-2.13 (m, 2H), 1.98-1.89 (d, 2H), 1.68-1.55 (m, 6H), 1.47-1.33 (m, 2H).

Following a procedure analogous to that described for Example 132, using in step ii the appropriate heterocycle, Examples 133-137 have been prepared.

### Examples 133-137

### Example 133: 8-{4-[(4-{[4-(pentafluoro-λ⁶-sulfanyl)phenyl]amino}piperidin-1-yl)sulfonyl]phenyl}-1,3,8-triazaspiro[4.5]decane-2,4-dione

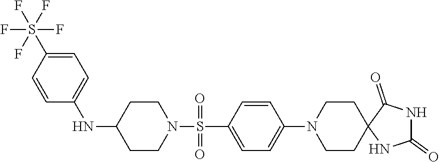 [SMILES: O=C1NC(=O)C2(CCN(c3ccc(S(=O)(=O)N4CCC(Nc5ccc(S(F)(F)(F)(F)F)cc5)CC4)cc3)CC2)N1]

MS(ES⁺) m/z 610.4 (M+H)⁺

Building block: step ii: 1,3,8-triazaspiro[4.5]decane-2,4-dione.

### Example 134: 8-{4-[(4-{[4-(pentafluoro-λ⁶-sulfanyl)phenyl]amino}piperidin-1-yl)sulfonyl]phenyl}-2,8-diazaspiro[4.5]decan-1-one

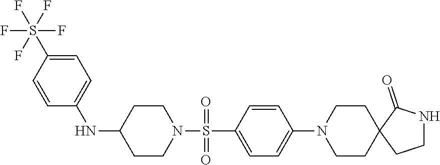 [SMILES: O=C1NCCC12CCN(c1ccc(S(=O)(=O)N3CCC(Nc4ccc(S(F)(F)(F)(F)F)cc4)CC3)cc1)CC2]

MS(ES⁺) m/z 595.3 (M+H)⁺

Building block: step ii: 2,8-diazaspiro[4.5]decan-1-one.

### Example 135: 8-{4-[(4-{[4-(pentafluoro-λ⁶-sulfanyl)phenyl]amino}piperidin-1-yl)sulfonyl]phenyl}-3-oxa-1,8-diazaspiro[4.5]decan-2-one

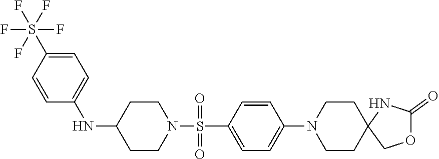 [SMILES: O=C1NC2(CCN(c3ccc(S(=O)(=O)N4CCC(Nc5ccc(S(F)(F)(F)(F)F)cc5)CC4)cc3)CC2)CO1]

MS(ES⁺) m/z 597.3 (M+H)⁺

Building block: step ii: 3-oxa-1,8-diazaspiro[4.5]decan-2-one.

### Example 136: 1-(4-{2-oxa-7-azaspiro[3.5]nonan-7-yl}benzenesulfonyl)-N-[4-(pentafluoro-λ⁶-sulfanyl)phenyl]piperidin-4-amine

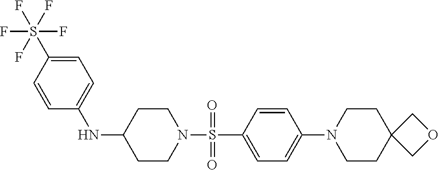 [SMILES: O=S(=O)(c1ccc(N2CCC3(CC2)COC3)cc1)N1CCC(Nc2ccc(S(F)(F)(F)(F)F)cc2)CC1]

MS(ES⁺) m/z 568.3 (M+H)⁺

Building block: step ii: 2-oxa-7-azaspiro[3.5]nonane.

### Example 137: 1-(4-{8-oxa-3-azabicyclo[3.2.1]octan-3-yl}benzenesulfonyl)-N-[4-(pentafluoro-λ⁶-sulfanyl)phenyl]piperidin-4-amine

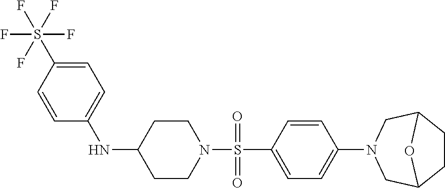 [SMILES: O=S(=O)(c1ccc(N2CC3CCC(C2)O3)cc1)N1CCC(Nc2ccc(S(F)(F)(F)(F)F)cc2)CC1]

MS(ES⁺) m/z 554.3 (M+H)⁺

Building block: step ii: 8-oxa-3-azabicyclo[3.2.1]octane.

Following a procedure analogous to that described for Example 24, using in step ii the appropriate aryl halide, Example 138-140 have been prepared.

### Examples 138-140

### Example 138: 4-fluoro-N-methyl-4′-[(4-{[4-(pentafluoro-λ⁶-sulfanyl)phenyl]amino}piperidin-1-yl)sulfonyl]-[1,1′-biphenyl]-3-carboxamide

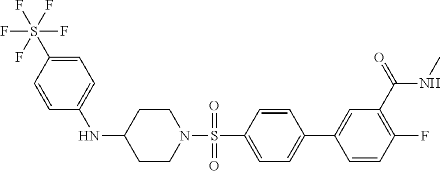 [SMILES: CNC(=O)c1cc(-c2ccc(S(=O)(=O)N3CCC(Nc4ccc(S(F)(F)(F)(F)F)cc4)CC3)cc2)ccc1F]

MS(ES⁺) m/z 594.4 (M+H)⁺

Building block: Step ii: 5-bromo-2-fluoro-N-methylbenzamide.

### Example 139: 3-fluoro-N-methyl-4′-[(4-{[4-(pentafluoro-λ⁶-sulfanyl)phenyl]amino}piperidin-1-yl)sulfonyl]-[1,1′-biphenyl]-4-carboxamide

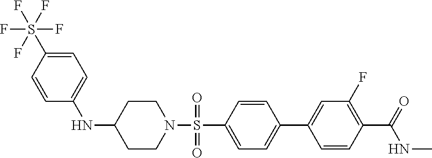 [SMILES: CNC(=O)c1ccc(-c2ccc(S(=O)(=O)N3CCC(Nc4ccc(S(F)(F)(F)(F)F)cc4)CC3)cc2)cc1F]

MS(ES⁺) m/z 594.4 (M+H)⁺

Building block: Step ii: 4-bromo-2-fluoro-N-methylbenzamide.

### Example 140: N-[4-(pentafluoro-λ⁶-sulfanyl)phenyl]-1-{4-[3-(propan-2-yl)-[1,2,4]triazolo[4,3-a]pyrazin-6-yl]benzenesulfonyl}piperidin-4-amine

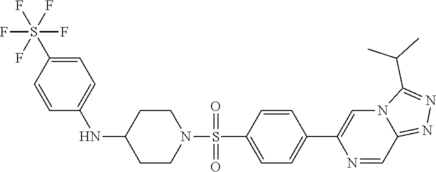 [SMILES: CC(C)c1nnc2cnc(-c3ccc(S(=O)(=O)N4CCC(Nc5ccc(S(F)(F)(F)(F)F)cc5)CC4)cc3)cn12]

MS(ES⁺) m/z 603.4 (M+H)⁺

Building block: Step ii: 6-bromo-3-(propan-2-yl)-[1,2,4]triazolo[4,3-a]pyrazine.

Following a procedure analogous to that described for Example 33, using in step ii the appropriate aryl halide, Example 141-161 have been prepared.

### Example 141-161

Example 141: N-(propan-2-yl)-4′-{[trans-4-{[4-(pentafluoro-λ⁶-sulfanyl)phenyl]amino}cyclohexyl]sulfonyl}-[1,1′-biphenyl]-3-carboxamide

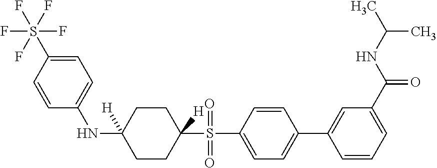 [SMILES: CC(C)NC(=O)c1cccc(-c2ccc(S(=O)(=O)[C@H]3CC[C@H](Nc4ccc(S(F)(F)(F)(F)F)cc4)CC3)cc2)c1]

MS(ES⁺) m/z 603.3 (M+H)⁺

Building block: Step ii: 3-bromo-N-(propan-2-yl)benzamide.

### Example 142: 4-(pentafluoro-λ⁶-sulfanyl)-N-[trans-4-(4-{3-methyl-1H-pyrazolo[3,4-c]pyridin-5-yl}benzenesulfonyl)cyclohexyl]aniline

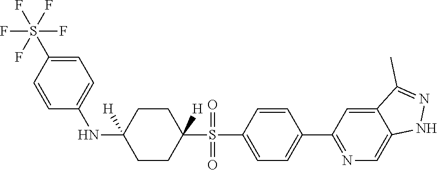 [SMILES: Cc1n[nH]c2cnc(-c3ccc(S(=O)(=O)[C@H]4CC[C@H](Nc5ccc(S(F)(F)(F)(F)F)cc5)CC4)cc3)cc12]

MS(ES⁺) m/z 573.3 (M+H)⁺

Building block: Step ii: 5-bromo-3-methyl-1H-pyrazolo[3,4-c]pyridine.

### Example 143: N,N-dimethyl-6-(4-{[trans-4-{[4-(pentafluoro-λ⁶-sulfanyl)phenyl]amino}cyclohexyl]sulfonyl}phenyl)imidazo[1,2-a]pyridin-3-amine

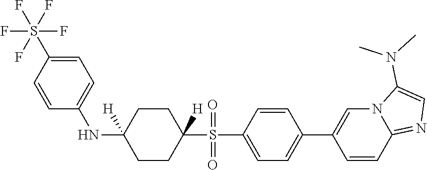 [SMILES: CN(C)c1cnc2ccc(-c3ccc(S(=O)(=O)[C@H]4CC[C@H](Nc5ccc(S(F)(F)(F)(F)F)cc5)CC4)cc3)cn12]

MS(ES⁺) m/z 601.4 (M+H)⁺

Building block: Step ii: 6-bromo-N,N-dimethylimidazo[1,2-a]pyridin-3-amine.

### Example 144: N-cyclopropyl-4′-{[trans-4-{[4-(pentafluoro-λ⁶-sulfanyl)phenyl]amino}cyclohexyl]sulfonyl}-[1,1′-biphenyl]-3-carboxamide

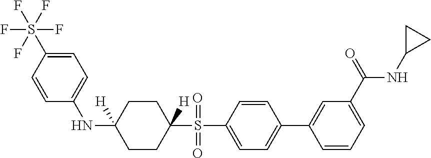 [SMILES: O=C(NC1CC1)c1cccc(-c2ccc(S(=O)(=O)[C@H]3CC[C@H](Nc4ccc(S(F)(F)(F)(F)F)cc4)CC3)cc2)c1]

MS(ES⁺) m/z 601.4 (M+H)⁺

Building block: Step ii: 3-bromo-N-cyclopropylbenzamide.

### Example 145: 4-(pentafluoro-λ⁶-sulfanyl)-N-[trans-4-(4-{5H-pyrrolo[3,2-d]pyrimidin-2-yl}benzenesulfonyl)cyclohexyl]aniline

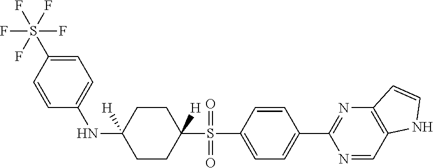 [SMILES: O=S(=O)(c1ccc(-c2ncc3[nH]ccc3n2)cc1)[C@H]1CC[C@H](Nc2ccc(S(F)(F)(F)(F)F)cc2)CC1]

MS(ES⁺) m/z 559.4 (M+H)⁺

Building block: Step ii: 2-chloro-5H-pyrrolo[3,2-d]pyrimidine.

### Example 146: 4-(pentafluoro-λ⁶-sulfanyl)-N-[trans-4-(4-{1H-pyrrolo[2,3-c]pyridin-5-yl}benzenesulfonyl)cyclohexyl]aniline

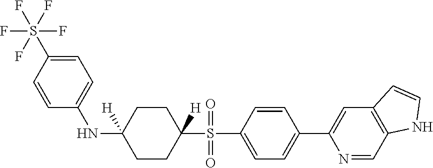 [SMILES: O=S(=O)(c1ccc(-c2cc3cc[nH]c3cn2)cc1)[C@H]1CC[C@H](Nc2ccc(S(F)(F)(F)(F)F)cc2)CC1]

MS(ES⁺) m/z 558.4 (M+H)⁺

Building block: Step ii: 5-chloro-1H-pyrrolo[2,3-c]pyridine.

### Example 147: 4-(pentafluoro-λ⁶-sulfanyl)-N-[trans-4-(4-{1H-pyrazolo[3,4-c]pyridin-5-yl}benzenesulfonyl)cyclohexyl]aniline

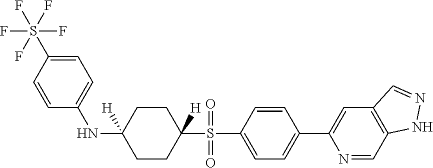 [SMILES: O=S(=O)(c1ccc(-c2cc3cn[nH]c3cn2)cc1)[C@H]1CC[C@H](Nc2ccc(S(F)(F)(F)(F)F)cc2)CC1]

MS(ES⁺) m/z 559.4 (M+H)⁺

Building block: Step ii: 5-bromo-1H-pyrazolo[3,4-c]pyridine.

### Example 148: 4-(4-{[trans-4-{[4-(pentafluoro-λ⁶-sulfanyl)phenyl]amino}cyclohexyl]sulfonyl}phenyl)thiophene-2-carboxamide

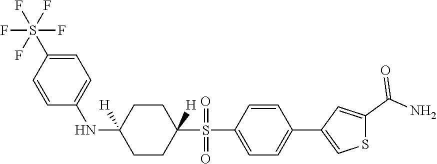 [SMILES: NC(=O)c1cc(-c2ccc(S(=O)(=O)[C@H]3CC[C@H](Nc4ccc(S(F)(F)(F)(F)F)cc4)CC3)cc2)cs1]

MS(ES⁺) m/z 567.3 (M+H)⁺

Building block: Step ii: 4-bromothiophene-2-carboxamide.

### Example 149: 4-(pentafluoro-λ⁶-sulfanyl)-N-[trans-4-(4-{8-methylimidazo[1,2-a]pyridin-6-yl}benzenesulfonyl)cyclohexyl]aniline

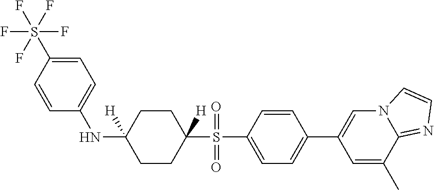 [SMILES: Cc1cc(-c2ccc(S(=O)(=O)[C@H]3CC[C@H](Nc4ccc(S(F)(F)(F)(F)F)cc4)CC3)cc2)cn2ccnc12]

MS(ES⁺) m/z 572.3 (M+H)⁺

Building block: Step ii: 6-bromo-8-methylimidazo[1,2-a]pyridine.

### Example 150: 4-(pentafluoro-λ⁶-sulfanyl)-N-[trans-4-(4-{3-methyl-1H-pyrazolo[3,4-b]pyridin-5-yl}benzenesulfonyl)cyclohexyl]aniline

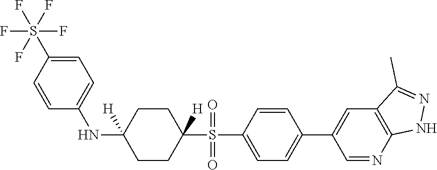 [SMILES: Cc1n[nH]c2ncc(-c3ccc(S(=O)(=O)[C@H]4CC[C@H](Nc5ccc(S(F)(F)(F)(F)F)cc5)CC4)cc3)cc12]

MS(ES⁺) m/z 573.3 (M+H)⁺

Building block: Step ii: 5-bromo-3-methyl-1H-pyrazolo[3,4-b]pyridine.

### Example 151: 4-(pentafluoro-λ⁶-sulfanyl)-N-[trans-4-{4-[8-(trifluoromethyl)imidazo[1,2-a]pyridin-6-yl]benzenesulfonyl}cyclohexyl]aniline

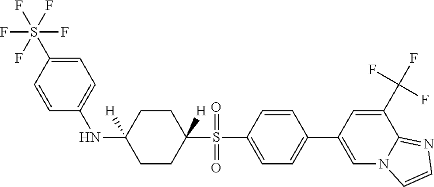 [SMILES: O=S(=O)(c1ccc(-c2cc(C(F)(F)F)c3nccn3c2)cc1)[C@H]1CC[C@H](Nc2ccc(S(F)(F)(F)(F)F)cc2)CC1]

MS(ES⁺) m/z 623.4 (M+H)⁺

Building block: Step ii: 6-bromo-8-(trifluoromethyl)imidazo[1,2-a]pyridine.

### Example 152: 4-(pentafluoro-λ⁶-sulfanyl)-N-[trans-4-(4-{8-fluoroimidazo[1,2-a]pyridin-6-yl}benzenesulfonyl)cyclohexyl]aniline

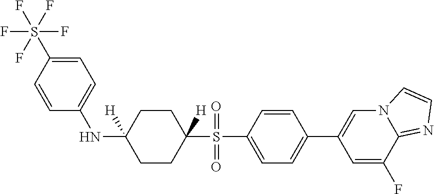 [SMILES: O=S(=O)(c1ccc(-c2cc(F)c3nccn3c2)cc1)[C@H]1CC[C@H](Nc2ccc(S(F)(F)(F)(F)F)cc2)CC1]

MS(ES⁺) m/z 576.4 (M+H)⁺

Building block: Step ii: 6-bromo-8-fluoroimidazo[1,2-a]pyridine.

### Example 153 ( ): 2-(4-{[trans-4-{[4-(pentafluoro-λ⁶-sulfanyl)phenyl]amino}cyclohexyl]sulfonyl}phenyl)-4H,5H,6H,7H-thieno[3,2-c]pyridin-4-one

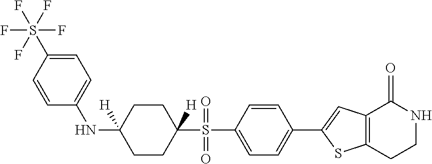 [SMILES: O=C1NCCc2sc(-c3ccc(S(=O)(=O)[C@H]4CC[C@H](Nc5ccc(S(F)(F)(F)(F)F)cc5)CC4)cc3)cc21]

MS(ES⁺) m/z 593.4 (M+H)⁺

Building block: Step ii: 2-bromo-4H,5H,6H,7H-thieno[3,2-c]pyridin-4-one.

### Example 154: 7-(4-{[trans-4-{[4-(pentafluoro-λ⁶-sulfanyl)phenyl]amino}cyclohexyl]sulfonyl}phenyl)-1H,2H-pyrrolo[1,2-a]pyrazin-1-one

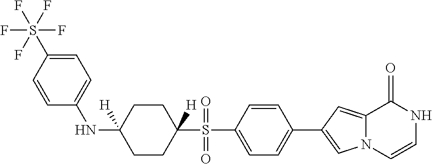 [SMILES: O=c1[nH]ccn2cc(-c3ccc(S(=O)(=O)[C@H]4CC[C@H](Nc5ccc(S(F)(F)(F)(F)F)cc5)CC4)cc3)cc12]

MS(ES⁺) m/z 574.4 (M+H)⁺

Building block: Step ii: 7-bromo-1H,2H-pyrrolo[1,2-a]pyrazin-1-one.

### Example 155: 8-(4-{[trans-4-{[4-(pentafluoro-λ⁶-sulfanyl)phenyl]amino}cyclohexyl]sulfonyl}phenyl)-1H,2H,3H,4H,5H-pyrrolo[1,2-a][1,4]diazepin-1-one

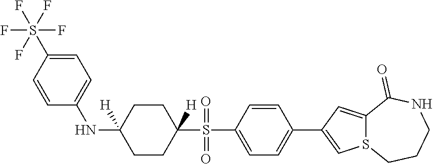

MS(ES⁺) m/z 590.3 (M+H)⁺

Building block: Step ii: 8-bromo-1H,2H,3H,4H,5H-pyrrolo[1,2-a][1,4]diazepin-1-one.

### Example 156: 7-(4-{[trans-4-{[4-(pentafluoro-λ⁶-sulfanyl)phenyl]amino}cyclohexyl]sulfonyl}phenyl)-1H,2H,3H,4H-pyrrolo[1,2-a]pyrazin-1-one

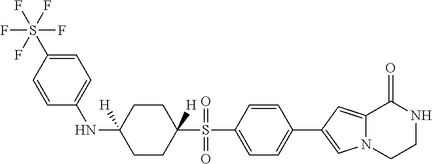 [SMILES: O=C1NCCn2cc(-c3ccc(S(=O)(=O)[C@H]4CC[C@H](Nc5ccc(S(F)(F)(F)(F)F)cc5)CC4)cc3)cc21]

MS(ES⁺) m/z 576.3 (M+H)⁺

Building block: Step ii: 7-bromo-1H,2H,3H,4H-pyrrolo[1,2-a]pyrazin-1-one.

### Example 157: 6-(4-{[trans-4-{[4-(pentafluoro-λ⁶-sulfanyl)phenyl]amino}cyclohexyl]sulfonyl}phenyl)imidazo[1,2-a]pyridine-8-carboxamide

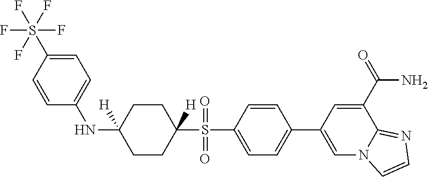 [SMILES: NC(=O)c1cc(-c2ccc(S(=O)(=O)[C@H]3CC[C@H](Nc4ccc(S(F)(F)(F)(F)F)cc4)CC3)cc2)cn2ccnc12]

MS(ES⁺) m/z 601.3 (M+H)⁺

Building block: Step ii: 6-bromoimidazo[1,2-a]pyridine-8-carboxamide.

### Example 158: 4-(4-{[trans-4-{[4-(pentafluoro-λ⁶-sulfanyl)phenyl]amino}cyclohexyl]sulfonyl}phenyl)furan-2-carboxamide

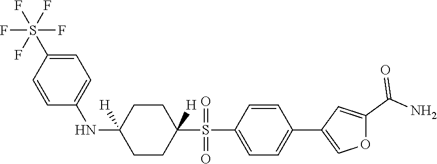 [SMILES: NC(=O)c1cc(-c2ccc(S(=O)(=O)[C@H]3CC[C@H](Nc4ccc(S(F)(F)(F)(F)F)cc4)CC3)cc2)co1]

MS(ES⁺) m/z 551.3 (M+H)⁺

Building block: Step ii: 4-bromofuran-2-carboxamide.

### Example 159: 6-(4-{[trans-4-{[4-(pentafluoro-λ⁶-sulfanyl)phenyl]amino}cyclohexyl]sulfonyl}phenyl)imidazo[1,2-a]pyridin-8-amine

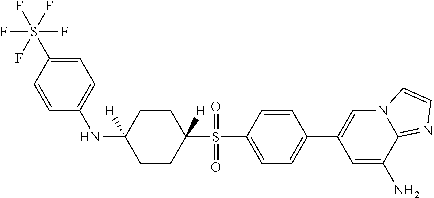 [SMILES: Nc1cc(-c2ccc(S(=O)(=O)[C@H]3CC[C@H](Nc4ccc(S(F)(F)(F)(F)F)cc4)CC3)cc2)cn2ccnc12]

MS(ES⁺) m/z 573.3 (M+H)⁺

Building block: Step ii: 6-bromoimidazo[1,2-a]pyridin-8-amine.

### Example 160: 6-(4-{[trans-4-{[4-(pentafluoro-λ⁶-sulfanyl)phenyl]amino}cyclohexyl]sulfonyl}phenyl)imidazo[1,2-a]pyridine-8-carbonitrile

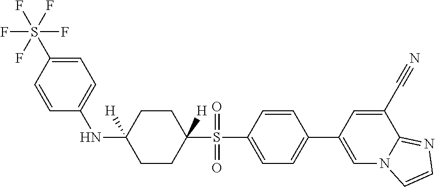 [SMILES: N#Cc1cc(-c2ccc(S(=O)(=O)[C@H]3CC[C@H](Nc4ccc(S(F)(F)(F)(F)F)cc4)CC3)cc2)cn2ccnc12]

MS(ES⁺) m/z 583.3 (M+H)⁺

Building block: Step ii: 6-bromoimidazo[1,2-a]pyridine-8-carbonitrile.

### Example 161: 4-(pentafluoro-λ⁶-sulfanyl)-N-[trans-4-(4-{3-[(2S)-pyrrolidin-2-yl]-[1,2,4]triazolo[4,3-a]pyridin-6-yl}benzenesulfonyl)cyclohexyl]aniline

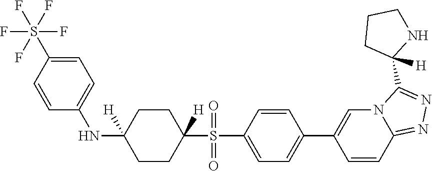 [SMILES: O=S(=O)(c1ccc(-c2ccc3nnc([C@@H]4CCCN4)n3c2)cc1)[C@H]1CC[C@H](Nc2ccc(S(F)(F)(F)(F)F)cc2)CC1]

MS(ES⁺) m/z 628.4 (M+H)⁺

Building block: Step ii: (9H-fluoren-9-yl)methyl (2S)-2-{6-bromo-[1,2,4]triazolo[4,3-a]pyridin-3-yl}pyrrolidine-1-carboxylate.

Following a procedure analogous to that described for Example 102, using in step ii the appropriate aryl halide, Example 162 has been prepared.

### Example 162: {4-[3-(propan-2-yl)-[1,2,4]triazolo[4,3-a]pyrazin-6-yl]phenyl}[trans-4-{[4-(pentafluoro-λ⁶-sulfanyl)phenyl]amino}cyclohexyl](imino)-λ⁶-sulfanone

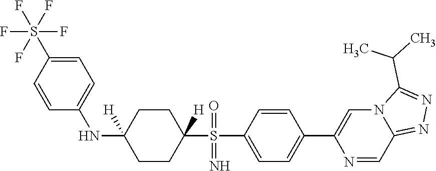 [SMILES: CC(C)c1nnc2cnc(-c3ccc(S(=N)(=O)[C@H]4CC[C@H](Nc5ccc(S(F)(F)(F)(F)F)cc5)CC4)cc3)cn12]

MS(ES⁺) m/z 601.3 (M+H)⁺

Building block: Step ii: 6-bromo-3-(propan-2-yl)-[1,2,4]triazolo[4,3-a]pyrazine.

Following a procedure analogous to that described for Examples 126 and 127, using appropriate starting materials and suitable Chiralpak column, Examples 163-164 have been prepared.

### Examples 163-164

### Example 163: (+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone

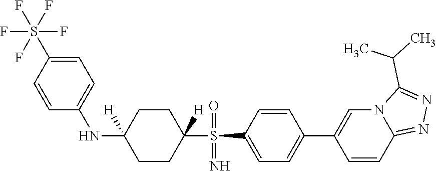 [SMILES: CC(C)c1nnc2ccc(-c3ccc([S@](=N)(=O)[C@H]4CC[C@H](Nc5ccc(S(F)(F)(F)(F)F)cc5)CC4)cc3)cn12]

Chiral Column: Chiralpak AD-H. [α]_{D}²⁰°=+2.9°; MS(ES⁺) m/z 600.4 (M+H)⁺.

### Example 164: (−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone

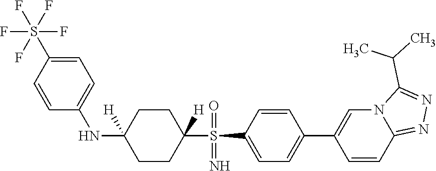 [SMILES: CC(C)c1nnc2ccc(-c3ccc([S@](=N)(=O)[C@H]4CC[C@H](Nc5ccc(S(F)(F)(F)(F)F)cc5)CC4)cc3)cn12]

Chiral Column: Chiralpak AD-H. [α]_{D}²⁰°=−3.4°; MS(ES⁺) m/z 600.4 (M+H)⁺.

### Example 165: N-[4-(pentafluoro-λ⁶-sulfanyl)phenyl]-1-(4-{3-[(3R)-pyrrolidin-3-yl]-[1,2,4]triazolo[4,3-a]pyridin-6-yl}benzenesulfonyl)piperidin-4-amine

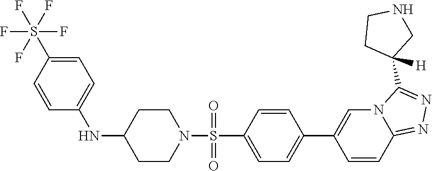 [SMILES: O=S(=O)(c1ccc(-c2ccc3nnc([C@@H]4CCNC4)n3c2)cc1)N1CCC(Nc2ccc(S(F)(F)(F)(F)F)cc2)CC1]

i) Following a procedure analogous to that described for Example 24, using in step ii, tert-butyl (3R)-3-{6-bromo-[1,2,4]triazolo[4,3-a]pyridin-3-yl}pyrrolidine-1-carboxylate, tert-butyl (3R)-3-(6-{4-[(4-{[4-(pentafluoro-λ⁶-sulfanyl)phenyl]amino}piperidin-1-yl)sulfonyl]phenyl}-[1,2,4]triazolo[4,3-a]pyridin-3-yl)pyrrolidine-1-carboxylate (200 mg) has been prepared as a yellow solid.
ii) To a solution of the product obtained in the previous step (200 mg) in ethyl acetate (10 mL) was added a 5N HCl solution in 2-propanol (10 mL) and the reaction mixture was stirred overnight at room temperature. After completion, the reaction mixture was concentrated under reduced pressure. The yellow oil (123 mg) was purified on C18, using 10% to 80% CH₃CN in water as the eluent giving the title compound N-[4-(pentafluoro-λ⁶-sulfanyl)phenyl]-1-(4-{3-[(3R)-pyrrolidin-3-yl]-[1,2,4]triazolo[4,3-a]pyridin-6-yl}benzenesulfonyl)piperidin-4-amine (75 mg) as a white solid. MS(ES⁺) m/z 629.3 (M+H)⁺

¹H NMR (400 MHz, DMSO) δ 8.92-8.88 (m, 1H), 8.13-8.08 (m, 2H), 7.92-7.86 (m, 3H), 7.81-7.75 (m, 1H), 7.51-7.44 (m, 2H), 6.63-6.57 (d, J=8.8 Hz, 2H), 6.50-6.44 (d, 1H), 4.01-3.88 (m, 1H), 3.67-3.57 (m, 2H), 3.40-3.34 (m, 3H), 3.16-3.01 (m, 2H), 3.01-2.91 (m, 1H), 2.62-2.54 (m, 2H), 2.35-2.24 (m, 1H), 2.17-2.07 (m, 1H), 2.02-1.92 (m, 2H), 1.52-1.39 (m, 2H).

Following a procedure analogous to that described for Example 165, using in step i the appropriate aryl halide, Example 166 has been prepared.

### Example 166: N-[4-(pentafluoro-λ⁶-sulfanyl)phenyl]-1-(4-{3-[(3S)-pyrrolidin-3-yl]-[1,2,4]triazolo[4,3-a]pyridin-6-yl}benzenesulfonyl)piperidin-4-amine

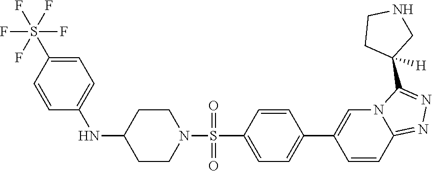 [SMILES: O=S(=O)(c1ccc(-c2ccc3nnc([C@H]4CCNC4)n3c2)cc1)N1CCC(Nc2ccc(S(F)(F)(F)(F)F)cc2)CC1]

MS(ES⁺) m/z 629.4 (M+H)⁺

Building block: Step i: tert-butyl (3S)-3-{6-bromo-[1,2,4]triazolo[4,3-a]pyridin-3-yl}pyrrolidine-1-carboxylate.

### Example 167: 1-{4-[3-(azetidin-3-yl)-[1,2,4]triazolo[4,3-a]pyridin-6-yl]benzenesulfonyl}-N-[4-(pentafluoro-λ⁶-sulfanyl)phenyl]piperidin-4-amine

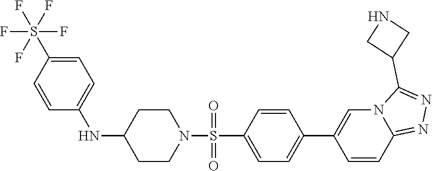 [SMILES: O=S(=O)(c1ccc(-c2ccc3nnc(C4CNC4)n3c2)cc1)N1CCC(Nc2ccc(S(F)(F)(F)(F)F)cc2)CC1]

i) Following a procedure analogous to that described for Example 24, using in step ii, benzyl 3-{6-bromo-[1,2,4]triazolo[4,3-a]pyridin-3-yl}azetidine-1-carboxylate, benzyl 3-(6-{4-[(4-{[4-(pentafluoro-λ⁶-sulfanyl)phenyl]amino}piperidin-1-yl)sulfonyl]phenyl}-[1,2,4]triazolo[4,3-a]pyridin-3-yl)azetidine-1-carboxylate (244 mg) has been prepared as a yellow solid.
ii) A solution of the product obtained in the previous step (244 mg) in TFA (1 mL) was stirred for 3 hours at 65° C. After completion, the reaction mixture was concentrated under reduced pressure. The yellow oil (123 mg) was purified on C18, using 10% to 60% CH₃CN in water as the eluent giving the title compound 1-{4-[3-(azetidin-3-yl)-[1,2,4]triazolo[4,3-a]pyridin-6-yl]benzenesulfonyl}-N-[4-(pentafluoro-λ⁶-sulfanyl)phenyl]piperidin-4-amine (22 mg) as a white solid. MS(ES⁺) m/z 615.4 (M+H)⁺

¹H NMR (400 MHz, DMSO) δ 9.15-9.11 (s, 1H), 9.01-8.97 (s, 1H), 8.86-8.80 (t, J=1.4 Hz, 1H), 8.12-8.04 (m, 2H), 7.98-7.93 (dd, J=9.7, 1.0 Hz, 1H), 7.93-7.88 (m, 2H), 7.88-7.83 (dd, J=9.6, 1.6 Hz, 1H), 7.52-7.44 (m, 2H), 6.64-6.57 (d, J=9.0 Hz, 2H), 6.50-6.45 (s, 1H), 4.88-4.75 (p, J=8.3 Hz, 1H), 4.53-4.40 (m, 4H), 3.65-3.59 (m, 2H), 3.42-3.29 (s, 1H), 2.63-2.54 (m, 2H), 2.01-1.94 (m, 2H), 1.52-1.40 (m, 2H).

### Example 168: 4-(pentafluoro-λ⁶-sulfanyl)-N-[trans-4-{4-[3-(azetidin-3-yl)-[1,2,4]triazolo[4,3-a]pyridin-6-yl]benzenesulfonyl}cyclohexyl]aniline

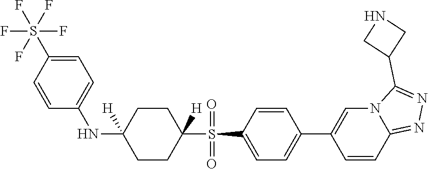

i) Following a procedure analogous to that described for Example 33, using in step ii, benzyl 3-{6-bromo-[1,2,4]triazolo[4,3-a]pyridin-3-yl}azetidine-1-carboxylate, benzyl 3-[6-(4-{[trans-4-{[4-(pentafluoro-λ⁶-sulfanyl)phenyl]amino}cyclohexyl]sulfonyl}phenyl)-[1,2,4]triazolo[4,3-a]pyridin-3-yl]azetidine-1-carboxylate (170 mg) has been prepared as a yellow solid.
ii) A solution of the product obtained in the previous step (170 mg) in TFA (1 mL) was stirred for 3 hours at 65° C. After completion, the reaction mixture was concentrated under reduced pressure. The yellow oil (123 mg) was purified on C18, using 10% to 60% CH₃CN in water as the eluent giving the title compound 4-(pentafluoro-λ⁶-sulfanyl)-N-[trans-4-{4-[3-(azetidin-3-yl)-[1,2,4]triazolo[4,3-a]pyridin-6-yl]benzenesulfonyl}cyclohexyl]aniline (30 mg) as a white solid. MS(ES⁺) m/z 614.4 (M+H)⁺

¹H NMR (400 MHz, DMSO) δ 9.15-9.11 (s, 1H), 9.01-8.96 (s, 1H), 8.86-8.83 (t, J=1.4 Hz, 1H), 8.13-8.05 (m, 2H), 8.04-7.98 (m, 2H), 7.97-7.93 (dd, J=9.6, 1.0 Hz, 1H), 7.90-7.83 (dd, J=9.7, 1.7 Hz, 1H), 7.52-7.45 (m, 2H), 6.65-6.58 (d, J=9.0 Hz, 2H), 6.46-6.41 (s, 1H), 4.87-4.74 (p, J=8.3 Hz, 1H), 4.53-4.42 (m, 4H), 3.45-3.34 (m, 1H), 3.34-3.19 (m, 1H), 2.08-1.92 (m, 4H), 1.63-1.50 (m, 2H), 1.26-1.14 (m, 2H).

Following a procedure analogous to that described for Example 102, using in step ii the appropriate aryl halide, Example 169-170 has been prepared.

### Example 169: {4-[3-(difluoromethyl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone

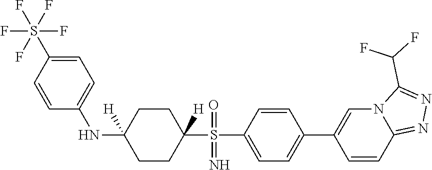 [SMILES: N=S(=O)(c1ccc(-c2ccc3nnc(C(F)F)n3c2)cc1)[C@H]1CC[C@H](Nc2ccc(S(F)(F)(F)(F)F)cc2)CC1]

MS(ES⁺) m/z 608.3 (M+H)⁺

Building block: Step ii: 6-bromo-3-(difluoromethyl)-[1,2,4]triazolo[4,3-a]pyridine.

### Example 170: (4-{3-methyl-[1,2,4]triazolo[4,3-a]pyrazin-6-yl}phenyl) [trans-4-{[4-(pentafluoro-λ⁶-sulfanyl)phenyl]amino}cyclohexyl](imino)-λ⁶-sulfanone

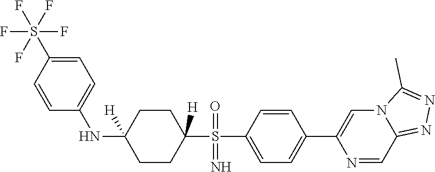 [SMILES: Cc1nnc2cnc(-c3ccc(S(=N)(=O)[C@H]4CC[C@H](Nc5ccc(S(F)(F)(F)(F)F)cc5)CC4)cc3)cn12]

MS(ES⁺) m/z 573.3 (M+H)⁺

Building block: Step ii: 6-chloro-3-methyl-[1,2,4]triazolo[4,3-a]pyrazine.

### Example 171: {4-[3-(propan-2-yl)-[1,2,4]triazolo[4,3-a]pyridin-6-yl]phenyl}[trans-4-{[5-(pentafluoro-λ⁶-sulfanyl)pyridin-2-yl]amino}cyclohexyl](imino)-λ⁶-sulfanone

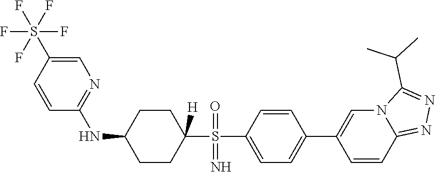 [SMILES: CC(C)c1nnc2ccc(-c3ccc(S(=N)(=O)[C@H]4CC[C@H](Nc5ccc(S(F)(F)(F)(F)F)cn5)CC4)cc3)cn12]

MS(ES⁺) m/z 601.3 (M+H)⁺

Building block: Step ii: 6-bromo-3-(propan-2-yl)-[1,2,4]triazolo[4,3-a]pyridine.

### Example 172: 5-(pentafluoro-λ⁶-sulfanyl)-N-(1-{4-[3-(propan-2-yl)-[1,2,4]triazolo[4,3-a]pyridin-6-yl]benzenesulfonyl}piperidin-4-yl)pyridin-2-amine

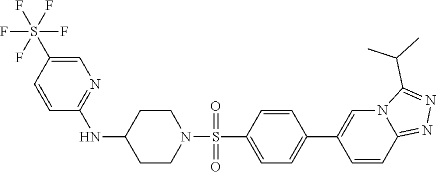 [SMILES: CC(C)c1nnc2ccc(-c3ccc(S(=O)(=O)N4CCC(Nc5ccc(S(F)(F)(F)(F)F)cn5)CC4)cc3)cn12]

i) Under a nitrogen atmosphere, tert-butyl 4-aminopiperidine-1-carboxylate (217 mg), 2-chloro-5-(pentafluoro-λ⁶-sulfanyl)pyridine (249 mg) and NaOtBu (145 mg) were added to a solution of Pd(OAc)₂ (29 mg) and Josiphos SL J009-1 (58 mg) in THF (10 mL). The reaction mixture was stirred for 18 hours at 100° C. and after cooling to room temperature, the reaction mixture was poured into water and the product was extracted into CH₂Cl₂. The combined organic layers were dried over a phase separation filter, and concentrated under reduced pressure. The obtained orange solid was purified on SiO₂, using 0% to 100% ethyl acetate in heptane as the eluent, to give tert-butyl 4-{[5-(pentafluoro-λ⁶-sulfanyl)pyridin-2-yl]amino}piperidine-1-carboxylate (247 mg) as a white solid.
ii) To a solution of the product obtained in the previous step (247 mg) in ethyl acetate (5 mL), was added at 0° C., a 6N HCl solution in 2-propanol (2.5 mL). The reaction mixture was stirred overnight at room temperature. The reaction mixture was concentrated under reduced pressure giving 5-(pentafluoro-λ⁶-sulfanyl)-N-(piperidin-4-yl)pyridin-2-amine hydrochloride (230 mg) as a white solid, which was used in the next step without further purification.
iii) To a solution of the product obtained in the previous step (131 mg) and Et₃N (0.14 mL) in CH₂Cl₂ (4 mL) was added 4-bromobenzenesulfonyl chloride (87 mg). The reaction mixture was stirred overnight at room temperature. The reaction mixture was quenched by pouring it into water. The product was extracted into CH₂Cl₂ and the combined organic layers were dried a phase separation filter and concentrated under vacuo. The obtained yellow solid was purified on SiO₂, using 0% to 100% ethyl acetate in heptane as the eluent, to give N-[1-(4-bromobenzenesulfonyl)piperidin-4-yl]5-(pentafluoro-λ⁶-sulfanyl)pyridin-2-amine (306 mg) as an white solid.
iv) Following a procedure described for Example 1, step ii, the product obtained in the previous step (50 mg) and [3-(propan-2-yl)-[1,2,4]triazolo[4,3-a]pyridin-6-yl]boronic acid (100 mg) were converted to the title compound 5-pentafluoro-λ⁶-sulfanyl)-N-(1-{4-[3-propan-2-yl)-[1,2,4]triazolo[4,3-a]pyridin-6-yl]benzenesulfonyl}piperidin-4-yl)pyridin-2-amine (10 mg) as a white solid. MS(ES⁺) m/z 603.3 (M+H)⁺.

¹H NMR (400 MHz, DMSO) δ 8.87-8.82 (t, J=1.3 Hz, 1H), 8.39-8.34 (d, J=2.8 Hz, 1H), 8.13-8.06 (m, 2H), 7.92-7.84 (m, 3H), 7.81-7.74 (m, 2H), 7.53-7.47 (d, J=7.3 Hz, 1H), 6.55-6.48 (d, J=9.4 Hz, 1H), 3.87-3.68 (m, 1H), 3.64-3.56 (m, 3H), 2.64-2.55 (m, 2H), 2.01-1.94 (m, 2H), 1.59-1.47 (m, 2H), 1.46-1.44 (s, 3H), 1.43-1.41 (s, 3H).

Following a procedure analogous to that described for Example 172, using in step iv the appropriate boronic ester/acid, Example 173-174 have been prepared.

### Example 173: N-[1-(4-{imidazo[1,2-a]pyridin-6-yl}benzenesulfonyl)piperidin-4-yl]-5-(pentafluoro-λ⁶-sulfanyl)pyridin-2-amine

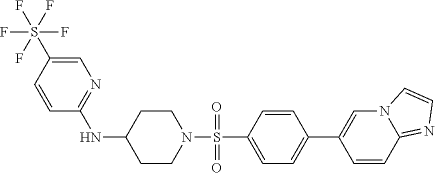 [SMILES: O=S(=O)(c1ccc(-c2ccc3nccn3c2)cc1)N1CCC(Nc2ccc(S(F)(F)(F)(F)F)cn2)CC1]

MS(ES⁺) m/z 560.4 (M+H)⁺

Building block: Step iv: 6-(4,4,5,5-tetramethyl-1,3,2-dioxaborolan-2-yl)imidazo[1,2-a]pyridine.

### Example 174: N-[1-(4-{3-methyl-[1,2,4]triazolo[4,3-a]pyridin-6-yl}benzenesulfonyl)piperidin-4-yl]-5-(pentafluoro-λ⁶-sulfanyl)pyridin-2-amine

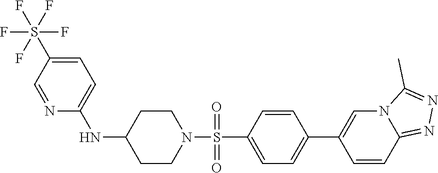 [SMILES: Cc1nnc2ccc(-c3ccc(S(=O)(=O)N4CCC(Nc5ccc(S(F)(F)(F)(F)F)cn5)CC4)cc3)cn12]

MS(ES⁺) m/z 575.4 (M+H)⁺

Building block: Step iv: {3-methyl-[1,2,4]triazolo[4,3-a]pyridin-6-yl}boronic acid.

### Example 175: 4-{4-[(4-{[5-(pentafluoro-λ⁶-sulfanyl)pyridin-2-yl]amino}piperidin-1-yl)sulfonyl]phenyl}pyridine-2-carboxamide

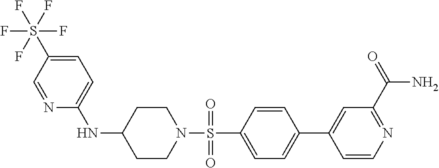 [SMILES: NC(=O)c1cc(-c2ccc(S(=O)(=O)N3CCC(Nc4ccc(S(F)(F)(F)(F)F)cn4)CC3)cc2)ccn1]

MS(ES⁺) m/z 564.4 (M+H)⁺

Building block: Step iv: 4-(4,4,5,5-tetramethyl-1,3,2-dioxaborolan-2-yl)pyridine-2-carboxamide.

Following a procedure analogous to that described for Example 33, using in step ii the appropriate aryl halide, Example 176 has been prepared.

### Example 176: 4-(pentafluoro-λ⁶-sulfanyl)-N-[trans-4-{4-[3-(difluoromethyl)-[1,2,4]triazolo[4,3-a]pyridin-6-yl]benzenesulfonyl}cyclohexyl]aniline

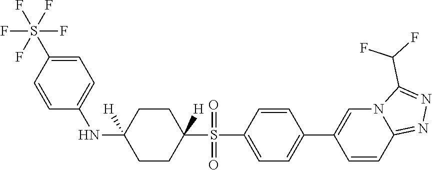 [SMILES: O=S(=O)(c1ccc(-c2ccc3nnc(C(F)F)n3c2)cc1)[C@H]1CC[C@H](Nc2ccc(S(F)(F)(F)(F)F)cc2)CC1]

MS(ES⁺) m/z 609.3 (M+H)⁺

Building block: Step ii: 3-bromo-N-(propan-2-yl)benzamide.

Following a procedure analogous to that described for Example 1, using in step ii the appropriate boronic acid/ester, Example 177 has been prepared.

### Example 177: 4-{4-[(4-{[4-(pentafluoro-λ⁶-sulfanyl)phenyl]amino}piperidin-1-yl)sulfonyl]phenyl}pyridine-2-carboxamide

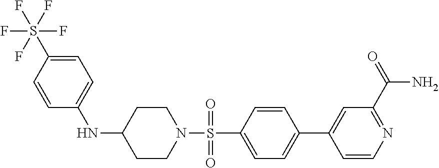 [SMILES: NC(=O)c1cc(-c2ccc(S(=O)(=O)N3CCC(Nc4ccc(S(F)(F)(F)(F)F)cc4)CC3)cc2)ccn1]

MS(ES⁺) m/z 563.3 (M+H)⁺

Building block: Step ii: 4-(4,4,5,5-tetramethyl-1,3,2-dioxaborolan-2-yl)pyridine-2-carboxamide.

### Example 178: 5-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]pyridin-2-amine

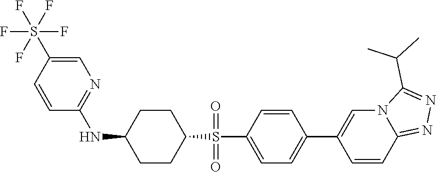 [SMILES: CC(C)c1nnc2ccc(-c3ccc(S(=O)(=O)[C@H]4CC[C@H](Nc5ccc(S(F)(F)(F)(F)F)cn5)CC4)cc3)cn12]

i) A solution of methanesulfonyl chloride (12 mL) in CH₂Cl₂ (100 mL) was added dropwise to a solution of tert-butyl N-[(1s,4s)-4-hydroxycyclohexyl]carbamate (25 g) and triethyl amine (21 mL) in CH₂Cl₂ (100 mL) at 0° C. The reaction mixture was stirred overnight at room temperature. After completion the reaction mixture was quenched by pouring into a saturated aqueous NaHCO₃ solution. The layers were separated, and the aqueous layers were extracted with ethyl acetate. The combined organic layers were washed brine, dried over MgSO₄, filtered, and concentrated under reduced pressure, to give tert-butyl N-[(1s,4s)-4-(methanesulfonyloxy)cyclohexyl]carbamate (36.3 g) as an off white solid. The product was used in the next step without further purification.
ii) 4-bromobenzene-1-thiol (39.5 g) was added to a suspension of the product obtained in the previous step (34.1 g) and cesium carbonate (94.5 g) in acetone (550 mL). The reaction mixture was stirred overnight at 60° C. After cooling to room temperature, the reaction mixture was filtered, and the solids were washed with ethyl acetate. The filtrate was concentrated under reduced pressure and the obtained oil was purified on SiO₂, using 0% to 60% ethyl acetate in heptane as the eluent, giving tert-butyl N-[(1r,4r)-4-[(4-bromophenyl)sulfanyl]cyclohexyl]carbamate (25.4 g) as a white solid.
iii) At 0° C., 3-chloroperbenzoic acid (56.5 g) was added to a solution of the product obtained in the previous step (25.3 g) in ethyl acetate (250 mL) and the reaction mixture was stirred overnight at room temperature. The reaction mixture was washed with a saturated NaHCO₃ solution, and the combined organic layers were washed with brine, dried over MgSO₄, filtered, and concentrated under reduced pressure, giving tert-butyl N-[(1r,4r)-4-(4-bromobenzenesulfonyl)cyclohexyl]carbamate (23 g) as a white solid, which was used in the next step without further purification.
iv) To a suspension of the product obtained in the previous step (2 g), bis(pinacolato)diboron (1.7 g) and potassium acetate (1.1 g) in cyclopentyl methyl ether (15 mL), purged with N₂ gas, was added PdCl₂(dppf)·CH₂Cl₂ (166 mg). The reaction mixture was heated for 2 hours at 100° C. in a microwave. After cooling to room temperature, the reaction mixture was poured into water and the product was extracted into ethyl acetate. The organic layer was washed with brine, dried over MgSO₄ and concentrated under reduced pressure giving 5-(pentafluoro-λ⁶-sulfanyl)-N-[(1r,4r)-4-[4-(4,4,5,5-tetramethyl-1,3,2-dioxaborolan-2-yl)benzenesulfonyl]cyclohexyl]pyridin-2-amine as a brown oil which was used in the next step without further purification.
v) Under a nitrogen atmosphere, Pd(PPh₃)₄ (248 mg) was added to a solution of the product obtained in the previous step (2 g), 6-bromo-3-(propan-2-yl)-[1,2,4]triazolo[4,3-a]pyridine hydrochloride (1.43 g) and NaHCO₃ (1.44 g) in a mixture of 1,4-dioxane (40 mL) and water (10 mL). The reaction mixture was stirred for 2 hours at 110° C. in a microwave. After cooling to room temperature, the reaction mixture was poured into water and the product was extracted into ethyl acetate. The combined organic layers were washed with water, brine, dried over MgSO₄, filtered, and concentrated under reduced pressure. The obtained brown oil was purified on C18, using 10% to 100% CH₃CN in water as the eluent, to give tert-butyl N-[(1r,4r)-4-{4-[3-(propan-2-yl)-[1,2,4]triazolo[4,3-a]pyridin-6-yl]benzenesulfonyl}cyclohexyl]carbamate (2.2 g) as a white solid.
vi) To a solution of the product obtained in the previous step (1.2 g) in ethyl acetate (10 mL), was added at 0° C., a 6N HCl solution in 2-propanol (4.8 mL). The reaction mixture was stirred overnight at room temperature. The reaction mixture was concentrated under reduced pressure giving (1r,4r)-4-{4-[3-(propan-2-yl)-[1,2,4]triazolo[4,3-a]pyridin-6-yl]benzenesulfonyl}cyclohexan-1-amine hydrochloride (1.1 g) as a brown solid, which was used in the next step without further purification.
vii) Under a nitrogen atmosphere, the product obtained in the previous step (159 mg), 2-chloro-5-(pentafluoro-λ⁶-sulfanyl)pyridine (99 mg) and NaOtBu (55 mg) were added to a solution of Pd(OAc)₂ (12 mg) and Josiphos SL J009-1 (24 mg) in THF (20 mL). The reaction mixture was stirred for 18 hours at 100° C. and after cooling to room temperature, the reaction mixture was poured into water and the product was extracted into CH₂Cl₂. The combined organic layers were dried over a phase separation filter, and concentrated under reduced pressure. The obtained brown oil was purified on SiO₂, using 0% to 70% ethyl acetate in heptane as the eluent, to give the title compound 5-(pentafluoro-λ⁶-sulfanyl)-N-[(1r,4r)-4-{4-[3-(propan-2-yl)-[1,2,4]triazolo[4,3-a]pyridin-6-yl]benzenesulfonyl}cyclohexyl]pyridin-2-amine (11 mg) as a white solid. MS(ES⁺) m/z 602.3 (M+H)⁺.

¹H NMR (400 MHz, DMSO) δ 8.92-8.84 (t, J=1.4 Hz, 1H), 8.45-8.37 (d, J=2.8 Hz, 1H), 8.17-8.10 (m, 2H), 8.03-7.96 (m, 2H), 7.91-7.85 (m, 1H), 7.82-7.73 (m, 2H), 7.50-7.43 (d, J=7.4 Hz, 1H), 6.55-6.47 (d, J=9.3 Hz, 1H), 3.79-3.70 (dt, J=13.8, 6.9 Hz, 1H), 3.69-3.60 (s, 1H), 3.46-3.35 (m, 1H), 2.11-1.98 (t, J=12.3 Hz, 4H), 1.54-1.46 (d, J=14.0 Hz, 2H), 1.45-1.43 (s, 3H), 1.43-1.41 (s, 3H), 1.35-1.20 (m, 2H).

Following a procedure analogous to that described for Example 178, using in step v the appropriate aryl halide, Example 179 has been prepared.

### Example 179: 5-(pentafluoro-λ⁶-sulfanyl)-N-[trans-4-(4-{3-methyl-[1,2,4]triazolo[4,3-a]pyridin-6-yl}benzenesulfonyl)cyclohexyl]pyridin-2-amine

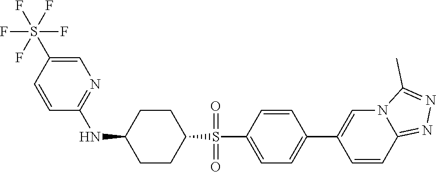 [SMILES: Cc1nnc2ccc(-c3ccc(S(=O)(=O)[C@H]4CC[C@H](Nc5ccc(S(F)(F)(F)(F)F)cn5)CC4)cc3)cn12]

MS(ES⁺) m/z 574.4 (M+H)⁺

Building block: Step v: 6-bromo-3-methyl-[1,2,4]triazolo[4,3-a]pyridine.

### Example 180: 5-(pentafluoro-λ⁶-sulfanyl)-N-[trans-4-(4-{imidazo[1,2-a]pyridin-6-yl}benzenesulfonyl)cyclohexyl]pyridin-2-amine

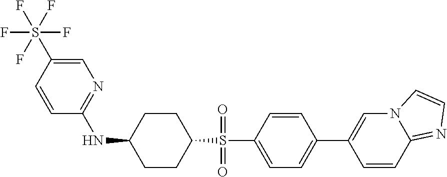 [SMILES: O=S(=O)(c1ccc(-c2ccc3nccn3c2)cc1)[C@H]1CC[C@H](Nc2ccc(S(F)(F)(F)(F)F)cn2)CC1]

i) Under a nitrogen atmosphere, Pd(PPh₃)₄ (33 mg) was added to a solution of tert-butyl N-[(1r,4r)-4-(4-bromobenzenesulfonyl)cyclohexyl]carbamate 232 mg, example 178, step iii), {imidazo[1,2-a]pyridin-6-yl}boronic acid (88 mg) and NaHCO₃ (179 mg) in a mixture of 1,4-dioxane (4 mL) and water (1 mL). The reaction mixture was stirred for 16 hours at 110° C. in a microwave. After cooling to room temperature, the reaction mixture was poured into water and the product was extracted into ethyl acetate. The combined organic layers were washed with water, brine, dried over MgSO₄, filtered, and concentrated under reduced pressure. The obtained brown oil was purified on C18, using 10% to 90% CH₃CN in water as the eluent, to give tert-butyl N-[(1r,4r)-4-(4-{imidazo[1,2-a]pyridin-6-yl}benzenesulfonyl)cyclohexyl]carbamate (71 mg) as a beige foam.
ii) To a solution of the product obtained in the previous step (71 mg) in ethyl acetate (2 mL), was added at 0° C., a 6N HCl solution in 2-propanol (3.1 mL). The reaction mixture was stirred overnight at room temperature. The reaction mixture was concentrated under reduced pressure giving (1r,4r)-4-(4-{imidazo[1,2-a]pyridin-6-yl}benzenesulfonyl)cyclohexan-1-amine (63 mg) as a white solid, which was used in the next step without further purification.
iii) Under a nitrogen atmosphere, the product obtained in the previous step (63 mg), 2-chloro-5-(pentafluoro-λ⁶-sulfanyl)pyridine (47 mg) and NaOtBu (25 mg) were added to a solution of Pd(OAc)₂ (4 mg) and Josiphos SL J009-1 (12 mg) in THF (4 mL). The reaction mixture was stirred for 18 hours at 100° C. and after cooling to room temperature, the reaction mixture was poured into water and the product was extracted into CH₂Cl₂. The combined organic layers were dried over a phase separation filter, and concentrated under reduced pressure. The obtained brown oil was purified on SiO₂, using 0% to 70% ethyl acetate in heptane as the eluent, to give the title compound 5-(pentafluoro-λ⁶-sulfanyl)-N-[(1r,4r)-4-(4-{imidazo[1,2-a]pyridin-6-yl}benzenesulfonyl)cyclohexyl]pyridin-2-amine (3 mg) as a white solid. MS(ES⁺) m/z 559.3 (M+H)⁺.

¹H NMR (400 MHz, DMSO) δ 9.15-9.09 (t, J=1.5 Hz, 1H), 8.42-8.37 (d, J=2.7 Hz, 1H), 8.06-8.02 (m, 2H), 8.02-8.00 (t, J=0.8 Hz, 1H), 7.99-7.94 (m, 2H), 7.78-7.74 (dd, J=9.3, 2.9 Hz, 1H), 7.74-7.64 (m, 3H), 7.49-7.42 (d, J=7.5 Hz, 1H), 6.55-6.45 (d, J=9.4 Hz, 1H), 3.75-3.61 (s, 1H), 3.40-3.36 (m, 1H), 2.09-1.98 (m, 4H), 1.56-1.40 (m, 2H), 1.33-1.25 (m, 2H).

Following a procedure analogous to that described for Example 180, using in step i the appropriate boronic acid/ester, Example 181 has been prepared.

### Example 181: 5-(pentafluoro-λ⁶-sulfanyl)-N-[trans-4-{4-[8-methyl-3-(propan-2-yl)-[1,2,4]triazolo[4,3-a]pyridin-6-yl]benzenesulfonyl}cyclohexyl]pyridin-2-amine

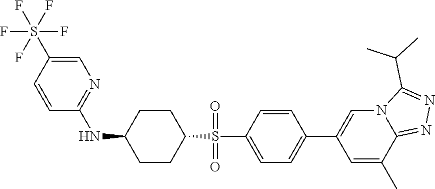 [SMILES: Cc1cc(-c2ccc(S(=O)(=O)[C@H]3CC[C@H](Nc4ccc(S(F)(F)(F)(F)F)cn4)CC3)cc2)cn2c(C(C)C)nnc12]

MS(ES⁺) m/z 616.4 (M+H)⁺

Building block: Step i: [8-methyl-3-(propan-2-yl)-[1,2,4]triazolo[4,3-a]pyridin-6-yl]boronic acid.

### Example 182: Determination of CCR6 Activity

### Human CCR6-CRE Reporter Assay

293FT cells were transfected with 2 constructs using TransIT-293 (Mirus). The first construct (pGL4.29, Promega) expressed the luciferase reporter gene luc2P in response to the binding of cAMP-induced CREB to the CREelement in its promoter. The second construct contained the open reading frame of human CCR6 in the pUNO1 backbone under the control of an enhanced CMV promoter (InVivoGen). 48 h after transfection, cells were harvested and diluted to 750,000 cells/ml in culture medium (DMEM, 10% FBS, 1× pen/strep (Gibco 15140)). Compounds were 2.5-fold serially diluted in DMSO. Further dilutions were made in assay medium (DMEM, 10% FBS, 1× pen/strep, 6 μM Forskolin, 6 nM CCL20). 4 μl of the compound solution was added to a white MW384 plate, followed by 20 μl of the cell suspension. Final DMSO concentration in the assay was 0.1%. The MW384 plate was placed in an incubator at 37° C., 5% CO2 for 5 h. Luciferase activity was determined by addition of 24 μl of a 2.5× diluted BriteLite luciferase solution (Perkin Elmer), followed by luminescence measurement using a VICTOR Plate reader (Perkin Elmer). To deselect compounds that did not specifically bind CCR6, the assay was also performed with transfected 293FT cells in which CCR6 was replaced with an empty vector using the same backbone. The relative −Log IC50 was determined with GraphPad Prism 9 using a four-parameter dose-response model.

### CHO-K1 CCR6 Chemotaxis Assay Towards CCL20

Cells overexpressing human CCR6 were used in the chemotaxis assay (DiscoverX, cAMP Hunter™ CHO-K1 CCR6 Gi Cell Line). Growth medium of these cells consisted of DMEM/F12, non-essential Amino acids (1×NEAA, Gibco 11140050), 10% FBS and 1× pen/strep (Gibco 15140). For the chemotaxis assay, Corning Transwell plates were used with a pore size of 8 m (Corning 351164). Cells were harvested using trypsin and diluted to 4*10\hat{ }6 cells/ml in chemotaxis medium (DMEM, 0.25% BSA, pen/strep). Compounds were four-fold serially diluted in DMSO. Further dilutions were made in chemotaxis medium. Cells were incubated with test compounds for 30 min at 37° C. prior to initiation of the assay (2*10\hat{ }6 cells/ml). Final DMSO concentration in the assay was 0.2%. Wells of the Transwell plates were filled with 200 μl chemotaxis medium containing 100 ng/ml CCL20 (R&D systems 360-MP). 50 μl of the cell/compound suspension was added to the inserts of the Transwell plates (100,000 cells/insert). The Transwell plates were placed in an incubator at 37° C., 5% CO2 for 4 h. After 4 h, the number of migrated cells was quantified using CellTiter Glo (Promega). Percentage inhibition values were calculated based on the low and high signal obtained with 0.2% DMSO in chemotaxis medium without and with CCL20, respectively. The relative −Log IC50 was determined with GraphPad Prism 9 using a four-parameter dose-response model.

### CD4+ T Cells Chemotaxis Assay Towards CCL20

Human CD4+ T cells were isolated from buffy coats of healthy donors using a CD4+ T Cell Isolation Kit (Miltenyi Biotec130-096-533). Isolated T cells were stimulated overnight with anti-CD3 and anti-CD28 antibodies (Biolegend 300314/302934) in growth medium (RPMI1640, 10% heat-inactivated FBS, 1× pen/strep (Gibco 15140)). For the chemotaxis assay, Corning Transwell plates were used with a pore size of 5 m (Corning 3388). T cells were counted and diluted to 6*10\hat{ }6 cells/ml in chemotaxis medium (RPMI1640, 1% BSA, pen/strep). Compounds were four-fold serially diluted in DMSO. Further dilutions were made in chemotaxis medium. Cells were incubated with test compounds for 30 min at 37° C. prior to initiation of the assay (3*10\hat{ }6 cells/ml). Final DMSO concentration in the assay was 0.2%. Wells of the Transwell plates were filled with 200 μl chemotaxis medium containing 150 ng/ml CCL20 (R&D systems 360-MP). 50 μl of the cell/compound suspension was added to the inserts of the Transwell plates (150,000 cells/insert). The Transwell plates were placed in an incubator at 37° C., 5% CO2 for 3 h. After 3 h, the number of migrated cells was quantified using CellTiter Glo (Promega). Percentage inhibition values were calculated based on the low and high signal obtained with 0.2% DMSO in chemotaxis medium without and with CCL20, respectively. The relative −Log IC50 was determined with GraphPad Prism 9 using a four-parameter dose-response model.

The tables below show the data for selected compounds:

TABLE 1
CCR6-CRE reporter assay

| Example Number | CCR6-CRE reporter |
|---|---|
| 1 | 6.9 |
| 2 | 7.2 |
| 3 | 7.3 |
| 4 | 7.3 |
| 5 | 6.3 |
| 6 | 7.4 |
| 7 | 7.4 |
| 8 | 5.9 |
| 9 | 7.1 |
| 10 | 7.1 |
| 11 | 7.0 |
| 12 | 7.1 |
| 13 | 7.1 |
| 14 | 6.7 |
| 15 | 7.1 |
| 16 | 7.2 |
| 17 | 6.1 |
| 18 | 6.8 |
| 19 | 7.2 |
| 20 | 7.0 |
| 21 | 7.5 |
| 22 | 7.4 |
| 23 | 7.3 |
| 24 | 6.7 |
| 25 | 7.4 |
| 26 | 7.3 |
| 27 | 7.2 |
| 28 | 7.5 |
| 29 | 7.4 |
| 30 | 7.3 |
| 31 | 7.2 |
| 32 | 7.0 |
| 33 | 7.2 |
| 34 | 6.7 |
| 35 | 7.1 |
| 36 | 6.9 |
| 37 | 7.2 |
| 38 | 7.4 |
| 39 | 6.6 |
| 40 | 7.0 |
| 41 | 6.8 |
| 42 | 6.6 |
| 43 | 6.8 |
| 44 | 6.8 |
| 45 | 5.8 |
| 46 | 6.6 |
| 47 | 7.2 |
| 48 | 7.4 |
| 49 | 7.1 |
| 50 | 6.9 |
| 51 | 7.2 |
| 52 | 7.4 |
| 53 | 7.1 |
| 54 | 7.3 |
| 55 | 7.5 |
| 56 | 7.2 |
| 57 | 6.7 |
| 58 | 6.7 |
| 59 | 6.8 |
| 60 | 6.8 |
| 61 | 6.9 |
| 62 | 6.4 |
| 63 | 7.4 |
| 64 | 7.3 |
| 65 | 7.4 |
| 66 | 7.2 |
| 67 | 6.2 |
| 68 | 6.0 |
| 69 | 6.1 |
| 70 | 5.9 |
| 71 | 6.0 |
| 72 | 5.8 |
| 73 | 6.3 |
| 74 | 6.3 |
| 75 | 6.0 |
| 76 | 6.1 |
| 77 | 6.3 |
| 78 | 5.8 |
| 79 | 5.8 |
| 80 | 5.7 |
| 81 | 6.9 |
| 82 | 6.7 |
| 83 | 6.5 |
| 84 | 7.3 |
| 85 | 7.0 |
| 86 | 6.9 |
| 87 | 6.7 |
| 88 | 6.1 |
| 89 | 6.8 |
| 90 | 6.8 |
| 91 | 6.9 |
| 92 | 6.7 |
| 93 | 6.4 |
| 94 | 6.3 |
| 95 | 6.6 |
| 96 | 6.8 |
| 97 | 6.0 |
| 98 | 6.7 |
| 99 | 6.7 |
| 100 | 6.1 |
| 101 | 6.5 |
| 102 | 6.9 |
| 103 | 5.9 |
| 104 | 6.4 |
| 105 | 6.4 |
| 106 | 5.8 |
| 107 | 5.8 |
| 108 | 6.3 |
| 109 | 5.7 |
| 110 | 6.5 |
| 111 | 6.0 |
| 112 | 6.4 |
| 113 | 6.5 |
| 114 | 6.4 |
| 115 | 6.4 |
| 116 | 6.7 |
| 117 | 6.6 |
| 118 | 7.1 |
| 119 | 6.5 |
| 120 | 7.2 |
| 121 | 6.6 |
| 122 | 6.4 |
| 123 | 6.6 |
| 124 | 6.7 |
| 125 | 6.7 |
| 126 | 6.9 |
| 127 | 6.5 |
| 128 | 6.9 |
| 129 | 6.7 |
| 130 | 6.7 |
| 131 | 6.5 |
| 132 | 7.1 |
| 133 | 7.4 |
| 134 | 6.8 |
| 135 | 6.9 |
| 136 | 6.9 |
| 137 | 6.7 |
| 138 | 7.2 |
| 139 | 7.2 |
| 140 | 7.2 |
| 141 | 7.1 |
| 142 | 6.6 |
| 143 | 7.0 |
| 144 | 7.3 |
| 145 | 5.8 |
| 146 | 6.1 |
| 147 | 6.7 |
| 148 | 7.3 |
| 149 | 7.2 |
| 150 | 7.2 |
| 151 | 7.2 |
| 152 | 7.0 |
| 153 | 6.8 |
| 154 | 6.9 |
| 155 | 6.8 |
| 156 | 6.8 |
| 157 | 7.1 |
| 158 | 7.0 |
| 159 | 7.2 |
| 160 | 7.3 |
| 161 | 7.1 |
| 162 | 7.0 |
| 163 | 7.1 |
| 164 | 6.9 |
| 165 | 6.1 |
| 166 | 6.1 |
| 167 | 6.6 |
| 168 | 6.6 |
| 169 | 6.5 |
| 170 | 6.3 |
| 171 | 7.0 |
| 172 | 7.7 |
| 173 | 7.5 |
| 174 | 7.6 |
| 175 | 7.7 |
| 176 | 6.9 |
| 177 | 7.1 |
| 178 | 7.2 |
| 179 | 7.1 |
| 180 | 7.0 |
| 181 | 7.6 |

TABLE 2
CHO-K1 CCR6 chemotaxis assay

| Example Number | CHO-K1 CCR6 chemotaxis |
|---|---|
| 1 | 7.1 |
| 6 | 7.6 |
| 10 | 8.3 |
| 11 | 7.6 |
| 13 | 8.3 |
| 14 | 5.9 |
| 15 | 7.2 |
| 16 | 7.9 |
| 18 | 6.7 |
| 19 | 6.4 |
| 20 | 6.4 |
| 21 | 7.9 |
| 22 | 7.0 |
| 23 | 7.3 |
| 24 | 6.3 |
| 26 | 7.0 |
| 27 | 7.2 |
| 28 | 8.3 |
| 29 | 7.3 |
| 30 | 6.6 |
| 33 | 8.1 |
| 34 | 7.4 |
| 35 | 8.4 |
| 36 | 5.8 |
| 37 | 8.4 |
| 39 | 7.1 |
| 41 | 7.1 |
| 42 | 7.8 |
| 43 | 6.5 |
| 44 | 6.8 |
| 47 | 6.1 |
| 48 | 7.4 |
| 49 | 6.3 |
| 50 | 7.0 |
| 51 | 8.1 |
| 58 | 7.8 |
| 59 | 7.8 |
| 60 | 8.1 |
| 61 | 7.5 |
| 62 | |
| 63 | 7.9 |
| 66 | 7.4 |
| 69 | 6.1 |
| 73 | 6.2 |
| 74 | 6.1 |
| 76 | 6.3 |
| 83 | 7.3 |
| 84 | 6.9 |
| 85 | 6.9 |
| 86 | 7.1 |
| 87 | 6.9 |
| 89 | 7.1 |
| 90 | 7.2 |
| 92 | 5.8 |
| 95 | 6.3 |
| 96 | 6.3 |
| 98 | 6.6 |
| 99 | 6.3 |
| 101 | 6.1 |
| 102 | 6.8 |
| 105 | 6.2 |
| 118 | 6.4 |
| 121 | 6.8 |
| 123 | 6.7 |
| 124 | 6.9 |
| 126 | 6.9 |
| 127 | 6.6 |
| 128 | 6.6 |
| 129 | 6.5 |
| 138 | 7.7 |
| 139 | 8.0 |
| 142 | 7.6 |
| 147 | 7.4 |
| 152 | 8.0 |
| 153 | 7.8 |
| 155 | 7.5 |
| 156 | 7.4 |
| 157 | 8.6 |
| 158 | 8.0 |
| 159 | 7.9 |
| 162 | 7.2 |
| 167 | 6.6 |
| 169 | 7.1 |
| 170 | 5.7 |
| 171 | |
| 172 | 8.1 |
| 173 | 8.4 |
| 174 | 8.3 |
| 175 | 8.0 |
| 176 | 8.0 |
| 177 | 7.1 |
| 178 | 8.0 |
| 179 | 8.0 |
| 180 | 7.6 |

TABLE 3
CD4+ T cells chemotaxis assay

| Example Number | CD4+ T-cell chemotaxis |
|---|---|
| 2 | 8.3 |
| 3 | 7.8 |
| 4 | 9.1 |
| 6 | 9.2 |
| 7 | 9.3 |
| 9 | 8.0 |
| 10 | 8.7 |
| 11 | 8.4 |
| 12 | 7.8 |
| 18 | 8.2 |
| 20 | 7.5 |
| 25 | 8.3 |
| 26 | 7.8 |
| 27 | 8.3 |
| 32 | 8.4 |
| 34 | 7.2 |
| 35 | 9.3 |
| 38 | 9.2 |
| 40 | 8.0 |
| 41 | 8.3 |
| 50 | 7.6 |
| 51 | 8.7 |
| 56 | 7.8 |
| 77 | 6.9 |
| 81 | 6.8 |
| 82 | 6.5 |
| 85 | 7.6 |
| 86 | 7.1 |
| 87 | 6.8 |
| 89 | 7.4 |
| 90 | 7.6 |
| 91 | 7.5 |
| 102 | 7.3 |
| 125 | 6.9 |
| 130 | 7.3 |
| 131 | 6.6 |
| 132 | 7.7 |
| 133 | 8.4 |
| 134 | 8.0 |
| 135 | 7.6 |
| 136 | 6.7 |
| 137 | 6.9 |
| 140 | 8.6 |
| 148 | 8.3 |
| 150 | 9.0 |
| 151 | 8.2 |
| 154 | 8.1 |
| 160 | 8.4 |
| 161 | 8.1 |
| 162 | 7.1 |
| 163 | 7.8 |
| 164 | 6.7 |
| 168 | 7.0 |
| 170 | 5.0 |
| 172 | 9.0 |
| 173 | 8.6 |
| 174 | 8.6 |
| 175 | 8.3 |
| 176 | 8.8 |
| 177 | 7.9 |
| 178 | 9.1 |
| 179 | 8.2 |
| 180 | 8.1 |

### ASPECTS

Aspect 1. A compound of formula (I)

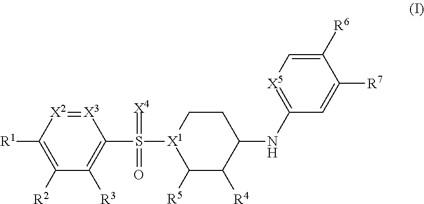

wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a};
R^{1a} is C₁₋₆alkyl, cyano, —NR^{1b}R^{1c}, —C₁₋₆alkyl-NR^{1b}R^{1c}, oxo, —CONR^{1b}R^{1c}, —PO(Me)₂, hydroxy, C₃₋₆cycloalkyl, —C₁₋₆alkyl-C₁₋₆alkoxy, C₃₋₆heterocyclyl, haloC₁₋₆alkyl, halo, C₁₋₆alkoxy;
R^{1b} is hydrogen, C₃₋₆cycloalkyl, or C₁₋₆alkyl;
R^{1c} is hydrogen, C₃₋₆cycloalkyl, or C₁₋₆alkyl;
R² is hydrogen or halogen;
R³ is hydrogen or halogen;
R⁴ is hydrogen, C₁₋₆alkyl, or halogen;
R⁵ is hydrogen, halogen, or C₁₋₆alkyl;
R⁶ is hydrogen or —SF₅, wherein R⁶ and R⁷ must be different;
R⁷ is hydrogen or —SF₅, wherein R⁶ and R⁷ must be different;
or a pharmaceutically acceptable salt thereof.

Aspect 2. The compound of aspect 1 wherein
R^{1a} is C₁₋₆alkyl, cyano, oxo, —CONR^{1b}R^{1c}, —PO(Me)₂, hydroxy, C₃₋₆cycloalkyl, —C₁₋₆alkylC₁₋₆alkoxy, haloC₁₋₆alkyl, halo, C₁₋₆alkoxy;
R^{1b} is hydrogen or C₁₋₆alkyl;
R^{1c} is hydrogen or C₁₋₆alkyl;
X⁵ is CH;
R⁵ is hydrogen;
or a pharmaceutically acceptable salt thereof.

Aspect 3. The compound of aspects 1 or 2, or a pharmaceutically acceptable salt thereof, wherein X¹ is CH.

Aspect 4. The compound of aspects 1 or 2, or a pharmaceutically acceptable salt thereof, wherein X¹ is N.

Aspect 5. The compound of any one of aspects 1 to 4, or a pharmaceutically acceptable salt thereof, wherein X² is CH.

Aspect 6. The compound of any one of aspects 1 to 4, or a pharmaceutically acceptable salt thereof, wherein X² is N.

Aspect 7. The compound of any one of aspects 1 to 6, or a pharmaceutically acceptable salt thereof, wherein X³ is CH.

Aspect 8. The compound of any one of aspects 1 to 6, or a pharmaceutically acceptable salt thereof, wherein X³ is N.

Aspect 9. The compound of any one of aspects 1 to 8, or a pharmaceutically acceptable salt thereof, wherein X⁴ is O.

Aspect 10. The compound of any one of aspects 1 to 8, or a pharmaceutically acceptable salt thereof, wherein X⁴ is NH.

Aspect 11. The compound of any one of aspects 1 or 3-10, or a pharmaceutically acceptable salt thereof, wherein X⁵ is N.

Aspect 12. The compound of any one of aspects 1 to 10, or a pharmaceutically acceptable salt thereof, wherein X⁵ is CH.

Aspect 13. The compound of any one of aspects 1-2, 4-5, 7, 9 or 12, or a pharmaceutically acceptable salt thereof, wherein X¹ is N, X² is CH, X³ is CH, X⁴ is O, and X⁵ is CH.

Aspect 14. The compound of any one of aspects 1-3, 5, 7, 9 or 12, or a pharmaceutically acceptable salt thereof, wherein X¹ is CH, X² is CH, X³ is CH, X⁴ is O, and X⁵ is CH.

Aspect 15. The compound of any one of aspects 1-2, 4, 6-7, 9 or 12, or a pharmaceutically acceptable salt thereof, wherein X¹ is N, X² is N, X³ is CH, X⁴ is O, and X⁵ is CH.

Aspect 16. The compound of any one of aspects 1-2, 4-5, 8-9 or 12, or a pharmaceutically acceptable salt thereof, wherein X¹ is N, X² is CH, X³ is N, X⁴ is O, and X⁵ is CH.

Aspect 17. The compound of any one of aspects 1-3, 5, 7, 10 or 12, or a pharmaceutically acceptable salt thereof, wherein X¹ is CH, X² is CH, X³ is CH, X⁴ is NH, and X⁵ is CH.

Aspect 18. The compound of any one of aspects 1-2, 4-5, 7, 10 or 12, or a pharmaceutically acceptable salt thereof, wherein X¹ is N, X² is CH, X³ is CH, X⁴ is NH, and X⁵ is CH.

Aspect 19. The compound of any one of aspects 1 to 18, or a pharmaceutically acceptable salt thereof, wherein R¹ is azaspirononanyl, triazaspirodecanyl, diazaspirodecanyl, oxadiazaspirodecanyl, oxaazaspirononanyl, oxaazabicyclooctanyl, pyrazolopyridinyl, pyrrolopyrimidinyl, pyrrolopyridinyl, thiophenyl, thienopyridinyl, pyrrolopyrazinyl, pyrrolodiazepinyl, furanyl, indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, or spirocyclopropaneindolinyl, wherein azaspirononanyl, triazaspirodecanyl, diazaspirodecanyl, oxadiazaspirodecanyl, oxaazaspirononanyl, oxaazabicyclooctanyl, pyrazolopyridinyl, pyrrolopyrimidinyl, pyrrolopyridinyl, thiophenyl, thienopyridinyl, pyrrolopyrazinyl, pyrrolodiazepinyl, furanyl, indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, and spirocyclopropaneindolinyl are optionally substituted with one or more R^{1a}.

Aspect 20. The compound of any one of aspects 1 to 19, or a pharmaceutically acceptable salt thereof, wherein R¹ is indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, or spirocyclopropaneindolinyl, wherein indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, and spirocyclopropaneindolinyl are optionally substituted with one or more R^{1a}.

Aspect 21. The compound of any one of aspects 1 to 20, or a pharmaceutically acceptable salt thereof, wherein R¹ is indazolyl, indolyl, pyridyl, triazolopyridinyl, phenyl, or imidazopyridinyl, wherein indazolyl, indolyl, pyridyl, triazolopyridinyl, phenyl, and imidazopyridinylare optionally substituted with one or more R^{1a}.

Aspect 22. The compound of any one of aspects 1 to 21, or a pharmaceutically acceptable salt thereof, wherein R¹ is methylindazolyl, cyano-indolyl, cyano-indazolyl, oxo-isoindolinyl, Carbamoylpyridyl, methyl-triazolopyridinyl, imidazopyridinyl, cyano-imidazopyridinyl, imidazopyrazinyl, methylimidazopyridinyl, carbamoylimidazopyridinyl, isopropyl-triazolopyridinyl, triazolopyridinyl, carbamoylindazolyl, oxo-indolinyl, carbamoyl-triazolopyridinyl, cyano-triazolopyridinyl, (dimethylcarbamoyl)phenyl, methylimidazopyrazinyl, (dimethylcarbamoyl)pyridyl, (methylcarbamoyl)phenyl, (methylcarbamoyl)pyridyl, oxo-triazolopyridinyl, carbamoylphenyl, isopropyl-triazolopyridazinyl, oxo-dihydro-isoquinolinyl, carbamoylpyridazinyl, dimethylphosphorylphenyl, isopropyl-triazolopyrazinyl, oxo-pyridinyl, oxo-pyrrolotriazinyl, hydroxypyridyl, (dimethylcarbamoyl)pyridyl, oxo-spirocyclopropaneindolineyl, methoxyazaspirononanyl, oxo-triazaspirodecanyl, oxo-diazaspirodecanyl, oxo-oxadiazaspirodecanyl, oxaazaspirononanyl, oxaazabicyclooctanyl, carbamoylfluorophenyl, propanyltriazolopyrazinyl, isopropycarbamoylphenyl, methylpyrazolopyridinyl, N,N-dimethylamineimidazopyridinyl, cyclopropylcarbamoylphenyl, pyrrolopyrimidinyl, pyrrolopyridinyl, pyrazolopyridinyl, carbamoylthiophenyl, methylimidazopyridinyl, (trifluoromethyl)imidazopyridinyl, fluoroimidazopyridinyl, oxo-thienopyridinyl, oxo-pyrrolopyrazinyl, oxo-pyrrolodiazepinyl, carbamoylimidazopyridinyl, furancarboxamide, cyano-imidazopyridinyl, pyrrolidinyl-triazolopyridinyl, propanyl-triazolopyrazinyl, or azetidinyl-triazolopyridinyl.

Aspect 23. The compound of any one of aspects 1 to 22, or a pharmaceutically acceptable salt thereof, wherein R¹ is methylindazolyl, cyanoindolyl, cyanoindazolyl, oxoisoindolinyl, carbamoylpyridyl, methyl-triazolopyridinyl, imidazopyridinyl, cyanoimidazopyridinyl, imidazopyrazinyl, methylimidazopyridinyl, carbamoylimidazopyridinyl, isopropyl-triazolopyridinyl, triazolopyridinyl, carbamoylindazolyl, oxoindolinyl, carbamoyl-triazolopyridinyl, methyl-triazolopyridinyl, methyl-triazolopyridinyl, cyano-triazolopyridinyl, (dimethylcarbamoyl)phenyl, methylimidazopyrazinyl, (dimethylcarbamoyl)pyridyl, (methylcarbamoyl)phenyl, (methylcarbamoyl)pyridyl, oxo-triazolopyridinyl, carbamoylphenyl, carbamoylpyridyl, isopropyl-triazolopyridazinyl, oxo-dihydro-isoquinolinyl, carbamoylpyridazinyl, dimethylphosphorylphenyl, oxoisoindolinyl, oxoindolinyl, oxo-dihydro-isoquinolinyl, isopropyl-triazolopyrazinyl, oxo-pyridinyl, oxo-dihydro-isoquinolinyl, oxo-pyrrolotriazinyl, hydroxypyridyl, methyl-triazolopyridinyl, imidazopyridinyl, triazolopyridinyl, carbamoylphenyl, carbamoylpyridyl, (dimethylcarbamoyl)pyridyl, oxospirocyclopropaneindolineyl.

Aspect 24. The compound of any one of aspects 1 to 23, or a pharmaceutically acceptable salt thereof, wherein R¹ is methyl-indazolyl, cyano-indolyl, carbamoyl-pyridyl, methyl-imidazopyridinyl, methyl-triazolopyridinyl, isopropyl-triazolopyridinyl, cyano-indazolyl, (methylcarbamoyl)phenyl, imidazopyridinyl, cyanoimidazopyridinyl.

Aspect 25. The compound of any one of aspects 1 to 24, or a pharmaceutically acceptable salt thereof, wherein R^{1a} is methoxy, fluoro, —CONH[(CH(CH3)₂], —CONH(cyclopropyl), dimethylamino, —CF₃, pyrrolidinyl, azetidinyl, methyl, cyano, oxo, —CONH₂, isopropyl, —CON(Me)₂, —CH₂—O—CH₃, —CONH(Me), —PO(Me)₂, —OH.

Aspect 26. The compound of any one of aspects 1 to 25, or a pharmaceutically acceptable salt thereof, wherein R^{1a} is methyl, cyano, oxo, —CONH₂, isopropyl, —CON(Me)₂, —CH₂—O—CH₃, —CONH(Me), —PO(Me)₂, —OH.

Aspect 27. The compound of any one of aspects 1 to 26, or a pharmaceutically acceptable salt thereof, wherein R^{1a} is methyl, cyano, —CONH₂, isopropyl, —CONH(Me).

Aspect 28. The compound of any one of aspects 1 to 27, or a pharmaceutically acceptable salt thereof, wherein R² is hydrogen, fluorine, or —CF₃.

Aspect 29. The compound of any one of aspects 1 to 28, or a pharmaceutically acceptable salt thereof, wherein R² is hydrogen.

Aspect 30. The compound of any one of aspects 1 to 29, or a pharmaceutically acceptable salt thereof, wherein R³ is hydrogen, fluorine, chlorine, or —CF₃.

Aspect 31. The compound of any one of aspects 1 to 30, or a pharmaceutically acceptable salt thereof, wherein R³ is hydrogen.

Aspect 32. The compound of any one of aspects 1 to 31, or a pharmaceutically acceptable salt thereof, wherein R⁴ is hydrogen or fluorine.

Aspect 33. The compound of any one of aspects 1 to 32, or a pharmaceutically acceptable salt thereof, wherein R⁴ is hydrogen.

Aspect 34. The compound of any one of aspects 1 to 33, or a pharmaceutically acceptable salt thereof, wherein R⁶ is —SF₅.

Aspect 35. The compound of any one of aspects 1 to 34, or a pharmaceutically acceptable salt thereof, wherein R⁷ is hydrogen.

Aspect 36. The compound of any one of aspects 1 to 35, or a pharmaceutically acceptable salt thereof, wherein R⁵ is hydrogen.

Aspect 37. The compound of any one of aspects 1-3, 5-12, 14, 17 or 19-36 wherein the compound is of formula (I′)

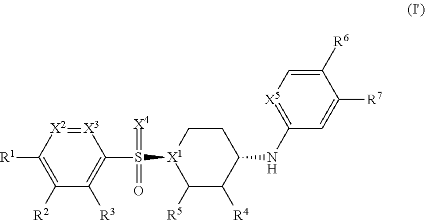

or a pharmaceutically acceptable salt thereof wherein R¹, R², R³, R⁴, R⁵, R⁶, R⁷, X², X³, X⁴, and X⁵ are as defined in any one of aspects 1-3, 5-12, 14, 17, or 19-36 and X¹ is CH.

Aspect 38. The compound of any one of aspects 1-5, 7, 9-10, 12-37, or a pharmaceutically acceptable salt thereof, wherein
X¹ is CH or N,
X² is CH,
X³ is CH,
X⁴ is O or NH,
X⁵ is CH,
R¹ is indazolyl, indolyl, pyridyl, triazolopyridinyl, phenyl, or imidazopyridinyl, wherein indazolyl, indolyl, pyridyl, triazolopyridinyl, phenyl, and imidazopyridinylare optionally substituted with one or more R^{1a}.
R^{1a} is methyl, cyano, —CONH₂, isopropyl, —CONH(Me),
R² is hydrogen,
R³ is hydrogen,
R⁴ is hydrogen,
R⁵ is hydrogen,
R⁶ is —SF₅,
R⁷ is hydrogen.

Aspect 39. The compound of any one of aspects 1-5, 7, 9-10, 12-38, or a pharmaceutically acceptable salt thereof, wherein
X¹ is CH or N,
X² is CH,
X³ is CH,
X⁴ is O or NH,
X⁵ is CH,
R¹ is methyl-indazolyl, cyano-indolyl, carbamoyl-pyridyl, methyl-imidazopyridinyl, methyl-triazolopyridinyl, isopropyl-triazolopyridinyl, cyano-indazolyl, (methylcarbamoyl)phenyl, imidazopyridinyl, cyanoimidazopyridinyl,
R² is hydrogen,
R³ is hydrogen,
R⁴ is hydrogen,
R⁵ is hydrogen,
R⁶ is —SF₅,
R⁷ is hydrogen.

Aspect 40. The compound of any one of aspects 1 to 39, or a pharmaceutical acceptable salt thereof, selected from:
1-(4-{imidazo[1,2-a]pyridin-6-yl}benzenesulfonyl)-N-[4-(pentafluoro-λ⁶-sulfanyl)phenyl]piperidin-4-amine
1-[4-(3-methyl-2H-indazol-5-yl)benzenesulfonyl]-N-[4-(pentafluoro-λ⁶-sulfanyl)phenyl]piperidin-4-amine
5-{4-[(4-{[4-(pentafluoro-λ⁶-sulfanyl)phenyl]Amino}piperidin-1-yl)sulfonyl]phenyl}-1H-indole-3-carbonitrile
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[cis-4-[4-(3-methyl-2H-indazol-5-yl)benzenesulfonyl]cyclohexyl]aniline
4-(pentafluoro-λ⁶-sulfanyl)-N-[trans-4-[4-(3-methyl-2H-indazol-5-yl)benzenesulfonyl]cyclohexyl]aniline
5-(4-{[trans-4-{[4-(pentafluoro-λ⁶-sulfanyl)phenyl]Amino}cyclohexyl]sulfonyl}phenyl)-1H-indole-3-carbonitrile
5-(4-{[cis-4-{[4-(pentafluoro-λ⁶-sulfanyl)phenyl]Amino}cyclohexyl]sulfonyl}phenyl)-1H-indole-3-carbonitrile
6-(4-{[trans-4-{[4-(pentafluoro-λ⁶-sulfanyl)phenyl]Amino}cyclohexyl]sulfonyl}phenyl)-2,3-dihydro-1H-isoindol-1-one
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(pentafluoro-λ⁶-sulfanyl)-N-[trans-4-(4-{imidazo[1,2-a]pyridin-6-yl}benzenesulfonyl)cyclohexyl]aniline
4-(pentafluoro-λ⁶-sulfanyl)-N-[trans-4-(4-{3-methylimidazo[1,2-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-indol-2-one
N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide
N-methyl-4′-{[trans-4-{[4-(pentafluoro-λ⁶-sulfanyl)phenyl]Amino}cyclohexyl]sulfonyl}-[1,1′-biphenyl]-4-carboxamide
6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile
4-(pentafluoro-λ⁶-sulfanyl)-N-[cis-4-(4-{imidazo[1,2-a]pyridin-6-yl}benzenesulfonyl)cyclohexyl]aniline
N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonyl}phenyl)pyridine-2-carboxamide
N-methyl-4′-{[trans-4-{[4-(pentafluoro-λ⁶-sulfanyl)phenyl]Amino}cyclohexyl]sulfonyl}-[1,1′-biphenyl]-3-carboxamide
N-methyl-5-(4-{[trans-4-{[4-(pentafluoro-λ⁶-sulfanyl)phenyl]Amino}cyclohexyl]sulfonyl}phenyl)pyridine-2-carboxamide
4′-{[trans-4-{[4-(pentafluoro-λ⁶-sulfanyl)phenyl]Amino}cyclohexyl]sulfonyl}-[1,1′-biphenyl]-3-carboxamide
1-(4-{imidazo[1,2-a]pyrazin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-)⁶-sulfanyl)phenyl]piperidin-4-amine
N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
N-[4-(pentafluoro-λ⁶-sulfanyl)phenyl]-1-{4-[3-(propan-2-yl)imidazo[1,2-a]pyridin-6-yl]benzenesulfonyl}piperidin-4-amine
1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine
1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile
6-(4-{[trans-4-{[4-(pentafluoro-λ⁶-sulfanyl)phenyl]Amino}cyclohexyl]sulfonyl}phenyl)imidazo[1,2-a]pyridine-3-carboxamide
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-(4-{[1,2,4]triazolo[4,3-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-1H-indazole-3-carboxamide
5-(4-{[trans-4-{[4-(pentafluoro-λ⁶-sulfanyl)phenyl]Amino}cyclohexyl]sulfonyl}phenyl)-1H-indazole-3-carbonitrile
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide
4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline
4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile
4-(pentafluoro-λ⁶-sulfanyl)-N-[trans-4-(4-{2-methylimidazo[1,2-a]pyrazin-6-yl}benzenesulfonyl)cyclohexyl]aniline
4-(pentafluoro-λ⁶-sulfanyl)-N-[trans-4-(4-{imidazo[1,2-a]pyrazin-6-yl}benzenesulfonyl)cyclohexyl]aniline
N,N-dimethyl-6-(4-{[cis-4-{[4-(pentafluoro-λ⁶-sulfanyl)phenyl]Amino}cyclohexyl]sulfonyl}phenyl)pyridine-3-carboxamide
N,N-dimethyl-6-(4-{[trans-4-{[4-(pentafluoro-λ⁶-sulfanyl)phenyl]Amino}cyclohexyl]sulfonyl}phenyl)pyridine-3-carboxamide
N-methyl-6-(4-{[trans-4-{[4-(pentafluoro-λ⁶-sulfanyl)phenyl]Amino}cyclohexyl]sulfonyl}phenyl)pyridine-3-carboxamide
N-methyl-4-(4-{[trans-4-{[4-(pentafluoro-λ⁶-sulfanyl)phenyl]Amino}cyclohexyl]sulfonyl}phenyl)pyridine-2-carboxamide
6-(4-{[trans-4-{[4-(pentafluoro-λ⁶-sulfanyl)phenyl]Amino}cyclohexyl]sulfonyl}phenyl)pyridine-3-carboxamide
4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
4-(4-{[trans-4-{[4-(pentafluoro-λ⁶-sulfanyl)phenyl]Amino}cyclohexyl]sulfonyl}phenyl)-1,2-dihydropyridin-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one
5-(4-{[trans-4-{[4-(pentafluoro-λ⁶-sulfanyl)phenyl]Amino}cyclohexyl]sulfonyl}phenyl)pyridin-2-ol
4-(pentafluoro-λ⁶-sulfanyl)-N-[trans-4-{[4′-(dimethylphosphoryl)-[1,1′-biphenyl]-4-yl]sulfonyl}cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-isoindol-1-one
6-(4-{[trans-4-{[4-(pentafluoro-λ⁶-sulfanyl)phenyl]Amino}cyclohexyl]sulfonyl}phenyl)-2,3-dihydro-1H-indol-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)pyridazine-3-carboxamide
4-(pentafluoro-λ⁶-sulfanyl)-N-[trans-4-{4-[3-(propan-2-yl)imidazo[1,2-a]pyridin-6-yl]benzenesulfonyl}cyclohexyl]aniline
4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline
5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one
6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one
1-(2-fluoro-4-{3-methylimidazo[1,2-a]pyridin-6-yl}benzenesulfonyl)-N-[4-(pentafluoro-λ⁶-sulfanyl)phenyl]piperidin-4-amine
1-(3-fluoro-4-{3-methylimidazo[1,2-a]pyridin-6-yl}benzenesulfonyl)-N-[4-(pentafluoro-λ⁶-sulfanyl)phenyl]piperidin-4-amine
1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine
1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine
6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(3-fluoro-4-{imidazo[1,2-a]pyridin-6-yl}benzenesulfonyl)-N-[4-(pentafluoro-λ⁶-sulfanyl)phenyl]piperidin-4-amine
6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(2-fluoro-4-{imidazo[1,2-a]pyridin-6-yl}benzenesulfonyl)-N-[4-(pentafluoro-λ⁶-sulfanyl)phenyl]piperidin-4-amine
1-[(5-{imidazo[1,2-a]pyridin-6-yl}pyridin-2-yl)sulfonyl]-N-[4-(pentafluoro-λ⁶-sulfanyl)phenyl]piperidin-4-amine
6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile
1-[(6-{imidazo[1,2-a]pyridin-6-yl}pyridin-3-yl)sulfonyl]-N-[4-(pentafluoro-λ⁶-sulfanyl)phenyl]piperidin-4-amine
6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile
1-(2-chloro-4-{imidazo[1,2-a]pyridin-6-yl}benzenesulfonyl)-N-[4-(pentafluoro-λ⁶-sulfanyl)phenyl]piperidin-4-amine
6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[3-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[3-(pentafluoro-λ⁶-sulfanyl)phenyl]piperidin-4-amine
4-(4-{[(3S,4S)-3-fluoro-4-{[4-(pentafluoro-λ⁶-sulfanyl)phenyl]Amino}piperidin-1-yl]sulfonyl}phenyl)pyridine-2-carboxamide
N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
5-(4-{[trans-4-{[4-(pentafluoro-λ⁶-sulfanyl)phenyl]Amino}cyclohexyl]sulfonimidoyl}phenyl)-1H-indole-3-carbonitrile
[4-(3-methyl-1H-indazol-5-yl)phenyl][trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
4-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide
(4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone
[trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone
N-methyl-4′-{[trans-4-{[4-(pentafluoro-λ⁶-sulfanyl)phenyl]Amino}cyclohexyl]sulfonimidoyl}-[1,1′-biphenyl]-4-carboxamide
4′-{[trans-4-{[4-(pentafluoro-λ⁶-sulfanyl)phenyl]Amino}cyclohexyl]sulfonimidoyl}-[1,1′-biphenyl]-4-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide
N-methyl-4′-{[trans-4-{[4-(pentafluoro-λ⁶-sulfanyl)phenyl]Amino}cyclohexyl]sulfonimidoyl}-[1,1′-biphenyl]-3-carboxamide
4′-{[trans-4-{[4-(pentafluoro-λ⁶-sulfanyl)phenyl]Amino}cyclohexyl]sulfonimidoyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide
N-methyl-5-(4-{[trans-4-{[4-(pentafluoro-λ⁶-sulfanyl)phenyl]Amino}cyclohexyl]sulfonimidoyl}phenyl)pyridine-2-carboxamide
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
(4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone
N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide
N-methyl-6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide
N-methyl-4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
5-(4-{[trans-4-{[4-(pentafluoro-λ⁶-sulfanyl)phenyl]Amino}cyclohexyl]sulfonimidoyl}phenyl)-2,3-dihydro-1H-indol-2-one
6-(4-{[trans-4-{[4-(pentafluoro-λ⁶-sulfanyl)phenyl]Amino}cyclohexyl]sulfonimidoyl}phenyl)-2,3-dihydro-1H-indol-2-one
{4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one
5-{4-[(4-{[4-(pentafluoro-λ⁶-sulfanyl)phenyl]Amino}piperidin-1-yl)sulfonimidoyl]phenyl}-1H-indole-3-carbonitrile
N-methyl-5-{4-[(4-{[4-(pentafluoro-λ⁶-sulfanyl)phenyl]Amino}piperidin-1-yl)sulfonimidoyl]phenyl}pyridine-2-carboxamide
4′-[(4-{[4-(pentafluoro-λ⁶-sulfanyl)phenyl]Amino}piperidin-1-yl)sulfonimidoyl]-[1,1′-biphenyl]-4-carbonitrile
5-{4-[(4-{[4-(pentafluoro-λ⁶-sulfanyl)phenyl]Amino}piperidin-1-yl)sulfonimidoyl]phenyl}-2,3-dihydro-1H-indol-2-one
(4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone
4-{4-[(4-{[4-(pentafluoro-λ⁶-sulfanyl)phenyl]Amino}piperidin-1-yl)sulfonimidoyl]phenyl}pyridine-2-carboxamide
(+)-5-(4-{[trans-4-{[4-(pentafluoro-λ⁶-sulfanyl)phenyl]Amino}cyclohexyl]sulfonimidoyl}phenyl)-1H-indole-3-carbonitrile
(−)-5-(4-{[trans-4-{[4-(pentafluoro-λ⁶-sulfanyl)phenyl]Amino}cyclohexyl]sulfonimidoyl}phenyl)-1H-indole-3-carbonitrile
(+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(+)-4-(4-{[trans-4-{[4-(pentafluoro-λ⁶-sulfanyl)phenyl]Amino}cyclohexyl]sulfonimidoyl}phenyl)pyridine-2-carboxamide
(−)-4-(4-{[trans-4-{[4-(pentafluoro-λ⁶-sulfanyl)phenyl]Amino}cyclohexyl]sulfonimidoyl}phenyl)pyridine-2-carboxamide
1-(4-{2-methoxy-7-azaspiro[3.5]nonan-7-yl}benzenesulfonyl)-N-[4-(pentafluoro-λ⁶-sulfanyl)phenyl]piperidin-4-amine,
8-{4-[(4-{[4-(pentafluoro-λ⁶-sulfanyl)phenyl]amino}piperidin-1-yl)sulfonyl]phenyl}-1,3,8-triazaspiro[4.5]decane-2,4-dione,
8-{4-[(4-{[4-(pentafluoro-λ⁶-sulfanyl)phenyl]amino}piperidin-1-yl)sulfonyl]phenyl}-2,8-diazaspiro[4.5]decan-1-one,
8-{4-[(4-{[4-(pentafluoro-λ⁶-sulfanyl)phenyl]amino}piperidin-1-yl)sulfonyl]phenyl}-3-oxa-1,8-diazaspiro[4.5]decan-2-one,
1-(4-{2-oxa-7-azaspiro[3.5]nonan-7-yl}benzenesulfonyl)-N-[4-(pentafluoro-λ⁶-sulfanyl)phenyl]piperidin-4-amine,
1-(4-{8-oxa-3-azabicyclo[3.2.1]octan-3-yl}benzenesulfonyl)-N-[4-(pentafluoro-λ⁶-sulfanyl)phenyl]piperidin-4-amine,
4-fluoro-N-methyl-4′-[(4-{[4-(pentafluoro-λ⁶-sulfanyl)phenyl]amino}piperidin-1-yl)sulfonyl]-[1,1′-biphenyl]-3-carboxamide,
3-fluoro-N-methyl-4′-[(4-{[4-(pentafluoro-λ⁶-sulfanyl)phenyl]amino}piperidin-1-yl)sulfonyl]-[1,1′-biphenyl]-4-carboxamide,
N-[4-(pentafluoro-λ⁶-sulfanyl)phenyl]-1-{4-[3-(propan-2-yl)-[1,2,4]triazolo[4,3-a]pyrazin-6-yl]benzenesulfonyl}piperidin-4-amine,
N-(propan-2-yl)-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{3-methyl-1H-pyrazolo[3,4-c]pyridin-5-yl}benzenesulfonyl)cyclohexyl]aniline,
N,N-dimethyl-6-(4-{[trans-4-{[4-(pentafluoro-λ⁶-sulfanyl)phenyl]amino}cyclohexyl]sulfonyl}phenyl)imidazo[1,2-a]pyridin-3-amine,
N-cyclopropyl-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{5H-pyrrolo[3,2-d]pyrimidin-2-yl}benzenesulfonyl)cyclohexyl]aniline,
4-(pentafluoro-λ⁶-sulfanyl)-N-[trans-4-(4-{1H-pyrrolo[2,3-c]pyridin-5-yl}benzenesulfonyl)cyclohexyl]aniline,
4-(pentafluoro-λ⁶-sulfanyl)-N-[trans-4-(4-{1H-pyrazolo[3,4-c]pyridin-5-yl}benzenesulfonyl)cyclohexyl]aniline,
4-(4-{[trans-4-{[4-(pentafluoro-λ⁶-sulfanyl)phenyl]amino}cyclohexyl]sulfonyl}phenyl)thiophene-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
4-(pentafluoro-λ⁶-sulfanyl)-N-[trans-4-(4-{3-methyl-1H-pyrazolo[3,4-b]pyridin-5-yl}benzenesulfonyl)cyclohexyl]aniline,
4-(pentafluoro-λ⁶-sulfanyl)-N-[trans-4-{4-[8-(trifluoromethyl)imidazo[1,2-a]pyridin-6-yl]benzenesulfonyl}cyclohexyl]aniline,
4-(pentafluoro-λ⁶-sulfanyl)-N-[trans-4-(4-{8-fluoroimidazo[1,2-a]pyridin-6-yl}benzenesulfonyl)cyclohexyl]aniline,
2-(4-{[trans-4-{[4-(pentafluoro-λ⁶-sulfanyl)phenyl]amino}cyclohexyl]sulfonyl}phenyl)-4H,5H,6H,7H-thieno[3,2-c]pyridin-4-one,
7-(4-{[trans-4-{[4-(pentafluoro-λ⁶-sulfanyl)phenyl]amino}cyclohexyl]sulfonyl}phenyl)-1H,2H-pyrrolo[1,2-a]pyrazin-1-one,
8-(4-{[trans-4-{[4-(pentafluoro-λ⁶-sulfanyl)phenyl]amino}cyclohexyl]sulfonyl}phenyl)-1H,2H,3H,4H,5H-pyrrolo[1,2-a][1,4]diazepin-1-one,
7-(4-{[trans-4-{[4-(pentafluoro-λ⁶-sulfanyl)phenyl]amino}cyclohexyl]sulfonyl}phenyl)-1H,2H,3H,4H-pyrrolo[1,2-a]pyrazin-1-one,
6-(4-{[trans-4-{[4-(pentafluoro-λ⁶-sulfanyl)phenyl]amino}cyclohexyl]sulfonyl}phenyl)imidazo[1,2-a]pyridine-8-carboxamide,
4-(4-{[trans-4-{[4-(pentafluoro-λ⁶-sulfanyl)phenyl]amino}cyclohexyl]sulfonyl}phenyl)furan-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
6-(4-{[trans-4-{[4-(pentafluoro-λ⁶-sulfanyl)phenyl]amino}cyclohexyl]sulfonyl}phenyl)imidazo[1,2-a]pyridine-8-carbonitrile,
4-(pentafluoro-λ⁶-sulfanyl)-N-[trans-4-(4-{3-[(2R)-pyrrolidin-2-yl]-[1,2,4]triazolo[4,3-a]pyridin-6-yl}benzenesulfonyl)cyclohexyl]aniline,
{4-[3-(propan-2-yl)-[1,2,4]triazolo[4,3-a]pyrazin-6-yl]phenyl}[trans-4-{[4-(pentafluoro-λ⁶-sulfanyl)phenyl]amino}cyclohexyl](imino)-λ⁶-sulfanone,
(+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
(−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
N-[4-(pentafluoro-λ⁶-sulfanyl)phenyl]-1-(4-{3-[(3R)-pyrrolidin-3-yl]-[1,2,4]triazolo[4,3-a]pyridin-6-yl}benzenesulfonyl)piperidin-4-amine,
N-[4-(pentafluoro-λ⁶-sulfanyl)phenyl]-1-(4-{3-[(3S)-pyrrolidin-3-yl]-[1,2,4]triazolo[4,3-a]pyridin-6-yl}benzenesulfonyl)piperidin-4-amine,
1-{4-[3-(azetidin-3-yl)-[1,2,4]triazolo[4,3-a]pyridin-6-yl]benzenesulfonyl}-N-[4-(pentafluoro-λ⁶-sulfanyl)phenyl]piperidin-4-amine,
4-(pentafluoro-λ⁶-sulfanyl)-N-[trans-4-{4-[3-(azetidin-3-yl)-[1,2,4]triazolo[4,3-a]pyridin-6-yl]benzenesulfonyl}cyclohexyl]aniline;
{4-[3-(difluoromethyl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone;
(4-{3-methyl-[1,2,4]triazolo[4,3-a]pyrazin-6-yl}phenyl)[trans-4-{[4-(pentafluoro-λ⁶-sulfanyl)phenyl]amino}cyclohexyl](imino)-λ⁶-sulfanone;
{4-[3-(propan-2-yl)-[1,2,4]triazolo[4,3-a]pyridin-6-yl]phenyl}[trans-4-{[5-(pentafluoro-)⁶-sulfanyl)pyridin-2-yl]amino}cyclohexyl](imino)-λ⁶-sulfanone;
5-(pentafluoro-λ⁶-sulfanyl)-N-(1-{4-[3-(propan-2-yl)-[1,2,4]triazolo[4,3-a]pyridin-6-yl]benzenesulfonyl}piperidin-4-yl)pyridin-2-amine;
N-[1-(4-{imidazo[1,2-a]pyridin-6-yl}benzenesulfonyl)piperidin-4-yl]-5-(pentafluoro-λ⁶-sulfanyl)pyridin-2-amine;
N-[1-(4-{3-methyl-[1,2,4]triazolo[4,3-a]pyridin-6-yl}benzenesulfonyl)piperidin-4-yl]-5-(pentafluoro-λ⁶-sulfanyl)pyridin-2-amine;
4-{4-[(4-{[5-(pentafluoro-λ⁶-sulfanyl)pyridin-2-yl]amino}piperidin-1-yl)sulfonyl]phenyl}pyridine-2-carboxamide;
4-(pentafluoro-λ⁶-sulfanyl)-N-[trans-4-{4-[3-(difluoromethyl)-[1,2,4]triazolo[4,3-a]pyridin-6-yl]benzenesulfonyl}cyclohexyl]aniline;
4-{4-[(4-{[4-(pentafluoro-λ⁶-sulfanyl)phenyl]amino}piperidin-1-yl)sulfonyl]phenyl}pyridine-2-carboxamide;
5-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]pyridin-2-amine;
5-(pentafluoro-λ⁶-sulfanyl)-N-[trans-4-(4-{3-methyl-[1,2,4]triazolo[4,3-a]pyridin-6-yl}benzenesulfonyl)cyclohexyl]pyridin-2-amine;
5-(pentafluoro-λ⁶-sulfanyl)-N-[trans-4-(4-{imidazo[1,2-a]pyridin-6-yl}benzenesulfonyl)cyclohexyl]pyridin-2-amine;
or
5-(pentafluoro-λ⁶-sulfanyl)-N-[trans-4-{4-[8-methyl-3-(propan-2-yl)-[1,2,4]triazolo[4,3-a]pyridin-6-yl]benzenesulfonyl}cyclohexyl]pyridin-2-amine.

Aspect 41. The compound of any one of aspects 1 to 40, or a pharmaceutical acceptable salt thereof, selected from:
1-(4-{imidazo[1,2-a]pyridin-6-yl}benzenesulfonyl)-N-[4-(pentafluoro-λ⁶-sulfanyl)phenyl]piperidin-4-amine
1-[4-(3-methyl-2H-indazol-5-yl)benzenesulfonyl]-N-[4-(pentafluoro-λ⁶-sulfanyl)phenyl]piperidin-4-amine
5-{4-[(4-{[4-(pentafluoro-λ⁶-sulfanyl)phenyl]Amino}piperidin-1-yl)sulfonyl]phenyl}-1H-indole-3-carbonitrile
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[cis-4-[4-(3-methyl-2H-indazol-5-yl)benzenesulfonyl]cyclohexyl]aniline
4-(pentafluoro-λ⁶-sulfanyl)-N-[trans-4-[4-(3-methyl-2H-indazol-5-yl)benzenesulfonyl]cyclohexyl]aniline
5-(4-{[trans-4-{[4-(pentafluoro-λ⁶-sulfanyl)phenyl]Amino}cyclohexyl]sulfonyl}phenyl)-1H-indole-3-carbonitrile
5-(4-{[cis-4-{[4-(pentafluoro-λ⁶-sulfanyl)phenyl]Amino}cyclohexyl]sulfonyl}phenyl)-1H-indole-3-carbonitrile
6-(4-{[trans-4-{[4-(pentafluoro-λ⁶-sulfanyl)phenyl]Amino}cyclohexyl]sulfonyl}phenyl)-2,3-dihydro-1H-isoindol-1-one
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(pentafluoro-λ⁶-sulfanyl)-N-[trans-4-(4-{imidazo[1,2-a]pyridin-6-yl}benzenesulfonyl)cyclohexyl]aniline
4-(pentafluoro-λ⁶-sulfanyl)-N-[trans-4-(4-{3-methylimidazo[1,2-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-indol-2-one
N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide
N-methyl-4′-{[trans-4-{[4-(pentafluoro-λ⁶-sulfanyl)phenyl]Amino}cyclohexyl]sulfonyl}-[1,1′-biphenyl]-4-carboxamide
6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile
4-(pentafluoro-λ⁶-sulfanyl)-N-[cis-4-(4-{imidazo[1,2-a]pyridin-6-yl}benzenesulfonyl)cyclohexyl]aniline
N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonyl}phenyl)pyridine-2-carboxamide
N-methyl-4′-{[trans-4-{[4-(pentafluoro-λ⁶-sulfanyl)phenyl]Amino}cyclohexyl]sulfonyl}-[1,1′-biphenyl]-3-carboxamide
N-methyl-5-(4-{[trans-4-{[4-(pentafluoro-λ⁶-sulfanyl)phenyl]Amino}cyclohexyl]sulfonyl}phenyl)pyridine-2-carboxamide
4′-{[trans-4-{[4-(pentafluoro-λ⁶-sulfanyl)phenyl]Amino}cyclohexyl]sulfonyl}-[1,1′-biphenyl]-3-carboxamide
1-(4-{imidazo[1,2-a]pyrazin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-)⁶-sulfanyl)phenyl]piperidin-4-amine
N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
N-[4-(pentafluoro-λ⁶-sulfanyl)phenyl]-1-{4-[3-(propan-2-yl)imidazo[1,2-a]pyridin-6-yl]benzenesulfonyl}piperidin-4-amine
1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine
1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile
6-(4-{[trans-4-{[4-(pentafluoro-λ⁶-sulfanyl)phenyl]Amino}cyclohexyl]sulfonyl}phenyl)imidazo[1,2-a]pyridine-3-carboxamide
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-(4-{[1,2,4]triazolo[4,3-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-1H-indazole-3-carboxamide
5-(4-{[trans-4-{[4-(pentafluoro-λ⁶-sulfanyl)phenyl]Amino}cyclohexyl]sulfonyl}phenyl)-1H-indazole-3-carbonitrile
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide
4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline
4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile
4-(pentafluoro-λ⁶-sulfanyl)-N-[trans-4-(4-{2-methylimidazo[1,2-a]pyrazin-6-yl}benzenesulfonyl)cyclohexyl]aniline
4-(pentafluoro-λ⁶-sulfanyl)-N-[trans-4-(4-{imidazo[1,2-a]pyrazin-6-yl}benzenesulfonyl)cyclohexyl]aniline
N,N-dimethyl-6-(4-{[cis-4-{[4-(pentafluoro-λ⁶-sulfanyl)phenyl]Amino}cyclohexyl]sulfonyl}phenyl)pyridine-3-carboxamide
N,N-dimethyl-6-(4-{[trans-4-{[4-(pentafluoro-λ⁶-sulfanyl)phenyl]Amino}cyclohexyl]sulfonyl}phenyl)pyridine-3-carboxamide
N-methyl-6-(4-{[trans-4-{[4-(pentafluoro-λ⁶-sulfanyl)phenyl]Amino}cyclohexyl]sulfonyl}phenyl)pyridine-3-carboxamide
N-methyl-4-(4-{[trans-4-{[4-(pentafluoro-λ⁶-sulfanyl)phenyl]Amino}cyclohexyl]sulfonyl}phenyl)pyridine-2-carboxamide
6-(4-{[trans-4-{[4-(pentafluoro-λ⁶-sulfanyl)phenyl]Amino}cyclohexyl]sulfonyl}phenyl)pyridine-3-carboxamide
4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
4-(4-{[trans-4-{[4-(pentafluoro-λ⁶-sulfanyl)phenyl]Amino}cyclohexyl]sulfonyl}phenyl)-1,2-dihydropyridin-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one
5-(4-{[trans-4-{[4-(pentafluoro-λ⁶-sulfanyl)phenyl]Amino}cyclohexyl]sulfonyl}phenyl)pyridin-2-ol
4-(pentafluoro-λ⁶-sulfanyl)-N-[trans-4-{[4′-(dimethylphosphoryl)-[1,1′-biphenyl]-4-yl]sulfonyl}cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-isoindol-1-one
6-(4-{[trans-4-{[4-(pentafluoro-λ⁶-sulfanyl)phenyl]Amino}cyclohexyl]sulfonyl}phenyl)-2,3-dihydro-1H-indol-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)pyridazine-3-carboxamide
4-(pentafluoro-λ⁶-sulfanyl)-N-[trans-4-{4-[3-(propan-2-yl)imidazo[1,2-a]pyridin-6-yl]benzenesulfonyl}cyclohexyl]aniline
4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline
5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one
6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one
1-(2-fluoro-4-{3-methylimidazo[1,2-a]pyridin-6-yl}benzenesulfonyl)-N-[4-(pentafluoro-λ⁶-sulfanyl)phenyl]piperidin-4-amine
1-(3-fluoro-4-{3-methylimidazo[1,2-a]pyridin-6-yl}benzenesulfonyl)-N-[4-(pentafluoro-λ⁶-sulfanyl)phenyl]piperidin-4-amine
1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine
1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine
6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(3-fluoro-4-{imidazo[1,2-a]pyridin-6-yl}benzenesulfonyl)-N-[4-(pentafluoro-λ⁶-sulfanyl)phenyl]piperidin-4-amine
6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(2-fluoro-4-{imidazo[1,2-a]pyridin-6-yl}benzenesulfonyl)-N-[4-(pentafluoro-λ⁶-sulfanyl)phenyl]piperidin-4-amine
1-[(5-{imidazo[1,2-a]pyridin-6-yl}pyridin-2-yl)sulfonyl]-N-[4-(pentafluoro-λ⁶-sulfanyl)phenyl]piperidin-4-amine
6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile
1-[(6-{imidazo[1,2-a]pyridin-6-yl}pyridin-3-yl)sulfonyl]-N-[4-(pentafluoro-λ⁶-sulfanyl)phenyl]piperidin-4-amine
6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile
1-(2-chloro-4-{imidazo[1,2-a]pyridin-6-yl}benzenesulfonyl)-N-[4-(pentafluoro-λ⁶-sulfanyl)phenyl]piperidin-4-amine
6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[3-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[3-(pentafluoro-λ⁶-sulfanyl)phenyl]piperidin-4-amine
4-(4-{[(3S,4S)-3-fluoro-4-{[4-(pentafluoro-λ⁶-sulfanyl)phenyl]Amino}piperidin-1-yl]sulfonyl}phenyl)pyridine-2-carboxamide
N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
5-(4-{[trans-4-{[4-(pentafluoro-λ⁶-sulfanyl)phenyl]Amino}cyclohexyl]sulfonimidoyl}phenyl)-1H-indole-3-carbonitrile
[4-(3-methyl-1H-indazol-5-yl)phenyl][trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
4-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide
(4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone
[trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone
N-methyl-4′-{[trans-4-{[4-(pentafluoro-λ⁶-sulfanyl)phenyl]Amino}cyclohexyl]sulfonimidoyl}-[1,1′-biphenyl]-4-carboxamide
4′-{[trans-4-{[4-(pentafluoro-λ⁶-sulfanyl)phenyl]Amino}cyclohexyl]sulfonimidoyl}-[1,1′-biphenyl]-4-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide
N-methyl-4′-{[trans-4-{[4-(pentafluoro-λ⁶-sulfanyl)phenyl]Amino}cyclohexyl]sulfonimidoyl}-[1,1′-biphenyl]-3-carboxamide
4′-{[trans-4-{[4-(pentafluoro-λ⁶-sulfanyl)phenyl]Amino}cyclohexyl]sulfonimidoyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide
N-methyl-5-(4-{[trans-4-{[4-(pentafluoro-λ⁶-sulfanyl)phenyl]Amino}cyclohexyl]sulfonimidoyl}phenyl)pyridine-2-carboxamide
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
(4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone
N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide
N-methyl-6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide
N-methyl-4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
5-(4-{[trans-4-{[4-(pentafluoro-λ⁶-sulfanyl)phenyl]Amino}cyclohexyl]sulfonimidoyl}phenyl)-2,3-dihydro-1H-indol-2-one
6-(4-{[trans-4-{[4-(pentafluoro-λ⁶-sulfanyl)phenyl]Amino}cyclohexyl]sulfonimidoyl}phenyl)-2,3-dihydro-1H-indol-2-one
{4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one
5-{4-[(4-{[4-(pentafluoro-λ⁶-sulfanyl)phenyl]Amino}piperidin-1-yl)sulfonimidoyl]phenyl}-1H-indole-3-carbonitrile
N-methyl-5-{4-[(4-{[4-(pentafluoro-λ⁶-sulfanyl)phenyl]Amino}piperidin-1-yl)sulfonimidoyl]phenyl}pyridine-2-carboxamide
4′-[(4-{[4-(pentafluoro-λ⁶-sulfanyl)phenyl]Amino}piperidin-1-yl)sulfonimidoyl]-[1,1′-biphenyl]-4-carbonitrile
5-{4-[(4-{[4-(pentafluoro-λ⁶-sulfanyl)phenyl]Amino}piperidin-1-yl)sulfonimidoyl]phenyl}-2,3-dihydro-1H-indol-2-one
(4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone
4-{4-[(4-{[4-(pentafluoro-λ⁶-sulfanyl)phenyl]Amino}piperidin-1-yl)sulfonimidoyl]phenyl}pyridine-2-carboxamide
(+)-5-(4-{[trans-4-{[4-(pentafluoro-λ⁶-sulfanyl)phenyl]Amino}cyclohexyl]sulfonimidoyl}phenyl)-1H-indole-3-carbonitrile
(−)-5-(4-{[trans-4-{[4-(pentafluoro-λ⁶-sulfanyl)phenyl]Amino}cyclohexyl]sulfonimidoyl}phenyl)-1H-indole-3-carbonitrile
(+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(+)-4-(4-{[trans-4-{[4-(pentafluoro-λ⁶-sulfanyl)phenyl]Amino}cyclohexyl]sulfonimidoyl}phenyl)pyridine-2-carboxamide
(−)-4-(4-{[trans-4-{[4-(pentafluoro-λ⁶-sulfanyl)phenyl]Amino}cyclohexyl]sulfonimidoyl}phenyl)pyridine-2-carboxamide
1-(4-{2-methoxy-7-azaspiro[3.5]nonan-7-yl}benzenesulfonyl)-N-[4-(pentafluoro-λ⁶-sulfanyl)phenyl]piperidin-4-amine,
8-{4-[(4-{[4-(pentafluoro-λ⁶-sulfanyl)phenyl]amino}piperidin-1-yl)sulfonyl]phenyl}-1,3,8-triazaspiro[4.5]decane-2,4-dione,
8-{4-[(4-{[4-(pentafluoro-λ⁶-sulfanyl)phenyl]amino}piperidin-1-yl)sulfonyl]phenyl}-2,8-diazaspiro[4.5]decan-1-one,
8-{4-[(4-{[4-(pentafluoro-λ⁶-sulfanyl)phenyl]amino}piperidin-1-yl)sulfonyl]phenyl}-3-oxa-1,8-diazaspiro[4.5]decan-2-one,
1-(4-{2-oxa-7-azaspiro[3.5]nonan-7-yl}benzenesulfonyl)-N-[4-(pentafluoro-λ⁶-sulfanyl)phenyl]piperidin-4-amine,
1-(4-{8-oxa-3-azabicyclo[3.2.1]octan-3-yl}benzenesulfonyl)-N-[4-(pentafluoro-λ⁶-sulfanyl)phenyl]piperidin-4-amine,
4-fluoro-N-methyl-4′-[(4-{[4-(pentafluoro-λ⁶-sulfanyl)phenyl]amino}piperidin-1-yl)sulfonyl]-[1,1′-biphenyl]-3-carboxamide,
3-fluoro-N-methyl-4′-[(4-{[4-(pentafluoro-λ⁶-sulfanyl)phenyl]amino}piperidin-1-yl)sulfonyl]-[1,1′-biphenyl]-4-carboxamide,
N-[4-(pentafluoro-λ⁶-sulfanyl)phenyl]-1-{4-[3-(propan-2-yl)-[1,2,4]triazolo[4,3-a]pyrazin-6-yl]benzenesulfonyl}piperidin-4-amine,
N-(propan-2-yl)-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{3-methyl-1H-pyrazolo[3,4-c]pyridin-5-yl}benzenesulfonyl)cyclohexyl]aniline,
N,N-dimethyl-6-(4-{[trans-4-{[4-(pentafluoro-λ⁶-sulfanyl)phenyl]amino}cyclohexyl]sulfonyl}phenyl)imidazo[1,2-a]pyridin-3-amine,
N-cyclopropyl-4′-{[trans-4-{[4-(pentafluoro-λ⁶-sulfanyl)phenyl]amino}cyclohexyl]sulfonyl}-[1,1′-biphenyl]-3-carboxamide,
4-(pentafluoro-λ⁶-sulfanyl)-N-[trans-4-(4-{5H-pyrrolo[3,2-d]pyrimidin-2-yl}benzenesulfonyl)cyclohexyl]aniline,
4-(pentafluoro-λ⁶-sulfanyl)-N-[trans-4-(4-{1H-pyrrolo[2,3-c]pyridin-5-yl}benzenesulfonyl)cyclohexyl]aniline,
4-(pentafluoro-λ⁶-sulfanyl)-N-[trans-4-(4-{1H-pyrazolo[3,4-c]pyridin-5-yl}benzenesulfonyl)cyclohexyl]aniline,
4-(4-{[trans-4-{[4-(pentafluoro-λ⁶-sulfanyl)phenyl]amino}cyclohexyl]sulfonyl}phenyl)thiophene-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
4-(pentafluoro-λ⁶-sulfanyl)-N-[trans-4-(4-{3-methyl-1H-pyrazolo[3,4-b]pyridin-5-yl}benzenesulfonyl)cyclohexyl]aniline,
4-(pentafluoro-λ⁶-sulfanyl)-N-[trans-4-{4-[8-(trifluoromethyl)imidazo[1,2-a]pyridin-6-yl]benzenesulfonyl}cyclohexyl]aniline,
4-(pentafluoro-λ⁶-sulfanyl)-N-[trans-4-(4-{8-fluoroimidazo[1,2-a]pyridin-6-yl}benzenesulfonyl)cyclohexyl]aniline,
2-(4-{[trans-4-{[4-(pentafluoro-λ⁶-sulfanyl)phenyl]amino}cyclohexyl]sulfonyl}phenyl)-4H,5H,6H,7H-thieno[3,2-c]pyridin-4-one,
7-(4-{[trans-4-{[4-(pentafluoro-λ⁶-sulfanyl)phenyl]amino}cyclohexyl]sulfonyl}phenyl)-1H,2H-pyrrolo[1,2-a]pyrazin-1-one,
8-(4-{[trans-4-{[4-(pentafluoro-λ⁶-sulfanyl)phenyl]amino}cyclohexyl]sulfonyl}phenyl)-1H,2H,3H,4H,5H-pyrrolo[1,2-a][1,4]diazepin-1-one,
7-(4-{[trans-4-{[4-(pentafluoro-λ⁶-sulfanyl)phenyl]amino}cyclohexyl]sulfonyl}phenyl)-1H,2H,3H,4H-pyrrolo[1,2-a]pyrazin-1-one,
6-(4-{[trans-4-{[4-(pentafluoro-λ⁶-sulfanyl)phenyl]amino}cyclohexyl]sulfonyl}phenyl)imidazo[1,2-a]pyridine-8-carboxamide,
4-(4-{[trans-4-{[4-(pentafluoro-λ⁶-sulfanyl)phenyl]amino}cyclohexyl]sulfonyl}phenyl)furan-2-carboxamide,
4-(pentafluoro-λ⁶-sulfanyl)-N-[trans-4-(4-{8-methylimidazo[1,2-a]pyridin-6-yl}benzenesulfonyl)cyclohexyl]aniline,
6-(4-{[trans-4-{[4-(pentafluoro-λ⁶-sulfanyl)phenyl]amino}cyclohexyl]sulfonyl}phenyl)imidazo[1,2-a]pyridine-8-carbonitrile,
4-(pentafluoro-λ⁶-sulfanyl)-N-[trans-4-(4-{3-[(2R)-pyrrolidin-2-yl]-[1,2,4]triazolo[4,3-a]pyridin-6-yl}benzenesulfonyl)cyclohexyl]aniline,
{4-[3-(propan-2-yl)-[1,2,4]triazolo[4,3-a]pyrazin-6-yl]phenyl}[trans-4-{[4-(pentafluoro-λ⁶-sulfanyl)phenyl]amino}cyclohexyl](imino)-λ⁶-sulfanone,
(+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
(−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone,
N-[4-(pentafluoro-λ⁶-sulfanyl)phenyl]-1-(4-{3-[(3R)-pyrrolidin-3-yl]-[1,2,4]triazolo[4,3-a]pyridin-6-yl}benzenesulfonyl)piperidin-4-amine,
N-[4-(pentafluoro-λ⁶-sulfanyl)phenyl]-1-(4-{3-[(3S)-pyrrolidin-3-yl]-[1,2,4]triazolo[4,3-a]pyridin-6-yl}benzenesulfonyl)piperidin-4-amine,
1-{4-[3-(azetidin-3-yl)-[1,2,4]triazolo[4,3-a]pyridin-6-yl]benzenesulfonyl}-N-[4-(pentafluoro-λ⁶-sulfanyl)phenyl]piperidin-4-amine, or
4-(pentafluoro-λ⁶-sulfanyl)-N-[trans-4-{4-[3-(azetidin-3-yl)-[1,2,4]triazolo[4,3-a]pyridin-6-yl]benzenesulfonyl}cyclohexyl]aniline.

Aspect 42. The compound of any one of aspects 1 to 41, or a pharmaceutically acceptable salt thereof, selected from:
1-(4-{imidazo[1,2-a]pyridin-6-yl}benzenesulfonyl)-N-[4-(pentafluoro-λ⁶-sulfanyl)phenyl]piperidin-4-amine
1-[4-(3-methyl-2H-indazol-5-yl)benzenesulfonyl]-N-[4-(pentafluoro-λ⁶-sulfanyl)phenyl]piperidin-4-amine
5-{4-[(4-{[4-(pentafluoro-λ⁶-sulfanyl)phenyl]Amino}piperidin-1-yl)sulfonyl]phenyl}-1H-indole-3-carbonitrile
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[cis-4-[4-(3-methyl-2H-indazol-5-yl)benzenesulfonyl]cyclohexyl]aniline
4-(pentafluoro-λ⁶-sulfanyl)-N-[trans-4-[4-(3-methyl-2H-indazol-5-yl)benzenesulfonyl]cyclohexyl]aniline
5-(4-{[trans-4-{[4-(pentafluoro-λ⁶-sulfanyl)phenyl]Amino}cyclohexyl]sulfonyl}phenyl)-1H-indole-3-carbonitrile
5-(4-{[cis-4-{[4-(pentafluoro-λ⁶-sulfanyl)phenyl]Amino}cyclohexyl]sulfonyl}phenyl)-1H-indole-3-carbonitrile
6-(4-{[trans-4-{[4-(pentafluoro-λ⁶-sulfanyl)phenyl]Amino}cyclohexyl]sulfonyl}phenyl)-2,3-dihydro-1H-isoindol-1-one
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(pentafluoro-λ⁶-sulfanyl)-N-[trans-4-(4-{imidazo[1,2-a]pyridin-6-yl}benzenesulfonyl)cyclohexyl]aniline
4-(pentafluoro-λ⁶-sulfanyl)-N-[trans-4-(4-{3-methylimidazo[1,2-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-indol-2-one
N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide
N-methyl-4′-{[trans-4-{[4-(pentafluoro-λ⁶-sulfanyl)phenyl]Amino}cyclohexyl]sulfonyl}-[1,1′-biphenyl]-4-carboxamide
6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile
4-(pentafluoro-λ⁶-sulfanyl)-N-[cis-4-(4-{imidazo[1,2-a]pyridin-6-yl}benzenesulfonyl)cyclohexyl]aniline
N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonyl}phenyl)pyridine-2-carboxamide
N-methyl-4′-{[trans-4-{[4-(pentafluoro-λ⁶-sulfanyl)phenyl]Amino}cyclohexyl]sulfonyl}-[1,1′-biphenyl]-3-carboxamide
N-methyl-5-(4-{[trans-4-{[4-(pentafluoro-λ⁶-sulfanyl)phenyl]Amino}cyclohexyl]sulfonyl}phenyl)pyridine-2-carboxamide
4′-{[trans-4-{[4-(pentafluoro-λ⁶-sulfanyl)phenyl]Amino}cyclohexyl]sulfonyl}-[1,1′-biphenyl]-3-carboxamide
1-(4-{imidazo[1,2-a]pyrazin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine
N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
N-[4-(pentafluoro-λ⁶-sulfanyl)phenyl]-1-{4-[3-(propan-2-yl)imidazo[1,2-a]pyridin-6-yl]benzenesulfonyl}piperidin-4-amine
1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine
1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile
6-(4-{[trans-4-{[4-(pentafluoro-λ⁶-sulfanyl)phenyl]Amino}cyclohexyl]sulfonyl}phenyl)imidazo[1,2-a]pyridine-3-carboxamide
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-(4-{[1,2,4]triazolo[4,3-a]pyridin-6-yl}benzenesulfonyl)cyclohexyl]aniline
5-(4-{[trans-4-{[4-(pentafluoro-λ⁶-sulfanyl)phenyl]Amino}cyclohexyl]sulfonyl}phenyl)-1H-indazole-3-carboxamide
5-(4-{[trans-4-{[4-(pentafluoro-λ⁶-sulfanyl)phenyl]Amino}cyclohexyl]sulfonyl}phenyl)-1H-indazole-3-carbonitrile
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide
4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline
4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile
4-(pentafluoro-λ⁶-sulfanyl)-N-[trans-4-(4-{2-methylimidazo[1,2-a]pyrazin-6-yl}benzenesulfonyl)cyclohexyl]aniline
4-(pentafluoro-λ⁶-sulfanyl)-N-[trans-4-(4-{imidazo[1,2-a]pyrazin-6-yl}benzenesulfonyl)cyclohexyl]aniline
N,N-dimethyl-6-(4-{[cis-4-{[4-(pentafluoro-λ⁶-sulfanyl)phenyl]Amino}cyclohexyl]sulfonyl}phenyl)pyridine-3-carboxamide
N,N-dimethyl-6-(4-{[trans-4-{[4-(pentafluoro-λ⁶-sulfanyl)phenyl]Amino}cyclohexyl]sulfonyl}phenyl)pyridine-3-carboxamide
N-methyl-6-(4-{[trans-4-{[4-(pentafluoro-λ⁶-sulfanyl)phenyl]Amino}cyclohexyl]sulfonyl}phenyl)pyridine-3-carboxamide
N-methyl-4-(4-{[trans-4-{[4-(pentafluoro-λ⁶-sulfanyl)phenyl]Amino}cyclohexyl]sulfonyl}phenyl)pyridine-2-carboxamide
6-(4-{[trans-4-{[4-(pentafluoro-λ⁶-sulfanyl)phenyl]Amino}cyclohexyl]sulfonyl}phenyl)pyridine-3-carboxamide
4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
4-(4-{[trans-4-{[4-(pentafluoro-λ⁶-sulfanyl)phenyl]Amino}cyclohexyl]sulfonyl}phenyl)-1,2-dihydropyridin-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one
6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one
5-(4-{[trans-4-{[4-(pentafluoro-λ⁶-sulfanyl)phenyl]Amino}cyclohexyl]sulfonyl}phenyl)pyridin-2-ol
4-(pentafluoro-λ⁶-sulfanyl)-N-[trans-4-{[4′-(dimethylphosphoryl)-[1,1′-biphenyl]-4-yl]sulfonyl}cyclohexyl]aniline
5-(4-{[trans-4-{[4-(pentafluoro-λ⁶-sulfanyl)phenyl]Amino}cyclohexyl]sulfonyl}phenyl)-2,3-dihydro-1H-isoindol-1-one
6-(4-{[trans-4-{[4-(pentafluoro-λ⁶-sulfanyl)phenyl]Amino}cyclohexyl]sulfonyl}phenyl)-2,3-dihydro-1H-indol-2-one
7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonyl}phenyl)pyridazine-3-carboxamide
4-(pentafluoro-λ⁶-sulfanyl)-N-[trans-4-{4-[3-(propan-2-yl)imidazo[1,2-a]pyridin-6-yl]benzenesulfonyl}cyclohexyl]aniline
4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline
5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one
6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one
1-(2-fluoro-4-{3-methylimidazo[1,2-a]pyridin-6-yl}benzenesulfonyl)-N-[4-(pentafluoro-λ⁶-sulfanyl)phenyl]piperidin-4-amine
1-(3-fluoro-4-{3-methylimidazo[1,2-a]pyridin-6-yl}benzenesulfonyl)-N-[4-(pentafluoro-λ⁶-sulfanyl)phenyl]piperidin-4-amine
1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine
1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine
6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(3-fluoro-4-{imidazo[1,2-a]pyridin-6-yl}benzenesulfonyl)-N-[4-(pentafluoro-λ⁶-sulfanyl)phenyl]piperidin-4-amine
6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(2-fluoro-4-{imidazo[1,2-a]pyridin-6-yl}benzenesulfonyl)-N-[4-(pentafluoro-λ⁶-sulfanyl)phenyl]piperidin-4-amine
1-[(5-{imidazo[1,2-a]pyridin-6-yl}pyridin-2-yl)sulfonyl]-N-[4-(pentafluoro-λ⁶-sulfanyl)phenyl]piperidin-4-amine
6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile
1-[(6-{imidazo[1,2-a]pyridin-6-yl}pyridin-3-yl)sulfonyl]-N-[4-(pentafluoro-λ⁶-sulfanyl)phenyl]piperidin-4-amine
6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile
1-(2-chloro-4-{imidazo[1,2-a]pyridin-6-yl}benzenesulfonyl)-N-[4-(pentafluoro-λ⁶-sulfanyl)phenyl]piperidin-4-amine
6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile
6-{4-[(4-{[3-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[3-(pentafluoro-λ⁶-sulfanyl)phenyl]piperidin-4-amine
4-(4-{[(3S,4S)-3-fluoro-4-{[4-(pentafluoro-λ⁶-sulfanyl)phenyl]Amino}piperidin-1-yl]sulfonyl}phenyl)pyridine-2-carboxamide
N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine
5-(4-{[trans-4-{[4-(pentafluoro-λ⁶-sulfanyl)phenyl]Amino}cyclohexyl]sulfonimidoyl}phenyl)-1H-indole-3-carbonitrile
[4-(3-methyl-1H-indazol-5-yl)phenyl][trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
4-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide
(4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone
[trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone
N-methyl-4′-{[trans-4-{[4-(pentafluoro-λ⁶-sulfanyl)phenyl]Amino}cyclohexyl]sulfonimidoyl}-[1,1′-biphenyl]-4-carboxamide
4′-{[trans-4-{[4-(pentafluoro-λ⁶-sulfanyl)phenyl]Amino}cyclohexyl]sulfonimidoyl}-[1,1′-biphenyl]-4-carboxamide
N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide
N-methyl-4′-{[trans-4-{[4-(pentafluoro-λ⁶-sulfanyl)phenyl]Amino}cyclohexyl]sulfonimidoyl}-[1,1′-biphenyl]-3-carboxamide
4′-{[trans-4-{[4-(pentafluoro-λ⁶-sulfanyl)phenyl]Amino}cyclohexyl]sulfonimidoyl}-[1,1′-biphenyl]-3-carboxamide
N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide
N-methyl-5-(4-{[trans-4-{[4-(pentafluoro-λ⁶-sulfanyl)phenyl]Amino}cyclohexyl]sulfonimidoyl}phenyl)pyridine-2-carboxamide
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
(4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone
(4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone
N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide
N-methyl-6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)pyridine-2-carboxamide
N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide
N-methyl-4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)pyridine-3-carboxamide
5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one
6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one
5-(4-{[trans-4-{[4-(pentafluoro-λ⁶-sulfanyl)phenyl]Amino}cyclohexyl]sulfonimidoyl}phenyl)-2,3-dihydro-1H-indol-2-one
6-(4-{[trans-4-{[4-(pentafluoro-λ⁶-sulfanyl)phenyl]Amino}cyclohexyl]sulfonimidoyl}phenyl)-2,3-dihydro-1H-indol-2-one
{4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone
6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one
5-{4-[(4-{[4-(pentafluoro-λ⁶-sulfanyl)phenyl]Amino}piperidin-1-yl)sulfonimidoyl]phenyl}-1H-indole-3-carbonitrile
N-methyl-5-{4-[(4-{[4-(pentafluoro-λ⁶-sulfanyl)phenyl]Amino}piperidin-1-yl)sulfonimidoyl]phenyl}pyridine-2-carboxamide
4′-[(4-{[4-(pentafluoro-λ⁶-sulfanyl)phenyl]Amino}piperidin-1-yl)sulfonimidoyl]-[1,1′-biphenyl]-4-carbonitrile
5-{4-[(4-{[4-(pentafluoro-λ⁶-sulfanyl)phenyl]Amino}piperidin-1-yl)sulfonimidoyl]phenyl}-2,3-dihydro-1H-indol-2-one
(4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone
4-{4-[(4-{[4-(pentafluoro-λ⁶-sulfanyl)phenyl]Amino}piperidin-1-yl)sulfonimidoyl]phenyl}pyridine-2-carboxamide
(+)-5-(4-{[trans-4-{[4-(pentafluoro-λ⁶-sulfanyl)phenyl]Amino}cyclohexyl]sulfonimidoyl}phenyl)-1H-indole-3-carbonitrile
(−)-5-(4-{[trans-4-{[4-(pentafluoro-λ⁶-sulfanyl)phenyl]Amino}cyclohexyl]sulfonimidoyl}phenyl)-1H-indole-3-carbonitrile
(+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
(+)-4-(4-{[trans-4-{[4-(pentafluoro-λ⁶-sulfanyl)phenyl]Amino}cyclohexyl]sulfonimidoyl}phenyl)pyridine-2-carboxamide
(−)-4-(4-{[trans-4-{[4-(pentafluoro-λ⁶-sulfanyl)phenyl]Amino}cyclohexyl]sulfonimidoyl}phenyl)pyridine-2-carboxamide.

Aspect 43. The compound of any one of aspects 1 to 42, or a pharmaceutically acceptable salt thereof, selected from:
1-(4-{3-methyl-[1,2,4]triazolo[4,3-a]pyridin-6-yl}benzenesulfonyl)-N-[4-(pentafluoro-)⁶-sulfanyl)phenyl]piperidin-4-amine
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
N-methyl-4′-{[trans-4-{[4-(pentafluoro-λ⁶-sulfanyl)phenyl]Amino}cyclohexyl]sulfonyl}-[1,1′-biphenyl]-4-carboxamide
4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
5-(4-{[trans-4-{[4-(pentafluoro-λ⁶-sulfanyl)phenyl]Amino}cyclohexyl]sulfonyl}phenyl)-1H-indazole-3-carbonitrile
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(4-{[trans-4-{[4-(pentafluoro-λ⁶-sulfanyl)phenyl]Amino}cyclohexyl]sulfonyl}phenyl)pyridine-2-carboxamide
5-(4-{[trans-4-{[4-(pentafluoro-λ⁶-sulfanyl)phenyl]Amino}cyclohexyl]sulfonyl}phenyl)-1H-indole-3-carbonitrile
4-(pentafluoro-λ⁶-sulfanyl)-N-[trans-4-[4-(3-methyl-2H-indazol-5-yl)benzenesulfonyl]cyclohexyl]aniline
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[trans-4-(4-{3-methylimidazo[1,2-a]pyridin-6-yl}benzenesulfonyl)cyclohexyl]aniline
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[4-(pentafluoro-λ⁶-sulfanyl)phenyl]piperidin-4-amine;
5-(pentafluoro-λ⁶-sulfanyl)-N-(1-{4-[3-(propan-2-yl)-[1,2,4]triazolo[4,3-a]pyridin-6-yl]benzenesulfonyl}piperidin-4-yl)pyridin-2-amine;
N-[1-(4-{imidazo[1,2-a]pyridin-6-yl}benzenesulfonyl)piperidin-4-yl]-5-(pentafluoro-λ⁶-sulfanyl)pyridin-2-amine;
or
N-[1-(4-{3-methyl-[1,2,4]triazolo[4,3-a]pyridin-6-yl}benzenesulfonyl)piperidin-4-yl]-5-(pentafluoro-λ⁶-sulfanyl)pyridin-2-amine.

Aspect 44. The compound of any one of aspects 1 to 43, or a pharmaceutically acceptable salt thereof, selected from:
1-(4-{3-methyl-[1,2,4]triazolo[4,3-a]pyridin-6-yl}benzenesulfonyl)-N-[4-(pentafluoro-)⁶-sulfanyl)phenyl]piperidin-4-amine
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone
N-methyl-4′-{[trans-4-{[4-(pentafluoro-λ⁶-sulfanyl)phenyl]Amino}cyclohexyl]sulfonyl}-[1,1′-biphenyl]-4-carboxamide
4-(4-{[trans-4-{[4-(pentafluoro-λ⁶-sulfanyl)phenyl]Amino}cyclohexyl]sulfonimidoyl}phenyl)pyridine-2-carboxamide
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile
5-(4-{[trans-4-{[4-(pentafluoro-λ⁶-sulfanyl)phenyl]Amino}cyclohexyl]sulfonyl}phenyl)-1H-indazole-3-carbonitrile
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
4-(4-{[trans-4-{[4-(pentafluoro-λ⁶-sulfanyl)phenyl]Amino}cyclohexyl]sulfonyl}phenyl)pyridine-2-carboxamide
5-(4-{[trans-4-{[4-(pentafluoro-λ⁶-sulfanyl)phenyl]Amino}cyclohexyl]sulfonyl}phenyl)-1H-indole-3-carbonitrile
4-(pentafluoro-λ⁶-sulfanyl)-N-[trans-4-[4-(3-methyl-2H-indazol-5-yl)benzenesulfonyl]cyclohexyl]aniline
5-{4-[(4-{[4-(pentafluoro-λ⁶-sulfanyl)phenyl]Amino}piperidin-1-yl)sulfonyl]phenyl}-1H-indazole-3-carbonitrile
4-(pentafluoro-λ⁶-sulfanyl)-N-[trans-4-(4-{3-methylimidazo[1,2-a]pyridin-6-yl}benzenesulfonyl)cyclohexyl]aniline
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile
1-(4-{imidazo[1,2-a]pyridin-6-yl}benzenesulfonyl)-N-[4-(pentafluoro-λ⁶-sulfanyl)phenyl]piperidin-4-amine.

Aspect 45. The compound of any one of aspects 1 to 44, or a pharmaceutically acceptable salt thereof, selected from:
4-(4-{[trans-4-{[4-(pentafluoro-λ⁶-sulfanyl)phenyl]Amino}cyclohexyl]sulfonyl}phenyl)pyridine-2-carboxamide
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline
(+)-4-(4-{[trans-4-{[4-(pentafluoro-λ⁶-sulfanyl)phenyl]Amino}cyclohexyl]sulfonimidoyl}phenyl)pyridine-2-carboxamide
(+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone
(−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone

Aspect 46. A process of preparation of a compound of formula (I) or (I′) according to any one of aspects 1 to 45, or a pharmaceutically acceptable salt thereof, wherein X¹ is N and X⁴ is O, comprising:
reacting compound of formula (VI), wherein R⁶, R⁷, and X⁵ are as defined in any one of aspects 1 to 45,

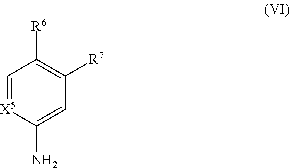

with compound of formula (VII), wherein R⁵ and R⁴ are as defined in any one of aspects 1 to 45,

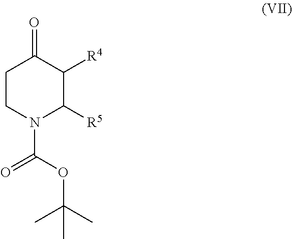

to form compound of formula (VIII), wherein R⁴, R⁵, X⁵, R⁶, R⁷ are as defined in any one of aspects 1 to 45,

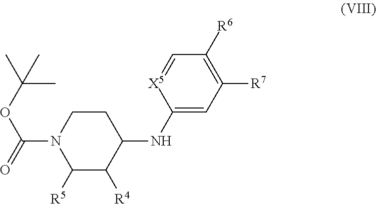

reacting said compound of formula (VIII) with acid to form compound of formula (IX), wherein R⁴, R⁵, X⁵, R⁶, R⁷ are as defined in any one of aspects 1 to 45,

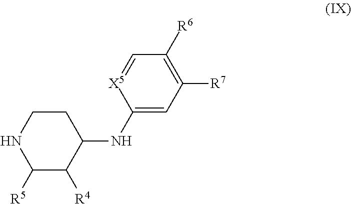

reacting said compound of formula (IX) with compound of formula (XI), wherein X², X³, R² and R³ are as defined in any one of aspects 1 to 45,

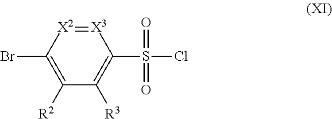

to form compound of formula (III), wherein R², R³, R⁴, R⁶, R⁷, R⁵, X², X⁵ and X³ are as defined in any one of aspects 1 to 45,

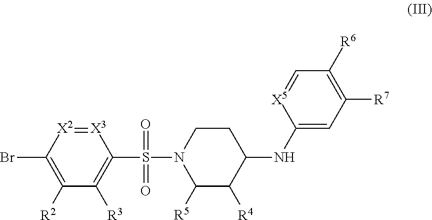

reacting said compound of formula (III) with compound of formula (X) wherein R¹ is as defined in any one of aspects 1 to 45, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

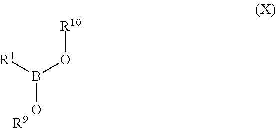

to form compound of formula (I).

Aspect 47. A process of preparation of a compound of formula (I) or (I′) according to any one of aspects 1 to 45, or a pharmaceutically acceptable salt thereof wherein X¹ is CH and X⁴ is O, comprising:
reacting compound of formula (XII), wherein R⁵ and R⁴ are as defined in any one of aspects 1 to 45,

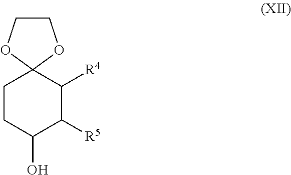

with CH₃SO₂Cl to form compound of formula (XIII), wherein R⁵ and R⁴ are as defined in any one of aspects 1 to 45,

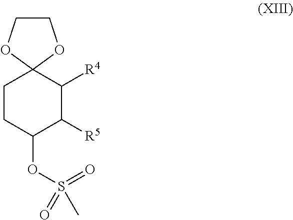

reacting said compound of formula (XIII) with compound of formula (XIV), wherein R³, R², X², and X³ are as defined in any one of aspects 1 to 45,

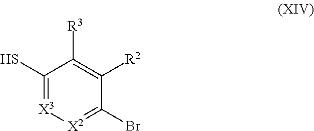

to form compound of formula (XV), wherein R³, R², R¹, R⁴, X², and X³ are as defined in any one of aspects 1 to 45,

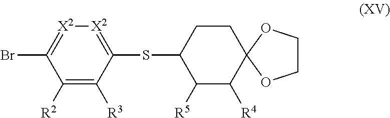

reacting said compound of formula (XV) with an oxidant to form compound of formula (XVI), wherein R³, R², R⁵, R⁴, X², and X³ are as defined in any one of aspects 1 to 45,

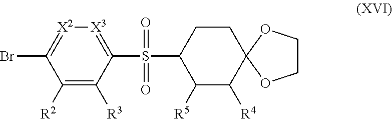

reacting said compound of formula (XVI) with an acid to form compound of formula (XVII), wherein R³, R², R⁵, R⁴, X², and X³ are as defined in any one of aspects 1 to 45,

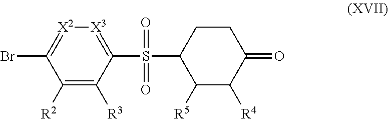

reacting said compound of formula (XVII) with compound of formula (VI), wherein R⁶, R⁷, and X⁵ are as defined in any one of aspects 1 to 45,

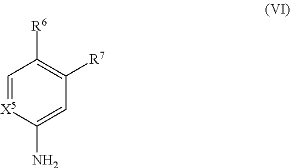

to form compound of formula (XVIII), wherein R², R³, R⁴, R⁶, R⁷, R⁵, X², X⁵ and X³ are as defined in any one of aspects 1 to 45,

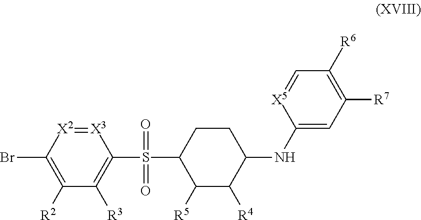

reacting said compound of formula (XVIII) with compound of formula (X) wherein R¹ is as defined in any one of aspects 1 to 45, and R⁹ and R¹⁰ are independently selected from C₁₋₆ alkyl, or R⁹ and R¹⁰ taken together with the oxygen atom to which they are attached, form a 3- to 14-membered heterocyclyl, optionally substituted with one or more, in particular four, C₁₋₆alkyl,

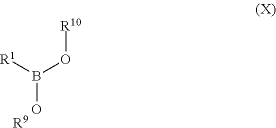

to form compound of formula (I).

Aspect 48. A process of preparation of a compound of formula (I) or (I′) according to any one of aspects 1 to 45, or a pharmaceutically acceptable salt thereof wherein X¹ is CH and X⁴ is NH, comprising:
reacting compound of formula (XV), wherein R³, R², R⁵, R⁴, X², and X³ are as defined in any one of aspects 1 to 45,

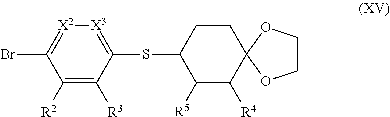

with an acid to form compound of formula (XX), wherein R³, R², R⁵, R⁴, X², and X³ are as defined in any one of aspects 1 to 45,

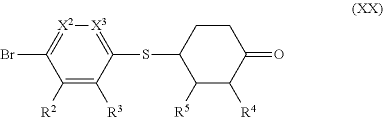

reacting said compound of formula (XX) with compound of formula (VI), wherein R⁶, R⁷, and X⁵ are as defined in any one of aspects 1 to 45,

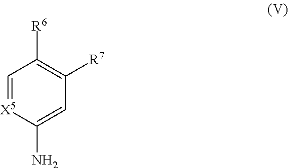

to form compound of formula (XXI), wherein R², R³, R⁴, R⁶, R⁷, R⁵, X², X⁵ and X³ are as defined in any one of aspects 1 to 45,

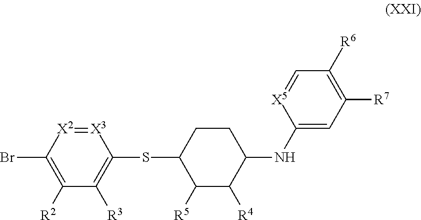

reacting said compound of formula (XXI) with ammonium carbamate and (diacetoxyiodo)benzene to form compound of formula (XXII), wherein R², R³, R⁴, R⁶, R⁷, R⁵, X², X⁵ and X³ are as defined in any one of aspects 1 to 45,

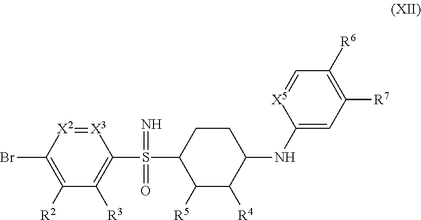

reacting said compound of formula (XXII) with compound of formula (X) wherein R¹ is as defined in any one of aspects 1 to 45, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

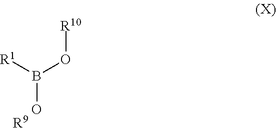

to form compound of formula (I).

Aspect 49. A process of preparation of a compound of formula (I) or (I′) according to any one of aspects 1 to 45, or a pharmaceutically acceptable salt thereof wherein X¹ is N and X⁴ is NH, comprising:
reacting compound of formula (IX), wherein R⁴, R⁵, X⁵, R⁶, R⁷ are as defined in any one of aspects 1 to 45,

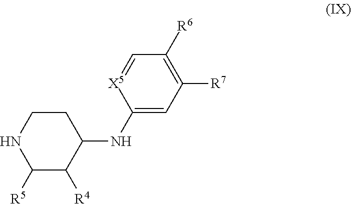

with compound of formula (XIV), wherein R² and R³ are as defined in any one of aspects 1 to 45,

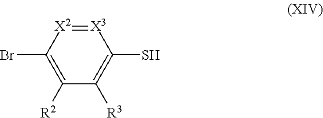

to form compound of formula (XXIV), wherein R², R³, R⁴, R⁶, R⁷, R⁵, X², X⁵ and X³ are as defined in any one of aspects 1 to 45,

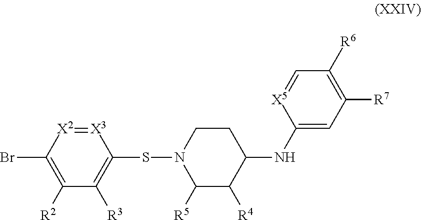

reacting said compound of formula (XXIV) with ammonium carbamate and (diacetoxyiodo)benzene to form compound of formula (XXV), wherein R², R³, R⁴, R⁶, R⁷, R⁵, X², X⁵ and X³ are as defined in any one of aspects 1 to 45,

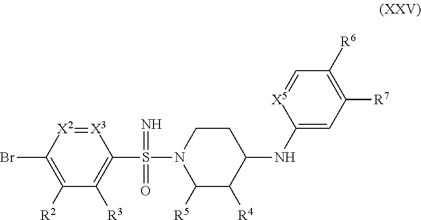

reacting said compound of formula (XXV) with di-tert-butyl dicarbonate and NaH to form compound of formula (XXVI), wherein R², R³, R⁴, R⁶, R⁷, R⁵, X², X⁵ and X³ are as defined in any one of aspects 1 to 45,

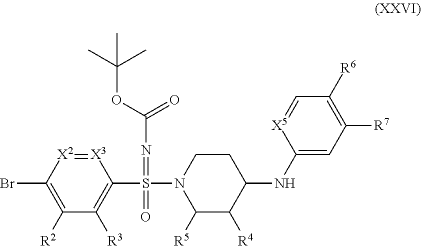

reacting said compound of formula (XXVI) with compound of formula (X), wherein R¹ is as defined in any one of aspects 1 to 45, and R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

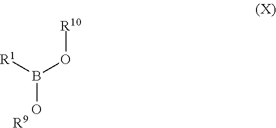

to form compound of formula (XXVII), wherein R¹, R², R³, R⁴, R⁶, R⁷, R⁵, X², X⁵ and X³ are as defined in any one of aspects 1 to 45,

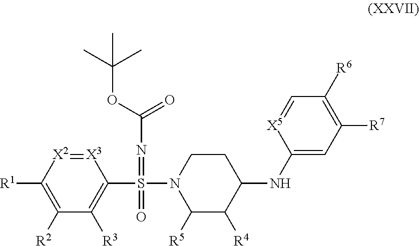

reacting said compound of formula (XXVII) with acid to form compound of formula (I).

Aspect 50. A compound according to any one of aspects 1 to 45, or a pharmaceutically acceptable salt thereof, when manufactured according to the process of any one of the aspects 46 to 49.

Aspect 51. A compound according to any one of aspects 1 to 45, or a pharmaceutically acceptable salt thereof, for use as therapeutically active substance.

Aspect 52. A pharmaceutical composition comprising a compound according to any one of aspects 1 to 45, or a pharmaceutically acceptable salt thereof, and a pharmaceutically acceptable excipient.

Aspect 53. The pharmaceutical composition according to aspect 52, further comprising an additional therapeutic agent.

Aspect 54. A compound according to any one of aspects 1 to 45, or a pharmaceutically acceptable salt thereof, for use in the treatment, prevention and/or delay of progression of an inflammatory autoimmune disease.

Aspect 55. A compound according to any one of aspects 1 to 45, or a pharmaceutically acceptable salt thereof, for use in the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus and multiple sclerosis.

Aspect 56. A compound according to any one of aspects 1 to 45, or a pharmaceutically acceptable salt thereof, for use in the treatment, prevention and/or delay of progression of psoriatic diseases, asthma, Crohn's disease, ulcerative colitis, rheumatoid arthritis, systemic lupus erythematosus and multiple sclerosis.

Aspect 57. The use of a compound according to any one of aspects 1 to 45, or a pharmaceutically acceptable salt thereof, for the preparation of a medicament for the treatment, prevention and/or delay of an inflammatory autoimmune disease.

Aspect 58. The use of a compound according to any one of aspects 1 to 45, or a pharmaceutically acceptable salt thereof, for the preparation of a medicament for the treatment, prevention and/or delay of psoriatic diseases, asthma, Crohn's disease, ulcerative colitis, rheumatoid arthritis, systemic lupus erythematosus and multiple sclerosis.

Aspect 59. A method for the treatment, prevention and/or delay of progression of an inflammatory autoimmune disease, which method comprises administering a therapeutically effective amount of a compound according to any one of aspects 1 to 45, or a pharmaceutically acceptable salt thereof.

Aspect 60. A method for the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus or multiple sclerosis, which method comprises administering a therapeutically effective amount of a compound according to any one of aspects 1 to 45, or a pharmaceutically acceptable salt thereof.

1. A compound of formula (I) 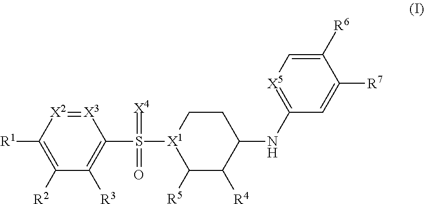
wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a};
R^{1a} is selected from C₁₋₆alkyl, cyano, —NR^{1b}R^{1c}, —C₁₋₆alkyl-NR^{1b}R^{1c}, oxo, —CONR^{1b}R^{1c}, —PO(Me)₂, hydroxy, C₃₋₆cycloalkyl, —C₁₋₆alkyl-C₁₋₆alkoxy, C₃₋₆heterocyclyl, haloC₁₋₆alkyl, halo, and C₁₋₆alkoxy;
R^{1b} is hydrogen, C₃₋₆cycloalkyl, or C₁₋₆alkyl;
R^{1c} is hydrogen, C₃₋₆cycloalkyl, or C₁₋₆alkyl;
R² is hydrogen or halogen;
R³ is hydrogen or halogen;
R⁴ is hydrogen, C₁₋₆alkyl, or halogen;
R⁵ is hydrogen, halogen, or C₁₋₆alkyl;
R⁶ is hydrogen or —SF₅, wherein R⁶ is different from R⁷; and
R⁷ is hydrogen or —SF₅, wherein R⁷ is different from R⁶;
or a pharmaceutically acceptable salt thereof.

2. The compound of claim 1 wherein
R^{1a} is selected from C₁₋₆alkyl, cyano, oxo, —CONR^{1b}R^{1c}, —PO(Me)₂, hydroxy, C₃₋₆cycloalkyl, —C₁₋₆alkylC₁₋₆alkoxy, haloC₁₋₆alkyl, halo, and C₁₋₆alkoxy;
R^{1b} is hydrogen or C₁₋₆alkyl;
R^{1c} is hydrogen or C₁₋₆alkyl;
X⁵ is CH; and
R⁵ is hydrogen;
or a pharmaceutically acceptable salt thereof.

3-12. (canceled)

13. The compound of claim 1, or a pharmaceutically acceptable salt thereof, wherein X¹ is N, X² is CH, X³ is CH, X⁴ is O, and X⁵ is CH.

14. The compound of claim 1, or a pharmaceutically acceptable salt thereof, wherein X¹ is CH, X² is CH, X³ is CH, X⁴ is O, and X⁵ is CH.

15. The compound of claim 1, or a pharmaceutically acceptable salt thereof, wherein X¹ is N, X² is N, X³ is CH, X⁴ is O, and X⁵ is CH.

16. The compound of claim 1, or a pharmaceutically acceptable salt thereof, wherein X¹ is N, X² is CH, X³ is N, X⁴ is O, and X⁵ is CH.

17. The compound of claim 1, or a pharmaceutically acceptable salt thereof, wherein X¹ is CH, X² is CH, X³ is CH, X⁴ is NH, and X⁵ is CH.

18. The compound of claim 1, or a pharmaceutically acceptable salt thereof, wherein X¹ is N, X² is CH, X³ is CH, X⁴ is NH, and X⁵ is CH.

19. The compound of claim 1, or a pharmaceutically acceptable salt thereof, wherein R¹ is azaspirononanyl, triazaspirodecanyl, diazaspirodecanyl, oxadiazaspirodecanyl, oxaazaspirononanyl, oxaazabicyclooctanyl, pyrazolopyridinyl, pyrrolopyrimidinyl, pyrrolopyridinyl, thiophenyl, thienopyridinyl, pyrrolopyrazinyl, pyrrolodiazepinyl, furanyl, indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, or spirocyclopropaneindolinyl, wherein azaspirononanyl, triazaspirodecanyl, diazaspirodecanyl, oxadiazaspirodecanyl, oxaazaspirononanyl, oxaazabicyclooctanyl, pyrazolopyridinyl, pyrrolopyrimidinyl, pyrrolopyridinyl, thiophenyl, thienopyridinyl, pyrrolopyrazinyl, pyrrolodiazepinyl, furanyl, indazolyl, indolyl, isoindolinyl, triazolopyridinyl, imidazopyridinyl, imidazopyrazinyl, indolinyl, phenyl, pyridyl, triazolopyridazinyl, isoquinolinyl, pyridazinyl, triazolopyrazinyl, pyrrolotriazinyl, and spirocyclopropaneindolinyl are optionally substituted with one or more R^{1a}.

20-21. (canceled)

22. The compound of claim 1, or a pharmaceutically acceptable salt thereof, wherein R¹ is methylindazolyl, cyano-indolyl, cyano-indazolyl, oxo-isoindolinyl, Carbamoylpyridyl, methyl-triazolopyridinyl, imidazopyridinyl, cyano-imidazopyridinyl, imidazopyrazinyl, methylimidazopyridinyl, carbamoylimidazopyridinyl, isopropyl-triazolopyridinyl, triazolopyridinyl, carbamoylindazolyl, oxo-indolinyl, carbamoyl-triazolopyridinyl, cyano-triazolopyridinyl, (dimethylcarbamoyl)phenyl, methylimidazopyrazinyl, (dimethylcarbamoyl)pyridyl, (methylcarbamoyl)phenyl, (methylcarbamoyl)pyridyl, oxo-triazolopyridinyl, carbamoylphenyl, isopropyl-triazolopyridazinyl, oxo-dihydro-isoquinolinyl, carbamoylpyridazinyl, dimethylphosphorylphenyl, isopropyl-triazolopyrazinyl, oxo-pyridinyl, oxo-pyrrolotriazinyl, hydroxypyridyl, (dimethylcarbamoyl)pyridyl, oxo-spirocyclopropaneindolineyl, methoxyazaspirononanyl, oxo-triazaspirodecanyl, oxo-diazaspirodecanyl, oxo-oxadiazaspirodecanyl, oxaazaspirononanyl, oxaazabicyclooctanyl, carbamoylfluorophenyl, propanyltriazolopyrazinyl, isopropycarbamoylphenyl, methylpyrazolopyridinyl, N,N-dimethylamineimidazopyridinyl, cyclopropylcarbamoylphenyl, pyrrolopyrimidinyl, pyrrolopyridinyl, pyrazolopyridinyl, carbamoylthiophenyl, methylimidazopyridinyl, (trifluoromethyl)imidazopyridinyl, fluoroimidazopyridinyl, oxo-thienopyridinyl, oxo-pyrrolopyrazinyl, oxo-pyrrolodiazepinyl, carbamoylimidazopyridinyl, furancarboxamide, cyano-imidazopyridinyl, pyrrolidinyl-triazolopyridinyl, propanyl-triazolopyrazinyl, or azetidinyl-triazolopyridinyl.

23-24. (canceled)

25. The compound of claim 1, or a pharmaceutically acceptable salt thereof, wherein R^{1a} is selected from methoxy, fluoro, —CONH[(CH(CH3)₂], —CONH(cyclopropyl), dimethylamino, —CF₃, pyrrolidinyl, azetidinyl, methyl, cyano, oxo, —CONH₂, isopropyl, —CON(Me)₂, —CH₂—O—CH₃, —CONH(Me), —PO(Me)₂, and —OH.

26-36. (canceled)

37. The compound of claim 1, wherein the compound is of formula (I′) 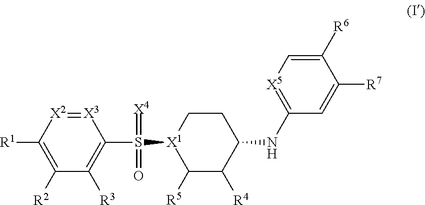
or a pharmaceutically acceptable salt thereof, wherein R¹, R², R³, R⁴, R⁵, R⁶, R⁷, X², X³, X⁴, and X⁵ are as defined in claim 1, and X¹ is CH.

38. The compound of claim 1, or
a pharmaceutically acceptable salt thereof, wherein
X¹ is CH or N,
X² is CH,
X³ is CH,
X⁴ is O or NH,
X⁵ is CH,
R¹ is indazolyl, indolyl, pyridyl, triazolopyridinyl, phenyl, or imidazopyridinyl, wherein indazolyl, indolyl, pyridyl, triazolopyridinyl, phenyl, and imidazopyridinyl are optionally substituted with one or more R^{1a},
R^{1a} is selected from methyl, cyano, —CONH₂, isopropyl, and —CONH(Me),
R² is hydrogen,
R³ is hydrogen,
R⁴ is hydrogen,
R⁵ is hydrogen,
R⁶ is —SF₅, and
R⁷ is hydrogen.

39. (canceled)

40. The compound of claim 1, or a pharmaceutical acceptable salt thereof, selected from:
1-(4-{imidazo[1,2-a]pyridin-6-yl}benzenesulfonyl)-N-[4-(pentafluoro-λ⁶-sulfanyl)phenyl]piperidin-4-amine;
1-[4-(3-methyl-2H-indazol-5-yl)benzenesulfonyl]-N-[4-(pentafluoro-λ⁶-sulfanyl)phenyl]piperidin-4-amine;
5-{4-[(4-{[4-(pentafluoro-λ⁶-sulfanyl)phenyl]Amino}piperidin-1-yl)sulfonyl]phenyl}-1H-indole-3-carbonitrile;
5-{4-[(4-{[4-(pentafluoro-λ⁶-sulfanyl)phenyl]Amino}piperidin-1-yl)sulfonyl]phenyl}-1H-indazole-3-carbonitrile;
4-(pentafluoro-λ⁶-sulfanyl)-N-[cis-4-[4-(3-methyl-2H-indazol-5-yl)benzenesulfonyl]cyclohexyl]aniline;
4-(pentafluoro-λ⁶-sulfanyl)-N-[trans-4-[4-(3-methyl-2H-indazol-5-yl)benzenesulfonyl]cyclohexyl]aniline;
5-(4-{[trans-4-{[4-(pentafluoro-λ⁶-sulfanyl)phenyl]Amino}cyclohexyl]sulfonyl}phenyl)-1H-indole-3-carbonitrile;
5-(4-{[cis-4-{[4-(pentafluoro-λ⁶-sulfanyl)phenyl]Amino}cyclohexyl]sulfonyl}phenyl)-1H-indole-3-carbonitrile;
6-(4-{[trans-4-{[4-(pentafluoro-λ⁶-sulfanyl)phenyl]Amino}cyclohexyl]sulfonyl}phenyl)-2,3-dihydro-1H-isoindol-1-one;
4-(4-{[trans-4-{[4-(pentafluoro-λ⁶-sulfanyl)phenyl]Amino}cyclohexyl]sulfonyl}phenyl)pyridine-2-carboxamide;
4-(pentafluoro-λ⁶-sulfanyl)-N-[trans-4-(4-{3-methyl-[1,2,4]triazolo[4,3-a]pyridin-6-yl}benzenesulfonyl)cyclohexyl]aniline;
4-(pentafluoro-λ⁶-sulfanyl)-N-[trans-4-(4-{imidazo[1,2-a]pyridin-6-yl}benzenesulfonyl)cyclohexyl]aniline;
4-(pentafluoro-λ⁶-sulfanyl)-N-[trans-4-(4-{3-methylimidazo[1,2-a]pyridin-6-yl}benzenesulfonyl)cyclohexyl]aniline;
5-(4-{[trans-4-{[4-(pentafluoro-λ⁶-sulfanyl)phenyl]Amino}cyclohexyl]sulfonyl}phenyl)-2,3-dihydro-1H-indol-2-one;
N,N-dimethyl-4′-{[trans-4-{[4-(pentafluoro-λ⁶-sulfanyl)phenyl]Amino}cyclohexyl]sulfonyl}-[1,1′-biphenyl]-4-carboxamide;
N-methyl-4′-{[trans-4-{[4-(pentafluoro-λ⁶-sulfanyl)phenyl]Amino}cyclohexyl]sulfonyl}-[1,1′-biphenyl]-4-carboxamide;
6-(4-{[cis-4-{[4-(pentafluoro-λ⁶-sulfanyl)phenyl]Amino}cyclohexyl]sulfonyl}phenyl)imidazo[1,2-a]pyridine-3-carbonitrile;
4-(pentafluoro-λ⁶-sulfanyl)-N-[cis-4-(4-{imidazo[1,2-a]pyridin-6-yl}benzenesulfonyl)cyclohexyl]aniline;
N,N-dimethyl-4′-{[trans-4-{[4-(pentafluoro-λ⁶-sulfanyl)phenyl]Amino}cyclohexyl]sulfonyl}-[1,1′-biphenyl]-3-carboxamide;
N,N-dimethyl-5-(4-{[trans-4-{[4-(pentafluoro-λ⁶-sulfanyl)phenyl]Amino}cyclohexyl]sulfonyl}phenyl)pyridine-2-carboxamide;
N-methyl-4′-{[trans-4-{[4-(pentafluoro-λ⁶-sulfanyl)phenyl]Amino}cyclohexyl]sulfonyl}-[1,1′-biphenyl]-3-carboxamide;
N-methyl-5-(4-{[trans-4-{[4-(pentafluoro-λ⁶-sulfanyl)phenyl]Amino}cyclohexyl]sulfonyl}phenyl)pyridine-2-carboxamide;
4′-{[trans-4-{[4-(pentafluoro-λ⁶-sulfanyl)phenyl]Amino}cyclohexyl]sulfonyl}-[1,1′-biphenyl]-3-carboxamide;
1-(4-{imidazo[1,2-a]pyrazin-6-yl}benzenesulfonyl)-N-[4-(pentafluoro-λ⁶-sulfanyl)phenyl]piperidin-4-amine;
6-{4-[(4-{[4-(pentafluoro-λ⁶-sulfanyl)phenyl]Amino}piperidin-1-yl)sulfonyl]phenyl}imidazo[1,2-a]pyridine-3-carbonitrile;
6-{4-[(4-{[4-(pentafluoro-λ⁶-sulfanyl)phenyl]Amino}piperidin-1-yl)sulfonyl]phenyl}-[1,2,4]triazolo[4,3-a]pyridine-3-carboxamide;
1-(4-{3-methyl-[1,2,4]triazolo[4,3-a]pyridin-6-yl}benzenesulfonyl)-N-[4-(pentafluoro-λ⁶-sulfanyl)phenyl]piperidin-4-amine;
N-[4-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine;
N-[4-(pentafluoro-λ⁶-sulfanyl)phenyl]-1-{4-[3-(propan-2-yl)imidazo[1,2-a]pyridin-6-yl]benzenesulfonyl}piperidin-4-amine;
1-{4-[3-(methoxymethyl)-[1,2,4]triazolo[4,3-a]pyridin-6-yl]benzenesulfonyl}-N-[4-(pentafluoro-λ⁶-sulfanyl)phenyl]piperidin-4-amine;
1-(4-{3-cyclopentyl-[1,2,4]triazolo[4,3-a]pyridin-6-yl}benzenesulfonyl)-N-[4-(pentafluoro-λ⁶-sulfanyl)phenyl]piperidin-4-amine;
6-{4-[(4-{[4-(pentafluoro-λ⁶-sulfanyl)phenyl]Amino}piperidin-1-yl)sulfonyl]phenyl}-2H,3H-[1,2,4]triazolo[4,3-a]pyridin-3-one;
6-(4-{[trans-4-{[4-(pentafluoro-λ⁶-sulfanyl)phenyl]Amino}cyclohexyl]sulfonyl}phenyl)imidazo[1,2-a]pyridine-3-carbonitrile;
6-(4-{[trans-4-{[4-(pentafluoro-λ⁶-sulfanyl)phenyl]Amino}cyclohexyl]sulfonyl}phenyl)imidazo[1,2-a]pyridine-3-carboxamide;
4-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]aniline;
4-(pentafluoro-λ⁶-sulfanyl)-N-[trans-4-(4-{[1,2,4]triazolo[4,3-a]pyridin-6-yl}benzenesulfonyl)cyclohexyl]aniline;
5-(4-{[trans-4-{[4-(pentafluoro-λ⁶-sulfanyl)phenyl]Amino}cyclohexyl]sulfonyl}phenyl)-1H-indazole-3-carboxamide;
5-(4-{[trans-4-{[4-(pentafluoro-λ⁶-sulfanyl)phenyl]Amino}cyclohexyl]sulfonyl}phenyl)-1H-indazole-3-carbonitrile;
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carboxamide;
4-(pentafluoro-λ⁶-sulfanyl)-N-[trans-4-(4-{2-methyl-[1,2,4]triazolo[1,5-a]pyridin-6-yl}benzenesulfonyl)cyclohexyl]aniline;
4-(pentafluoro-λ⁶-sulfanyl)-N-[trans-4-(4-{2-methyl-[1,2,4]triazolo[1,5-a]pyridin-7-yl}benzenesulfonyl)cyclohexyl]aniline;
6-(4-{[trans-4-{[4-(pentafluoro-λ⁶-sulfanyl)phenyl]Amino}cyclohexyl]sulfonyl}phenyl)-[1,2,4]triazolo[4,3-a]pyridine-3-carbonitrile;
4-(pentafluoro-λ⁶-sulfanyl)-N-[trans-4-(4-{2-methylimidazo[1,2-a]pyrazin-6-yl}benzenesulfonyl)cyclohexyl]aniline;
4-(pentafluoro-λ⁶-sulfanyl)-N-[trans-4-(4-{imidazo[1,2-a]pyrazin-6-yl}benzenesulfonyl)cyclohexyl]aniline;
N,N-dimethyl-6-(4-{[cis-4-{[4-(pentafluoro-λ⁶-sulfanyl)phenyl]Amino}cyclohexyl]sulfonyl}phenyl)pyridine-3-carboxamide;
N,N-dimethyl-6-(4-{[trans-4-{[4-(pentafluoro-λ⁶-sulfanyl)phenyl]Amino}cyclohexyl]sulfonyl}phenyl)pyridine-3-carboxamide;
N-methyl-6-(4-{[trans-4-{[4-(pentafluoro-λ⁶-sulfanyl)phenyl]Amino}cyclohexyl]sulfonyl}phenyl)pyridine-3-carboxamide;
N-methyl-4-(4-{[trans-4-{[4-(pentafluoro-λ⁶-sulfanyl)phenyl]Amino}cyclohexyl]sulfonyl}phenyl)pyridine-2-carboxamide;
6-(4-{[trans-4-{[4-(pentafluoro-λ⁶-sulfanyl)phenyl]Amino}cyclohexyl]sulfonyl}phenyl)pyridine-3-carboxamide;
4-(pentafluoro-λ⁶-sulfanyl)-N-[trans-4-{4-[3-(propan-2-yl)-[1,2,4]triazolo[4,3-b]pyridazin-6-yl]benzenesulfonyl}cyclohexyl]aniline;
4-(pentafluoro-λ⁶-sulfanyl)-N-[trans-4-{4-[3-(propan-2-yl)-[1,2,4]triazolo[4,3-a]pyrazin-6-yl]benzenesulfonyl}cyclohexyl]aniline;
4-(4-{[trans-4-{[4-(pentafluoro-λ⁶-sulfanyl)phenyl]Amino}cyclohexyl]sulfonyl}phenyl)-1,2-dihydropyridin-2-one;
7-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one;
6-(4-{[trans-4-{[4-(pentafluoro-λ⁶-sulfanyl)phenyl]Amino}cyclohexyl]sulfonyl}phenyl)-1,2,3,4-tetrahydroisoquinolin-3-one;
6-(4-{[trans-4-{[4-(pentafluoro-λ⁶-sulfanyl)phenyl]Amino}cyclohexyl]sulfonyl}phenyl)-3H,4H-pyrrolo[2,1-f][1,2,4]triazin-4-one;
5-(4-{[trans-4-{[4-(pentafluoro-λ⁶-sulfanyl)phenyl]Amino}cyclohexyl]sulfonyl}phenyl)pyridin-2-ol
4-(pentafluoro-λ⁶-sulfanyl)-N-[trans-4-{[4′-(dimethylphosphoryl)-[1,1′-biphenyl]-4-yl]sulfonyl}cyclohexyl]aniline;
5-(4-{[trans-4-{[4-(pentafluoro-λ⁶-sulfanyl)phenyl]Amino}cyclohexyl]sulfonyl}phenyl)-2,3-dihydro-1H-isoindol-1-one;
6-(4-{[trans-4-{[4-(pentafluoro-λ⁶-sulfanyl)phenyl]Amino}cyclohexyl]sulfonyl}phenyl)-2,3-dihydro-1H-indol-2-one;
7-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one;
6-(4-{[trans-4-{[4-(pentafluoro-λ⁶-sulfanyl)phenyl]Amino}cyclohexyl]sulfonyl}phenyl)-1,2,3,4-tetrahydroisoquinolin-1-one;
6-(4-{[trans-4-{[4-(pentafluoro-λ⁶-sulfanyl)phenyl]Amino}cyclohexyl]sulfonyl}phenyl)pyridazine-3-carboxamide;
4-(pentafluoro-λ⁶-sulfanyl)-N-[trans-4-{4-[3-(propan-2-yl)imidazo[1,2-a]pyridin-6-yl]benzenesulfonyl}cyclohexyl]aniline;
4-(pentafluoro-λ⁶-sulfanyl)-N-[trans-4-{4-[8-methyl-3-(propan-2-yl)imidazo[1,2-a]pyridin-6-yl]benzenesulfonyl}cyclohexyl]aniline;
5′-(4-{[trans-4-{[4-(pentafluoro-λ⁶-sulfanyl)phenyl]amino}cyclohexyl]sulfonyl}phenyl)-1′,2′-dihydrospiro[cyclopropane-1,3′-indol]-2′-one;
6-(4-{[trans-4-{[4-(pentafluoro-λ⁶-sulfanyl)phenyl]Amino}cyclohexyl]sulfonyl}phenyl)-2H,3H-[1,2,4]triazolo[4,3-a]pyridin-3-one;
1-(2-fluoro-4-{3-methylimidazo[1,2-a]pyridin-6-yl}benzenesulfonyl)-N-[4-(pentafluoro-λ⁶-sulfanyl)phenyl]piperidin-4-amine;
1-(3-fluoro-4-{3-methylimidazo[1,2-a]pyridin-6-yl}benzenesulfonyl)-N-[4-(pentafluoro-λ⁶-sulfanyl)phenyl]piperidin-4-amine;
1-[(6-{3-methylimidazo[1,2-a]pyridin-6-yl}pyridin-3-yl)sulfonyl]-N-[4-(pentafluoro-λ⁶-sulfanyl)phenyl]piperidin-4-amine;
1-[(5-{3-methylimidazo[1,2-a]pyridin-6-yl}pyridin-2-yl)sulfonyl]-N-[4-(pentafluoro-λ⁶-sulfanyl)phenyl]piperidin-4-amine;
6-{2-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile;
1-(3-fluoro-4-{imidazo[1,2-a]pyridin-6-yl}benzenesulfonyl)-N-[4-(pentafluoro-λ⁶-sulfanyl)phenyl]piperidin-4-amine;
6-{3-fluoro-4-[(4-{[4-(pentafluoro-λ⁶-sulfanyl)phenyl]Amino}piperidin-1-yl)sulfonyl]phenyl}imidazo[1,2-a]pyridine-3-carbonitrile;
1-(2-fluoro-4-{imidazo[1,2-a]pyridin-6-yl}benzenesulfonyl)-N-[4-(pentafluoro-λ⁶-sulfanyl)phenyl]piperidin-4-amine;
1-[(5-{imidazo[1,2-a]pyridin-6-yl}pyridin-2-yl)sulfonyl]-N-[4-(pentafluoro-λ⁶-sulfanyl)phenyl]piperidin-4-amine;
6-{5-[(4-{[4-(pentafluoro-λ⁶-sulfanyl)phenyl]Amino}piperidin-1-yl)sulfonyl]pyridin-2-yl}imidazo[1,2-a]pyridine-3-carbonitrile;
1-[(6-{imidazo[1,2-a]pyridin-6-yl}pyridin-3-yl)sulfonyl]-N-[4-(pentafluoro-λ⁶-sulfanyl)phenyl]piperidin-4-amine;
6-{6-[(4-{[4-(pentafluoro-λ⁶-sulfanyl)phenyl]Amino}piperidin-1-yl)sulfonyl]pyridin-3-yl}imidazo[1,2-a]pyridine-3-carbonitrile;
1-(2-chloro-4-{imidazo[1,2-a]pyridin-6-yl}benzenesulfonyl)-N-[4-(pentafluoro-λ⁶-sulfanyl)phenyl]piperidin-4-amine;
6-{3-chloro-4-[(4-{[4-(pentafluoro-λ⁶-sulfanyl)phenyl]Amino}piperidin-1-yl)sulfonyl]phenyl}imidazo[1,2-a]pyridine-3-carbonitrile;
6-{4-[(4-{[3-(pentafluoro-λ⁶-sulfanyl)phenyl]Amino}piperidin-1-yl)sulfonyl]phenyl}imidazo[1,2-a]pyridine-3-carbonitrile;
1-(4-{imidazo[1,2-a]pyridin-6-yl}benzenesulfonyl)-N-[3-(pentafluoro-λ⁶-sulfanyl)phenyl]piperidin-4-amine;
4-(4-{[(3 S,4S)-3-fluoro-4-{[4-(pentafluoro-λ⁶-sulfanyl)phenyl]Amino}piperidin-1-yl]sulfonyl}phenyl)pyridine-2-carboxamide;
N-[3-(pentafluoro-λ⁶-sulfanyl)phenyl]-1-{4-[3-(propan-2-yl)-[1,2,4]triazolo[4,3-a]pyridin-6-yl]benzenesulfonyl}piperidin-4-amine;
5-(4-{[trans-4-{[4-(pentafluoro-λ⁶-sulfanyl)phenyl]Amino}cyclohexyl]sulfonimidoyl}phenyl)-1H-indole-3-carbonitrile;
[4-(3-methyl-1H-indazol-5-yl)phenyl][trans-4-{[4-(pentafluoro-λ⁶-sulfanyl)phenyl]Amino}cyclohexyl](imino)-λ⁶-sulfanone;
(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone;
(4-{imidazo[1,2-a]pyridin-6-yl}phenyl)[cis-4-{[4-(pentafluoro-λ⁶-sulfanyl)phenyl]Amino}cyclohexyl](imino)-λ⁶-sulfanone;
(4-{3-methylimidazo[1,2-a]pyridin-6-yl}phenyl)[trans-4-{[4-(pentafluoro-λ⁶-sulfanyl)phenyl]Amino}cyclohexyl](imino)-λ⁶-sulfanone;
6-(4-{[trans-4-{[4-(pentafluoro-λ⁶-sulfanyl)phenyl]Amino}cyclohexyl]sulfonimidoyl}phenyl)imidazo[1,2-a]pyridine-3-carbonitrile;
4-(4-{[trans-4-{[4-(pentafluoro-λ⁶-sulfanyl)phenyl]Amino}cyclohexyl]sulfonimidoyl}phenyl)pyridine-2-carboxamide;
N,N-dimethyl-4′-{[trans-4-{[4-(pentafluoro-λ⁶-sulfanyl)phenyl]Amino}cyclohexyl]sulfonimidoyl}-[1,1′-biphenyl]-4-carboxamide;
(4-{imidazo[1,2-a]pyridin-7-yl}phenyl)[trans-4-{[4-(pentafluoro-λ⁶-sulfanyl)phenyl]Amino}cyclohexyl](imino)-λ⁶-sulfanone;
[trans-4-{[4-(pentafluoro-λ⁶-sulfanyl)phenyl]Amino}cyclohexyl](4-{[1,2,4]triazolo[1,5-a]pyridin-7-yl}phenyl)(imino)-λ⁶-sulfanone;
N-methyl-4′-{[trans-4-{[4-(pentafluoro-λ⁶-sulfanyl)phenyl]Amino}cyclohexyl]sulfonimidoyl}-[1,1′-biphenyl]-4-carboxamide;
4′-{[trans-4-{[4-(pentafluoro-λ⁶-sulfanyl)phenyl]Amino}cyclohexyl]sulfonimidoyl}-[1,1′-biphenyl]-4-carboxamide;
N,N-dimethyl-4′-{[trans-4-{[4-(pentafluoro-λ⁶-sulfanyl)phenyl]Amino}cyclohexyl]sulfonimidoyl}-[1,1′-biphenyl]-3-carboxamide;
N-methyl-4′-{[trans-4-{[4-(pentafluoro-λ⁶-sulfanyl)phenyl]Amino}cyclohexyl]sulfonimidoyl}-[1,1′-biphenyl]-3-carboxamide;
4′-{[trans-4-{[4-(pentafluoro-λ⁶-sulfanyl)phenyl]Amino}cyclohexyl]sulfonimidoyl}-[1,1′-biphenyl]-3-carboxamide;
N,N-dimethyl-5-(4-{[trans-4-{[4-(pentafluoro-λ⁶-sulfanyl)phenyl]Amino}cyclohexyl]sulfonimidoyl}phenyl)pyridine-2-carboxamide;
N-methyl-5-(4-{[trans-4-{[4-(pentafluoro-λ⁶-sulfanyl)phenyl]Amino}cyclohexyl]sulfonimidoyl}phenyl)pyridine-2-carboxamide;
{4-[3-(propan-2-yl)-[1,2,4]triazolo[4,3-a]pyridin-6-yl]phenyl}[trans-4-{[4-(pentafluoro-λ⁶-sulfanyl)phenyl]Amino}cyclohexyl](imino)-λ⁶-sulfanone;
(4-{2-methylimidazo[1,2-a]pyrazin-6-yl}phenyl)[trans-4-{[4-(pentafluoro-λ⁶-sulfanyl)phenyl]Amino}cyclohexyl](imino)-λ⁶-sulfanone;
(4-{3-methyl-[1,2,4]triazolo[4,3-a]pyridin-7-yl}phenyl)[trans-4-{[4-(pentafluoro-λ⁶-sulfanyl)phenyl]Amino}cyclohexyl](imino)-λ⁶-sulfanone;
(4-{3-methyl-[1,2,4]triazolo[4,3-a]pyridin-6-yl}phenyl)[trans-4-{[4-(pentafluoro-λ⁶-sulfanyl)phenyl]Amino}cyclohexyl](imino)-λ⁶-sulfanone;
N,N-dimethyl-6-(4-{[trans-4-{[4-(pentafluoro-λ⁶-sulfanyl)phenyl]Amino}cyclohexyl]sulfonimidoyl}phenyl)pyridine-3-carboxamide;
N-methyl-6-(4-{[trans-4-{[4-(pentafluoro-λ⁶-sulfanyl)phenyl]Amino}cyclohexyl]sulfonimidoyl}phenyl)pyridine-3-carboxamide;
5-(4-{[trans-4-{[4-(pentafluoro-λ⁶-sulfanyl)phenyl]Amino}cyclohexyl]sulfonimidoyl}phenyl)pyridine-2-carboxamide;
N,N-dimethyl-4-(4-{[trans-4-{[4-(pentafluoro-λ⁶-sulfanyl)phenyl]Amino}cyclohexyl]sulfonimidoyl}phenyl)pyridine-2-carboxamide;
N-methyl-4-(4-{[trans-4-{[4-(pentafluoro-λ⁶-sulfanyl)phenyl]Amino}cyclohexyl]sulfonimidoyl}phenyl)pyridine-2-carboxamide;
6-(4-{[trans-4-{[4-(pentafluoro-λ⁶-sulfanyl)phenyl]Amino}cyclohexyl]sulfonimidoyl}phenyl)pyridine-3-carboxamide;
5-(4-{[trans-4-{[4-(pentafluoro-λ⁶-sulfanyl)phenyl]Amino}cyclohexyl]sulfonimidoyl}phenyl)-2,3-dihydro-1H-isoindol-1-one;
6-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one;
7-(4-{[trans-4-{[4-(pentafluoro-λ⁶-sulfanyl)phenyl]Amino}cyclohexyl]sulfonimidoyl}phenyl)-1,2,3,4-tetrahydroisoquinolin-1-one;
6-(4-{[trans-4-{[4-(pentafluoro-λ⁶-sulfanyl)phenyl]Amino}cyclohexyl]sulfonimidoyl}phenyl)-2,3-dihydro-1H-isoindol-1-one;
5-(4-{[trans-4-{[4-(pentafluoro-λ⁶-sulfanyl)phenyl]Amino}cyclohexyl]sulfonimidoyl}phenyl)-2,3-dihydro-1H-indol-2-one;
6-(4-{[trans-4-{[4-(pentafluoro-λ⁶-sulfanyl)phenyl]Amino}cyclohexyl]sulfonimidoyl}phenyl)-2,3-dihydro-1H-indol-2-one;
{4-[3-(propan-2-yl)imidazo[1,2-a]pyridin-6-yl]phenyl}[trans-4-{[4-(pentafluoro-λ⁶-sulfanyl)phenyl]Amino}cyclohexyl](imino)-λ⁶-sulfanone;
6-(4-{[trans-4-{[4-(pentafluoro-λ⁶-sulfanyl)phenyl]Amino}cyclohexyl]sulfonimidoyl}phenyl)-2H,3H-[1,2,4]triazolo[4,3-a]pyridin-3-one;
5-{4-[(4-{[4-(pentafluoro-λ⁶-sulfanyl)phenyl]Amino}piperidin-1-yl)sulfonimidoyl]phenyl}-1H-indole-3-carbonitrile;
N-methyl-5-{4-[(4-{[4-(pentafluoro-λ⁶-sulfanyl)phenyl]Amino}piperidin-1-yl)sulfonimidoyl]phenyl}pyridine-2-carboxamide;
4′-[(4-{[4-(pentafluoro-λ⁶-sulfanyl)phenyl]Amino}piperidin-1-yl)sulfonimidoyl]-[1,1′-biphenyl]-4-carbonitrile;
5-{4-[(4-{[4-(pentafluoro-λ⁶-sulfanyl)phenyl]Amino}piperidin-1-yl)sulfonimidoyl]phenyl}-2,3-dihydro-1H-indol-2-one;
(4-{imidazo[1,2-a]pyridin-6-yl}phenyl)(4-{[4-(pentafluoro-λ⁶-sulfanyl)phenyl]Amino}piperidin-1-yl)(imino)-λ⁶-sulfanone;
4-{4-[(4-{[4-(pentafluoro-λ⁶-sulfanyl)phenyl]Amino}piperidin-1-yl)sulfonimidoyl]phenyl}pyridine-2-carboxamide;
(+)-5-(4-{[trans-4-{[4-(pentafluoro-λ⁶-sulfanyl)phenyl]Amino}cyclohexyl]sulfonimidoyl}phenyl)-1H-indole-3-carbonitrile;
(−)-5-(4-{[trans-4-{[4-(pentafluoro-λ⁶-sulfanyl)phenyl]Amino}cyclohexyl]sulfonimidoyl}phenyl)-1H-indole-3-carbonitrile;
(+)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone;
(−)-(4-{imidazo[1,2-a]pyridin-6-yl}phenyl)[trans-4-{[4-(pentafluoro-λ⁶-sulfanyl)phenyl]Amino}cyclohexyl](imino)-λ⁶-sulfanone;
(+)-4-(4-{[trans-4-{[4-(pentafluoro-λ⁶-sulfanyl)phenyl]Amino}cyclohexyl]sulfonimidoyl}phenyl)pyridine-2-carboxamide;
(−)-4-(4-{[trans-4-{[4-(pentafluoro-λ⁶-sulfanyl)phenyl]Amino}cyclohexyl]sulfonimidoyl}phenyl)pyridine-2-carboxamide;
1-(4-{2-methoxy-7-azaspiro[3.5]nonan-7-yl}benzenesulfonyl)-N-[4-(pentafluoro-λ⁶-sulfanyl)phenyl]piperidin-4-amine;
8-{4-[(4-{[4-(pentafluoro-λ⁶-sulfanyl)phenyl]amino}piperidin-1-yl)sulfonyl]phenyl}-1,3,8-triazaspiro[4.5]decane-2,4-dione;
8-{4-[(4-{[4-(pentafluoro-λ⁶-sulfanyl)phenyl]amino}piperidin-1-yl)sulfonyl]phenyl}-2,8-diazaspiro[4.5]decan-1-one;
8-{4-[(4-{[4-(pentafluoro-λ⁶-sulfanyl)phenyl]amino}piperidin-1-yl)sulfonyl]phenyl}-3-oxa-1,8-diazaspiro[4.5]decan-2-one;
1-(4-{2-oxa-7-azaspiro[3.5]nonan-7-yl}benzenesulfonyl)-N-[4-(pentafluoro-λ⁶-sulfanyl)phenyl]piperidin-4-amine;
1-(4-{8-oxa-3-azabicyclo[3.2.1]octan-3-yl}benzenesulfonyl)-N-[4-(pentafluoro-λ⁶-sulfanyl)phenyl]piperidin-4-amine;
4-fluoro-N-methyl-4′-[(4-{[4-(pentafluoro-λ⁶-sulfanyl)phenyl]amino}piperidin-1-yl)sulfonyl]-[1,1′-biphenyl]-3-carboxamide;
3-fluoro-N-methyl-4′-[(4-{[4-(pentafluoro-λ⁶-sulfanyl)phenyl]amino}piperidin-1-yl)sulfonyl]-[1,1′-biphenyl]-4-carboxamide;
N-[4-(pentafluoro-λ⁶-sulfanyl)phenyl]-1-{4-[3-(propan-2-yl)-[1,2,4]triazolo[4,3-a]pyrazin-6-yl]benzenesulfonyl}piperidin-4-amine;
N-(propan-2-yl)-4′-{[trans-4-{[4-(pentafluoro-λ⁶-sulfanyl)phenyl]amino}cyclohexyl]sulfonyl}-[1,1′-biphenyl]-3-carboxamide;
4-(pentafluoro-λ⁶-sulfanyl)-N-[trans-4-(4-{3-methyl-1H-pyrazolo[3,4-c]pyridin-5-yl}benzenesulfonyl)cyclohexyl]aniline;
N,N-dimethyl-6-(4-{[trans-4-{[4-(pentafluoro-λ⁶-sulfanyl)phenyl]amino}cyclohexyl]sulfonyl}phenyl)imidazo[1,2-a]pyridin-3-amine;
N-cyclopropyl-4′-{[trans-4-{[4-(pentafluoro-λ⁶-sulfanyl)phenyl]amino}cyclohexyl]sulfonyl}-[1,1′-biphenyl]-3-carboxamide;
4-(pentafluoro-λ⁶-sulfanyl)-N-[trans-4-(4-{5H-pyrrolo[3,2-d]pyrimidin-2-yl}benzenesulfonyl)cyclohexyl]aniline;
4-(pentafluoro-λ⁶-sulfanyl)-N-[trans-4-(4-{1H-pyrrolo[2,3-c]pyridin-5-yl}benzenesulfonyl)cyclohexyl]aniline;
4-(pentafluoro-λ⁶-sulfanyl)-N-[trans-4-(4-{1H-pyrazolo[3,4-c]pyridin-5-yl}benzenesulfonyl)cyclohexyl]aniline;
4-(4-{[trans-4-{[4-(pentafluoro-λ⁶-sulfanyl)phenyl]amino}cyclohexyl]sulfonyl}phenyl)thiophene-2-carboxamide;
4-(pentafluoro-λ⁶-sulfanyl)-N-[trans-4-(4-{8-methylimidazo[1,2-a]pyridin-6-yl}benzenesulfonyl)cyclohexyl]aniline;
4-(pentafluoro-λ⁶-sulfanyl)-N-[trans-4-(4-{3-methyl-1H-pyrazolo[3,4-b]pyridin-5-yl}benzenesulfonyl)cyclohexyl]aniline;
4-(pentafluoro-λ⁶-sulfanyl)-N-[trans-4-{4-[8-(trifluoromethyl)imidazo[1,2-a]pyridin-6-yl]benzenesulfonyl}cyclohexyl]aniline;
4-(pentafluoro-λ⁶-sulfanyl)-N-[trans-4-(4-{8-fluoroimidazo[1,2-a]pyridin-6-yl}benzenesulfonyl)cyclohexyl]aniline;
2-(4-{[trans-4-{[4-(pentafluoro-λ⁶-sulfanyl)phenyl]amino}cyclohexyl]sulfonyl}phenyl)-4H,5H,6H,7H-thieno[3,2-c]pyridin-4-one;
7-(4-{[trans-4-{[4-(pentafluoro-λ⁶-sulfanyl)phenyl]amino}cyclohexyl]sulfonyl}phenyl)-1H,2H-pyrrolo[1,2-a]pyrazin-1-one;
8-(4-{[trans-4-{[4-(pentafluoro-λ⁶-sulfanyl)phenyl]amino}cyclohexyl]sulfonyl}phenyl)-1H,2H,3H,4H,5H-pyrrolo[1,2-a][1,4]diazepin-1-one;
7-(4-{[trans-4-{[4-(pentafluoro-λ⁶-sulfanyl)phenyl]amino}cyclohexyl]sulfonyl}phenyl)-1H,2H,3H,4H-pyrrolo[1,2-a]pyrazin-1-one;
6-(4-{[trans-4-{[4-(pentafluoro-λ⁶-sulfanyl)phenyl]amino}cyclohexyl]sulfonyl}phenyl)imidazo[1,2-a]pyridine-8-carboxamide;
4-(4-{[trans-4-{[4-(pentafluoro-λ⁶-sulfanyl)phenyl]amino}cyclohexyl]sulfonyl}phenyl)furan-2-carboxamide;
4-(pentafluoro-λ⁶-sulfanyl)-N-[trans-4-(4-{8-methylimidazo[1,2-a]pyridin-6-yl}benzenesulfonyl)cyclohexyl]aniline;
6-(4-{[trans-4-{[4-(pentafluoro-λ⁶-sulfanyl)phenyl]amino}cyclohexyl]sulfonyl}phenyl)imidazo[1,2-a]pyridine-8-carbonitrile;
4-(pentafluoro-λ⁶-sulfanyl)-N-[trans-4-(4-{3-[(2R)-pyrrolidin-2-yl]-[1,2,4]triazolo[4,3-a]pyridin-6-yl}benzenesulfonyl)cyclohexyl]aniline;
{4-[3-(propan-2-yl)-[1,2,4]triazolo[4,3-a]pyrazin-6-yl]phenyl}[trans-4-{[4-(pentafluoro-λ⁶-sulfanyl)phenyl]amino}cyclohexyl](imino)-λ⁶-sulfanone;
(+)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone;
(−)-{4-[3-(propan-2-yl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone;
N-[4-(pentafluoro-λ⁶-sulfanyl)phenyl]-1-(4-{3-[(3R)-pyrrolidin-3-yl]-[1,2,4]triazolo[4,3-a]pyridin-6-yl}benzenesulfonyl)piperidin-4-amine;
N-[4-(pentafluoro-λ⁶-sulfanyl)phenyl]-1-(4-{3-[(3 S)-pyrrolidin-3-yl]-[1,2,4]triazolo[4,3-a]pyridin-6-yl}benzenesulfonyl)piperidin-4-amine;
1-{4-[3-(azetidin-3-yl)-[1,2,4]triazolo[4,3-a]pyridin-6-yl]benzenesulfonyl}-N-[4-(pentafluoro-λ⁶-sulfanyl)phenyl]piperidin-4-amine;
4-(pentafluoro-λ⁶-sulfanyl)-N-[trans-4-{4-[3-(azetidin-3-yl)-[1,2,4]triazolo[4,3-a]pyridin-6-yl]benzenesulfonyl}cyclohexyl]aniline;
{4-[3-(difluoromethyl)-[1,2,4]triazolo[4,3-a]pyridin-6-yl]phenyl}[trans-4-{[4-(pentafluoro-λ⁶-sulfanyl)phenyl]amino}cyclohexyl](imino)-λ⁶-sulfanone;
(4-{3-methyl-[1,2,4]triazolo[4,3-a]pyrazin-6-yl}phenyl)[trans-4-{[4-(pentafluoro-λ⁶-sulfanyl)phenyl]amino}cyclohexyl](imino)-λ⁶-sulfanone;
{4-[3-(propan-2-yl)-[1,2,4]triazolo[4,3-a]pyridin-6-yl]phenyl}[trans-4-{[5-(pentafluoro-λ⁶-sulfanyl)pyridin-2-yl]amino}cyclohexyl](imino)-λ⁶-sulfanone;
5-(pentafluoro-λ⁶-sulfanyl)-N-(1-{4-[3-(propan-2-yl)-[1,2,4]triazolo[4,3-a]pyridin-6-yl]benzenesulfonyl}piperidin-4-yl)pyridin-2-amine;
N-[1-(4-{imidazo[1,2-a]pyridin-6-yl}benzenesulfonyl)piperidin-4-yl]-5-(pentafluoro-λ⁶-sulfanyl)pyridin-2-amine;
N-[1-(4-{3-methyl-[1,2,4]triazolo[4,3-a]pyridin-6-yl}benzenesulfonyl)piperidin-4-yl]-5-(pentafluoro-λ⁶-sulfanyl)pyridin-2-amine;
4-{4-[(4-{[5-(pentafluoro-λ⁶-sulfanyl)pyridin-2-yl]amino}piperidin-1-yl)sulfonyl]phenyl}pyridine-2-carboxamide;
4-(pentafluoro-λ⁶-sulfanyl)-N-[trans-4-{4-[3-(difluoromethyl)-[1,2,4]triazolo[4,3-a]pyridin-6-yl]benzenesulfonyl}cyclohexyl]aniline;
4-{4-[(4-{[4-(pentafluoro-λ⁶-sulfanyl)phenyl]amino}piperidin-1-yl)sulfonyl]phenyl}pyridine-2-carboxamide;
5-(pentafluoro-λ⁶-sulfanyl)-N-[trans-4-{4-[3-(propan-2-yl)-[1,2,4]triazolo[4,3-a]pyridin-6-yl]benzenesulfonyl}cyclohexyl]pyridin-2-amine;
5-(pentafluoro-λ⁶-sulfanyl)-N-[trans-4-(4-{3-methyl-[1,2,4]triazolo[4,3-a]pyridin-6-yl}benzenesulfonyl)cyclohexyl]pyridin-2-amine;
5-(pentafluoro-λ⁶-sulfanyl)-N-[trans-4-(4-{imidazo[1,2-a]pyridin-6-yl}benzenesulfonyl)cyclohexyl]pyridin-2-amine; and
5-(pentafluoro-λ⁶-sulfanyl)-N-[trans-4-{4-[8-methyl-3-(propan-2-yl)-[1,2,4]triazolo[4,3-a]pyridin-6-yl]benzenesulfonyl}cyclohexyl]pyridin-2-amine.

41-45. (canceled)

46. A process of preparation of a compound of formula (I) or (I′) according to claim 1, or a pharmaceutically acceptable salt thereof, wherein X¹ is N and X⁴ is O, comprising:
reacting compound of formula (VI), wherein R⁶, R⁷, and X⁵ are as defined in claim 1 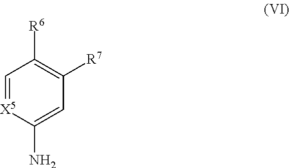
with compound of formula (VII), wherein R⁵ and R⁴ are as defined in claim 1 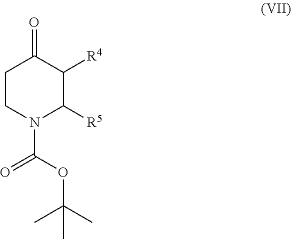
to form compound of formula (VIII), wherein R⁴, R⁵, X⁵, R⁶, R⁷ are as defined in claim 1 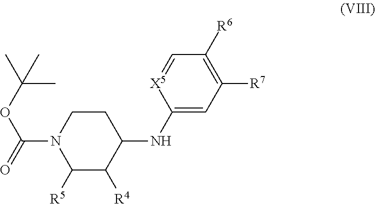
reacting said compound of formula (VIII) with acid to form compound of formula (IX), wherein R⁴, R⁵, X⁵, R⁶, R⁷ are as defined in claim 1 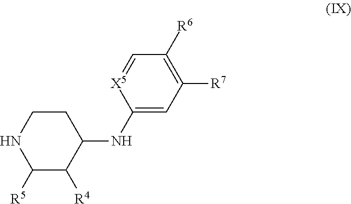
reacting said compound of formula (IX) with compound of formula (XI), wherein X², X³, R² and R³ are as defined in claim 1 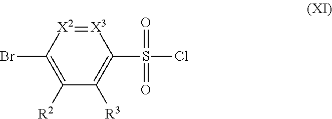
to form compound of formula (III), wherein R², R³, R⁴, R⁶, R⁷, R⁵, X², X⁵ and X³ are as defined in claim 1 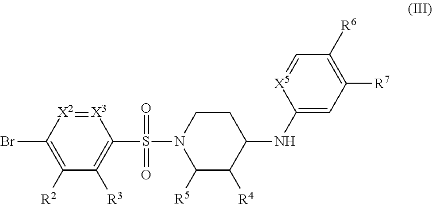
reacting said compound of formula (III) with compound of formula (X) wherein R¹ is as defined in claim 1 and R⁹ and R¹⁰ are each independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, 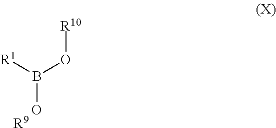
to form compound of formula (I).

47-49. (canceled)

50. A compound according to claim 1, or a pharmaceutically acceptable salt thereof, when manufactured according to the process of claim 46.

51. (canceled)

52. A pharmaceutical composition comprising a compound according to claim 1, or a pharmaceutically acceptable salt thereof, and a pharmaceutically acceptable excipient.

53. The pharmaceutical composition according to claim 52, further comprising an additional therapeutic agent.

54-58. (canceled)

59. A method for the treatment, prevention and/or delay of progression of an inflammatory autoimmune disease, which method comprises administering a therapeutically effective amount of a compound according to claim 1, or a pharmaceutically acceptable salt thereof.

60. A method for the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus or multiple sclerosis, which method comprises administering a therapeutically effective amount of a compound according to claim 1, or a pharmaceutically acceptable salt thereof.
